(19) United States
(12) Patent Application Publication
(10) Pub. No.: US 2021/0221785 A1
(43) Pub. Date: Jul. 22, 2021

(54) AMINO-PYRIMIDONYL DERIVATIVES, A PROCESS FOR THEIR PREPARATION AND PHARMACEUTICAL COMPOSITIONS CONTAINING THEM

(71) Applicants: LES LABORATOIRES SERVIER, SURESNES CEDEX (FR); VERNALIS (R&D) LTD, Cambridge (GB)

(72) Inventors: Csaba WÉBER, Pilisszentlászló (HU); András KOTSCHY, Törökbálint (HU); Attila VASAS, Fót (HU); Árpád KISS, Budakalász (HU); Balázs MOLNÁR, Isaszeg (HU); Alba MACIAS, Cambridge (GB); Andrea FIUMANA, Ely (GB); Nicolas DAVIES, Cambridge (GB); James Brooke MURRAY, Linton (GB); Emilie SELLIER, Cambridge (GB); Didier DEMARLES, Checy (FR); Lisa IVANSCHITZ, Massy (FR); Olivier GENESTE, Rueil-Malmaison (FR)

(21) Appl. No.: 17/254,482

(22) PCT Filed: Jul. 4, 2019
(86) PCT No.: PCT/EP2019/068050

§ 371 Date: Dec. 21, 2020

(30) Foreign Application Priority Data
Jul. 5, 2018 (FR) 1856218
Mar. 28, 2019 (EP) 19305398.0
May 27, 2019 (EP) 19305667.8

(51) Int. Cl. C07D 401/06; C07D 401/14; A61K 35/17; A61K 45/06; A61K 31/506

(52) U.S. Cl. CPC C07D 401/06; C07D 401/14; A61K 31/506; A61K 45/06; A61K 35/17

(57) ABSTRACT

Compounds of formula (I):

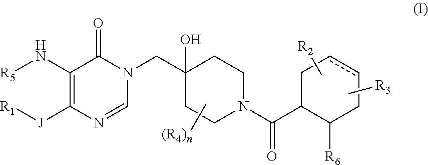

wherein R₁, R₂, R₃, R₄, R₅, R₆, J and n are as defined in the description.

The present invention relates to new amino-pyrimidonyl derivatives, to a process for their preparation and to pharmaceutical compositions containing them.

The compounds of the present invention are new and have very valuable pharmacological characteristics in the field of apoptosis and oncology.

Ubiquitination is a process controlling essential cellular functions such as protein turnover and homeostasis, protein activation and localisation. Ubiquitin is a 76 amino acids polypeptide which is covalently attached to postranslationnaly modified protein substrates via an isopeptide bond. Deubiquinating enzymes Willis) are in majority cysteine proteases that cleave the ubiquitin-ubiquitin bond or ubiquitin-protein bond at the Cter glycine of Ubiquitin. Approximately 100 DUBs regulate the thousands ubiquitinated proteins and then some redundancy of deubiquitinase substrates regulation are observed.

Dysregulation of DUBs have been associated with several diseases such as neurodegenerative and infectious, diseases (Edelman et al., Expert Rev. Mol. Med. 2011, 13, 1-17) and human malignancies (Pal et al., Cancer Res. 2014, 74, 4955-4966). Accordingly. overexpression of DUBs or increase of their activity have been associated to numerous types of cancers (Luise et al., Plos One 2011, 6, e15891: Rolen et al., Mol. Carcinog. 2006, 45, 260-269) and poor prognosis.

Ubiquitin Specific Protease 7 (USP7), also known as Herpes-virus-Associated Ubiquitin-Specific Protease (HAUSP), belongs to the deubiquitinating family. USP7 has been reported to stabilize numerous oncogenes involved in survival and proliferations via cell cycle progression, apoptosis. DNA repair, DNA replication and epigenetic factors regulation (Nicholson et al., Cell Biochem. Biopys. 2011, 60, 61-68). In addition, USP7 has been shown to regulate immune response via inflammation and Treg modulation (Van Loosdregt et al., Immunity 2013, 39, 259-27; Colleran et al., Proc Natl. Acad. Sci. USA 2013, 110, 618-623; Wang et al., EBio Medicine 2016, 99-112: Wang et al., PLoS One 2017, 12, e018977). USP7 has also been implicated in other pathologic states such as neurodevelopmental disorder (Hao et al., Mol. Cell 2015, 59, 956-969) and viral infection (Holowaty et al., Biochem. Soc. Trans. 2004, 32, 731-732).

USP7 overexpression has been associated with late stages of cancers and poor prognosis in neuroblastoma, myeloma, prostate, colon and breast cancers. Numerous USP7 inhibitors have been recently published in the literature (Turnbull et al., Nature 2017, 550, 481-486; Kategaya et al., Nature 2017, 550, 534-538; Gavory et al., Nat. Chem. Biol. 2018, 14, 118-125: O'Dowd et al., ACS Med. Chem. Lett. 2018, 9, 238-243; Pozhidaeva et al., Cell Chem. Biol. 2017, 24, 1501-1512; Lamberto et al., Cell Chem. Biol. 2017, 24, 1490-1500) and, particularly, pyrimidonyl derivatives claimed as USP7 inhibitors have been disclosed in PCT/GB2017/053175. However. PCT/GB2017/053175 shows that 5,6-disubstituted pyrimidonyl derivatives provide compounds with weakest affinity on USP7. Despite an intense research in the field, no USP7 inhibitors have entered the clinic (Kemp et al., Progress in Medicinal Chemistry 2016, 55, 149-192, Wu et al., J. Med. Chem. 2018, 61, 422-443). There is, therefore, a therapeutic need for compounds that inhibit the activity of the protein USP7.

In addition to being new and very potent on their target, the compounds of the present invention have pro-apoptotic and/or anti-proliferative properties making it possible to use them in pathologies involving a defect in apoptosis, such as, for example, in the treatment of cancer and of immune and auto-immune diseases.

The present invention relates more especially to compounds of formula (I):

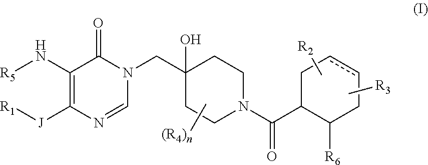

wherein:
J represents an oxygen atom or a sulphur atom,
R₁ represents a cycloalkyl group, a heterocycloalkyl group, an aryl group, or a heteroaryl group,
R₂ represents a hydrogen atom, a halogen atom, a hydroxy group, or a linear or branched (C₁-C₆)alkoxy group,
R₃ represents a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₂-C₆)alkynyl-R₇ group, a cycloalkyl group, an aryl group, a heteroaryl group, an aryl(C₁-C₆)alkyl group, or a heteroaryl(C₁-C₆)alkyl group,
R₄ represents a hydrogen atom or a halogen atom,
R₅ represents a hydrogen atom, a linear or branched (C₁-C₆)alkyl group, a linear or branched halo(C₁-C₆)alkyl group, or an aryl(C₁-C₆)alkyl group,
R₆ represents an aryl group or a heteroaryl group,
R₇ represents a cycloalkyl group, an aryl group, a heteroaryl group, or a —Y₁—OR′ group,
n is an integer equal to 0, 1 or 2.
[symbol] means a single bond or a double bond

it being understood that:
“aryl” means a phenyl, naphthyl, or indanyl group,
“heteroaryl” means any mono- or fused bi-cyclic group composed of from 5 to 10 ring members, having at least one aromatic moiety and containing from 1 to 3 heteroatoms selected from oxygen, sulphur and nitrogen,
“cycloalkyl” means any mono- or fused bi-cyclic non-aromatic carbocyclic group containing from 3 to 7 ring members,
“heterocycloalkyl” means any non-aromatic mono- or fused bi-cyclic group containing from 3 to 10 ring members, and containing from 1 to 3 heteroatoms selected from) oxygen, sulphur and nitrogen,

it being possible for the aryl, heteroaryl, cycloalkyl and heterocycloalkyl groups so defined to be substituted by from I to 4 groups selected from linear or branched (C₁-C₆)alkyl, linear or branched (C₂-C₆)alkenyl, linear or branched (C₂-C₆)alkynyl, linear or branched halo(C₁-C₆)alkyl, —Y₂—OR′, —Y₂—NR′R″, —Y₂—S(O)ₘ—R′, oxo (or N-oxide where appropriate), pentafluorosulfide, nitro, —Y₂—CN, —C(O)—R′, —C(O)—OR′, —O—C(O)—R′, —Y₂—C(O)—NR′R″, —Y₂—NR′—C(O)—R″, —Y₂—NR′—C(O)—OR″, halogen, cyclopropyl and —Y₂-heterocycloalkyl,

it being understood that:
Y₁ and Y₂ independently of one another represent a bond, a linear or branched (C₁-C₄)alkylene group, or a linear or branched halo(C₁-C₄)alkylene group.
R′ and R″ independently of one another represent a hydrogen atom. a linear or branched (C₁-C₆)alkyl group. a linear or branched (C₁-C₆)alkenyl group, a linear or branched (C₂-C₆)alkynyl group, a linear or branched (C₁-C₆)alkoxy group, a linear or branched halo(C₁-C₆)alkyl, a linear or branched hydroxy(C₁-C₆)alkyl group, a linear or branched (C₁-C₆)alkoxy(C₁-C₆)alkyl group, a formyl group, a phenyl group, a benzyl group, a cyclopropyl group, a cyclopropylmethyl group,
or the pair (R′, R″) form together with the nitrogen atom carrying them a non-aromatic ting composed of from 5 to 7 ring members, which may contain in addition to the nitrogen a second heteroatom selected from oxygen and nitrogen, it being understood that the nitrogen in question may be substituted by from 1 to 2 groups representing a hydrogen atom, or a linear or branched (C₁-C₆)alkyl group,
m is an integer equal to 0, 1 and 2,

their enantiomers, diastereoisomers, and addition salts thereof with a pharmaceutically acceptable acid or base.

In a preferred embodiment of the invention, the present invention relates to compounds of formula (I) wherein:
R₁ represents an aryl group or a heteroaryl group,
R₂ represents a halogen atom, a hydroxy group or a linear or branched (C₁-C₆)alkoxy group,
R₃ represents a halogen atom, a linear or branched (C₁-C₆)alkyl group, a linear or branched (C₂-C₆)alkynyl group, a linear or branched (C₂-C₆)alkynyl-R₇ group, an aryl group, an aryl(C₁-C₆)alkyl group, or a heteroaryl(C₁-C₆)alkyl group,
R₄ represents a hydrogen atom or a halogen atom,
R₅ represents a hydrogen atom, a linear or branched (C₁-C₆)alkyl group, a linear or branched halo(C₁-C₆)alkyl group, or an aryl(C₁-C₆)alkyl group,
R₆ represents an aryl group,
R₇ represents a cycloalkyl group, an aryl group, or a heteroaryl group,

it being possible for the aryl and heteroaryl groups so defined to be substituted by from 1 to 4 groups selected from linear or branched (C₁-C₆)alkyl, linear or branched halo(C₁-C₄)alkyl, —Y₂—OR′, —Y₂—NR′R″, pentafluorosulfide, —Y₂—CN, —C(O)—R′, —C(O)—OR′, —Y₂—C(O)—NR′R″, halogen and —Y₂-heterocycloalkyl,

it being understood that Y₂ is as defined for formula (I) and R′ and R″ independently of one another represent a hydrogen atom, a linear or branched (C₁-C₆)alkyl group, a linear or branched halo(C₁-C₆)alkyl, a linear or branched hydroxy(C₁-C₆)alkyl group, a formyl group, a phenyl group, a benzyl group, or a cyclopropyl group,

or the pair (R′, R″) form together with the nitrogen atom carrying them a non-aromatic ring composed of from 5 to 7 ring members, which may contain in addition to the nitrogen a second heteroatom selected from oxygen and nitrogen, it being understood that the nitrogen in question may be substituted by from 1 to 2 groups representing a hydrogen atom or a linear or branched (C₁-C₆)alkyl group.

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyrovic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Among the heteroaryl groups there may be mentioned, without implying any limitation, pyrrolyl, furyl, thienyl, thiazolyl, isothiazolyl, oxazolyl, isoxazolyl, pyrazolyl, imidazolyl, pyridinyl, pyrazinyl, pyridazinyl, pyrimidinyl, pyridinonyl, indolyl, dihydroindolyl, dihydroisoindolyl, indazolyl, dihydrocyclopentathienyl, benzothienyl, tetrahydrobenzothienyl, benzofuranyl, imidazopyridinyl, imidazopyrazinyl, benzotriazolyl, benzodioxolyl, dihydrobenzodioxinyl, quinolinyl, isoquinolinyl, tetrahydroquinolinyl, tetrahydroisoquinolinyl, quinoxalinyl, dihydroquinoxalinyl, dihydrothienodioxinyl, quinatolinonyl, pyrrolopyridazinyl, pyrazolopyrazinyl, pyrrolopyridinyl, dihydropyrrolizinyl, tetrahydroindolizinyl, etc.

Among the cycloalkyl groups there may be mentioned, without implying any limitation, cyclopropyl, cyclobutyl, cyclopentyl, cyclohexyl, etc.

Among the heterocycloalkyl groups there may be mentioned, without implying any limitation, pyrrolidinyl, tetrahydropyranyl, piperidinyl, piperazinyl, morpholinyl, etc.

Advantageously, the compounds of formula (I) display a trans configuration as follows:

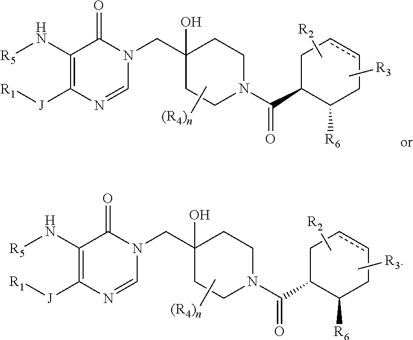

Preferably, the compounds of formula (I) display a trans configuration as follows:

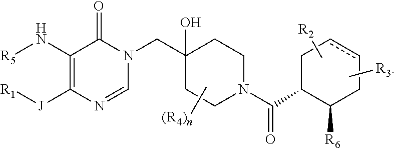

In another embodiment, when R₄ represents a halogen atom and n is an integer equal to 1 or 2, a new asymmetric carbon can be created providing two possible isomers as follows:

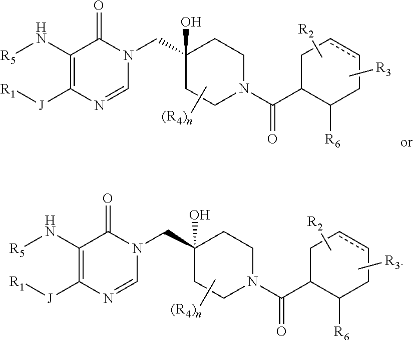

Preferably, when R₄ represents a halogen atom and n is an integer equal to 2, having the following formula:

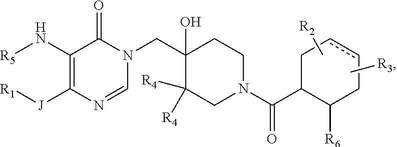

the preferred isomer has the S-configuration as follows:

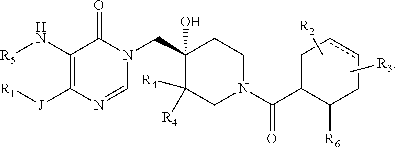

Preferably, [symbol] is a single bond.

J advantageously represents an oxygen atom.

R₁ preferably represents an aryl group or a heteroaryl group. More preferably, R₁ represents a phenyl group, an indanyl group, a benzodioxolyl group, a tetrahydroisoquinolyl group, an isoindolinyl group, an indazolyl group, a thiazolyl group, a pyridinyl group, a pyrrolopyridinyl group or a pyrimidinyl group. Even more preferably, R₁ represents a phenyl group. In a preferred embodiment of the invention, R₁ represents a phenyl group which is substituted by from 1 to 2 groups selected from linear or branched (C₁-C₆)alkyl; linear or branched halo(C₁-C₆)alkyl; —Y₂—OR′; —Y₂—NR′R″; pentafluorosulfide; —Y₂—CN; —C(O)—R′; —C(O)—OR′ wherein R′ represents a linear or branched (C₁-C₆)alkyyl; —Y₂—C(O)—NR′R″; halogen; and —Y₂-heterocycloalkyl. In another preferred embodiment of the invention, R₁ represents a phenyl group which is substituted by from 1 to 2 groups selected from linear or branched (C₁-C₆)alkyl, linear or branched halo(C₁-C₆)alkyl, —Y₂—OR′, —Y₂—NR′R″; —Y₂—CN, —C(O)—R′, halogen and —Y₂-heterocycloalkyl. More advantageously, R₁ represents a phenyl group which is substituted by from 1 to 2 groups selected from —Y₂—OR′, —Y₂—NR′R′R″, halogen, pyrrolidinyl, —Y₂-piperidinyl and —Y₂-morpholinyl. Even more advantageously, R₁ represents a phenyl group which is substituted by from 1 to 2 groups selected from hydroxy, methoxy —Y₂—NR′R″, fluorine, chlorine, pyrrolidinyl and piperidinyl.

R₂ preferably represents a halogen atom, a hydroxy group, or a linear or branched (C₁-C₆)alkoxy group. More preferably, R₂ represents a fluorine atom, a hydroxy group, or a methoxy group. Even more preferably, R₂ represents a fluorine atom.

R₃ preferably represents a halogen atom, a linear or branched (C₁-C₆)alkyl, a linear or branched (C₂-C₆)alkynyl group, a linear or branched (C₂-C₆)alkynyl-R₇ group, an aryl group, an aryl(C₁-C₆)alkyl group, or a heteroaryl(C₁-C₆)alkyl group. More preferably, R₃ represents a fluorine atom; a phenyl group; a benzyl group; a —C≡CH group; a —C≡C—R₇ group wherein R₇ represents a cycloalkyl group, an aryl group or a heteroaryl group; or a heteroaryl(C₁-C₆)alkyl group wherein the heteroaryl ring is selected from pyridinyl, pyrimidinyl, pyrazinyl, pyridazinyl, thiazolyl or imidazolyl. Even more preferably, R₃ represents a fluorine atom; a phenyl group; a benzyl group; a —C≡C—R₇ group wherein R₇ represents a cycloalkyl group, an aryl group or a heteroaryl group; or a heteroaryl(C₁-C₆)alkyl group wherein the heteroaryl ring is selected from pyridinyl, pyrimidinyl, pyrazinyl, pyridazinyl, thiazolyl or imidazolyl. In another preferred embodiment, R₃ represents a fluorine atom, in another preferred embodiment, R₃ represents a —C≡C—R₇ group wherein R₇ represents a cyclopropyl group, a phenyl group, a imidazolyl group, a pyridinyl group, a pyrimidinyl group, a pyrazinyl group, or a pyridazinyl group. In another preferred embodiment, R₃ represents a —C≡C—R₇ group wherein R₇ represents a heteroaryl group selected from imidazolyl, pyridinyl, pyrimidinyl, pyrazinyl or pyridazinyl. In another preferred embodiment, R₃ represents a —C≡C—R₇ group wherein R₇ represents a pyridinyl group, a pyrimidinyl group, a pyrazinyl group, or a pyridazinyl group.

Advantageously, R₂ and R₃ are geminal groups. More advantageously, R₂ and R₃ are geminal groups and R₂ and R₃ represent a fluorine atom (also called a gem-difluoro group).

In another preferred embodiment, R₂ and R₃ are seminal groups wherein R₂ represents a halogen atom or a linear or branched (C₁-C₆)alkoxy group and R₃ represents a —C≡C—R₇ group wherein R₇ represents a heteroaryl group selected from imidazolyl, pyridinyl, pyrimidinyl, pyrazinyl or pyridazinyl.

More preferably, R₂ and R₃ are geminal groups wherein R₂ represents a halogen atom, more preferably a fluorine atom, and R₃ represents a —C≡C—R₇ group wherein R₇ represents a pyridinyl group. a pyrazinyl group or a pyridazinyl group.

In another preferred embodiment, R₂ and R₃ are geminal groups wherein R₂ represents a linear or branched (C₁-C₆)alkoxy group, more preferably a methoxy group, and R₃ represents a —C≡C—R₇ group wherein R₇ represents a pyridinyl group, a pyrimidinyl group, a pyrazinyl group or a pyridazinyl group.

Advantageously, R₄ represents a hydrogen atom or a fluorine atom. More preferably, R₄ represents a hydrogen atom. In another embodiment, when R₄ represents a fluorine atom and n is equal to 2. both fluorine atoms preferably represent a gem-difluoro group.

Preferably, R₅ represents a hydrogen atom.

R₆ preferably represents an aryl group or a heteroaryl group selected from pyridinyl, thienyl, oxazolyl, pyrarolyl, thiazolyl, or furyl. More preferably, R₆ represents an aryl group, even more preferably, a phenyl group.

R₇ preferably represents a cycloalkyl group, an aryl group, or a heteroaryl group. More preferably, R₇ represents a cyclopropyl group, a phenyl group, a imidazolyl group, a pyridinyl group, a pyrimidinyl group, a pyrazinyl group, or a pyridazinyl group. Even more preferably, R₇ represents an imidazolyl group, a pyridinyl group, a pyrimidinyl group, a pyrazinyl group, or a pyridazinyl group. More advantageously, R₇ represents a pyridinyl group, a pyrimidinyl group, a pyrazinyl group, or a pyridazinyl group. In another preferred embodiment, R₇ represents a cycloalkyl group. an aryl group, or a heteroaryl group which is substituted by from 1 to 2 groups selected from halogen atom, linear or branched (C₁-C₆)alkyl group, linear or branched (C₁-C₆)alkoxy group, or amino group. More preferably. R₇ represents a cycloalkyl group, an aryl group, or a heteroaryl group which is substituted by from 1 to 2 groups selected from a fluorine atom, a methyl group, a methoxy group, or an amino group.

In a preferred embodiment of the invention, the present invention relates to compounds of formula (I-a):

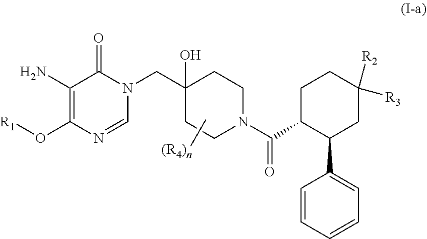

wherein R₁, R₂, R₃, R₄ and n are as defined for formula (I).

In a preferred embodiment of the invention, the present invention relates to compounds of formula (I-a):

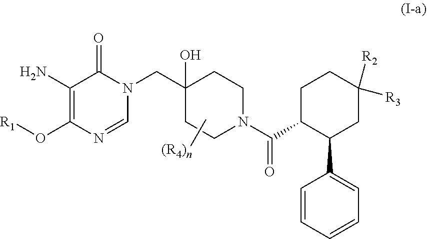

wherein R₁ represents a phenyl group which may be substituted by from 1 to 2 groups selected from linear or branched (C₁-C₆)alkyl, linear or branched halo(C₁-C₆)alkyl, —Y₂—OR′, —Y₂—NR′R″, pentafluorosulfide, —Y₂—CN, —C(O)—R′, —C(O)—OR′ wherein R′ represents a linear or branched (C₁-C₆)alkyl, —Y₂—C(O)—NR′R″, halogen and —Y₂-heterocycloalkyl, and R₂, R₃, R₄ and n are as defined for formula (I).

In a more preferred embodiment of the invention, the present invention relates to compounds of formula (I-a):

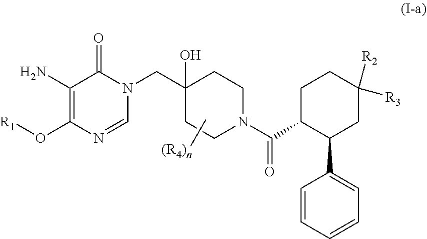

wherein R₁ represents a phenyl group which may be substituted by from 1 to 2 groups selected from —Y₂—OR′, —Y₂—NR′R″, halogen, pyrrolidinyl, —Y₂-piperidinyl and —Y₂-morpholinyl, and R₂, R₃, R₄ and n are as defined for formula (I).

Among the preferred compounds or the invention there may be mentioned:
5-amino-3-[[1-[(1R,2R)-4,4-difluoro-2-phenyl-cyclohexanecarbonyl]-4-hydroxy-4-piperidyl]methyl]-6-(3-hydroxy-5-methoxy-phenoxy)pyrimidin-4-one;
5-amino-3-({1-[(1R,2R)-4,4-difluoro-2-phenylcyclohexane-1-carbonyl]-4-hydroxypiperidin-4-yl}methyl)-6-[4-(pyrrolidin-2-yl)phenoxy]pyrimidin-4(3H)-one;
5-amino-3({1-[(1R,2R)-4,4-difluoro-2-phenylcyclohexane-1-carbonyl]-4-hydroxypiperidin-4-yl}methyl)-6-(4-fluoro-3-hydroxyphenoxy)pyrimidin-4(3H)-one;
5-amino-3-[[1-[(1R,2R)-4,4-difluoro-2-phenyl-cyclohexanecarbonyl]-4-hydroxy-4-piperidyl]methyl]-6-[3-(2-piperidyl)phenoxy]pyrimidin-4-one;
5-amino-6-[4-(1-aminoethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-6-[4-(aminomethyl)phenoxy]-3-({1-[(1R,2R)-4,4-difluoro-2-phenylcyclohexane-1 -carbonyl]-4-hydroxypiperidin-4-yl}methyl)pyrimidin-4(3H)-one;
5-amino-3-({1-[(1R,2R)-4,4-difluoro-2-phenylcyclohexane-1-carbonyl]-4-hydroxypiperidin-4-yl}methyl)-6-{4-[(methylamino)methyl]phenoxy}pyrimidin-4(3H)-one;
5-amino-6-[3-(aminomethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-3-[[1-[(1R,2R)-4,4-difluoro-2-phenyl-cyclohexanecarbonyl]-4-hydroxy-4-piperidyl]methyl]-6-(3-hydroxyphenoxy)pyrimidin-4-one;
5-amino-6-[4-(aminomethyl)-3-fluorophenoxy]-3-({1-[(1R₂R)-4,4-difluoro-2-phenylcyclohexane-1-carbonyl]-4-hydroxypiperidin-4-yl}methyl)pyrimidin-4(3H)-one;
5-amino-6-[4-(aminomethyl)-3-chlorophenoxy]-3-({[1-[(1R,2R)-4,4-difluoro-2-phenylcyclohexane-1-carbonyl]-4-hydroxypiperidin-4-yl}methyl)pyrimidin-4(3H)-one;
5-amino-6-{4-[(tert-butylamino)methyl]phenoxy}-3-({1-[(1R ,2R)-4,4-difluoro-2-phenylcyclohexane-1-carbonyl]-4-hydroxypiperidin-4-yl}; methyl)pyrimidin-4(3H)-one;
5-amino-6-[4-(aminomethyl)phenoxy]-3-({4S)-1-[(1R,2R)-4,4-difluoro-2-phenylcyclohexane-1-carbonyl]-3,3-difluoro-4-hydroxypiperidin-4-yl}methyl)pyrimidin-4(3H)-one;
5-amino-3-({(4S)-1-[(1R,2R)-4,4-difluoro-2-phenylcyclohexane-1-carbonyl]-3,3-difluoro-4-hydroxypiperidin-4-yl}methyl)-6-(3-hydroxyphenoxy)pyrimidin-4(3H)-one;
5-amino-3-({(4S)-1-[(1R,2R)-4,4-difluoro-2-phenylcyclohexane-1-carbonyl]-3,3-difluoro-4-hydroxypiperidin-4-yl}methyl)-6-(4-fluoro-3-hydroxyphenoxy)pyrimidin-4(3H)-one;
5-amino-3-({1-[(1R,2R)-4,4-difluoro-2-phenylcyclohexane-1-carbonyl]-4-hydroxypiperidin-4-yl}methyl)-6-[4-(piperidin-2-yl)phenoxy]pyrimidin-4(3H)-one;
5-amino-3-[(1-{[(1R,2R,4R)-4-fluoro-2-phenyl-4-[2-(pyridin-3-yl)ethynyl]cyclohexyl]carbonyl]-4-hydroxypiperidin-4-yl}methyl]-6-(4-fluorophenoxy)pyrimidin-4-one;
5-amino-3-[(1-{[(1R,2R,4R)-4-fluoro-2-phenyl-4-[2-(pyridin-2-yl)ethynyl]cyclohexyl]carbonyl}-4-hydroxypiperidin-4-yl)methyl]-6-(4-fluorophenoxy)pyrimidin-4-one;
5-amino-3-[(1-{[(1R,2R,4R)-4-fluoro-4-[2-(5-fluoropyridin-2-yl)ethynyl]-2-phenylcyclohexyl]carbonyl}-4-hydroxypiperidin-4-yl)methyl]-6-(4-fluorophenoxy)pyrimidin-4-one;
5-amino-3-({1-[(1R,4S)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4-one;
5-amino-3-({1-[(1R,2R,4R)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4 -one;
5-amino-6-(4-fluorophenoxy)-3-[(4-hydroxy-1-{[(1R,2R,4R)-4-methoxy-2-phenyl-4-[2-(pyrimidin-5-yl)ethynyl]cyclohexyl]carbonyl}piperidin-4-yl)methyl]pyrimidin-4-one;
5-amino-3-[(1-{[(1R,2R,4R)-4-fluoro-2-phenyl-4-[2-(pyridazin-3-yl)ethynyl]cyclohexyl]carbonyl}-4-hydroxypiperidin-4-yl)methyl]-6-(4-fluorophenoxy)pyrimidin-4-one;
5-amino-6-(4-fluorophenoxy)-3-[(4-hydroxy-1-{[(1R,2R,4R)-4-methoxy-2-phenyl-4-[2-(pyridazin-3-yl)ethynyl]cyclohexyl]carbonyl}piperidin-4-yl)methyl]pyrimidin-4-one;
5-amino-[4-fluorophenoxy)-3-([{4-hydroxy-1-[(1R,2R,4R)-4-methoxy-2-phenyl-4-[2-pyrazin-2-yl)ethynyl]cyclohexanecarbonyl]piperidin-4-yl}methyl)-3,4-dihydropyrimidin-4-one;
5-amino-3-({1-[(1R,2R,4R)-4-fluoro-4-[2-(4-fluoropyridin-2-y)ethynyl]-2-phenylcyclohexanecarbonyl]-4-hydroxypiperidin-4-yl}methyl)-6-(4-fluorophenoxyl-3,4-dihydropyrimidin-4-one;
5-amino-3-{[(4S)-3,3-difluoro-1-[(1R,2R,4R)-4-fluoro-4-[2-(6-methylpyridazin-3-yl)ethynyl]-2-phenylcyclohexanecarbonyl]-4-hydroxypiperidin-4-yl]methyl}-6-(4-fluorophenoxy)-3,4-dihydropyrimidin-4-one;
5-amino-3-{[(4S)-3,3-difluoro-4-hydroxy-1-{[(1R,2R,4R)-4-methoxy-2-phenyl-4-[2-(pyridazin-3-yl)ethynyl]cyclohexyl]carbonyl}piperidin-4-yl]methyl}-6-(4-fluorophenoxy)pyrimidin-4-one;
5-amino-3-{[(4S)-3,3-difluoro-4-hydroxy-1-[(1R,2R,4R)-4-methoxy-2-phenyl-4-[2-(pyridin-2-yl)ethynyl]cyclohexanecarbonyl]piperidin-4-yl]methyl}-6-(4-fluorophenoxy)-3,4-dihydropyrimidin-4-one;
5-amino-3-{[(4S)-3,3-difluoro-1-{[(1R,2R,4R)-4-fluoro-2-phenyl-4-[2-(pyridazin-3-yl)ethynyl]cyclohexyl]carbonyl}-4-hydroxypiperidin-4-yl]methyl}-6-(4-fluorophenoxy)pyrimidin-4-one;
5-amino-3-{[(4S)-3,3-difluoro-4-hydroxy-1-[(1R,2R, 4R)-4-methoxy-4-[2-(5-methylpyridazin-3-yl)ethynyl]-2-phenylcyclohexanecarbonyl]piperidin-4-yl]methyl}-6-(4-fluorophenoxy)-1,4-dihydropyrimidin-4-one;
5-amino-3-{[(1R,2R,4R)-4-fluoro-4-[2-(5-methylpyridazin-3-yl)ethynyl]-2-phenylcyclohexanecarbonyl]-4-hydroxypiperidin4-yl]methyl}-6-(4-fluorophenoxy)-3,4-dihydropyrimidin-4-one;
5-amino-3-{[(4S)-3,3-difluoro-4-hydroxy-1-[(1R,2R, 4R)-4-methoxy-4-[2-(5-methoxypyridazin-3-yl)ethynyl]-2-phenylcyclohexanecarbonyl]piperidin-4-yl]methyl}-6-(4-fluorophenoxy)-3,4-dihydropyrimidin-4-one;
5-amino-3-{[(4S)-3,3-difluoro-4-hydroxy-1-[(1R,2R,4R)-4-methoxy-2-phenyl-4-[2-(pyrimidin-5-yl)ethynyl]cyclohexanecarbonyl]piperidin-4-yl]methyl}-6-(4-fluorophenoxy)-3,4-dihydropyrimidin-4-one.

The invention relates also to a process for the preparation of compounds of formula (I), which process is characterised in that there is used as starting material the compound of formula (II):

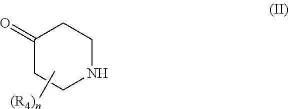

wherein R₄ and n are as defined for formula (I).

which is subjected to coupling with a compound of formula (III):

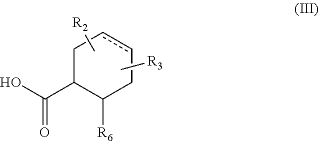

wherein R₂, R₃ and R₆ are as defined for formula (I),

to yield the compound of formula (IV):

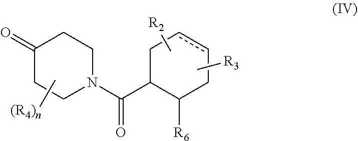

wherein R₂, R₃, R₄, R₆ and n are as defined hereinbefore.

compound of formula (IV) which is further converted to compound of formula (V):

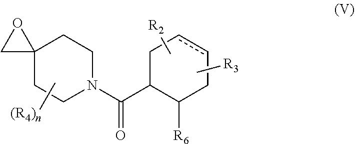

wherein R₂, R₃, R₄, R₆ and n are as defined hereinbefore,

compound of formula (V) which is further subjected to coupling with compound of formula (VI):

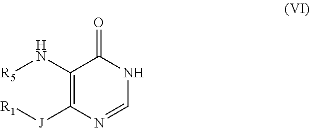

wherein R₁, R₅ and J are as defined for formula (I),

to yield the compound of formula (I), which may then be purified according to a conventional separation technique, which is convened, if desired, into its addition salts with a pharmaceutically acceptable acid or base and which is optionally separated into its isomers according to a conventional separation technique,

it being understood that at any moment considered appropriate during the course of the process described above, some groups (hydroxy, amino . . . ) of the starting reagents or of the synthesis intermediates can be protected, subsequently deprotected and functionalized, as required by the synthesis.

In another embodiment of the invention, compounds of formula (I) may be obtained using an alternative process, which process is characterized in that there is used as starting material the compound of formula (VII):

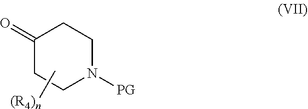

wherein R₄ and n are as defined for formula (I) and PG represents a protecting group of the amine function,

which is further converted to compound of formula (VIII):

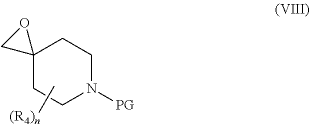

wherein R₄, PG and n are as defined hereinbefore,

compound of formula (VIII) which is subjected to coupling with compound of formula (VI):

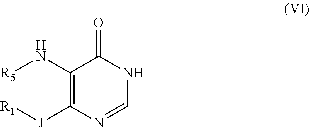

wherein R₁, R₅ and J are as defined for formula (I),

to yield the compound of formula (IX):

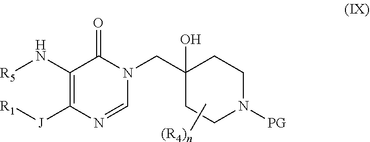

wherein R₁, R₄, R₅, J, PG and n are as defined for formula (I).

which is further subjected, after removing the protecting group or the amine function, to coupling with a compound of formula (III):

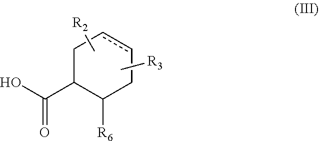

wherein R₂, R₃ and R₆ are as defined for formula (I),

to yield the compound of formula (I), which may then he purified according to a conventional separation technique, which is converted, if desired, into its addition salts with a pharmaceutically acceptable acid or base and which is optionally separated into its isomers according to a conventional separation technique,

it being understood that at any moment considered appropriate during the course of the process described above, some groups (hydroxy, amino . . . ) of the starting reagents or of the synthesis intermediates can be protected, subsequently deprotected and functionalized, as required by the synthesis.

The compounds of formulae (II), (III), (VI) and (VII) are either commercially available or can be obtained by the person skilled in the art using conventional chemical reactions described in the literature.

Pharmacological studies of the compounds of the invention have shown pro-apoptotic and/or to anti-proliferative properties. The ability to reactivate the apoptotic process in cancerous cells is of major therapeutic interest in the treatment of cancers and of immune and auto-immune diseases.

Among the cancer treatments envisaged there may be mentioned, without implying any limitation, treatment of cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer. More especially, the compounds according to the invention will be useful in the treatment of chemo-, targeted therapy- or radio-resistant cancers.

The present invention relates also to pharmaceutical compositions comprising at least one compound of formula (I) in combination with one or more pharmaceutically acceptable excipients. In particular, these pharmaceutical compositions are interesting for use as pro-apoptotic and/or anti-proliferative agents, particularly, in the treatment of cancers and of auto-immune and immune system diseases. Preferably, these pharmaceutical compositions can be used in the treatment of cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer.

Among the pharmaceutical compositions according to the invention there may be mentioned more especially those that are suitable for oral, parenteral, nasal, per- or trans-cutaneous, rectal, perlingual ocular or respiratory administration, especially tablets or dragees, sublingual tablets, sachets, paquets, capsules, glossettes, lozenges, suppositories, creams, ointments, dermal gels, and drinkable or injectable ampoules.

The pharmaceutical compositions according to the invention comprise one or more excipients or carriers selected from diluents, lubricants, binders, disintegration agents, stabilisers, preservatives, absorbents, colorants, sweeteners, flavourings etc.

By Way of Non-Limiting Example There May be Mentioned:
as diluents: lactose, dextrose, sucrose, mannitol, sorbitol, cellulose, glycerol,
as lubricants: silica, talc, stearic acid and its magnesium and calcium salts, polyethylene glycol.
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The dosage varies according to the sex, age and weight of the patient, the administration route, the nature of the therapeutic indication, or of any associated treatments, and ranges from 0.01 mg to 1 g per 24 hours in one or more administrations.

Furthermore, the present invention relates also to the combination of a compound of formula (I) with anti-cancer agents selected from genotoxic agents, mitotic poisons, anti-metabolites, proteasome inhibitors, kinase inhibitors. protein-protein interaction inhibitors, immunomodulators, E3 ligase inhibitors, chimeric antigen receptor T-cell therapy and antibodies, and also to pharmaceutical compositions comprising that type of combination and their use in the manufacture of medicaments for use in the treatment of cancers, particularly, cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas. ovarian cancer, non-small-cell lung cancer. prostate cancer, pancreatic cancer and small-cell lung cancer.

The combination of a compound of formula (I) with an anticancer agent may be administered simultaneously or sequentially. The administration route is preferably the oral route, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

The compounds of formula (I) may also be used in combination with radiotherapy in he treatment of cancer.

The following Preparations and Examples illustrate the invention but do not limit it in any way.

General Procedures for Preparations R2a-R2ce, R3a-R3ce, R4a-R4ce, R5a-R51 and EXAMPLES 1 to 90 and 104 to 121

All reagents obtained from commercial sources were used without further purification. Anhydrous solvents were obtained from commercial sources and used without further drying.

Flash chromatography was performed on ISCO CombiFlash Rf 200i with pre-packed silica-gel cartridges (RediSepaR:Rf Gold High Performance).

Thin layer chromatography was conducted with 5×10 cm plates coated with Merck Type 60 F254 silica-gel.

Microwave heating was performed in an Anton Parr MonoWave or CEM Discover® instrument.

Preparative HPLC purifications were performed on an HANBON NP7000 Liquid Chromatography system with a Gemini-NX® 5 μm C18, 250 mm×50 mm i.d. column running at a flow rate of 99.9 mL×min⁻¹ with UV diode array detection (210-400 nm) using 5 mM aqueous NH₄HCO₃ solution and MeCN as eluents unless specified otherwise.

Chiral Chromatography was performed on Daicel columns in the mixture of heptane and alcohols.

Analytical LC-MS: The compounds of the present invention were characterized by high performance liquid chromatography-mass spectroscopy (HPLC-MS) on Agilent HP1200 with Agilent 6140 quadrupole LC/MS, operating in positive or negative ion electrospray ionisation mode. Molecular weight scan range is 100 to 1350. Parallel UV detection was done at 210 nm and 254 nm. Samples were supplied as a 1 mM solution in acetonitrile, or in THF/H₂O (1:1) with 5 μL loop injection. LCMS analyses were performed on two instruments, one of which was operated with basic, and the other with acidic eluents.

Basic LCMS: Gemini-NX, 3 μm, C18. 50 mm×3,00 mm i.d. column at 23° C., at a flow rate of 1 mL×min⁻¹ using 5 mM ammonium bicarbonate (Solvent A) and acetonitrile (Solvent B) with a gradient starting from 100% Solvent A and finishing at 100% Solvent B over various/certain duration of time.

Acidic LCMS: ZORBAX Eclipse XDB-C18, 1.8 μm, 50 mm×4.6 mm i.d. column at 40° C., at a flow rate of 1 mL×min⁻¹ using 0.02% v/v aqueous formic acid (Solvent A) and 0.02% v/v formic acid in acetonitrile (Solvent B) with a gradient starting from 100% Solvent A and finishing at 100% Solvent B over various/certain duration of time.

¹H-NMR measurements were performed on Bruker Avance III 500 MHz spectrometer and Bruker Avance III 400 MHz spectrometer, using DMSO-d6 or CDCl₃ as solvent. ¹H NMR data is in the form of delta values, given in part per million (ppm), using the residual peak of the solvent (2.50 ppm for DMSO-d6 and 716 ppm for CDCl₃) as internal standard. Splitting patterns are designated as: s (singlet), d (doublet), t (triplet), q (quartet), qn (quintet), sept (septet), m (multiplet), brs (broad singlet), brd (broad doublet), bn (broad triplet), brq (broad quartet), brm (broad multiplet), vbrs (very broad singlet), br, (broad singlet or doublet), dd (doublet or doublets), td (triplet or doublets), dt (doublet of triplets), dq (doublet of quartet), ddd (doublet of doublet of doublets), dm (doublet of multiplets), tm (triplet of multiplets), qm (quartet of multiplets).

Combination gas chromatography and low resolution mass spectrometry were performed on Agilent 6850 gas chromatograph and Agilent 5975C mass spectrometer using 15 m×0.25 mm column with 0.25 μm HP-5MS coating and helium as carrier gas, Ion source: EI+, 70 eV, 230 C, quadrupole 150° C., interface: 300° C.

High resolution mass spectrometry was performed on JEOL AccuTOF MS instrument connected to Agilent 7693A gas chromatograph on Rxi-5Sil MS column 15 m×0.25 mm column and helium was used as carrier gas. Ion source: EI+, 70 eV, 200° C. interface: 250° C. HRMS were determined on a Shimadzu IT-TOF ion source temperature 200° C., ESI +/31 , ionization voltage: (+−)4.5 kV. Mass resolution min. 10000.

Elementary analyses were performed on a Thermo Flash EA 1112 Elemental Analyzer:

IUPAC chemical names were generated using ACD/Name 2015 Pack 2 (File Version N20E41, Build 75170, 19 Dec. 2014) or using ‘Structure to Name’ functionality within Accelrys Draw 4.2.

### LIST OF ABBREVIATIONS

| Abbreviation | Name |
|---|---|
| abs. | absolute |
| aq. | aqueous |
| Boc | teri-butoxycarbonyl |
| cc. | concentrated |
| DAST | diethylaminosulfur trifluoride |
| DBU | 1,8-diazabicyclo[5.4.0]undec-7-ene |
| DCM | methylene chloride |
| DEE | diethylether |
| DIPO | diisopropyl oxide |
| DMAP | 4-dimethylaminopyridine |
| DMF | dimethylformamide |
| DMSO | dimethylsulfoxide |
| EEO | ethyl ethanoate |
| eq. | equivalent |
| Et₃N•3HF | triethylamine trihydrofluoride |
| EtOAc | ethyl acetate |
| HATU | 1-[bis(dimethylamino)methylene]-1H-1,2,3- |
| | triazolo[4,5-b]pyridinium 3-oxid |
| | hexafluorophosphate |
| HBTU | 3-[bis(dimethylamino)methyliumyl]-3H- |
| | benzotriazol-1-oxide hexafluorophosphate |
| LC | liquid chromatography |
| MeCN | acetonitrile |
| MeOH | methanol |
| MSM | methylsulfinylmethane |
| MTBE | tert-butyl methylether |
| r.t. | room temperature |
| sat. | saturated |
| TBAF | tetra-n-butylammonium fluoride |
| TFA | trifluoroacetic acid |
| TCEP | tris(2-carboxyethyl)phosphine |
| THF | tetrahydrofurane |
| TMSCl | trimethylsilyl chloride |
| TMSOTf | trimethylsilyl triflate |
| XtalFluor-E ® | (diethylamino)difluorosulfoniuin tetrafluoroborate |
| XtalFluor-M ® | difluoro(morpholino)sulfonium tetrafluoroborate |

General Procedure 1

4-chloro-6-methoxy-5-nitro-pyrimidine (Preparation R1a: 1.0 eq.), the appropriate phenol (1.2 eq.), and potassium carbonate (1.2 eq.) were dissolved in acetonitrile. It was stirred at 80° C. till completion, then water was added to the reaction mixture. MeCN was evaporated. The residue extracted with DCM. The combined organic phase was dried over MgSO₄ and evaporated under reduced pressure to give R2a-R2ce.

General Procedure 2

Autoclave was charged with R2a-R2ce (1.0 eq.), Raney-nickel catalyst (10 w/w %) and 1.4-dioxane and then placed under a nitrogen atmosphere. After that it was filled with 10 bar H₂ gas. The reaction mixture was stirred in autoclave at r.t. for 20 hours. The reaction mixture was removed from the autoclave and filtered. The filtrate was purified by preparative LC (on C-18 Gemini-NX 5 μm column, 5 mM aqueous NH₄HCO₃-MeCN, gradient). Solvent was evaporated under reduced pressure to give R3a-R3ce.

General Procedure 3

R2a-R2ce (1.0 eq.), and tin(II) chloride dihydrate (3.5 eq.) were dissolved in 1,4-dioxane. The reaction mixture was stirred till completion at r.t. Then sat. NaHCO₃ solution and EtOAc were added, the suspension was filtered through Celite. washed with EtOAc, the layers were separated. The aqueous phase extracted with EtOAc. The Celite washed with DCM-MeOH. The organic phase evaporated to give R3a-R3ce.

General Procedure 4

R3a-R3ce (1.0 eq.) was dissolved in 1.4-dioxane, then 1N hydrochloric acid (3.0-5.0 eq.) was added. It was stirred at 95° C. till completion, then the reaction mixture concentrated under reduced pressure to give R4a-R4ce.

General Procedure 5

R4a-R4ce (1.0 eq.), R5a-R51 (1.0 eq.) and potassium carbonate (3.0 eq.) were dissolved in N,N-dimethylformamide. It was stirred at 70° C. till completion. The reaction mixture was directly injected through syringe filter to preparative HPLC (on C-18 Gemini-NX 5 μm column, 5 mM NH₄HCO₄ aqueous solution MeCN, gradient 5-90%). Fractions were collected and concentrated under reduced pressure, then dried in vacuum at 50° C. for overnight.

General Procedure 6: Boc Protection

The appropriate amine (1.0 eq.), tert-butoxycarbonyl tert-butyl carbonate (1.5 eq.), and sodium hydrogen carbonate (2.0 eq.) were dissolved in THF and water (1:1). It was stirred at r.t. till completion. The reaction mixture was extracted with EtOAc. The combined organic phase dried on MgSO₄ and the solvent was evaporated under reduced pressure to give the appropriate Boc protected amine.

General Procedure 7: Boc Deprotection

The appropriate Boc protected amine (1.0 eq.) was dissolved in 1.4-dioxane and 1N hydrochloric acid solution (5.0 eq.) was added. It was stirred at 70° C. till completion, then the solvents were evaporated under reduced pressure to give the appropriate amine derivative.

General Procedure 8

Step 1:

Corresponding aryl-carbaldehyde (1.0 eq.) and 1-(triphenyl-phosphanylidene)propan-2-one (1.2 eq.) were dissolved in DCM. The mixture was stirred at r.t. for 1-168 hours. The solvent was evaporated. The residue was purified by flash chromatography (hexane:EEO) to give the appropriate (E)-4-(aryl)but-3-en-2-one.

Step 2:

A solution of corresponding (E)-4-(aryl)but-3-en-2-one obtained in Step 1 above (2.1 eq.). triethylamine (1.5 eq.) and abs. DCM were cooled to −20° C. and TMSOTf (2.0 eq.) was added dropwise. The solution was stirred for 1 hour at this temperature. The mixture was washed with aq. NaHCO₃ solution (15 ml)3 times. The organic layer was dried over MgSO₄, then the solvent was evaporated in reduced pressure. The residue was used without further purification.

Step 3:

Corresponding (E)-((4-(aryl)buta-1,3-dien-2-yl)oxy)trimethylsilane obtained in Step 2 above (1.0 eq.) and ethyl acrylate (2.0 eq.) were dissolved in abs. toluene. The mixture was stirred at 120° C. for 1-2 days. The solvent was evaporated. The residue was dissolved in THF/1M aq. HCl 1:1 v/v mixture and stirred for 1 hour at 25° C. Then the emulsion was diluted with DEE and washed 3 times with NaHCO₃ solution and with brine. The organic layer was dried over MgSO₄ and then the solvent was evaporated under reduced pressure. The crude product was purified by flash chromatography (hexane:EEO) to give the corresponding ethyl 2-(aryl)-4-oxocyclohexane-1-carboxylate.

Step 4:

Oven-dried flask was inertized then filled with ethyl 2-(aryl)-4-oxocyclohexane-1-carboxylate obtained in Step 3 above (1.0 eq.) and abs. DCM (c=0.05M). The solution was cooled to 10° C. and DAST (5.0 eq.) was added dropwise. After that the reaction mixture was stirred for 3 hours at 25° C. The reaction mixture was quenched with aq. NaHCO₃ solution (25 ml), and the mixture was washed with aq. NaHCO₃ solution twice. The organic layer was dried over MgSO₄, and then the solvent was evaporated tinder reduced pressure. The crude product was purified by column chromatography (hexane:EEO) to give the corresponding ethyl 4,4-difluoro-2-(aryl)cyclohexane-1-carboxylate.

Step 5:

The corresponding ester obtained in Step 4 above was dissolved in the mixture of ethanol and water (5:1, v/v and lithium hydroxide hydrate (2.0-3.0 eq.) was added. It was stirred at r.t. for 44-435 hours.

Work-Up 1:

The reaction mixture was partially evaporated to water and isolated as lithium salt.

Work-Up 2:

The reaction mixture was evaporated to water, then 1N HCl was added. The obtained solid was filtered off.

Work-Up 3:

The reaction mixture was evaporated to water, 1N HCl was added, and then it was evaporated again. The residue was purified by preparative HPLC (on C-18 Gemini-NX 5 μm column, 5 mM aqueous NH₄HCO₃-MeCN, gradient).

Step 6:

The appropriate 4-piperidone hydrochloride hydrate. HBTU (1.6 eq.). 2-aryl-4,4-difluoroyclohexanecarboxylic acid (1.0 eq.) and N,N-diisopropylethylamine (5.0 eq.) were dissolved in MeCN (50 mL) and stirred till completion. After evaporation, the residue was dissolved in DCM and it was washed with 1N NaOH and then with 1N HCl and then with water. Organic layer was dried (MgSO₄) and evaporated. DIPO was added, solid compound was formed, which was filtered off to give the appropriate 1-(2-aryl-4,4-difluorocyclohexanecarbonyl)piperidin-4-one.

Step 7:

1-(2-Aryl-4,4-difluorocyclohexanecarbonyl)piperidin-4-one (1.0 eq.) and trimethylsulfoxonium-iodide (5.0 eq.) was charged into a round bottom flask and dissolved/suspended m MeCN and MTBE (1:1). NaOH (2.5 eq.) was dissolved in water and the obtained solution was added to the mixture and stirred at 60° C. for 6 hours. After the reaction completed, the reaction mixture was filtered through Celite, and washed with MTBE. Water was added to the solution, layers were separated, and the aqueous layer was extracted with MTBE. Combined organic layers were dried over MgSO₁ and after filtration evaporated to give the appropriate (2-aryl-4,4-difluorocyclohexyl)-(1-oxa-6-azaspiro[2.5]octan-6-yl)methanone.

### Preparation R2a: 6-methoxy-5-nitro-4-phenoxy-1,6-dihydropyrimidine

Using General Procedure 1 starting from Preparation R1a and phenol as reagents, Preparation R2a was obtained. ¹H-NMR (500 MHZ, dmso-d6) δ ppm 8.59 (s, 1H), 7.48 (m, 2H), 7.33 (tm, 1H), 7.27 (dm, 2H), 4.1 (s, 3H)

¹³C-NMR (125 MHz, dmso-d6) δ ppm 162.4, 161.1, 158.7, 152, 130.4, 126.8, 122.1, 56.6

### Preparation R2b: 4-(2-fluorophenoxy)-6-methoxy-5-nitro-pyrimidine

Using General Procedure 1 starting from Preparation R1a and 2-fluorophenol as reagents. Preparation R2b was obtained. HRMS calculated for C₁₁H₈FN₃O₄: 265.0499; found 265.04976 (M⁺ form).

¹H-NMR (400 MHz, dmso-d6) δ ppm 8.62 (s, 1H), 7.48 (dd, 1H), 7.43 (dd, 1H), 7.41 (m, 1H), 7.31 (m, 1H), 4.12 (s, 3H)

¹³C-NMR (125 MHz, dmso-d6) δ ppm 162.6, 160.3, 158.8, 153.9, 138.8, 128.8, 126, 124.5, 117.4, 56.8

### Preparation R2c: 4-methoxy-6-(4-methoxyphenoxy)pyrimidin-5-amine

Using General Procedure 1 starting from Preparation R1a and 4-methoxyphenol as reagents. Preparation R2c was obtained. HRMS calculated for C₁₂H₁₃N₃O₃: 247.0957; found 248.10318 ((M+H)⁺ form).

¹H-NMR (500 MHz, dmso-d6) δ ppm 7.72 (s, 1H, 7.06 (m, 2H), 6.94 (m, 2H), 4.79 (s, 2H), 3.94 (s, 3H), 335 (s, 3H)

¹³C-NMR (125 MHz, dmso-d6) δ ppm 157.7, 156.5, 155,4, 147.3, 142.9, 122.8, 116.7, 114.9

### Preparation R2d: 4-methoxy-6-(3-methoxyphenoxy)-5-nitro-pyrimidine

Using General Procedure 1 starting from Preparation R1a and 3-methoxyphenol as reagents. Preparation R2d was obtained. HRMS calculated for C₁₂H₁₁N₃O₅: 277.0699; found 278.0773 ((M+H)⁺ form).

¹H-NMR (500 MHz, dmso-d6) δ ppm 8.6 (s, 1H), 7.36 (t, 1H), 6.9 (dm, 1H), 6.89 (m, 1H), 6.83 (dm, 1H), 4.1 (s, 3H), 3.76 (s, 3H)

¹³C-NMR (125 MHz, dmso-d6) δ ppm 162.4, 161.1, 160.9, 158.7, 152.2, 130.7, 114, 112.7, 108

### Preparation R2e: 4-(4-fluorophenoxy)-6-methoxy-5-nitro-pyrimidine

Using General Procedure 1 starting from Preparation R1a and 4-fluorophenol as reagents. Preparation R2e was obtained. HRMS calculated for C₁₁H₁₁FN₃O₄: 265.0499; found 265.04956 (M⁺ form).

¹H-NMR (400 MHz, dmso-d6) δ ppm 8.59 (s, 1H), 7.33 (m, 2H), 7.33 (m, 2H), 4.1 (s, 3H)

¹³C-NMR (125 MHz, dmso-d6) δ ppm 162.4, 161.6, 158.6, 124.1, 117, 56.7

### Preparation R2f: 4-(3-fluorophenoxy)-6-methoxy-5-nitro-pyrimidine

Using General Procedure 1 starting from Preparation R1a and 3-fluorophenol as reagents. Preparation R2f was obtained. HRMS calculated for C₁₁H₈FN₃O₄: 265.0499; found 266.05704 ((M+H)⁺ form).

¹H-NMR (400 MHz, dmso-d6) δ ppm 8.62 (s, 1H), 7.52 (m, 1H), 7.31 (m, 1H), 7.2 (m, 1H), 7.16 (dd, 1H), 4.11 (s, 3H),

¹³C-NMR (125 MHz, dmso-d6) δ ppm 163, 162.5, 158.7, 152.8, 131,6, 118.4, 113.9, 110.3, 56.7.

### Preparation R2g: 4-(3,5-dimethoxyphenoxy)-6-methoxy-5-nitro-pyrimidine

Using General Procedure 1 starting from Preparation R1a and 3,5-dimethoxyphenol as reagents. Preparation R2g was obtained. HRMS calculated for C₁₃H₁₃N₃O₆: 307.0804; found 308.0882 ((M+H)⁺ form).

¹H-NMR (500 dmso-d6) δ ppm 8.61 (s, 1H), 6.47 (m, 2H), 6.45 (m, 1H), 4.1 (s, 3H), 3.74 (s, 6H)

¹³C-NMR (125 MHz, dmso-d6) δ ppm 162.4. 161.5, 161.1, 158.7, 153.6, 121, 100.6, 98.9, 56.6, 56.1.

### Preparation R2h: 4-(3,5-difluorophenoxy)-6-methoxy-5-nitro-pyrimidine

Using General Procedure 1 starting from Preparation R1a and 3,5-difluorophenol as reagents. Preparation R2h was obtained. HRMS calculated for C₁₁H₇F₂N₃O₄: 283.0405; found 284.0477 ((M+H)⁺ form).

¹H-NMR (500 MHz, dmso-d6) δ ppm 8.65 (s, 1H), 7.28 (t, 1H), 7.24 (d, 2H), 4.12 (s, 3H)

¹³C-NMR (125MHz, dmso-d6) δ ppm 163, 162.6, 160.5, 158.7, 153.3, 121, 107, 102.8, 56.8

### Preparation R2j: 4-(4-chlorophenoxy)-6-methoxy-5-nitro-pyrimidine

Using General Procedure 1 starting from Preparation R1a and 4-chlorophenol as reagents. Preparation R2j was obtained. HRMS calculated for C₁₁H₈ClN₃O₄: 281.0203; found 281.01978 (M⁺ form).

¹H-NMR (400 MHz, dmso-d6) δ ppm 8.6 (s, 1H) 7.54 (m, 2H), 7.34 (m, 2H), 4.1 (s, 3H),

¹³C-NMR (125 MHz, dmso-d6) δ ppm 162.5, 161, 158.7, 150.7, 131, 130.3, 124.1, 56.7.

### Preparation R2k: 4-(4-chloro-3-methoxy-phenoxy)-6-methoxy-5-nitro-pyrimidine

Using General Procedure 1 starting from Preparation R1a and 4-chloro-3-methoxy-phenol as reagents. Preparation R2k was obtained. HRMS calculated for C₁₂H₁₀ClN₃O₅: 311.0309; found 312.038 ((M+H) form).

¹H-NMR (400 MHz, dmso-d6) δ ppm 8.61 (s, 1H), 7.51 (d, 1H), 7.17 (d, 1H), 6.9 (dd, 1H), 4.11 (s, 3H), 3.83 (s, 3H),

¹³C-NMR (125 MHz, dmso-d6) δ ppm 162.4, 161, 158.7, 155.8, 151.6, 130.7, 119, 114.9, 107.7, 57, 56.7.

### Preparation R2l: 4-(3-chlorophenoxy)-6-methoxy-5-nitro-pyrimidine

Using General Procedure 1 starting from Preparation R1a and 3-chlorophenol as reagents. Preparation R2l was obtained. HRMS calculated for C₁₁H₈ClN₃O₄: 281.0203; found 282.0276((M+H)⁺ form),

¹H-NMR (400 MHz, dmso-d6) δ ppm 8.62 (s, 1H) 7.51 (t, 1H), 7.51 (brs, 1H), 7.42 (dm, 1H), 7.29 (dm, 1H), 4.11 (s, 3H)

¹³C-NMR (125 MHz, dmso-d6) δ ppm 162.5, 160.9, 158.7, 152.6, 134.1, 131.7, 127, 122.6, 121.1, 56.7

### Preparation R2n: 4-(1,3-benzodioxol-5-yloxy)-6-methoxy-5-nitro-pyrimidine

Using General Procedure 1 starting from Preparation R1a and 1,3-benzodioxol-5-ol as reagents. Preparation R2n was obtained. HRMS calculated for C₁₂H₉N₃O₆: 291.0491; found 292.057 ((M+H)⁻ form).

¹H-NMR (500 MHz, dmso-d6) δ ppm 8.59 (s, 1H), 6.97 (d, 1H), 6.05 (d, 1H) 6.7 ((id, 1H), 6.09 (s, 2H), 4.09 (s, 3H),

¹³C-NMR (125 MHz, dmso-d6) δ ppm 162.3, 161.4, 158.6, 148.9, 148.3, 146,1, 120.8, 114.5, 108.6, 104.4, 102.4, 56.6.

### Preparation R2o: 3-methoxy-5-(6-methoxy)-5-nitro-pyrimidin-4-yl)oxy-phenol

Using General Procedure 1 starting from Preparation R1a and 5-methoxybenzene-1,3-diol as reagents. Preparation R2o was obtained. HRMS calculated for C₁₂H₁₁N₃O₆: 293.0648; found 294.0718 (M+H)⁺ form).

¹H-NMR (500 MHz, dmso-d6) δ ppm 9.86 (s, 1H), 8.6 (s, 1H), 6.29/6.28/6.21 (t+t+t, 3H), 4.09 (s, 3H), 3.69 (s, 3H),

¹³C-NMR (125 MHz, dmso-d6) δ ppm 162.3, 161.5. 161, 159.6/153.4, 158.7, 101.8/99.8/99, 56.6, 55.8.

### Preparation R2p: 4-(4-fluoro-3-methoxy-phenoxy)-6-methoxy-5-nitro-pyrimidine

Using General Procedure 1 starting from Preparation R1a and 4-fluoro-3-methoxy-phenol as reagents. Preparation R1p was obtained. HRMS calculated for C₁₂H₁₀FN₃O₅: 295.0605; found 296.0675 ((M+H)⁺ form).

¹H-NMR (500 MHz, dmso-d6) δ ppm 8.84 (s, 1H), 7.33 (dd, 1H), 7.26 (dd, 1H), 6.91 (ddd, 1H), 3.81 (s, 3H), 3.81 (s, 3H)

¹³C-NMR (125 MHz, dmso-d6) δ ppm 159.1, 116.8. 113.8, 108.5. 56.9, 56.9

### Preparation R2r: 4-methoxy-5-nitro-6-[3-(trifluoromethoxy)phenoxy]pyrimidine

Using General Procedure 1 starting from Preparation R1a and 3-(trifluoromethoxy)phenol as reagents. Preparation R2r was obtained. HRMS calculated for C₁₂H₈F₃N₃O₅, 331.0416; found 332.0488 ((M+H)⁺ form).

¹H-NMR (500 MHz, dmso-d6) δ ppm 8.62 (s, 1H), 7.62 1H), 7.47 (s, 1H), 7.37 (m, 1H), 7.36 (m, 1H), 4.11 (s, 3H),

¹³C-NMR (125 MHz, dmso-d6) δ ppm 162.5, 160.8, 158.7, 152.6, 149.2, 131.8, 121.5, 121.4, 120.1, 119.4, 115.8, 56.7.

### Preparation R2s: 4-methoxy-6-(3-methylphenoxy)-5-nitro-pyrimidine

Using General Procedure 1 starting from Preparation R1a and m-cresol as reagents, Preparation R2s was obtained. HRMS calculated For C₁₂H₁₁N₃O₄: 261.075; found 262.0825 ((M+H)⁺ form).

¹H-NMR (500 MHz, dmso-d6) δ ppm 8.59 (s, 1H), 7.35 (t, 1H), 7.14 (dm, 1H), 7.09 (m, 1H), 7.05 (dm, 1H), 4.1 (s, 3H), 2.33 (s, 3H),

¹³C-NMR (125 MHz, dmso-d6) δ ppm 162.3, 161.1, 158.7, 140.2, 130, 127.5, 122.4, 121, 119, 56.6, 21.2.

### Preparation R2t: 4-(3-bromophenoxy)-6-methoxy-5-nitro-pyrimidine

Using General Procedure 1 starting from Preparation R1a and 3-bromophenol as reagents. Preparation R2t was obtained. HRMS calculated for C₁₁H₈BrN₃O₄324.9698; found 325.9771 ((M+H)⁺ form).

¹H-NMR (500 MHz, dmso-d6) δ ppm 8.62 (s, 1H), 7.63 (m, 1H), 7.55 (dm, 1H), 7.44 (t, 1H), 7.33 (dm, 1H), 4.11 (s, 3H),

13C-NMR (125 MHz, dmso-d6) δ ppm 1600.9, 162.5, 158.7, 132, 129.9, 125.4, 121.4, 120.9, 56.7.

### Preparation R2u: 4-methoxy-5-nitro-6-[3-(pentafluoro-λ⁶-sulfanyl)phenoxy]pyrimidine

Using General Procedure 1 starting from Preparation R1a and 3-(pentafluoro-λ⁶-sulfanyl)phenol as reagents. Preparation R2u was obtained. HRMS calculated for C₁₁H₈F₅N₃O₄S: 373.0156; found 374.022 ((M+H)⁺ form).

¹H-NMR (400 MHz, dmso-d6) δ ppm 8.62 (s, 1H), 8.02 (t, 1H), 7.74 (t, 1H), 7.66 (dd, 1H), 4.12 (s, 3H),

¹³C-NMR (125 MHz, dmso-d6) δ ppm 162.6, 160.9, 158.6, 151.7, 131.3, 126.8, 120.6, 56.7

¹⁹F-NMR (376 MHz, dmso-d6) δ ppm 86, 64.2.

### Preparation R2v: 4-methoxy-5-nitro-6-[3-(trifluoromethyl)phenoxy]pyrimidine

Using General Procedure 1 starting from Preparation R1a and 3-(trifluoromethyl)phenol as reagents. Preparation R2v was obtained. HRMS calculated for C₁₂H₈F₃N₃O₄: 315.0467; found 316.0545 ((M+H)⁺ form).

¹H-NMR (500 MHz, dmso-d6) δ ppm 8.62 (s, 1H), 7.79 (m, 1H), 7.72 (m, 1H), 7.72 (m, 1H), 7.65 (m, 1H), 4.12 (s, 3H),

¹³C-NMR (125 MHz, dmso-d6) δ ppm 162.5, 160.9, 158.7, 152.2, 131.7, 131, 126.6, 124, 123.7, 119.5, 56.7.

### Preparation R2w: [4-(6-methoxy-5-nitro-pyrimidin-4-yl)oxyphenyl]methanol

Using General Procedure 1 starting from Preparation R1a and 4-(hydroxymethyl)phenol as reagents. Preparation R2w was obtained. HRMS calculated for C₁₂H₁₁N₃O₅: 277.0699; found 278.0764 ((M+H)⁻ form).

¹H-NMR (400 MHz, dmso-d6) δ ppm 8.58 (s, 1H), 7.4 (d, 2H), 7.22 (d, 2H), 5.27 (t, 1H), 4.53 (d, 2H), 4.11 (s, 3H), ¹³C-NMR (125 MHz, dmso-d6) δ ppm 162.4, 161.3, 158.6, 150.5, 141.2, 128.4, 121.7, 120.9, 62.7, 56.6.

### Preparation R2z: 4-(6-methoxy-5-nitro-pyrimidin-4-yl)oxybenzonitrite

Using General Procedure 1 starting from Preparation R1a and4-hydroxybenzonitrile as reagents. Preparation R2z was obtained. HRMS calculated for C₁₂H₈N₄₄: 272.0546; found 273.0621 ((M+H)⁺ form).

¹H-NMR (400 MHz, dmso-d6) δ ppm 8.63 (s, 1H), 7.99 (d, 2H), 7.55 (d, 2H), 4.12 (s, 3H)

¹³C-NMR (125 MHz, dmso-d6) δ ppm 162.6, 160.6, 158.7, 155.4, 134.9, 123.5, 118,8, 109.9, 56.8

### Preparation R2aa: 3-(6-methoxy-5-nitro-pyrimidin-4-yl)oxybenzonitrile

Using General Procedure 1 starting from Preparation R1a and 3-hydroxybenzonitrile as reagents. Preparation R2aa was obtained. HRMS calculated for C₁₂H₈N₄O₄: 272.0546; found 272.05401 (M⁺ form).

¹H-NMR (400 MHz, dmso-d6) δ ppm 8.62 (s, 1H), 7.92 (m, 1H), 7.83 (m, 1H), 7.69 (m. 1H), 7.69 (m, 1H), 4.12 (s, 3H),

¹³C-NMR (125 MHz, dmso-d6) δ ppm 162.6, 160.7, 158.7, 152, 131.8, 130.9, 127.7, 126.1, 118.2, 113, 56.8.

### Preparation R2ab: tert-butyl 7-(6-methoxy-5-nitro-pyrimidin-4-yl)oxy-3,4-dihydro-1H-isoquinoline-2-carboxylate

Using General Procedure 1 starting from Preparation R1a and tert-butyl 7-hydroxy-3,4-dihydro-1H-isoquinoline-2-carboxylate as reagents. Preparation R2ab was obtained. HRMS calculated for C₁₉H₂₂N₄O₆: 402.1539; found 425.1421 ((M+Na) form).

¹H-NMR (400 MHz, dmso-d6) δ ppm 8.6 (s, 1H), 7.25 (d, 1H), 7.12 (d, 1H), 7.08 (dd 1H), 4.51 (brs, 2H), 4.11 (s, 3H), 3.57 (t, 2H), 2.8 (t, 2H), 1.43 (s, 9H),

¹³C-NMR (125 MHz, dmso-d6) δ ppm 162.4, 161.1, 158.7, 154.1, 150.2, 135.7, 133.3, 130.6, 120.2, 119.6, 79.5, 56.6, 45.4, 41.5, 28.6, 28.2.

### Preparation R2ac: methyl 3-(6-methoxy-5-nitro-pyrimidin-4-yl)oxybenzoate

Using General Procedure 1 starting from Preparation R1a and methyl 3-hydroxybenzoate as reagents. Preparation R2ac was obtained. HRMS calculated for C₁₃H₁₁N₃O₆: 305.0648; found 306.0714 ((M+H)⁺ form).

¹H-NMR (400 MHz, dmso-d6) δ ppm 8.6 (s, 1H), 7.93 (dd, 1H), 7.83 (1H), 7.65 (t, 1H), 7.61 (dd, 1H), 4.11 (s, 3H), 3.88 (s, 3H),

¹³C-NMR (125 MHz, dmso-d6) δ ppm 165.7, 162.5, 161, 158.6, 152, 131.9, 131, 127.6, 127.2, 122.8, 121, 56.7, 52.9.

### Preparation R2ad: [3-(6-methoxy-5-nitro-pyrimidin-4-yl)oxyphenyl]methanol

Using General Procedure 1 starting from Preparation R1a and 3-(hydroxymethyl)phenol as reagents. Preparation R2ad was obtained. HRMS calculated for C₁₂H₁₁N₃O₄: 2770699; found 278.077 ((M+H)⁺ form). ¹H-NMR (400 MHz, dmso-d6) δ ppm 8.6 (s, 1H), 7.43 (t, 1H), 7.27 (dd, 1H), 7.19 (t, 1H), 7.13 (dd, 1H), 5.33 (t, 1H), 4.54 (d, 2H), 411 (s, 3H),

¹³C-NMR (125 MHz, dmso-d6) δ ppm 162.4, 161.2, 158.7, 152, 145.5, 130, 124.6, 121, 120.3, 119.7, 62.7, 56.6.

### Preparation R2ae: 3-[4-(6-methoxy-5 nitro-pyrimidin-4-yl)oxyphenyl]propan-1-ol

Using General Procedure 1 starting from Preparation R1a and 4-(3-hydroxypropyl)phenol as reagents. Preparation R2ae was obtained. HRMS calculated for C₁₄H₁₅N₃O₅: 305.1012; found 306.1082 ((M+H)⁺ form).

¹H-NMR (400 MHz, dmso-d6) δ ppm 8.59 (s, 1H), 7.29 (dm, 2H), 7.17 (dm, 2H), 4.51 (t, 1H), 4.11 (s, 3H), 3.43 (m, 2H), 2.65 (t, 2H), 1.74 (quin, 2H),

¹³C-NMR (125 MHz, dmso-d6) δ ppm 162.3, 161.2, 158.6, 149.9, 140.8, 130.1, 121.8, 60.4, 56.6, 34.7, 31.5.

### Preparation R2af: 4-methoxy-5-nitro-6-phenylsulfanyl-pyrimidine

Using General Procedure 1 starting from Preparation R1a and benzenethiol as reagents, Preparation R2af was obtained. HRMS calculated for C₁₁H₉N₃O₃S: 263.0365; found 264.0434 ((M+H)⁺ form).

¹H-NMR (400 MHz, dmso-d6) δ ppm 8.61 (s, 1H), 7.63-7.44 (m, 5H), 4.07 (s, 3H),

¹³C-NMR (125 MHz, dmso-d6) δ ppm 165.4, 161.9, 158.3, 127.5, 56.4.

### Preparation R2ag: tert-butyl 6-(6-methoxy-5-nitro-pyrimidin-4-yl)oxy-3,4-dihydro-1H-isoquinoline-2-carboxylate

Using General Procedure 1 starting from Preparation R1a and tert-butyl 6-hydroxy-3,4-dihydro-1H-isoquinoline-2-carboxylate as reagents. Preparation R2ag was obtained. HRMS calculated for C₁₉H₂₂N₄O₆: 402.1539; found 347.0988 ((M+H-tBu)⁻ form).

¹H-NMR (500 MHz, dmso-d6) δ ppm 8.58 (s, 1H), 7.26 (m, 1H), 7.08 (m, 1H), 7.08 (m, 1H), 4.52 (brs, 2H), 4.1 (s, 3H), 3.55 (t, 2H ), 2.78 (t, 2H), 1.43 (s, 9H),

¹³C-NMR (125 MHz, dmso-d6) δ ppm 158.6, 128.2, 121.8, 119.9, 56.6, 45.3, 41, 28.6, 28.6.

### Preparation R2ah: tert-butyl 5-(6-methoxy-5-nitro-pyrimidin-4-yl)oxyisoindoline-2-carboxylate

Using General Procedure 1 starting from Preparation R1a and tert-butyl 5-hydroxyisoindoline-2-carboxylate as reagents. Preparation R2ah was obtained. HRMS calculated for C₁₈H₂₀N₄O₆: 388.1383; found 333.0826 ((M+H-tBu)⁺ form).

¹H-NMR (500 MHz, dmso-d6) δ ppm 8.58 (s, 1H), 7.41/7.4 (d, 1H), 7.26/7.24 (d, 1H), 7.18 (dd, 1H), 4.63-4.55 (brs, 4H), 4.1 (s, 3H), 1.46 (s, 9H),

¹³C-NMR (125 MHz, dmso-d6) δ ppm 158.7, 124.6, 121.3, 116.7, 56.6, 28.6.

### Preparation R2ai: 2-[4-(6-methoxy-5-nitro-pyrimidin-4-yl)oxyphenoxy]ethanol

Using General Procedure 1 starting from Preparation R1a and 4-(2-hydroxyethoxy)phenol as reagents. Preparation R2ai was obtained. HRMS calculated for C₁₃H₁₃N₃O₆: 307.0804; found 308.088 ((M+H)⁺ form).

¹H-NMR (500 MHz, dmso-d6) δ ppm 8.57 (s, 1H), 7.17 (dm, 2H), 7 (dm, 2H), 4.89 (t, 1H), 4.09 (s, 3H), 4 (t, 2H), 3.72 (q, 2H),

¹³C-NMR (125 MHz, dmso-d6) δ ppm 162.3, 161.,4 158.6, 145.2, 145.2, 123, 115.7, 70.4, 60, 56.6.

### Preparation R2ai: tert-butyl 2-[4-6-methoxy-5-nitro-pyrimidin-4-yl)oxyphenyl]pyrrolidine-1-carboxylate

4-Pyrrolidin-2-ylphenol, hydrogen bromide (1:1) (1 g, 4.0962 mmol), tert-butoxycarbonyl tert-butyl carbonate (1,5 eq., 6.1443 mmol) and sodium hydrogen carbonate (4.0 eq., 16.385 mmol) were dissolved in THF (10 mL) and water (10 mL). The reaction mixture was stirred at r.t. for 20 hours. The reaction mixture was extracted with EtOAc (3×10 ml). The organic layer was evaporated after drying with brine and MgSO₄. The residue was washed with diisopropyl ether and the solid compound was filtered off to give tert-butyl 2-(4-hydroxyphenyl)pyrrolidine-1-carboxylate as a crude product.

Using General Procedure 1 starting from Preparation R1a and tert-butyl 2-(4-hydroxyphenyl)pyrrolidine-1-carboxylate as reagents. Preparation R2aj was obtained. HRMS calculated for C₃₀H₂₄N₄O₆: 416.1696; found 439.581 ((M+Na)⁺ form).

¹H-NMR (500 MHz, dmso-d6) δ ppm 8.56 (s, 1H), 7.226 (m, 2H), 7.19 (m, 2H), 4.8 (brm, 1H), 4.12 (s, 3H),3.58-3.45 (m, 2H), 2.32/1.75 (m+m, 2H), 1.9-1.8 (m, 2H), 1.26 (brs, 9H),

¹³C-NMR (125 MHz, dmso-d6) δ ppm 158.5, 127.3, 121.5, 60.6, 56.5, 47.4, 35.7, 28.5, 23.4.

### Preparation R2al: 6-(6-methoxy-5-nitro-pyrimidin-4-yl)oxy-1H-indazole

Using General Procedure 1 starting from Preparation R1a and 1H-indazol-6-ol as reagents. Preparation R2al was obtained. HRMS calculated for C₁₂H₉N₅O₄: 287.0655; found 288.0729 ((M+H)⁺ form).

¹H-NMR (500 MHz, dmso-d6) δ ppm 13.21 (s, 1H), 8.58 (s, 1H), 8.12 (m, 1H), 7.83 (d, 1H), 7.45 (m, 1H), 7 (dd, 1H), 4.11 (s, 3H)

¹³C-NMR (125 MHz, dmso-d6) δ ppm 162.4, 161.4, 158.7, 150.4, 140.3, 134.2, 122.2, 121.6, 115.7, 103.1, 56.6

### Preparation R2am: 2-[4-(6-methoxy-5-nitro-pyrimidin-4-yl)oxyphenyl]ethanol

Using General Procedure 1 starting from Preparation R1a and 4-(2-hydroxyethyl)phenol as reagents. Preparation R2am was obtained. HRMS calculated for C₁₃H₁₃N₃O₅: 291.0855; found 292.093 ((M+H)⁺ form).

¹H-NMR (500 MHz, dmso-d6) δ ppm 8.58 (s, 1H), 7.3 (dm, 2H), 7.15 (dm, 2H), 4.61 (t, 1H), 4.1 (s, 3H), 3.62 (m, 2H), 2.74 (t, 2H)

¹³C-NMR (125 MHz, dmso-d6) δ ppm 162.3, 161.2, 158.6, 150.2, 138.3, 130.7, 121.6, 62.4, 56.6, 38.7

### Preparation R2an: 4-methoxy-5-nitro-6-[4-(2,2,2-trifluoroethyl)phenoxy]pyrimidine

Using General Procedure 1 starting from Preparation R1a and 4-(2,2,2-trifluoroethyl) phenol as reagents. Preparation R2an was obtained. HRMS calculated for C₁₃H₁₀F₃N₃O₄: 329.0623; found 330.0707 ((M+H)⁺ form).

¹H-NMR (500 MHz, dmso-d6) δ ppm 8.6 (s, 1H), 7.46 (m, 2H), 7.3 (m, 2H), 4.1 (s, 3H), 3.71 (q, 2H)

¹³C-NMR (125 MHz dmso-d6) δ ppm 158.7, 132.3, 126.8, 122.2, 38.2

### Preparation R2ao: 4-(4-(2,2-difluoroethyl)phenoxyl-6-methoxy-5-nitro-pyrimidine

Using General Procedure 1 starting from Preparation R1a and 4-(2,2-difluormethyl) phenol as reagents. Preparation R2ao was obtained. HRMS calculated for C₁₃H₁₁F₂N₃O₄: 311.0717; found 312.0786 ((M+H)⁺ form).

¹H-NMR (400 MHz, dmso-d6) δ ppm 8.59 (s, 1H), 7.4 (dm, 2H), 7.25 (dm, 2H), 6.28 (tt, 1H), 4.1 (s, 3H), 3.22 (td, 2H)

¹³C-NMR (125 MHz, dmso-d6) δ ppm 162.4, 161.1, 158.6, 151.1, 131.8, 131.4, 122.1, 117.4, 58.6, 39.4

### Preparation R2ap: 4-[4-(2-fluoroethyl)phenoxy]-6-methoxy-5-nitro-pyrimidine

Using General Procedure 1 starting from Preparation R1a and 4-(2-fluoroethyl)phenol as reagents. Preparation R2ap was obtained. HRMS calculated for C₁₃H₁₂FN₃O₄: 293.0812; found 294.0883 ((M+H)⁺ form).

¹H-NMR (500 MHz, dmso-d6) δ ppm 8.58 (s, 1H), 7.37 (dm, 2H), 7.21 (dm, 2H), 4.66 (dt, 2H), 4.1 (s, 3H), 3.01 (dt, 2H),

¹³C-NMR (125 MHz, dmso-d6) δ ppm 162.4, 161.2, 158.6, 150.6, 136.1, 130.8, 121.9, 120.9, 84.3, 56.6, 35.9

### Preparation R2aq: 4-[4-fluoro-3-(trifluoromethoxy)phenoxy]-6-methoxy-5-nitro-pyrimidine

Using General Procedure 1 starting from Preparation R1a and 4-fluoro-3-(trifluoromethoxy)phenol as reagents. Preparation R2aq was obtained. HRMS calculated for C₁₂H₂F₄N₃O₅: 349.0322; found 350.0392 ((M+H)⁺ form).

¹H-NMR (500 MHz, dmso-d6) δ ppm 8.63 (s, 1H), 7.75 (dm, 1H), 7.65 (t, 1H), 7.46 (dm, 1H), 4.11 (s, 3H),

¹³C-NMR (125 MHz, dmso-d6) δ ppm 162.5, 160.9, 158.7, 152.2, 147.7, 135.7, 123.5, 118.9, 118.7, 56.7.

### Preparation R2as: 4-(4-chloro-3-ethyl-phenoxy)-6-methoxy-5-nitro-pyrimidine

Using General Procedure 1 starting from Preparation R1a and 4-chloro-3-ethyl-phenol as reagents. Preparation R2as was obtained. HRMS calculated for C₁₃H₁₂ClN₃O₄: 309.0516; found 310.0589 ((M+H)⁺ form).

¹H-NMR (500 MHz, dmso-d6) δ ppm 8.6 (s, 1H), 7.53 (d, 1H), 7.33 (d, 1H), 7.2 (dd, 1H), 4.1 (s, 3H), 2.72 (q, 2H), 1.17(t, 3H)

¹³C-NMR (125 MHz, dmso-d6) δ ppm 162.5, 161.7, 161, 158.7, 150.8, 143.4, 130.8, 130.6, 123.3, 121.5, 56.7, 26.6, 14.1

### Preparation R2at: 4-(3-benzyloxyphenoxy)-6-methoxy-5-nitro-pyrimidine

Using General Procedure 1 starting from Preparation R1a and 3-benzyloxyphenol as reagents. Preparation R2at was obtained. HRMS calculated for C₁₈H₁₅N₃O₅: 353.1012; found 354.1084 (M+H⁺ form).

¹H-NMR (500 MHz, dmso-d6) δ ppm 8.6 (s, 1H), 7.48-7.3 (m, 5H), 7.37 (t, 1H), 6.99 (m, 1H), 6.98 (dm, 1H), 6.85 (dm, 1H), 5.1 (s, 2H), 4.1 (s, 3H),

¹³C-NMR (125 MHz, dmso-d6) δ ppm 158.7, 130.1, 114.2, 113.5, 108.8, 70, 56.6.

### Preparation R2au: 4-(6-methoxy-5-nitro-pyrimidin-4-yl)oxybenzaldehyde

Using General Procedure 1 starting from Preparation R1a and 4-hydroxybenzaldehyde as reagents. Preparation R2au was obtained. HRMS calculated for C₁₂H₉N₃O₅: 275.0542; found 276.0612 ((M+H)⁺ form).

¹H-NMR (500 MHz, dmso-d6) δ ppm 10.03 (s, 1H), 8.63 (s, 1H), 14.03 (dm, 2H), 7.53 (dm, 2H), 4.12 (s, 3H),

¹³C-NMR (125 MHz dmso-d6) δ ppm 192.4, 162.6, 160.7, 158.7, 156.4, 134.7, 131.9, 122.9, 121.2, 56.7.

### Preparation R2av: tert-butyl N-[(1R)-2,2,2-trifluoro-1-[4-(6-methoxy-5-nitro-pyrimidin-4-yl)oxyphenyl]ethyl]carbamate

4-[( 1R)-1-amino-2,2,2-trifluoro-ethyl]phenol, hydrochloride (850 mg, 3.7345 mmol), tert-butoxycarbonyl tert-butyl carbonate (1.5 eq., 5.6017 mmol), and sodium hydrogencarbonate (2.0 eq., 7.4689 mmol) were dissolved in THF (10 mL) and water (10 mL). The reaction mixture was stirred at r.t. for 20 hours. The reaction mixture was extracted with EtOAc (3×10 ml). The organic layer was evaporated after drying with brine and MgSO₄ to give tert-butyl N-[(1R)-2,2,2-trifluoro-1-(4-hydroxyphenyl)ethyl]carbamate as a crude product.

Using General Procedure 1 starting from Preparation R1a and tert-butyl N-[(1R)-2,2,2-trifluoro-1-(4-hydroxyphenyl)ethyl]carbamate as reagents. Preparation R2av was obtained. HRMS calculated for C₁₉H₁₉F₃N₄O₆: 444.1257; found 462.1587 ((M+NH₄)⁺ form).

¹H-NMR 1400 MHz, dmso-d6) δ ppm 8.91/8.6 (s, 1H, 8.42/8.4 (d, 1H), 7.7/7.62 (dm, 2H), 7.35/7.24 (dm, 2H), 5.51 (m, 1H), 4.12/4.11 (s, 3H), 1.41/1.4 (s, 9H)

¹³C-NMR (125 MHz, dmso-d6) δ ppm 162.5, 161, 159.1/158.6, 152.2, 132.1, 130.6/130.3, 122.2/122.1, 79.9, 57.1/56.7, 55, 28.5

### Preparation R2aw: tert-butyl N-[(1S-2,2,2-trifluoro-1-[4-(6-methoxy-5-nitro-pyrimidin-4-yl)oxyphenyl]ethyl]carbamate

4-[(1S)-1-1-amino-2,2,2-trifluoro-ethyl]phenol, hydrochloride (960 mg, 4.2177 mmol), tert-butoxycarbonyl tert-butyl carbonate (1.5 eq., 6.3266 mmol) and sodium hydrogencarbonate (2.0 eq., 8.4355 mmol) were dissolved in THF (10 mL), and water (10 mL). The reaction mixture was stirred at r.t for 20 hours. The reaction mixture was extracted with EtOAc (3×10 ml). The organic layer was evaporated after drying with brine and MgSO₄ to give tert-butyl N-[(1S)-2,2,2-trifluoro-1-(4-hydroxyphenyl) ethyl]carbamate.

Using General Procedure 1 starting from Preparation R1a and tert-butyl N-[(1S)-2,2,2-trifluoro-1-(4-hydroxyphenyl)ethyl]carbamate as reagents. Preparation R2aw was obtained. HRMS calculated for C₁₈H₁₉F₃N₄O₆: 444.1257; found 389.0703 ((M+H-C₄H₈)⁺ form).

### Preparation tert-butyl 2-[3-(6-methoxy-5-nitro-pyrimidin-4-yl)oxyphenyl]piperidine-1-carboxylate

3-(2-piperidyl)phenol, tert-butoxycarbonyl tert-butyl carbonate (1.5 eq.) and sodium hydrogencarbonate (4.0 eq.) were dissolved in THF (10 mL), and water (10 mL). The reaction mixture was stirred at r.t. for 20 hours. The reaction mixture was extracted with EtOAc (3×10 ml). The organic layer was evaporated after drying with brine and MgSO₄ to give tert-butyl 2-(3-hydroxyphenyl)pipendine-1-carboxylate.

Using General Procedure 1 starting from Preparation R1a and tert-butyl 2-(3-hydroxyphenyl)piperidine-1-carboxylate as reagents. Preparation R2az was obtained. HRMS calculated for C₂₁H₂₆N₄O₆⁻. 430.1852; found 453.1741 ((M+Na)⁺ form)

¹H-NMR (500 MHz, dmso-d6) δ ppm 8.58 (s, 1H), 7.47 (t, 1H), 7.16 (dm, 1H), 7.13 (dm, 1H), 7.05 (brs, 1H), 5.28 (br., 1H), 4.1 (s, 3H), 3.92/2.7 (d+td, 2H), 2.28/1.76 (d+tm, 2H), 1.54/1.38 (d+m, 2H), 1.54/1.24 (d+m, 2H), 1.37 (s, 9H),

¹³C-NMR (125 MHz, dmso-d6) δ ppm 162.4, 161.1, 158.6, 155.1, 152.4, 143.3, 130.5, 124.6, 120.1, 119.9, 79.5, 56.6, 53.2, 40.3, 28.5, 28.3, 25.2, 19.4

### Preparation R2bc: 5-(6-methoxy-5-nitro-pyrimidin-4-yl)oxyindan-2-ol

Using General Procedure 1 starting from Preparation R1a and indane-2,5-diol as reagents. Preparation R2bc was obtained. HRMS calculated for C₁₄H₁₃N₃O₅: 303.0855; found 304.0927 (M+H)⁺ form),

¹H-NMR (500 MHz, dmso-d6) δ ppm 8.57 (s, 1H), 7.26 (d, 1H), 7.09 (d, 1H), 6.98 (dd, 1H), 4.91 (d, 1H), 4.53 (m, 1H), 4.09 (s, 3.06/2.74 (dt+dt, 2H), 3.06/2.74 (dt+dt, 2H), ¹³C-NMR (125 MHz, dmso-d6) δ ppm 162.3, 161.4, 158.6, 150.7, 144.1, 140.3, 126, 120.9, 119.7, 118.4, 72, 56.6, 42.7, 42

### Preparation R2bf: 2-[3-(6-methoxy-5-nitro-pyrimidin-4-yl)oxyphenyl]propan-2-ol

Using General Procedure 1 starting from Preparation R1a and 3-(1-hydroxy-1-methyl-ethyl)phenol as reagents. Preparation R2bf was obtained. HRMS calculated for C₁₄H₁₅N₃O₅: 305.1012; found 306.1083 ((M+H)⁻ form).

¹H-NMR (500 MHz, dmso-d6) δ ppm 8.59 (s, 1H), 7.42-7.07 (m, 4H), 5.13 (br., 1H), 4.1 (s. 3H), 1.42 (s, 6H),

¹³C-NMR (125 MHz, dmso-d6) δ ppm 162.4, 161.2, 158.7, 153.6, 151.7, 129.6/123.1/119.6/118, 71, 56.6, 32.3.

### Preparation R2 bg: 2-fluoro-4-(6-methoxy-5-nitro-pyrimidin-4-yl)oxy-benzonitrile

Using General Procedure 1 starting from Preparation R1a and 2-fluoro-4-hydroxy-benzonitrile as reagents. Preparation R2bg was obtained. HRMS calculated for C₁₂H₇FN₄O₄: 290.0451; found 291.0522 ((M+H)⁺ form).

¹H-NMR (500 MHz, dmso-d6) δ ppm 8.66 (s, 1H), 8.09 (dd, 1H), 7.74 (dd, 1H), 7.44 (dm, 1H), 4.13 (s, 3H),

¹³C-NMR (125 MHz, dmso-d6) δ ppm 163.5, 162.7, 160.2, 158.8, 156.8, 135.6, 119.8, 114, 111.6, 98.7, 56.9.

### Preparation R2 bb: 2-chloro-4-(6-methoxy-5-nitro-pyrimidin-4-yl)oxy-benzonitrile

Using General Procedure 1 starting from Preparation R1a and 2-chloro-4-hydroxy-benzonitrile as reagents. Preparation R2bh was obtained. HRMS calculated for C₁₂H₇ClN₄O₄: 306.0156; found 307.0226 ((M+H)⁺ form).

¹H-NMR (500 MHz, dmso-d6) δ ppm 8.66 (s, 1H), 8.13 (d, 1H), 7.92 (d, 1H), 7.56 (dd, 1H), 4.12 (s, 3H),

¹³C-NMR (125 MHz, dmso-d6) δ ppm 162.7, 160.3, 158.8, 155.8, 137.1, 136.6, 124.3, 122.3, 116, 110.5, 56.8.

### Preparation R2bi: 2-[4-(6-methoxy-5-nitro-pyrimidin-4-yl)oxyphenyl]acetonitrile

Using General Procedure 1 starting from Preparation R1a and 2-(4-hydroxyphenyl)acetonitrile as reagents. Preparation R2bi was obtained. HRMS calculated for C₁₃H₁₀N₄O₄: 286.0702; found 287.077 ((M+H)⁺ form).

¹H-NMR (500 MHz, dmso-d6) δ ppm 8.58 (s, 1H), 7.45 (m, 214), 7.31 (m, 2H), 4.1 (s, 2H), 4.09 (s, 3H),

¹³C-NMR (125 MHz, dmso-d6) δ ppm 162.4, 161.1, 158.6, 151.3, 1301.2, 130.1, 122,7, 119.6, 56.6, 22.3

### Preparation R2bj: 3-[4-(6-methoxy-5-nitro-pyrimidin-4-yl)oxyphenyl]propanenitrile

Using General Procedure 1 starting from Preparation R1a and 3-(4-hydroxyphenyl)propanenitrile as reagents. Preparation R2bj was obtained. HRMS calculated for C₁₄H₁₂N₄O₄: 300.0858; found 300.08627 (M⁺ form).

¹H-NMR (500 MHz, dmso-d6) δ ppm 8.59 (s, 1H), 7.39 (m, 2H), 7.23 (m, 2H), 4.1 (s, 3H), 2.92 (m, 2H), 2.84 (m 2H),

¹³C-NMR (125 MHz, dmso-d6) δ ppm 158.6, 130.3, 122, 120.7, 56.6, 30.3, 18.6.

### Preparation R2bk: 3-chloro-4-(6-methoxy-5-nitro-pyrimidin-4-yl)oxy-benzonitrile

Using General Procedure 1 starting from Preparation R1a and 3-chloro-4-hydroxy-benzonitrile as reagents. Preparation R2bk was obtained. HRMS calculated for C₁₂H₇ClN₄O₄: 306.0156; found 307.0227 ((M+H)⁺ form).

¹H-NMR (500 MHz, dmso-d6) δ ppm 8.63 (s, 1H), 8.33 (d, 1H), 8 (dd, 1H), 7.76 (d, 1H), 4.13 (s, 3H),

¹³C-NMR (125 MHz, dmso-d6) δ ppm 162.7, 159.8, 158.8, 151.3, 135, 133.7, 127.6, 126, 117.5, 111.6, 57.

### Preparation R2bm: tert-butyl N-[1-[4-(6methoxy-5-nitro-pyrimidin-4-yl)oxyphenyl]ethyl]carbamate

Using General Procedure 1 starting from Preparation R1a and tert-butyl N-[1-(4-hydroxyphenyl)ethyl]carbamate as reagents. Preparation R2bm was obtained. HRMS calculated for C₁₈H₂₂N₄O₆: 390.1539; found 408.1877 ((M+NH₄)⁺ form).

¹H-NMR (500 MHz, dmso-d6) δ ppm 8.58 (s, 1H), 7.44 (d, 1H), 7.37 (m, 2H), 7.2 (m, 2H), 4.65 (m, 1H), 4.1 (s, 3H), 1.37 (s, 9H), 1.31 (d, 3H),

¹³C-NMR (125 MHz, dmso-d6) δ ppm 158.6, 127.6, 121.7, 56.6, 49.5, 28.7, 23.3.

### Preparation R2bn: tert-butyl N-[1-[4-(6-methoxy-5-nitro-pyrimidin-4-yl)oxyphenyl]propyl]carbamate

4-(1-Aminopropyl)phenol, hydrochloride (1:1) (1 g, 5.3286 mmol) tert-butoxycarbonyl tert-butyl carbonate (1.5 eq., 7.9929 mmol) and sodium hydrogen carbonate (3.0 eq., 15.9858 mmol) were dissolved in THF (10 mL), and water (10 mL). The reaction mixture was stirred at r.t. for 20 hours. The reaction mixture was extracted with EtOAc (3×10 ml). The organic layer was evaporated ager drying with brine and MgSO₄ to give tert-butyl N-[1-[4-(6-methoxy-5-nitro-pyrimidin-4-yl)oxyphenyl]propyl]carbamate.

Using General Procedure 1 starting from Preparation R1a and tert-butyl N-[1-[4-(6-methoxy-5-nitro-pyrimidin-4-yl)oxyphenyl]propyl]carbamate as reagents. Preparation R2bn was obtained. HRMS calculated for C₂₉H₂₄N₄O₆: 404.1696; found 422.2022 ((M+NH₄)⁺ form).

¹H-NMR (500 MHz, dmso-d6) δ ppm 8.59 (s, 1H), 7.4 (d, 1H), 7.35 (m, 2H), 7.2 (m, 2H), 4.39 (m, 1H), 4.1 (s, 3H), 1.66/1.61 (m+m, 2H), 1.37 (s, 3H), 0.83 (t, 3H),

¹³C-NMR (125 MHz, dmso-d6) δ ppm 158.7, 128.2, 121.7, 56.6, 55.8, 29.9, 28.7, 11.6.

### Preparation R2bq: 4-methoxy-5-nitro-6-(3-pyridyloxy)pyrimidine

Using General Procedure 1starting from Preparation R1a and pyridin-3-ol as reagents. Preparation R2bq was obtained. HRMS calculated for C₁₀H₈N₄O₄: 248.0546; found 249.0615 ((M+H)⁺ form)

¹H-NMR (500 MHz, dmso-d6) δ ppm 8.61 (s, 1H), 8.58 (d, 1H), 8.54 (dd, 1H), 7.82 (ddd, 1H), 7.55 (dd, 1H), 4.12 (s, 3H)

¹³C-NMR (125 MHz, dmso-d6) δ ppm 162.5, 160.9, 158,7, 148.8, 147.9, 143.8, 130.2, 125.1, 120.9, 56.7

### Preparation R2bs: 6(6-methoxy-5-nitro-pyrimidin-1H-pyrrolo[3,2-b]pyridine

Using General Procedure 1 starting from Preparation R1a and 1H-pyrrolo[3,2-b]pyridin-6-ol as reagents. Preparation R2bs was obtained. HRMS calculated for C₁₂H₉N₅O₁: 287.0655; found 288.0733 ((M+H)⁺ form).

¹H-NMR (500 MHz, dmso-d6) δ ppm 11.49 (br., 1H), 8.57 (s, 8.25 (d, 1H), 7.77 (dd, 1H), 7.71 (dd, 1H), 6.61 (m, 1H), 4.11 (s, 3H)

¹³C-NMR (125 MHz, dmso-d6) δ ppm 162.4, 161.7, 158.6, 145/143.6/128.3, 136.9, 131.2 120.9, 112.4, 102.2, 56.6

### Preparation R2bt: 4-(3-benzyloxy-4-chloro-phenoxy)-6-methoxy-5-nitro-pyrimidine

Using General Procedure 1 starting from Preparation R1a and 3-benzyloxy-4-chloro-phenol as reagents. Preparation R2bt was obtained. HRMS calculated for C₁₈H₁₄ClN₃O₅: 387.0622; found 388.0696 ((M+H)⁺ form).

¹H-NMR (500 MHz, dmso-d6) δ ppm 8.6 (s, 1H), 7.49-7.31 (m, 7.53 (d, 1H), 7.3 (d, 1H), 6.92 (dd, 1H), 5.18 (s, 2H), 4.11 (s, 3H)

¹³C-NMR (125 MHz, dmso-d6) δ ppm 158.7, 130.8, 115.2, 108.9, 70.8, 56.7

### Preparation R2bu: 4-(3-benzyloxy-4-methyl-phenoxy)-6-methoxy-5-nitro-pyrimidine

Using General Procedure 1 starting from Preparation R1a and 3-benzyloxy-4-methyl-phenol as reagents. Preparation R2bu was obtained. HRMS calculated for C₁₉H₁₇N₃O₅: 367.1168; found 368.1237 ((M+H)⁺ form).

¹H-NMR (500 MHz, dmso-d6) δ ppm 8.57 (s, 1H), 7.48-7.30 (m, 5H), 7.22 (dm, 1H), 7.01 (d, 1H), 6.75 (dd, 1H), 5.08 (s, 2H),4.1 (s, 3H), 2.2 (brs, 3H)

¹³C-NMR (125 MHz, dmso-d6) δ ppm 158.6, 131.1, 113.5, 106.3, 69.9, 56.6, 16.2

### Preparation R2bv: tert-butyl 4-[2-[4-(6-methoxy-5-nitro-pyrimidin-1-yl)oxyphenyl]ethyl]piperidine-1-carboxylate

4-[2-(4-piperidyl)ethyl]phenol (1 g, 4.871 mmol), tert-butoxycarbonyl tert-butyl carbonate (1.5 eq., 7.306 mmol) and sodium hydrogencarbonate (4.0 eq., 19.48 mmol) were dissolved in THF (10 mL) and water (10 mL). The reaction mixture was stirred at r.t. for 20 hours. Then water was added (30 ml) and it was extracted with EtOAc (3×30 ml). The organic layer was evaporated after drying with MgSO₄. The residue was washed with diisopropyl ether and the solid compound was filtered off to give tert-butyl 4-[2-(4-hydroxyphenyl)ethyl]piperidine-1-carboxylate as a crude product.

Using General Procedure 1 starting from Preparation R1a and tert-butyl 4-[2-(4-hydroxyphenyl)ethyl]piperidine-1-carboxylate as reagents. Preparation R2bv was obtained. HRMS calculated for C₂₄H₃₀N₄O₆: 458.2165; found 403.1608 ((M+H-C₄H₈)⁻ form).

¹H-NMR (500 MHz, dmso-d6) δ ppm 8.58 (s, 1H), 7.28 (m, 2H), 7.15 (m, 2H), 4.1 (s, 3H), 3.92/2.67 (m+m, 4H), 2.62 (m, 2H), 1.49/1.01 (m+m, 4H), 1.51 (m, 2H), 1.4 (m, 1H), 1.38 (s, 3H)

¹³C-NMR (125 MHz, dmso-d6) δ ppm 158.7, 130, 121.8, 56.6, 44, 38.6, 38.3, 35.3, 32.1, 32

### Preparation R2bw: 4-[[4-[(6-methoxy-5-nitro-1,4-dihydropyrimidin-4-yl)oxy]phenyl]methyl]morpholine

Using General Procedure 1 starting from Preparation R1a and 4-(morpholinomethyl)phenol as reagents. Preparation R2bw was obtained. HRMS calculated for C₁₆H₁₈N₄O₅: 346.1277; found 347.1351 ((M+H)⁺ form).

¹H-NMR (500 MHz, dmso-d6) δ ppm 8.59 (s, 1H), 7.39 (dm, 2H), 7.21 (dm, 2H), 4.1 (s, 3H), 3.58 (t, 4H), 3.48 (s, 2H), 2.36 (br., 4H)

¹³C-NMR (125 MHz, dmso-d6) δ ppm 162.4, 161.1, 158.7, 150.9, 136.5, 130.6, 121.7, 66.7, 61.2, 56.6, 53.6

### Preparation R2bx: tert-butyl 2-[2-[4-(6-methoxy-5-nitro-pyrimidin-4-yl)oxyphenyl]ethyl]piperidine-1-carboxylate

4-[2-(2-piperidyl)ethyl]phenol (1 g, 4.871 mmol), tert-butoxycarbonyl tert-butyl carbonate (1.5 eq., 7.306 mmol) and sodium hydrogencarbonate (4.0 eq., 19.48 mmol) were dissolved in THF (10 mL) and water (10 mL). The reaction mixture was stirred at r.t. for 20 hours. Then water was added (30 ml) and it was extracted with EtOAc (3×30 ml). The organic layer was evaporated after drying with MgSO₄. The residue was crystalized from the mixture of diisopropyl ether pentane, and the solid compound was filtered off to give tert-butyl 2-[2-(4-hydroxyphenyl)ethyl]piperidine-1-carboxylate as a crude product.

Using General Procedure 1 gaping front Preparation R1a and tert-butyl 2-[2-(4-hydroxyphenyl)ethyl]piperidine-1-carboxylate as reagents. Preparation R2bx was obtained. HRMS calculated for C₂₃H₃₀N₄O₆: 458.2165; found 481.20517 ((M+Na)⁺ form).

¹H-NMR (500 MHz, dmso-d6) δ ppm 8.57 (s, 1H), 7.3 (dm, 2H), 7.16 (dm, 2H), 4.16 (br., 1H), 4.1 (s, 3H), 3.85/2.81 (br.+br., 2H), 2.57/2.46 (m+m, 2H), 1.95/1.73 (m+m, 2H), 1.58/1.48 (m+m, 2H), 1.57/1.26 (m+m, 2H), 1.56/1.51 (m+m, 2H), 137 (s, 9H),

¹³C-NMR (125 MHz, dmso-d6) δ ppm 162.4, 161.2, 158.6, 156, 140.5, 130, 121.8, 78.8, 56.6, 50.2, 38.9, 31.9, 31.4, 28.6, 28.4, 15.6, 19

### Preparation R2bv: tert-butyl 2-[3-(6-methoxy-5-nitro-pyrimidin-4-yl)oxyphenyl]morpholine-4-carboxylate

3-morpholin-2-ylphenol (1 g, 5.579 mmol), tert-butoxycarbonyl tert-butyl carbonate (1.5 eq., 8.369 mmol) and sodium hydrogencarbonate (4.0 eq., 22.319 mmol) were dissolved in THF (10 mL) and water (10 mL). The reaction mixture was stirred at r.t. for 20 hours. Then water was added (30 ml) and it was extracted with EtOAc (3×30 ml). The organic layer was evaporated after drying with MgSO₄ to give tert-butyl 2-(3-hydroxyphenyl)morpholine-4-carboxylate as a crude product.

Using General Procedure 1 starting front Preparation R1a and tert-butyl 2-(3-hydroxyphenyl)morpholine-4-carboxylate as reagents. Preparation R2by was obtained. HRMS calculated for C₂₀H₂₄N₄O₇: 432.1645; found 455.153 ((M+Na)⁺ form).

¹H-NMR (400 MHz, dmso-d6) δ ppm 8.6 (s, 1H), 7.47 (t, 1H), 7.34 dm, 1H), 7.28 (t, 1H), 7.23 (dm, 1H), 4.45 (dd, 1H), 4.1 (s, 3H), 3.94/3.55 (m+m, 2H), 3.91/2.78 (brm, 2H), 3.78/2.97 (brm, 2H), 1.41 (s, 3H),

¹³C-NMR (100 MHz, dmso-d6) δ ppm 158.7, 130.3, 124.7, 121.6, 119.8, 76.5, 66.4, 56.6, 49.7, 43.2, 28.5

### Preparation R2bz: tert-butyl N-[(1R)-2,2,2-trifluoro-1-[4-(6-methoxy-5-nitro-pyrimidin-4-yl)oxyphenyl]ethyl]carbamate

4-[(1R)-1-amino-2,2,2-trifluoro-ethyl]phenol (850 mg, 3.734 mmol), tert-butoxycarbonyl tert-butyl carbonate (1.5 eq., 5.601 mmol) and sodium hydrogencarbonate (2.0 eq., 7.468 mmol) were dissolved in THF (10 mL) and water (10 mL). The reaction mixture was stirred at r.t. for 18 hours. It was extracted with EtOAc (3×10 ml). The organic layer was evaporated after drying with MgSO₄ to give tert-butyl N-[(1R)-2,2,2-trifluoro-1-(4-hydroxyphenyl)ethyl]carbamate as a crude product.

Using General Procedure 1 starting from Preparation R1a and tert-butyl N-[(1R)-2,2,2-trifluoro-1-(4-hydroxyphenyl)ethyl]carbamate as reagents. Preparation R2bz was obtained. HRMS calculated for C₁₈H₁₉F₃N₄O₆: 444.1257; found 462.1587 ((M+NH₄)⁺ form).

¹H-NMR (400 MHz, dmso-d6) δ ppm 8.91/8.6 (s, 1H), 8.42/8.4 (d, 1H), 7.7/7.62 (dm, 2H), 7.35/7.24 (dm, 2H), 5.51 (m, 1H), 4.12/4.11 (s, 3H), 1.41/1.4 (s, 9H)

¹³C-NMR (100 MHz, dmso-d6) δ ppm 162.5, 161, 159.1/158.6, 152.2, 132.1, 130.6/130.3, 122.2/122.1, 79.9, 57.1/56.7, 55, 28.5

### Preparation R2ca: 2-[4-(6-methoxy-5-nitro-pyrimidin-4-yl)oxyphenyl]acetonitrile

Using General Procedure 1 starting from Preparation R1a and 2-(4-hydroxyphenyl)acetonitrile as reagents. Preparation R2ca was obtained. HRMS calculated for C₁₃H₁₀N₄O₄: 286.0702; found 287.0770 ((M+H)⁺ form)

¹H-NMR (500 MHz, dmso-d6) δ ppm 8.58 (s, 1H), 7.45 (m, 2H), 7.31 (m, 2H), 4.1 (s, 2H), 4.09 (s, 3H)

¹³C-NMR (125 MHz, dmso-d6) δ ppm 162.4, 161.1, 158.6, 151.3, 130.2, 130.1, 122.7, 119.6, 56.6, 22.3

### Preparation R2cb: [2-fluoro-5-(6-methoxy-5-nitro-pyrimidin-4-yl)oxy-phenyl]methanol

Using General Procedure 1 starting from Preparation R1a and 4-fluoro-3-(hydroxymethyl)phenol as reagents. Preparation R2cb was obtained. HRMS calculated for C₁₂H₁₀FN₃O₅: 295.0605; found 296.06726 ((M+H)³⁰ form).

¹H-NMR (500 MHz, dmso-d6) δ ppm 8.59 (s, 1H), 7.32 (dd, 1H), 7.26 (t, 1H), 7.21 (ddd, 1H), 5.4 (t, 1H), 4.56 (d, 2H), 4.1 (s, 3H)

¹³C-NMR (125 MHz, dmso-d6) δ ppm 162.4, 161.2, 138.6, 157.5, 147.9, 131.5, 122.1, 122, 120.9, 116.5, 56.9, 56.6

### Preparation R2cc: 4-(4-fluoro-3-nitro-phenoxy)-6-methoxy-5-nitro-pyrimidine

Using General Procedure 1 starting from Preparation R1a and 4-fluoro-3-nitro-phenol as reagents. Preparation R2cc was obtained. HRMS calculated for C₁₁H₇FN₄O₆: 310.035; found 311.0429 ((M+H)⁺ form).

¹H-NMR (500 MHz, dmso-d6) δ ppm 8.63 (s, 1H), 8.25 (dd, 1H), 7.83 ddd, 1H), 7.74 (dd, 1H), 4.12 (s, 3H)

¹³C-NMR (125 MHz, dmso-d6) δ ppm 162.6, 160.8, 158.6, 153.1, 147.3, 137.5, 130.6, 120.9, 120.4, 120.2, 56.8

### Preparation R2cd: 4-(4-isopropenyl-3-methoxy-phenoxy)-6-methoxy-5-nitro-pyrimidine

Using General Procedure 1 starting from Preparation R1a and 4-bromo-3-methoxy-phenol as reagents. 4-(4-bromo-3-methoxy-phenoxy)-6-methoxy-5-nitro-pyrimidine was obtained.

4-(4-bromo-3-methoxy-phenoxy)-6-methoxy-5-nitro-pyrimidine (500 mg, 1.4 mmol), 2-isopropenyl-4,4,5,5-tetramethyl-1,3,2-dioxaborolane (2.8 mmol, 2.0 eq.), potassium carbonate (4.3 mmol. 3.07 eq.), tetrakis(triphenylphosphine)palladium(0) (0.14 mmol, 0.1 eq.) were dissolved in dry toluene. The resulted mixture was heated and stirred at 100° C. till completion. Then ethyl acetate and brine were added. The layers were separated. The organic layer was dried over magnesium sulfate and evaporated. It was purified by Flash chromatography (eluent:heptane:ethyl acetate=3:2) to give Preparation R2cd.

¹H-NMR (500 MHz, dmso-d6) δ ppm 8.61 (s, 1H), 7.21 (d, 1H), 6.96 (d, 1H), 6.81 (dd, 1H), 5.13/5.05 (m+m, 2H), 4.11 (s, 3H), 3.75 (s, 3H), 2.05 (dd, 3H)

¹³C-NMR (125 MHz, dmso-d6) δ ppm 158.7, 129.9, 116.2, 113.6. 105.9, 56.6, 56.3, 23.5

### Preparation R2ce; tert-butyl 2-[4-(6-methoxy-5-nitro-pyrimidin-4-yl)oxyphenyl]piperidine-1-carboxylate

4-(2-piperidyl)phenol hydrochloride(1.2 g, 5.615 mmol), tert-butoxycarbonyl tert-butyl carbonate (1.5 eq., 8.423 mmol) and sodium hydrogencarbonate (4.0 eq., 22.461 mmol) were dissolved in THF (15 mL) and water (15 mL). The reaction mixture was stirred at r.t. for 20 hours. Then water was added (30 ml) and it was extracted with EtOAc (3×30 ml). The organic layer was evaporated alter drying with MgSO₄. The residue was washed with mixture of diisopropyl ether and ethanol and the solid compound was filtered onto give tert-butyl 2-(4-hydroxyphenyl)piperidine-1-carboxylate as a crude product.

Using General Procedure 1 starting from Preparation R1a and tert-butyl 2-(4-hydroxyphenyl)piperidine-1-carboxylate as reagents. Preparation R2ce was obtained. HRMS calculated for C₂₁H₂₆N₄O₆: 430.1852; found 375.1292 ((M+H-C₄H_{R})⁺ form).

¹H-NMR (400 MHz, dmso-d6) δ ppm 8.6 (s, 1H), 7.27 (s, 2H), 7.27 (s, 2H), 5.29 (d, 1H), 4.1 (s, 3H), 3.94/2.72 (m+m, 2H), 2.38-1.18 (m, 6H), 1.4 (s, 9H)

¹³C-NMR (100 MHz, dmso-d6) δ ppm 158.8, 128.1, 122.2, 56.6, 53, 40.3, 28.6

### Preparation 123a: 6-methoxy-4-phenoxy-1,6-dihydropyrimidin-5-amine

Using General Procedure 2 starting from Preparation R2a as reagent. Preparation R3a was obtained. HRMS calculated for C₁₁H₁₀FN₃O₂: 217.0851; found 218.092 ((M+H)⁻ form).

¹H-NMR (500 dmso-d6) δ ppm 7.75 (s, 1H), 7.4 (m 2H), 7.19 (m, 1H), 7.12 (m, 2H), 4.85 (brs, 2H), 3.95 (s, 3H),

¹³C-NMR (125 MHz, dmso-d6) δ ppm 158, 154.7, 154.1, 142.9, 129.9, 124.9, 121.4, 117.3, 54.4.

### Preparation R3b: 4-(2-fluorophenoxy)-6-methoxy-pyrimidin-5-amine

Using General Procedure 2 starting from Preparation R2b as reagent. Preparation R3b was obtained. HRMS calculated for C₁₁H₁₀FN₃O₂: 235.0757; found 235.07507 (M⁺ form).

¹H-NMR (500 MHz, dmso-d6) δ ppm 7.71 (s, 1H), 7.35 (m, 1H), 7.31 (m, 1H), 7.28 (m, 1H), 7.24 (m, 1H), 4.92 (s, 2H ) 3.95 (s, 3H),

¹³C-NMR (125 MHz, dmso-d6) δ ppm 157.9, 154.5, 142.7, 140.9, 127, 125.5, 124.7, 117.1, 54.5.

### Preparation R3c: 4-methoxy-6-(4-methoxyphenoxy)pyrimidin-5-amine

Using General Procedure 2 starting from Preparation R2c as reagent. Preparation R3c was obtained. HRMS calculated for C₁₂H₁₃N₃O₃: 247.0957; found 248.10318 ((M+H)⁺ form).

¹H-NMR (500 MHz, dmso-d6) δ ppm 7.72 (s, 1H), 7.06 (m, 2H), 6.94 (m, 2H), 4.79 (s, 2H), 3.94 (s, 3H), 3.75 (s, 3H),

¹³C-NMR (125 MHz, dmso-d6) δ ppm 157.7, 156.5, 155.4, 147.3, 142.9, 122.8, 116.7, 114.9.

### Preparation R3d: 4-methoxy-6-(3-methoxyphenoxy)pyrimidin-5-amine

Using General Procedure 2 starting from Preparation R2d as reagent. Preparation R3d was obtained. HRMS calculated for C₁₂H₁₃N₃O₃: 247.0957; found 248.10317 ((M+H)⁺ form).

¹H-NMR (400 MHz, dmso-d6) δ ppm 7.76 (s, 1H), 7.29 (t, 1H), 6.77 (dd, H), 6.71 (t, 1H), 6.69 (dd, 1H), 4.83 (s, 2H), 3.95 (s, 3H), 3.74 (s,

¹³C-NMR (125 MHz, dmso-d6) δ ppm 160.7, 158.1, 155.2, 142.9, 130.3, 113.5, 110.6, 107.4, 55.8, 54.4.

### Preparation R3e: 4-(4-fluorophenoxy)-6-methoxy-pyrimidin-5-amine

Using General Procedure 2 starting from Preparation R2e as reagent. Preparation R3e was obtained. HRMS calculated for C₁₁H₁₀FN₃O₂: 235.0757; found 235.07503 (M⁺ form).

¹H-NMR (500 MHz, dmso-d6) δ ppm 7.74 (s, 1H), 7.23 (m, 2H), 7.18 (m, 2H), 4.86 (s, 2H), 3.95 (s, 3H),

¹³C-NMR (125 MHz, dmso-d6) δ ppm 159.2, 157.9, 154.9, 150.1, 142.9, 123.4, 116.4, 54.4.

### Preparation R3f: 4-(3-fluorophenoxy)-6-methoxy-pyrimidin-5-amine

Using General Procedure 2 starting from Preparation R2f as reagent. Preparation R3f was obtained. HRMS calculated for C₁₁H₁₀FN₃O₂: 235.0757; found 236.0824 ((M+H)⁺ form).

¹H-NMR (500 MHz, dmso-d6) δ ppm 7.78 (s, 1H), 7.43 (m, 1H), 7.08-7.01 (m, 1H), 7.08-7.01 (m, 1H), 6.99 (m, 1H), 4.92 (br., 2H), 3.96 (s, 3H),

¹³C-NMR (125 MHz, dmso-d6δ ppm 162.8, 158.2, 155.3, 154, 142.8, 131.2, 117.7, 116.8, 111.7, 108.5, 54.5.

### Preparation R3g: 4-(3,5-dimethoyphenoxy)-6-methoxy-pyrimidin-5-amine

Using General Procedure 2 starting from Preparation R2g as reagent. Preparation R3g was obtained. HRMS calculated for C₁₃H₁₅N₃O₄: 277.1063; found 278.1141 ((M+H)⁺ form).

¹H-NMR (500 MHz, dmso-d6) δ ppm 7.77 (s, 1H), 6.35 (t, 1H), 6.3 (d, 2H), 4.83 (br., 2H), 3.95 (s, 3H), 3.72 (s, 6H)

¹³C-NMR (125 MHz, dmso-d6) δ ppm 161.3, 58.1, 155.9, 154.4, 142.9, 117.5, 99.9, 97, 55.9, 54.4.

### Preparation R3h: 4-(3,5-difluorophenoxy)-6-methoxy-pyrimidin-5-amine

Using General Procedure 2 starting from Preparation R2h as reagent. Preparation R3h was obtained. HRMS calculated for C₁₁H₉F₂N₃O₂: 253.0663; found 254.0738 ((M+H)⁺ form).

¹H-NMR (400 MHz, dmso-d6) δ ppm 7.81 (s, 1H), 7.09 (m, 1H), 6.98 (m, 2H), 4.98 (br,. 2H), 3.96 (s, 3H),

¹³C-NMR (125 MHz, dmso-d6) δ ppm 163, 158.5, 156.2, 151.2, 142.8, 118.1, 105.2. 100.4, 54.5.

### Preparation R3i: 4-methoxy-N-methyl-6-phenoxy-pyrimidin-5-amine

To the solution (in THF) of 4,6-dichloro-N-methyl-pyrimidin-5-amine freshly prepared sodium phenoxide (1.1 eq.) in THF was added and it was stirred at r.t. for 40 hours. The reaction mixture was purified by Hanbon preparative HPLC (C18 Silica. Gemini NX 5 μm, 5 mM NH₄CO₃-MeCN) using gradient method 5-90%. Solvent was evaporated under reduced pressure to give 4-chloro-N-methyl-6-phenoxy-pyrimidin-5-amine as a crude product. It was dissolved in methanol and sodium methoxide (2.2 eq.) was added. The reaction mixture was heated and stirred at 50° C. for 3 hours. The reaction mixture was filtered and the filtrate was purified by Hanbon preparative HPLC (C18 Silica, Gemini NX 5 μm, 5 mM NH₄HCO₃MeCN) using gradient method 5-90%. Solvent was evaporated under reduced pressure to give Preparation R3i. HRMS calculated for C₁₂H₁₃N₃O₂: 231.1008; found 232.108 ((M+H⁾⁺ form).

¹H-NMR (400 MHz, dmso-d6) δ ppm 7.81 (s, 1H), 7.39 (m, 2H), 7.18 (tm 1H), 7.1 (dm, 2H), 4.8 (q, 1H), 3.95 (s, 3H),2.89 (d, 3H),

¹³C-NMR (125 MHz, dmso-d6) δ ppm 159.8, 155.7, 154.5, 144.3, 130, 124.7, 121, 120, 54.6, 33.1.

### Preparation R3j: 4-(4-chlorophenoxy)-6-methoxy-pyrimidin-5-amine

Using General Procedure 2 starting from Preparation R2j as reagent. Preparation R3j was obtained. HRMS calculated for C₁₁H₁₀ClN₃O₂: 251.0462; found 252.0523 ((M+H)⁺ form).

¹H-NMR (400 MHz, dmso-d6) δ ppm 7.76 (s, 1H), 7.45 (m, 2H), 7.18 (m, 2H), 4.9 (s, 2H), 3.95 (s, 3H).

¹³C-NMR (125 MHz, dmso-d6) δ ppm 158.1, 154.3, 153, 142.8, 129.8, 128.8, 123.3, 117.4, 54.4.

### Preparation R3k: 4-(4-chloro-3-methoxy-phenoxy)-6-methoxy-pyrimidin-5-amine

Using General Procedure 3 starting from Preparation R2k as reagent. Preparation R3k was obtained. HRMS calculated for C₁₂H₁₂ClN₃O₃: 281.0567; found 282.0637 ((M+H)⁺ form).

¹H-NMR (500 MHz, dmso-d₆) δ ppm 7.77 (s, 1H), 7.42 (d, 1H), 6.99 (d, 1H), 6.73 (dd, 1H), 4.89 (s, 2H), 3.95 (s, 3H), 3.82 (s, 3H).

¹³C-NMR (125 MHz, dmso-d6) δ ppm 158.1, 155.6, 154.3, 154, 142.8, 130.3, 117.4, 117, 114.1, 106.9.

### Preparation R3l: 4-(3-chlorophenoxy)-6-methoxy-pyrimidin-5-amine

Using General Procedure 3 starting from Preparation R2l as reagent. Preparation R3l was obtained. HRMS calculated for C₁₁H₁₀ClN₃O₂: 251.0462; found 252.0533 ((M+H)⁺ form).

¹H-NMR (400 MHz, dmso-d6) δ ppm 7.78 (s, 1H), 7.43 (t, 1H), 7.27 (dm, 1H), 7.26 (m, 1H), 7.13 (dm, 1H), 4.93 (brs, 2H), 3.96 (s, 3H)

¹³C-NMR (125 MHz, dmso-d6) δ ppm 158.3, 155.1, 153.9, 142.8, 131.4, 124.9, 121.5, 120.2, 54.6, 7.26

### Preparation R3n: 4-(1,3-benzodioxol-5-yloxy)-6-methoxy-pyrimidin-5-amine

Using General Procedure 2 starting from Preparation R2n as reagent. Preparation R3n was obtained. HRMS calculated for C₁₂H₁₁N₃O₄: 261.075; found 262.0819 ((M+H)⁺ form).

¹H-NMR (500 MHz, dmso-d6) δ ppm 7.74 (s, 1H), 6.9 (d, 1H), 6.8 (d, 1H), 6.39 (dd, 1H), 6.05 (s, 2H), 4.79 (br., 2H), 3.94 (s, 3H)

¹³C-NMR (125 MHz, dmso-d6) δ ppm 157.8, 155.3, 148.3, 148.1, 144.5, 142.9, 116.8, 114.1, 108.4, 104.3, 102, 54.4

Preparation R3o: 3-(5-amino-6-methoxy-pyrimidin-4-yl)oxy-5-methoxy-phenol

Using General Procedure 2 starting from Preparation R2o as reagent. Preparation R3o was obtained. HRMS calculated for C₁₂H₁₃N₃O₄: 263.0906; found 263.09017 (M⁺ form).

¹H-NMR (500 MHz, dmso-d6) δ ppm 7.78 (s, 1H), 6.16 (t, 1H), 6.14 (t, 1H), 6.08 (t, 1H), 4.81 (s, 2H), 3.95 (s, 3H), 3.67 (s, 3H).

¹³C-NMR (125 MHz, dmso-d6) δ ppm 161.3, 159.4, 158.1, 155.9, 154.4, 143, 117.6, 100.9, 98.2, 97.9, 55.6, 54.4.

### Preparation R3p: 4-(4-fluoro-3-methoxy-phenoxy)-6-methoxy-pyrimidin-5-amine

Using General Procedure 2 starting from Preparation R2p as reagent. Preparation R3p was obtained. HRMS calculated for C₁₂H₁₂FN₃O₃: 265.0863; found 266.0931 ((M+H)⁺ form).

¹H-NMR (500 MHz, dmso-d6) δ ppm 7.76 (s, 1H), 7.21 (dd, 1H), 7 (dd, 1H), 6.69 (dm, 1H), 4.85 (s, 2H), 3.95 (s, 3H), 3.8 (s, 3H).

¹³C-NMR (125 MHz, dmso-d6) δ ppm 142.9, 116.2, 113.3. 108.1.

### Preparation R3q: 4-methoxy-6-phenoxy-N-(2,2,2-trifluoroethyl)pyrimidin-5-amine

Preparation R3a and triethylamine (1.5 eq.) were dissolved in abs. THF and 2,2,2-trifluoroethyl-trifluoromethanesulfonate (1.2 eq.) was added. The reaction mixture was heated and stirred at 70° C. for 214 hours. The reaction mixture was purified by Hanbon preparative HPLC (C18 Silica, Gemini NX 5 μm, 5 mM NH₄HCO₃-MeCN) using gradient method 5-90% Solvent was evaporated under reduced pressure to give Preparation R3q. HRMS calculated for C₁₃H₁₂F₃N₃O₂: 299.0882; found 300.0946 ((M+H)⁺ form). ¹H-NMR (500 MHz, dmso-d6) δ ppm 7.91 (s, 1H), 7.42 (tm, 2H), 7.23 (tm, 1H), 7.11 (dm, 2H), 5.5 (t, 1H), 4.06 (m, 2H), 3.99 (s, 3H)

¹³C-NMR (125 MHz, dmso-d6) δ ppm 160, 157.1, 153.6, 146.1, 126.2, 125.3, 121.6, 115.6, 54.8, 45.6

### Preparation R3r: 4-methoxy-6-[3-(trifluoromethoxy)phenoxy]pyrimidin-5-amine

Using General Procedure 2 starting from Preparation R2r as reagent. Preparation R3r was obtained. HRMS calculated for C₁₂H₁₀F₃N₃O₃: 301.0674; found 302.0742 ((M+H)⁺ form).

¹H-NMR (500 ) MHz, dmso-d6) δ ppm 7.79 (s, 1H), 7.53 (m, 1H), 7.23-7.18 (m, 3H), 4.96 (br., 2H), 3.96 (s, 3H).

¹³C-NMR (125 MHz, dmso-d6) δ ppm 158.3, 153.8, 152.2, 149.2, 142.8, 131.3, 120.5, 120.4/117.2/114.4, 117.8, 54.5

### Preparation R3s: 4-methoxy-6(3-methylphenoxy)pyrimidin-5-amine

Using General Procedure 2 starting from Preparation R2s as reagent. Preparation R3s was obtained. HRMS calculated for C₁₃H₁₃N₃O₂: 231.1008; found 232.1083 ((M+H)⁺ form).

¹H-NMR (500 MHz, dmso-d6) δ ppm 7.75 (s, 1H), 7.27 (t, 1H), 7.01 (brd, 1H), 6.93 (br., 1H), 6.91 (dm, 1H), 4.82 (br., 2H), 3.95 (s, 3H), 2.31 (s, 3H).

¹³C-NMR (125 MHz, dmso-d6) δ ppm 158, 154.8, 154.2, 143, 139.6, 129.6, 125.5, 121.8, 118.4, 117.3, 54.4, 21.3.

### Preparation R3t: 4-(3-bromophenyl)-6-methoxy-pyrimidin-5-amine

Using General Procedure 2 starting from Preparation R2t as reagent. Preparation R3t was obtained. HRMS calculated for C₁₁H₁₀BrN₃O₂: 294.9956; found 296.00304 ((M+H)⁺ form).

¹H-NMR (500 MHz, dmso-d6) δ ppm 7.78 (s, 1H), 7.4 (m, 1H), 7.39 (m, 1H), 7.36 (m, 1H), 7.17 (m, 1H), 4.93 (s, 2H), 3.96 (s, 3H).

¹³C-NMR (125 MHz, dmso-d6) δ ppm 158.2, 154, 142.8, 131.7, 127.7, 124.2, 120.5, 117.7.

### Preparation R3u: 4-methoxy-6-[3-(pentafluoro-λ⁶-sulfanyl)phenoxy]pyrimidin-5-amine

Using General Procedure 2 starting from Preparation R2u as reagent. Preparation R3u was obtained. HRMS calculated for C₁₁H₁₀F₅N₃O₂S: 343.0414; found 344.0484 ((M+H)⁺ form).

¹H-NMR (500 MHz, dmso-d6) δ ppm 7.79 (s, 1H), 7.75 (dm, 1H), 7.72 (t, 1H), 7.65 (t, 1H), 7.5 (dm, 1H), 5.01 (s, 2H) 3.97 (s, 3H).

¹³C-NMR (125 MHz, dmso-d6) δ ppm 158.3, 154, 153.7, 153,6, 142.7, 130.8, 125.6, 122.2, 119, 117.8, 54.5.

### Preparation R3v: 4-methoxy-6-[3-(trifluoromethyl)phenoxy]pyrimidin-5-amine

Using General Procedure 2 starting from Preparation R2v as reagent. Preparation R3v was obtained. HRMS calculated for C₁₂H₁₀F₅N₃O₂: 285.0725; found 286.0796 ((M+H)⁺ form).

¹H-NMR (500 MHz, dmso-d6) δ ppm 7.79 (s, 1H), 7.64 (brt, 1H), 7.57 (dm, 1H), 7.52 (m, 1H), 7.48 (dm, 1H), 4.97 (br., 2H), 3.97 (s, 3H).

¹³C-NMR (125 MHz, dmso-d6) δ ppm 158.3, 154.5, 153.9, 142,8, 131.3, 130.7, 125.5, 124.3, 121.5, 118.1, 54.5.

### Preparation R3w: [1-(5-amino-6-methoxy-pyrimidin-4-yl)oxyphenyl]methanol

Using General Procedure 2 starting from Preparation R2w as reagent. Preparation R3w was obtained. HRMS calculated for C₁₂H₁₃N₃O₃: 247.0957; found 247.09514 (M⁺ (GCTOF) form).

¹H-NMR (500 MHz, dmso-d6) δ ppm 7.73 (s, 1H), 7.33 (dm, 2H), 7.07 (dm, 2H), 5.19 (t, 1H), 4.83 (brs, 2H), 4.49 (d, 2H), 3.95 (s, 3H).

¹C-NMR (125 MHz, dmso-d6) δ ppm 157.9, 155, 152.8, 142.9 142.9, 128, 121.2, 117.1, 62.9, 54.4.

### Preparation R3z: 4-(5-amino-6-methoxy-pyrimidin-4-yl)oxybenzonitrile

Using General Procedure 3 starting from Preparation R2z as reagent. Preparation R3z was obtained. HRMS calculated for C₁₂H₁₀N₄O₂: 242.0804; found 243.088 ((M+H)⁺ form).

¹H-NMR (500 MHz, dmso-d6) δ ppm 7.88 (dm, 2H), 7.81 (s, 1H), 7.32 (dm, 2H), 5.04 (br., 2H), 3.97 (s, 3H).

¹³C-NMR (125 MHz, dmso-d6) δ ppm 158.6, 158.1, 152.9, 142.8, 114.5, 121.6, 119.1, 118.5, 107, 54.6.

### Preparation R3aa: 3-(5-amino-6-methoxy-pyrimidin-4-yl)oxybenzonitrile

Using General Procedure 3 starting from Preparation R2aa as reagent. Preparation R3aa was obtained. HRMS calculated for C₁₂H₁₀N₄O₂: 242.0804; found 243.0874 ((M+H)⁺ form)

¹H-NMR (500 MHz, dmso-d6) δ ppm 7.78 (s, 1H), 7.71 (m, 1H), 7.68 (dm, 1H), 7.61 (t, 1H), 7.51 (dm, 1H), 4.98 (s, 2H), 3.96 (s, 3H).

¹³C-NMR (125 MHz, dmso-d6) δ ppm 142.7, 131.4, 128.8, 126.7, 124.9, 118.6, 54.5.

### Preparation R3ab: tert-butyl 7-(5-amino-6-methoxy-pyrimidin-4yl)oxy-3,4-dihydro-1H-isoquinoline-2-carboxylate

Using General Procedure 2 starting from Preparation R2ab as reagent, Preparation R3ab was obtained. HRMS calculated for C₁₉H₂₄N₄O₄: 372.1797; found 373.1876 ((M+H)⁺ form).

¹H-NMR (500 MHz, dmso-d6) δ ppm 7.7.4(s, 1H), 7.17 (d, 1H), 6.96 (d, 6.93 (dd, 1H), 4.81 (s, 2H), 4.48 (s, 2H), 3.95 (s, 3H), 3.56 (t, 2H), 2.76 (t, 2H), 1.42 (s, 9H).

¹³C-NMR (125 MHz, dmso-d6) δ ppm 142.9, 130, 119.8, 119.1, 54.4, 45.5, 41.7, 28.6, 28.1

### Preparation R3ac: methyl 3-(5-amino-6-methoxy-pyrimidin-4-yl)oxybenzoate

Using General Procedure 2 starling from Preparation R2ac as reagent. Preparation R3ac was obtained. HRMS calculated for C₁₃H₁₃N₃O₄: 275.0906; found 276.0977 ((M+H)⁺ form).

¹H-NMR (400 MHz, dmso-d6) δ ppm 7.79 (dm, 1H), 7.78 (s, 1H), 7.64 (m, 1H), 7.56 (t, 1H), 7.47 (dm, 1H), 4.95 (s, 2H), 3.97 (s, 3H), 3.85 (s, 3H).

¹³C-NMR (125 MHz, dmso-d6) δ ppm 141.8, 130.5, 126.3, 125.5, 121.6, 54.5, 52.8.

### Preparation R3ad: [3-(5-amino-6-methoxy-pyrimidin-4-yl)oxyphenyl]methanol

Using General Procedure 2 starting from Preparation R2ad as reagent, Preparation R3ad was obtained. HRMS calculated for C₁₂H₁₃N₃O₃: 247.0957; found 248.1034 ((M+H)⁺ form).

¹H-NMR (400 MHz, dmso-d6) δ ppm 7.75 (s, 1H), 734 (t, 1H), 7.13 (dm, 1H), 7.05 (m, 1H), 6.98 (dm, 1H), 5.26 (brt, 1H), 4.84 (brs, 2H), 4.5 (d, 2H), 3.95 (s, 3H).

¹³C-NMR (125 MHz, dmso-d6) δ ppm 143, 129.5, 122.7, 119.6, 119, 62.9, 54.4.

### Preparation R3ae: 3-[4-(5-amino-6-methoxy-pyrimidin-4-yl)oxyphenyl]propan-1-ol

Using General Procedure 2 starting from Preparation R2ae as reagent. Preparation R3ae was obtained. HRMS calculated for C₁₄H₁₇N₃O₃: 275.127; found 276.1348 (M+H)⁺ form).

¹H-NMR (400 MHz, dmso-d6) δ ppm 7.75 (s, 1H), 7.21 (dm, 2H), 7.03 (tm, 2H), 4.83 (brs, 2H), 4.49 (t, 1H), 3.95 (s, 3H), 3.43 (q, 2H), 2.62 (t, 2H), 1.73 (quin, 2H).

¹³C-NMR (125 MHz, dmso-d6) δ ppm 157.9, 155, 152, 142.9, 138,7, 129.7, 121.3, 117.1, 60.5, 54.4, 34.8, 31.5.

### Preparation R3af; 4-methoxy-6-phenylsulfanyl-pyrimidin-5-amine

Using General Procedure 2 starting from Preparation R2af as reagent. Preparation R3af was obtained. HRMS calculated for C₁₁H₁₁N₃OS: 233.0623; found 234.0703 ((M+H)⁺ form).

¹H-NMR (500 MHz, dmso-d6) δ ppm 7.94 (s, 1H), 7.38 (m, 4H), 7.34 (m, 1H), 5.13 (brs, 2H) 3.94 (s, 3H).

¹³C-NMR (125 MHz, dmso-d6) δ ppm 156.8, 145.1, 142.9, 132.6/129.7, 131.5, 129.5, 128.3, 54.4.

### Preparation R3ag: tert-butyl 6-(5-amino-6-methoxy-pyrimidin-4-yl)oxy-3,4-dihydro-1H-isoquinoline-2-carboxylate

Using General Procedure 2 starting from Preparation R2ag as reagent, Preparation R3ag was obtained. HRMS calculated for C₁₉H₂₄N₄O₄: 372.1797; found 373.1868 ((M+H)⁺ form).

¹H-NMR (400 MHz, dmso-d6) δ ppm 7.73 (s, 1H), 7.18 (d, 1H), 6.94 (dd, 1H), 6.93 (d, 1H), 4.82 (s, 2H), 4.49 (brs, 2H), 3.95 (s, 3H), 3.54 (t, 2H), 2.76 (t, 2H), 1.43 (s, 9H).

¹³C-NMR (125 MHz, dmso-d6) δ ppm 142.9, 127.7, 121.3, 119.6, 54.4, 45.2, 41.2, 28.7, 28.6.

### Preparation R3ah: tert-butyl 5-(5-amino-6-methoxy-pyrimidin-4-yl)oxyisoindoline-2-carboxylate

Using General Procedure 2 starting from Preparation R2ah as reagent, Preparation R3ah was obtained. HRMS calculated for C₁₈H₂₂N₄O₄: 358.1641; found 359.1713 ((M+H)⁺ form).

¹H-NMR (500 MHz, dmso-d6) δ ppm 7.74/7.73 (s/s, 7.33/7.32 ((d/d, 1H), 7.11 (d, 1H), 7.04/7.02 (dd/dd, 1H), 4.85 (s, 2H), 4.59/4.56 (brs+brs, 4H), 3.95 (s, 3H), 1.46/1.45 (s/s, 9H).

¹³C-NMR (125 MHz, dmso-d6) δ ppm 158, 154.9, 142.9, 139/138.1, 133.6/133.1, 124.1, 120.9, 116.2/116.1, 54.4, 52.3/52.2/51.9/51.8, 28.6.

### Preparation R3ai: 2-[4-(5-amino-6-methoxy-pyrimidin-4yl)oxyphenoxy]ethanol

Using General Procedure 2 starting from Preparation R2ai as reagent. Preparation R3ai was obtained. HRMS calculated for C₁₃H₁₅N₃O₄: 277.1063; found 278.1134 ((M+H)⁺ form). ¹H-NMR (400 MHz, dmso-d6) δ ppm 7.73 (s, 1H), 7.06 (d, 2H), 6.96 (d, 2H), 4.88 (t, 1H), 4.8 (brs, 2H), 3.99 (t, 2H), 3.95 (s, 3H), 3.72 (m, 2H).

¹³C-NMR (125 MHz, dmso-d6) δ ppm 157.7, 156, 155.4, 147.2, 142.9, 122.7, 115.5, 70.4, 60.1, 54.4

### Preparation R3ai: tert-butyl 2-[4-(5-amino-6-methoxy-pyrimidin-4-yl)oxyphenyl]pyrrolidine-1-carboxylate

Using General Procedure 2 starting from Preparation R2aj as reagent. Preparation R3aj was obtained. HRMS calculated for C₂₀N₂₆N₄O₄: 386.1954; found 387.2031 ((M+H)⁺ form).

¹H-NMR (500 MHz, dmso-d6) δ ppm 7.76 (s, 1H), 7.19 (m, 2H), 7.07 (m, 2H), 4.79 (m, 1H), 4.6 (s, 2H), 3.98 (s, 3H), 3.52/3.48 (m+m, 2H), 2.3/1.76 (m+m, 2H), 1.9-1.8 (m, 2H), 1.27 (brs, 9H),

¹³C-NMR (125 MHz, dmso-d6) δ ppm 143.2, 126.9, 120 9, 60.7, 54 3. 47.4, 35.6, 28.6, 23.4,

### Preparation R3al: 4-(1H-indazol-6-yloxy)-6-methoxy-pyrimidin-5-amine

Using General Procedure 2 starting from Preparation R2al as reagent. Preparation R3al was obtained. HRMS calculated for C₁₂H₁₁N₅O₂: 257.0913; found 258.0985 ((M+H)⁺ form)

¹H-NMR (500 MHz, dmso-d6) δ ppm 12.99 (s, 1H), 8.06 (s, 1H), 7.76 (s, 1H), 7.75 (d, 1H), 7.24 (d, 1H), 6.91 (d, 1H), 4.9 (brs, 2H), 3.96 (s, 3H).

¹³C-NMR (125 MHz, dmso-d6) δ ppm 158.1, 154.8, 152.9, 142.9, 140.7, 134, 121.6, 120.5, 117.5, 116, 101.5, 54.4.

### Preparation R3am: 2-[4-(5-amino-6-methoxy-pyrimidin-4-yl)oxyphenyl]ethanol

Using General Procedure 2 starting from Preparation R2am as reagent. Preparation R3am was obtained. HRMS calculated for C₁₃H₁₅N₃O₃: 261.1113; found 262.1186 ((M+H)⁺ form).

¹H-NMR (509 MHz, dmso-d6) δ ppm 7.73 (s, 1H), 7.23 (m, 2H), 7.02 (m, 2H), 4.81 (s, 2H), 4.66 (t, 1H), 3.95 (s, 3H), 3.61 (m, 2H), 2.72 (t, 2H).

¹³C-NMR (125 MHz, dmso-d6) δ ppm 142,9, 130.2, 121.2, 62.7, 54.4, 38.8,

Preparation R3an: 4-methoxy-6-[4-(2,2,2-trifluoroethyl)phenoxy]pyrimidin-5-amine

Using General Procedure 2 starting from Preparation R2an as reagent. Preparation R3an was obtained. HRMS calculated for C₁₃H₁₂F₃N₃O₂: 299.0882; found 299.08761 ((M⁻) form).

¹H-NMR (500 MHz, dmso-d6) δ ppm 7.76 (s, 1H), 7.37 (m, 2H) 7.13 (m, 2H), 4.87 (s, 2H), 3.96 (s, 3H), 3.65 (q, 2H)

¹³C-NMR (125 MHz, dmso-d6) δ ppm 158.1, 154.5, 153.9, 142.9, 131.9, 126.8, 126.8, 121.4, 117.5, 54.4, 38.2

### Preparation R3ao: 4-[4-(2,2-difluoroethyl)phenoxyl-6-methoxy-pyrimidin-5-amine

Using General Procedure 2 starting from Preparation R2ao as reagent, Preparation R3ao was obtained. HRMS calculated for C₁₃H₁₃F₂N₃O₂: 281.0976; found 282.1045 ((M+H)⁺ form).

¹H-NMR (400 MHz, dmso-d6) δ ppm 7.75 (s, 1H), 7.32 (dm, 2H), 7.1 (dm, 2H), 6.25 (tt, 1H), 4.85 (br., 2H), 3.95 (s, 3H), 3.18 (td, 2H)

¹³C-NMR (125 MHz, dmso-d6) δ ppm 142.9, 131.4, 121.4, 117.5, 66.8, 54.4

### Preparation R3ap: 4-[4-(2-fluoroethyl)phenoxy]-6-methoxy-pyrimidin-5-amine

Using General Procedure 2 starting from Preparation R2ap as reagent, Preparation R3ap was obtained. HRMS calculated for C₁₃H₁₄FN₃O₂: 263.107; found 264.1140 ((M+H)⁺ form).

¹H-NMR (400 MHz, dmso-d6) δ ppm 7.75 (s, 1H), 7.3 (d, 2H), 7.3 (d, 2H), 4.84 (s, 2H), 4.66 (dt, 2H), 3.96 (s, 3H), 2.99 (dt, 2H).

¹³C-NMR (125 MHz, dmso-d6) δ ppm 158, 154.9, 152.7, 142.9, 134, 130.4, 121.4, 117.2, 84.4, 54.4, 35.9

### Preparation R3aq: 4-[4-fluoro-3-(trifluoromethoxy)phenoxy]-6-methoxy-pyrimidin-5-amine

Using General Procedure 2 starting from Preparation R2aq as reagent, Preparation Ring was obtained. HRMS calculated for C₁₂H₉F₄N₃O₃: 319.058; found 319.05479 (M⁺ form).

¹H-NMR (500 MHz, dmso-d6) δ ppm 7.77 (s, 1H), 7.55 (dd, 11.1), 7.49 (dm, 1H), 7.29 (dm, 1H), 4.95 (s, 2H), 3.96 (s, 3H).

¹³C-NMR (125 MHz, dmso-d6) δ ppm 158.2, 154, 151, 150.2, 142.7, 135.5, 122.5, 120.4, 118.2, 117.7, 117.5, 54.5.

### Preparation R3as: 4-(4-chloro-3-ethyl-phenoxy)-6-methoxy-pyrimidin-5-amine

Using General Procedure 2 starting from Preparation R2as as reagent. Preparation R3as was obtained. HRMS calculated for C₁₃H₁₄ClN₃O₂: 279.0775; found 280.0842 ((M+H)⁺ form).

¹H-NMR (400 MHz, dmso-d6) δ ppm 7.77 (s, 1H), 7.42 (d, 1H), 7.15 (d, 1H), 7.01 (dd, 1H), 4.89 (s, 2H), 3.96 (s, 3H), 2.7 (q, 2H), 1.17 (t, 3H).

¹³C-NMR (125 MHz, dmso-d6) δ ppm 158.1, 154.4, 153.1, 142.8, 142.7, 130.3, 128.4, 122.6, 120.7, 117.4, 54.4, 26.6, 14.4.

### Preparation R3at: 4-(3-benzyloxyphenoxy)-6-methoxy-pyrimidin-5-amine

Using General Procedure 2 starting from Preparation R2at as reagent. Preparation R3at was obtained. HRMS calculated for C₁₈H₁₇N₃O₃: 323.127; found 324.1347 ((M+H)⁺ form).

¹H-NMR (400 MHz, dmso-d6) δ ppm 7.76 (s, 1H), 7.48-7.3 (m, 5H), 7.29 (t, 1H), 6.85 (dm, 1H), 6.8 (t, 1H), 6.7 (dm, 1H), 5.09 (s, 2H), 4.84 (s, 2H), 3.95 (s, 3H)

¹³C-NMR (125 MHz, dmso-d6) δ ppm 142.9, 130.4, 113.7, 111.4, 108.2, 69.9, 54.4.

### Preparation R3au: 4-(5-amino-6-methoxy-pyrimidin-4-yl)oxybenzaldehyde

Using General Procedure 2 starting from Preparation R2au as reagent, Preparation R3au was obtained. HRMS calculated for C₁₂H₁₁N₃O₃: 245.08; found 246.0873 ((M+H)⁻ form).

¹H-NMR (500 MHz, dmso-d6) δ ppm 9.97 (s, 1H), 7.95 (m, 2H), 7.81 (s, 1H), 7.32 (m, 2H), 5.02 (s, 2H), 3.97 (s, 3H).

¹³C-NMR (125 MHz, dmso-d δ ppm 192.3, 159.4, 158.6, 153.1, 142.8, 132.8, 131.8, 121, 118.5, 54.6.

### Preparation R3av: tert-butyl N-[(1R)-1-[4-(5-amino-6-methoxy-pyrimidin-4-yl) oxyphenyl]-2,2,2-trifluoro-ethyl]carbamate

Using General Procedure 2 starting from Preparation R2av as reagent, Preparation R3av was obtained. HRMS calculated for C₁₈H₂₁F₃N₄O₄: 414.1515; found 415.1578 ((M+H)⁺ form).

### Preparation R3aw: tert-butyl N-[(1S)-1-[4-(5-amino-6-methoxy-pyrimidin-4-yl)oxyphenyl]-2,2,2-trifluoro-ethyl]carbamate

Using General Procedure 2 starting from Preparation R2aw as reagent, Preparation R3aw was obtained. HRMS calculated for C₁₈H₂₁F₃N₄O₄: 414.1515; found 415.1586 ((M+H)⁺ form).

¹H-NMR (500 MHz, dmso-d6) δ ppm 8.39 (d, 1H), 7.76 (s, 1H), 7.59 (m, 2H), 7.16 (m, 2H), 5.44 (m, 1H), 4.88 (brs, 2H), 3.96 (s, 3H), 1.41 (s, 9H)

¹³C-NMR (125 MHz, dmso-d6) δ ppm 142.9, 130.2, 121.3, 55.3, 54.5, 28.5

### Preparation R3az: tert-butyl 2-[3-(5-amino-6-methoxy-pyrimidin-4-yl)oxyphenyl]piperidine-1-carboxylate

Using General Procedure 2 starting from Preparation R2az as reagent. Preparation R3az was obtained. HRMS calculated for C₂₁H₂₈N₄O₄: 400.2111; found 401.2183 ((M+H)⁺ form).

¹H-NMR (500 MHz, dmso-d6) δ ppm 7.74 (s, 1H), 7.39 (t, 1H), 7.03 (d, 1H), 7 (d, 1H), 6.88 (brs, 1H), 5.28 (br., 1H), 3.95 (s, 3H), 3.91/2.68 (d+t, 2H), 2.28/1.75 (d+tm, 2H), 1.54/1.4 (d+m, 2H), 1.54/1.25 (d+q, 2H), 1.36 (s, 9H).

¹³C-NMR (125 MHz, dmso-d6) δ ppm 158.1, 155, 154.7, 154,5, 142.9, 142.5, 130.1, 122.6, 119.3, 119.1, 79.4, 54.4, 53, 411.3, 28.5, 28.3, 25.3, 19.4

### Preparation R3bc: 5-(5-amino-6-methoxy-pyrimidin-4-yl)oxyindan-2-ol

Using General Procedure 2 starting from Preparation R2bc as reagent, Preparation R3bc was obtained. HRMS calculated for C₁₄H₁₅N₃O₃: 273.113; found 274.1188 ((M+H)⁻ form).

¹H-NMR (500 MHz, dmso-d6) δ ppm 7.73 (s, 1H), 7.19 (d, 1H), 6.95 (d, 1H), 6.86 (dd, 1H), 4.89 (d, 1H), 4.78 (br., 2H), 4.52 (m, 1H), 3.94 (s, 3H), 3.09-3,00+2.77-2.68 (m, 2H), 3.09-3.00+2.77-2.68 (m, 2H).

¹³C-NMR (125 MHz, dmso-d6) δ ppm 157.8, 155.3, 152.7, 143.5, 143, 138.2, 125.5, 119.5, 118, 117, 72.1, 54.4, 42.7, 41.9

### Preparation R3bf: 2-[3-(5-amino-6-methoxy-pyrimidin-4-yl)oxyphenyl]propan-2-ol

Using General Procedure 2 starting from Preparation R2bf as reagent. Preparation R3bf was obtained. HRMS calculated for C₁₄H₁₇N₃O₃: 275.127; found 276.1342 ((M+H)⁺ form). ¹H-NMR (500 MHz, dmso-d6) δ ppm 7.75 (s, 1H), 7.31 (t, 1H), 7.26 (dm, 1H), 7.2 (t, 1H), 6.93 (dm, 1H), 5.07 (s, 1H), 4.83 (s, 2H), 3.95 (s, 3H), 1.41 (s, 6H)

¹³C-NMR (125 MHz, dmso-d6) δ ppm 158, 154.9, 153.9, 153, 143, 129.1, 121.1, 118.9, 117.6, 71, 54.4, 32.3

### Preparation R3bg: 4-(5-amino-6-methoxy-pyrimidin4-yl)oxy-2-fluoro-benzonitrile

Using General Procedure 3 starting from Preparation R2bg as reagent. Preparation R3bg was obtained. HRMS calculated for C₁₂H₉FN₄O₂: 260.071; found 261.0779 ((M+H)⁺ form).

¹H-NMR (400 MHz, dmso-d6) δ ppm 7.95 (dd, 1H), 7.84 (s, 1H), 7.42 (dd, 1H), 7.18 (dm, 1H), 5.1 (s, 2H), 3.98 (s, 3H).

¹³C NMR (125 MHz, dmso-d6) δ ppm 163.8, 160, 158.9, 152.1, 142.7, 135.3, 117.6, 114.5, 109, 95.8, 54.7.

### Preparation R3bh: 4-(5-amino-6-methoxy-pyrimidin-4-yl)oxy-2-chloro-benzonitrile

Using General Procedure 3 starting from Preparation R2bh as reagent. Preparation R3bh was obtained. HRMS calculated for C₁₂H₉ClN₄O₂: 276.0414; found 277.0486 ((M+H)⁺ form).

¹H-NMR (400 MHz, dmso-d6) δ ppm 8.00 (d, 1H), 7.84 (s, 1H), 7.61 (d, 1H), 7.32 (dd, 1H), 5.1 (s, 2H), 3.98 (s, 3H).

¹³C-NMR (125 MHz, dmso-d6) δ ppm 158.9, 158.8, 152.2, 142.7, 137, 136.2, 122, 120.1, 116.4, 107.5, 54.7.

### Preparation R3bi: 2-[4-(5-amino-6-methoxy-pyrimidin-4-yl)oxyphenyl]acetonitrile

Using General Procedure 3 starting from Preparation R2bi as reagent. Preparation R3bi was obtained. HRMS calculated for C₁₃H₁₂N₄O₂: 256.096; found 257.1034 ((M+H)⁺form).

¹H-NMR (500 MHz, dmso-d6) δ ppm 7.74 (s, 1H), 7.37 (m, 2H), 7.15 (m, 2H), 4.87 (s, 2H), 4.04 (s, 2H), 3.95 (s, 3H).

¹³C-NMR (125 MHz, dmso-d6) δ ppm 158.1, 154.6, 153.5, 142.8, 129.7, 127.8, 122, 119.8, 54.4, 22.2.

### Preparation R3bj: 3-[4-(5-amino-6-methoxy-pyrimidin-1-yl)oyphenyl]propanenitrile

Using General Procedure 3 starting from Preparation R2bj as reagent. Preparation R3bj was obtained. HRMS calculated for C₁₄H₁₄N₄O₂: 270.1117; found 271.1192 ((M+H)⁺ form).

¹H-NMR (400 MHz, dmso-d6) δ ppm 7.75 (s, 1H), 7.31 (dm, 2H), 7.08 (dm, 2H), 4.83 (br., 2H), 3.95 (s, 3H), 2.89 (m, 2H), 2.82 (m, 2H).

¹³C-NMR (125 MHz, dmso-d6) δ ppm 158, 154.8, 152.9, 142.9, 135.4, 129.9, 121.5, 120.8, 54.4, 30.4, 18.8.

### Preparation R3bk: 4-(5-amino-6-methoxy-pyrimidin-4-yl)oxy-3-chloro-benzonitrile

Using General Procedure 3 starting from Preparation R2bk as reagent, Preparation R3bk was obtained. HRMS calculated for C₁₂H₉ClN₄O₂: 276.0414; found 277.0484 ((M+H)⁺ form).

¹H-NMR (500 MHz, dmso-d6) δ ppm 8.23 (d, 1H), 7.89 (dd, 1H), 7.73 (s, 1H), 7.48 (d, 1H), 5.05 (s, 2H), 3.97 (s, 3H).

¹³C-NMR (125 MHz, dmso-d6) δ ppm 158.4, 154, 153.2, 142.6, 134.7, 133.3, 127.3, 124.9, 117.9, 117.5, 109.3, 54.6

### Preparation R3bm: tert-butyl N-[1-[4-(5-amino-6-methoxy-pyrimidin-4-yl)oxyphenyl]ethyl]carbamate

Using General Procedure 2 starting from Preparation R2bm as reagent, Preparation R3bm was obtained. HRMS calculated for C₁₈H₂₄N₄O₄: 360.1797; found 361.1862 ((M+H)⁺ form).

¹H-NMR (500 MHz, dmso-d6) δ ppm 7.77 (s, 1H), 7.32 (m, 2H), 7.07 (m, 2H), 7.06 (brs, 1H), 4.64 (m, 1H), 4.59 (brs, 2H), 3.98 (s, 3H), 1.37 (s, 9H), 1.35 (d, 3H).

¹³C-NMR (12.5 MHz, dmso-d6) δ ppm 143.2, 127.3. 120.9, 54.3. 49.9. 28.8. 23.1.

### Preparation R3bn: tert-butyl N-[1-[4-(5-amino-6-methoxy-pyrimidin-4-yl)oxyphenyl]propyl]carbamate

Using General Procedure 2 starting from Preparation R2bn as reagent. Preparation R3bn was obtained. HRMS calculated for C₁₉H₂₆N₄O₄: 374.1954; found 375.2024 ((M+H)⁺ form).

¹H-NMR (500 MHz, dmso-d6) δ ppm 7.77 (s, 1H), 7.3 (m, 2H), 7.06 (m, 2H), 7.01 (brs, 1H), 4.6 (s, 2H), 4.38 (m, 1H), 3.98(s, 3H), 1.7/1.65 (m+m, 2H), 1.37 (s, 3H), 0.85 (t, 3H).

¹³C-NMR (125 MHz, dmso-d6) δ ppm 142.9, 127.8, 121.1, 56.2, 54.4, 30, 28.8, 11.

### Preparation R3bq: 4-methoxy-6-(3-pyridyloxy)pyrimidin-5-amine

Using General Procedure 2 starting from Preparation R2bq as reagent. Preparation R3bq was obtained. HRMS calculated for C₁₀H₁₀N₄O₂: 218.0804; found 219.0878 ((M+H) form).

¹H-NMR (400 MHz, dmso-d6) δ ppm 8.46 (d, 1H), 8.42 (dd, 1H), 7.76 (s, 1H), 7.63 (dm, 7.46 (dd, 1H), 4.97 (br., 2H), 3.96 (s, 3H)

¹³C-NMR (125 MHz, dmso-d6) δ ppm 158.2, 154.1, 150.7, 146, 143.4. 142.7, 129.1, 124.8, 117.4, 54.5

### Preparation R3bs: 4-methoxy-6-(1H-pyrrolo[3,2-b]pyridin-6-yloxy)pyrimidin-5-amine

Using General Procedure 2 starting from Preparation R2bs as reagent. Preparation R3bs was obtained. HRMS calculated for C₁₂H₁₁N₅O₂: 257.0913; found 258.0990 ((M+H)⁺ form).

¹H-NMR (500 MHz, dmso-d6) δ ppm 11.3 (brs, 1H), 8.19 (d, 1H), 7.72 (s, 1H), 7.63 (dd, 1H), 7.6 (dd, 1H), 6.56 (d, 1H), 4.91 (s, 2H), 3.96(s, 3H)

¹³C-NMR (125 MHz, dmso-d6) δ ppm 142.8, 137.5, 130.1, 111.8, 102.1

### Preparation R3bt: 4-(3-benzyloxy-4-chloro-phenoxy)-6-methoxy-pyrimidin-5-amine

Using General Procedure 2 starting from Preparation R2bt as reagent. Preparation R3bt was obtained. HRMS calculated for C₁₈H₁₆ClN₃O₃: 357.088; found 358.0962 ((M+H)⁺ form).

¹H-NMR (500 MHz, dmso-d6) δ ppm 7.76 (s, 1H), 7.51-7.29(m, 5H), 7.44 (d, 1H), 7.11 (d, 1H), 6.75 (dd, 1H), 5.18 (s, 2H), 4.89 (s, 2H), 3.96 (s, 3H)

¹³C-NMR (125 MHz, dmso-d6) δ ppm 142.8, 130.4, 114.5, 108.3, 70.6, 54.5

### Preparation R3bu: 4-(3-benzyloxy-4-methyl-phenoxy)-6-methoxy-pyrimidin-5-amine

Using General Procedure 2 starting from Preparation R2bu as reagent. Preparation R3bu was obtained. HRMS calculated for C₁₉H₁₉N₃O₃: 337.1426, found 338.1509 ((M+H)⁺ form).

¹H-NMR (500 MHz, dmso-d6) δ ppm 7.74 (s, 1H), 7.49-7.28 (m, 5H), 7.15 (d, 1H), 6.85 (d, 1H), 6.62 (dd, 1H), 5.08 (s, 2H), 4.8 (s, 2H), 3.95 (s, 3H), 2.19 (s, 3H)

¹³C-NMR (125 MHz, dmso-d6) δ ppm 142.9, 130.9, 113.2, 106.1, 69.7, 54.4, 16.1

### Preparation R3bv: tert-butyl 4-[2-[4-(5-amino-6-methoxy-pyrimidin-4-yl)oxyphenyl]ethyl]piperidine-1-carboxylate

Using General Procedure 2 starting from Preparation R2bv as reagent. Preparation R3bv was obtained. HRMS calculated for C₁₆H₂₀N₄O₃: 316,1535; found 317.1615 ((M+H)⁺ form).

¹H-NMR (500 MHz, dmso-d6) δ ppm 7.75 (s, 1H), 7.31 (dm, 2H), 7.07 (dm, 2H),4.83 (br., 2H), 3.95 (s, 3H), 3.57 (t, 4H), 3.45 (s, 2H), 2.35 (br., 4H)

¹³C-NMR (125 MHz, dmso-d6) δ ppm 158, 154.7, 153.1, 142.9, 134, 130.4, 121.1, 117.3, 66.7, 62.3, 54.4, 53.6

### Preparation R3bw: 4-methoxy-6-[4-(morpholinomethyl)pbenoxy]pyrimidin-5-amine

Using General Procedure 2 starting from Preparation R2bw as reagent, Preparation R3bw was obtained. HRMS calculated for C₁₆H₂₀N₄O₃: 316.1535; found 317.1615 ((M+H)⁺ form).

¹H-NMR (500 MHz, dmso-d6) δ ppm 7.75 (s, 1H), 7.31 (dm, 2H), 7.07 (dm, 2H), 4.83 (br., 2H), 3.95 (s, 3H), 3.57 (t, 4H), 3.45 (s, 2H), 2.35 (br., 4H)

¹³C-NMR (125 MHz, dmso-d6) δ ppm 158, 154.7, 153.1, 142.9, 134.3, 130.4, 121.1, 117.3, 66.7, 62.3, 54.4, 53.6

### Preparation R3bx: tert-butyl 2-[2-[4-(5-amino-6-methoxy-pyrimidin-4-yl)oxyphenyl]ethyl]piperidine-1-carboxylate

Using General Procedure 2 starting from Preparation R2bx as reagent. Preparation R3bx was obtained. HRMS calculated for C₂₃H₃₂H₄O₄: 428.2424; found 429.2486 ((M+H)⁺ form).

¹H-NMR (500 MHz, dmso-d6) δ ppm 7.73 (s, 1H), 7.23 (m, 2H), 7.02 (m, 2H), 4.81 (s, 2H), 4.15 (m, 1H), 3.95 (s, 3H), 3.86/2.8 (m+m, 2H), 2.54/2,44 (m+m, 2H), 1.93/1.72 (m+m, 2H), 1.64-1.19 (m, 6H), 1.38 (s, 3H).

¹³C-NMR (125 MHz, dmso-d6) δ ppm 142.9, 129.6, 121.4, 54.4, 50.2, 38.7, 31.9, 31.8, 28.6

### Preparation R3bv: tert-butyl 2-[3-(5-amino-6-methoxy-pyrimidin-4-yl)oxyphenyl]morpholine-4-carboxylate

Using General Procedure 2 starting from Preparation R2by as reagent, Preparation R3by was obtained. HRMS calculated for C₂₀H₂₆N₄O₅: 402.1903; found 403.197 ((M+H)⁺ form).

¹H-NMR (400 MHz, dmso-d6) δ ppm 7.76 (s, 1H), 7.39 (t, 1H), 7.2 (dm, 1H), 7.12 (t, 1H), 7.08 (dm, 1H), 4.86 (brs, 2H), 4.43 (dd, 1H), 4.00-2.62 (brm, 4H), 3.96 (s, 3H), 3.94/3.54 (m+m, 2H), 1.41 (s, 9H).

¹³C-NMR (100 MHz dmso-d6) δ ppm 142.9, 142.9, 129.9, 122.9, 120.8, 119.1, 76.7, 66.3, 54.5

### Preparation R3bz: tert-butyl N-[(1R)-1-[4-(5-amino-6-methoxy-pyrimidin-4-yl)oxyphenyl]-2,2,2-trifluoro-ethyl]carbamate

Using General Procedure 2 starting from Preparation R2bz as reagent. Preparation R3bz was obtained. HRMS calculated for C₁₈H₂₁F₃N₄O₄: 414.1515; found 415.1578 ((M+H)⁺ form).

¹H-NMR (500 MHz, dmso-d6) δ ppm 8.39 (d, 1H), 7.76 (s, 1H), 7.59 (m, 2H), 7.16 (m, 2H), 5.44 (m, 1H), 4.88 (brs, 2H), 3.96 (s, 3H), 1.41 (s, 9H)

¹³C-NMR (125 MHz, dmso-d6) δ ppm 142.9, 130.2, 121.3, 55.3, 54.5, 28.5

### Preparation R3ca: 2-[4-(5-amino-6-methoxy-pyrimidin-4yl)oxyphenyl]acetonitrile

Using General Procedure 3 starting from Preparation R2ca as reagent. Preparation R3ca was obtained. HRMS calculated for C₁₃H₁₂N₄O₂: 256.096; found 257.1034 ((M+H)⁺ form).

¹H-NMR (500 MHz, dmso-d6) δ ppm 7.74 (s, 1H), 7.37 (m, 2H), 7.15 (m, 2H), 4.87 (s, 2H), 4.04 (s, 2H), 3.95 (s, 3H)

¹³C-NMR (125 MHz, dmso-d6) δ ppm 158.1, 154.6, 153.5, 142.8, 129.7, 127.8, 122, 119.8, 54.4, 22.2

### Preparation R3cb: [5-(5-amino-6-methoxy-pyrimidin-4-yl)oxy-2-fluoro-phenyl]methanol

Using General Procedure 2 starting from Preparation R2cb as reagent, Preparation R3cb was obtained. HRMS calculated for C₁₃H₁₂N₄O₂: 265.0863; found 266.0935 ((M+H) form).

¹H-NMR (500 MHz, dmso-d6) δ ppm 7.75 (s, 1H), 7.18 (dt, 7.17 (t, 1H), 7.07 (ddd, 1H), 5.35 (t, 1H), 4.86 (s, 2H), 4.55 (d, 2H), 395 (s, 3H).

¹³C-NMR (125 MHz, dmso-d6) δ ppm 158, 156.5, 154.9, 150, 142.8, 130.9, 121.4, 121.4, 117.3, 116, 57.1, 54.4

### Preparation R3cc: 4-(3-amino-4-fluoro-phenoxy)-6-methoxy-pyrimidin-5-amine

Using General Procedure 2 starting from Preparation R2cc as reagent. Preparation R3cc was obtained. HRMS calculated for C₁₁H₁₁FN₄O₂: 250.0866; found 251.0938 ((M+H)⁺ form).

¹H-NMR (500 MHz, dmso-d6) δ ppm 7.75 (s, 1H), 6.96 (dd, 1H), 6.48 (dd, 1H), 6.23 (ddd, 1H), 5.26 (s, 2H), 4.78 (s, 2H), 3.94 (s, 3H)

¹³C-NMR (125 MHz, dmso-d6) δ ppm 157.9, 155, 150.5, 148, 143, 137.6, 117.1, 115.4, 108.9, 108.1, 54.4

### Preparation R3cd: 4-(4-isopropyl-3-methoxy-phenoxy)-6-methoxy-pyrimidin-5-amine

Using General Procedure 2 starting from Preparation R2cd as reagent, Preparation R3cd was obtained. HRMS calculated for C₁₅H₁₉N₃O₃: 289.1426; found) 289.14174 (M⁺ form).

¹H-NMR (500 MHz, dmso-d6) δ ppm 7.75 (s, 1H), 7.17 (d, 1H), 6.74 (d, 1H), 6.6 (dd, 1H), 4.8 (s, 2H), 3.95 (s, 3H), 3.75 (s, 3H), 3.21 (m, 1H), 1.16 (d, 6H)

¹³C-NMR (125 MHz, dmso-d6) δ ppm 157.9, 157.4, 155, 153, 143, 132.5, 126.4, 117.1, 113, 104.9, 56, 55.4, 26.4, 23.1

### Preparation R3ce: tert-butyl 2-[4-(5-amino-6-methoxy-pyrimidin-4-yl)oxyphenyl]piperidine-1-carboxylate

Using General Procedure 2 starting from Preparation R2ce as reagent. Preparation R3ce was obtained. HRMS calculated for C₂₁H₂₈N₄O₄: 400.2111; found 345 1564 ((M+H-C₄H₈)⁺ form).

¹H-NMR (500 MHz, dmso-d6) δ ppm 7.75 (s, 1H), 7.2 (m, 2H), 7.12 (m, 2H), 5.28 (brd, 1H), 4.84 (s, 2H), 3.95 (s, 3H), 3.94/2.71 (m+m, 2H), 1.4 (s, 9H)

¹³C-NMR (125 MHz, dmso-d6) δ ppm 142.9, 127.7, 121.5, 54.4, 52.9, 40.3, 28.5

### Preparation R4a: 5-amino-4-phenoxy-1H-pyrimidin-6-one

Using General Procedure 4 starting from Preparation R3a as reagent. Preparation R4a was obtained. HRMS calculated for C₁₀H₉N₃O₂: 203.0695; found 204.1177 ((M+H)⁺ form).

¹H-NMR (500 MHz, dmso-d6) δ ppm 12.45 (brs, 1H), 7.5 (s, 1H), 7.35 (m, 2H), 7.11 (m, 1H), 7.02 (m, 2H), 4.6 (s, 2H).

¹³C-NMR (125 MHz, dmso-d6) δ ppm 159.3, 147.3, 135.7, 129.8, 123.8, 121.7, 119.7.

### Preparation R4b: 5-amino-4-(2-fluorophenoxy)-1H-pyrimidin-6-one hydrochloride

Using General Procedure 4 starting from Preparation R3b as reagent. Preparation R4b was obtained. HRMS calculated for C₁₀H₈FN₅O₂: 221.0601; found 222.0673 ((M+H)⁻ form).

¹H-NMR (500 MHz, dmso-d6) δ ppm 13.12 (brs, 1H), 7.88 (s, 1H), 7.37 (m, 1H), 7.3 (m, 1H), 7.28 (m, 1H), 7.24 (m, 1H)

¹³C-NMR (125 MHz, dmso-d6) δ ppm 159.3, 155.3, 154.4, 143.8, 140.4, 127.2, 125.6, 124.1, 117.2.

### Preparation R4c: 5-amino-4(4-methoxyphenoxy)-1H-pyrimidin-6-one hydrochloride

Using General Procedure 4 starting from Preparation R3c as reagent. Preparation R4c was obtained. HRMS calculated for C₁₁H₁₁N₃O₃: 233.08; found 234.08709 ((M+H)⁺ form).

¹H-NMR (400 MHz, dmso-d6) δ ppm 13.04 (brs, 1H), 7.9 (s, 1H), 7.07 (m, 2H), 6.95 (m, 2H), 3.75 (s, 3H).

¹³C-NMR (125 MHz, dmso-d6) δ ppm 159.4, 156.8, 156.8, 146.8, 144.5, 122.5, 114.9, 55.9.

### Preparation R4d: 5-amino-4-(3-methoxyphenoxy)-1H-pyrimidin-6-one hydrochloride

Using General Procedure 4 starting from Preparation R3d as reagent. Preparation R₄d was obtained. HRMS calculated for C₁₁H₁₁N₃O₃: 233.08; found 234.6871 ((M+H)⁺ form).

¹H-NMR (500 MHz, dmso-d6) δ ppm 13.05 (br., 1H), 7.89 (s, 1H), 7.3 (t, 1H), 7.16 (br., 2H), 6.79 (dd, 1H), 6.71 (t, 1H), 6.69 (dd, 1H), 3.74 (s, 3H),

¹³C-NMR (125 MHz, dmso-d6) δ ppm 160.7, 159.5, 155.4, 154.8, 143.8, 130.4, 113.1, 110.8, 107.2, 55.8.

### Preparation R₄e: 5-amino-4-(4-fluorophenoxy)-1H-pyrimidin-6-one hydrochloride

Using General Procedure 4 starting from Preparation R₃e as reagent. Preparation R₄e was obtained HRMS calculated for C₁₀H₈FN₃O₂: 221.0601; found 222.0669 ((M+H)⁺ form).

¹H-NMR (500 MHz, dmso-d6) δ ppm 7.87 (s, 1H), 7.2 (m, 2H), 7.2 (m, 2H).

¹³C-NMR (125 MHz, dmso-d6) δ ppm 160.7, 159.5, 158.2, 149.8, 143.4. 122.9, 166.6.

### Preparation R₄f: 5-amino-4-(3-fluorophenoxy)-1H-pyrimidin-6-one hydrochloride

Using General Procedure 4 starting from Preparation R3f as reagent. Preparation R4f was obtained HRMS calculated for C₁₀H₈FN₃O₂: 221.0601; found 222.0672 ((M+H)⁺ form).

¹H-NMR (500 MHz, dmso-d6) δ ppm 13.01 (br., 1H), 7.85 (s, 1H), 7.43 (m, 1H), 7.43 (br., 2H), 7.07-7.01 (m, 1H), 7.07-7.01 (m, 1H), 6.98 (m, 1H).

¹³C-NMR (125 MHz, dmso-d6) δ ppm 162.9, 159.5, 155.1, 151.4, 142.5, 131.2, 116.8, 111.7, 108.5.

### Preparation R4g: 5-amino-4-(3,5-dimethoxyphenoxy)-1H-pyrimidin-6-one hydrochloride

Using General Procedure 4 starting from Preparation R3g as reagent. Preparation R4g was obtained. HRMS calculated for C₁₂H₁₃N₃O₄: 263.0906; found 263.0977 ((M+H)⁺ form).

¹H-NMR (500 MHz, dmso-d6) δ ppm 12.91 (brs, 1H), 7.82 (s, 1H), 6.34 (t, 1H), 6.27 (d, 2H), 3.72 (s, 6H).

¹³ C-NMR (125 MHz, dmso-d6) δ ppm 161.4, 159.5, 155.8, 153.5, 142.1, 99.4, 96.9, 55.9.

### Preparation R4h: 5-amino-4-(3,5-difluorophenoxy)-1H-pyrimidin-6-one hydrochloride

Using General Procedure 4 starting from Preparation R3h as reagent. Preparation R4h was obtained. HRMS calculated for C₁₀H₇F₂N₃O₂: 239.0506; found 240.0576 ((M+H)⁺ form).

¹H-NMR (400 MHz, dmso-d6) δ ppm 12.96 (br., 1H), 7.81 (s, 1H), 7.07 (m, 1H), 6.93 (m, 2H).

¹³C-NMR (125 MHz, dmso-d6) δ ppm 163, 159.6, 156.2, 151.3, 141.3, 104.5, 100.2.

### Preparation R4i: 5-(methylamino)-4-phenoxy-1H-pyrimidin-6-one hydrochloride

Using General Procedure 4 starting from Preparation R3i as reagent. Preparation R4i was obtained. HRMS calculated for C₁₁H₁₁N₃O₂: 217.0851; found 218.0924 ((M+H)⁺ form).

¹H-NMR (500 MHz, dmso-d6) δ ppm 13.27 (brs, 1H), 8.04 (s, 1H), 7.42 (m, 2H), 7.24 (m, 1H), 7.18 (m, 2H), 2.87 (s, 3H)

¹³C-NMR (125 MHz, dmso-d6) δ ppm 159.4, 157.2, 153.5, 146.7, 130.1, 125.5, 121.2, 112.1, 34.3.

### Preparation R4j: 5-amino-1-(4-chlorophenoxy)-1H-pyrimidin-6-one hydrochloride

Using General Procedure 4 starting from Preparation R3j as reagent. Preparation R4j was obtained. HRMS calculated for C₁₁H₈ClN₃O₂: 237.0305; found 238.0379 ((M+H)⁺ form).

¹H-NMR (500 MHz, dmso-d6) δ ppm 12.92 (brs, 1H), 7.8 (s, 1H), 7.44 (m, 2H), 7.14 (m, 2H).

¹³C-NMR (25 MHz, dmso-6) δ ppm 159.4, 153, 152.9, 141.8, 129.8, 128.8, 122.5.

### Preparation R4k: 5-amino-4-(4-chloro-3-methoxy-phenoxy)-1H-pyrimidin-6-one hydrochloride

Using General Procedure 4 starting from Preparation R3k as reagent. Preparation R4k was obtained. HRMS calculated for C₁₁H₁₀ClN₃O₃: 267.0411; found 2118.0481 ((M+H)⁺ form).

¹H-NMR (500 MHz, dmso-d6) δ ppm 12.87 (brs, 1H), 7.78 (s, 1H), 7.4 (d, 1H), 6.86 (d, 1H), 6.68 (dd, 1H), 3.82 (s, 3H).

¹³C-NMR (125 MHz, dmso-d6) δ ppm 141.1, 130.3, 113.2, 106.2, 56.8.

### Preparation R4l: 5-amino-4-(3-chlorophenoxy)-1H-pyrimidin-6-one

Using General Procedure 4 starting from Preparation R3l as reagent. Preparation R4l was obtained. HRMS calculated for C₁₀H₈ClN₃O₂: 237.0305; found 238.0376 ((M+H)⁺ form).

¹H-NMR (500 MHz, dmso-d6) δ ppm 12.97 (br., 1H), 7.83 (s, 1H), 7.42 (t, 1H). 7.26 (dm, 1H), 7.26 (dm, 1H), 7.23(t, 1H).

¹³C-NMR (125 MHz, dmso-d6) δ ppm 159.5, 154.9, 152.8, 142, 133.8, 131.4, 124.8, 120.8, 119.4.

### Preparation R4n: 5-amino-4-(1,3-benzodioxol-5-yloxy)-1H-pyrimidin-6-one hydrochloride

Using General Procedure 4 starting from Preparation R3n as reagent. Preparation R4n was obtained. HRMS calculated for C₁₁H₉N₃O₄: 247.0593; found 248.0666 ((M+H)⁺ form).

¹H-NMR (500 MHz, dmso-d6) δ ppm 12.79 (brs, 1H), 7.74 (s, 1H), 6.89 (d, 1H), 6.77 (d, 1H), 6.55 (dd, 1H), 6.04 (s, 2H).

¹³C-NMR (125 MHz, dmso-d6) δ ppm 140.9, 113.3, 108.4, 103.5, 102.

### Preparation R4o: 5-amino4-(3-hydroxy-5-methoxy-phenoxy)-1H-pyrimidin-6-one

Using General Procedure 4 starting from Preparation R3o as reagent. Preparation R4o was obtained. HRMS calculated for C₁₁H₁₁N₃O₄: 249.075; found 250.08193 ((M+H)⁺ form).

¹H-NMR (500 MHz, dmso-d6) δ ppm 12.44 (brs, 1H), 9.52 (s, 1H), 7.52 (s, 1H), 6.08 (t, 1H), 6.05 (t, 1H), 5.98 (t, 1H), 4.56 (s, 2H), 3.66(s, 3H).

¹³C-NMR (125 MHz, dmso-d6) δ ppm 161.4, 159.4, 159.4, 157, 146.9, 135.6, 122.9, 99.1, 96.9, 96.7, 55.6

### Preparation R4p: 5-amino-4-(4-fluoro-3-methoxy-phenoxy)-1H-pyrimidin-6-one hydrochloride

Using General Procedure 4 starting from Preparation R3p as reagent. Preparation R4p was obtained. HRMS calculated for C₁₁H₁₀FN₃O₃. 251.0706; found 252.0779 ((M+H)⁺ form).

¹H-NMR (500 MHz, dmso-d6) δ ppm 12.79 (br., 1H), 7.75 (s, 1H), 7.2 (dd, 1H), 6.97 (dd, 1H), 6.64 (ddd, 1H), 5.91 (br., 2H), 3.8 (s, 3H)

¹³C-NMR (125 MHz, dmso-d6) δ ppm 159.4, 152.3, 150.6, 148.9, 148, 140.5, 116.2, 112.3, 107.3, 56.7

### Preparation R4g: 4-phenoxy-5-(2,2,2-trifluoroethylamino)-1H-pyrimidin-6-one

Using General Procedure 4 starting from Preparation R3q as reagent. Preparation R4q was obtained. HRMS calculated for C₁₂H₁₀F₃N₃O₂: 285.0725; found 286.0801 ((M+H)⁺ form).

¹H-NMR (500 MHz, dmso-d6) δ ppm 12.64 (br., 1H), 7.64 (s, 1H), 7.37 (m, 2H), 7.15 (tm, 1H), 7.03 (m, 2H), 5.24 (br., 1H), 4.09 (brq. 2H).

¹³C-NMR (125 MHz, dmso-d6) δ ppm 159.8, 134.5, 150.1, 138.7, 130, 126.1, 124.4, 120.3, 119.5, 44.8.

Preparation R4r: 5-amino-4-[3-(trifluoromethy)phenoxy]-1H-pyrimidin-6-one hydrochloride

Using General Procedure 4 starling from Preparation R3r as reagent. Preparation R4r was obtained. HRMS calculated for C₁₁H₈F₃N₃O₃: 287.0518; found 288.0592 ((M+H)⁺ form).

¹H-NMR (500 MHz, dmso-d6) δ ppm 12.81 (brs., 1H), 7.73 (s, 1H) 7.51 (t, 1H) 7.17 (dm, 1H), 7.14 (dm, 1H), 7.14(m, 1H).

¹³C-NMR (125 MHz, dmso-d6) δ ppm 159.5, 155.5, 150.5, 149.2, 139.9, 121.5, 121.3, 119.3, 116.8, 113.3.

### Preparation R4s: 5-amino-4-(3-methylphenoxy)-1H-pyrimidin-6-one hydrochloride

Using General Procedure 4 starting from Preparation R3s as reagent. Preparation R4s was obtained. HRMS calculated for C₁₁H₁₁N₃O₂: 217.0851; found 218.0922 ((M+H)⁺ form).

1H-NMR (500 MHz, dmso-d6) δ ppm 13.06 (brs, 1H), 7.89 (s, 1H), 7.27 (t, 1H), 7.02 (dm, 1H), 6.93 (m, 1H), 6.91 (dm, 1H), 2.3 (s, 3H).

¹³ C-NMR (125 MHz, dmso-d6) δ ppm 159.4, 155.6, 153.7, 143.9, 139.7, 129.7, 125.8, 121.5, 118.1. 213.0.

### Preparation R4t: 5-amino-4-(3-bromophenoxy)-1H-pyrimidin-6-one

Using General Procedure 4 starting from Preparation R3t as reagent. Preparation R4t was obtained. HRMS calculated for C₁₀H₈BrN₃O₂: 280.98; found 281.98762 ((M+H) form).

¹H-NMR (500 MHz, dmso-d6) δ ppm 12.49 (brs, 1H), 7.53 (s. 1H) 7.31 (m, 1H), 7.31 (m, 1H), 7.24 (m, 1H), 7.06 (m, 1H), 4.71 (brs, 2H).

¹³C-NMR (125 MHz, dmso-d6) δ ppm 159.4, 146.3, 135.7, 131.1, 126.6, 122.3, 118.6.

### Preparation R4u: 5-amino-6-[3-(pentafluoro-λ⁶-sulfanyl)phenoxyl]pyrimidin-4(3H)-one hydrochloride

Using General Procedure 4 starting from Preparation R3u as reagent. Preparation R4u was obtained. HRMS calculated for C₁₀H₈F₅N₃O₂S: 329.0257; found 330.0321 ((M+H)⁺ form).

¹H-NMR (500 MHz, dmso-d6) δ ppm 12.77 (brs, 1H), 7.92 (m, 1H), 7.71 (m, 1H), 7.7 (s, 1H), 7.63 (m, 1H), 7.41 (dm, 1H)

¹³C-NMR (125 MHz, dmso-d6) δ ppm 159.5, 149.7, 139.1, 130.9, 124.3, 121.7, 117.9.

### Preparation R4v: 5-amino-4-[3-(trifluoromethyl)phenoxy]-1H-pyrimidin-6-one hydrochloride

Using General Procedure 4 starting from Preparation R3v as reagent. Preparation R4v was obtained. HRMS calculated for C₁₁H₈F₃N₃O₂: 271.0569; found 272.0634 ((M+H)⁺ form).

¹H-NMR (500 MHz, dmso-d6) δ ppm 12.73 (brs, 1H), 7.67 (s, 1H), 7.61 (t, 1H), 7.51 (dm, 1H), 7.42 (m, 1H), 7.39 (dm, 1H).

¹³C-NMR (125 MHz, dmso-d6) δ ppm 159.4, 155.1, 148.9, 138.3, 131.3, 130.6, 124.1, 120.8, 116.7.

### Preparation R4w: 5-amino-4-[4-(hydroxymethyl)phenoxy]-1H-pyrimidin-6-one

Using General Procedure 4 starting from Preparation R3w as reagent. Preparation R4w was obtained. HRMS calculated for C₁₁H₁₁N₃O₃: 231.08; found 234.0878 ((M+H)⁺ form),

¹H-NMR (400 MHz, dmso-d6) δ ppm 13 (brs, 1H), 7.85 (s, 1H), 7.33 (d, 2H), 7.07 (d, 2H), 44.8 (s, 2H)

¹³C-NMR (125 MHz, dmso-d6) δ ppm 159.4, 155.1, 152.4, 143.2, 139.5, 128.1, 120.8, 62.8

### Preparation R4z: 4-[(5-amino-6-oxo-1H-pyrimidin-4-yl)oxy]benzonitrile

Using General Procedure 4 starting from Preparation R3z as reagent. Preparation R4z was obtained. HRMS calculated for C₁₁H₅N₄O₂: 228.0647; found 229.0718 ((M+H)⁺ form).

¹H-NMR (500 MHz, dmso-d6) δ ppm 12.54 (s, 1H), 7.82 (dm, 2H), 7.35 (s, 1H), 7.18 (dm, 2H), 4.93 (s, 2H).

¹³C-NMR (125 MHz, dmso-d6) δ ppm 159.5, 159.1, 145.1, 135.6, 134.5, 123.3, 119.7, 119.3, 105.8.

### Preparation R4na: 3-[(5-amino-6-ozo-1H-pyrimidin-4-yl)oxy]benzonitrile hydrochloride

Using General Procedure 4 starting from Preparation R3aa as reagent. Preparation R4aa was obtained. HRMS calculated for C₁₁H₈N₄O₂: 228.0647; found 229.072 ((M+H)⁺ form).

¹H-NMR (500 MHz, dmso-d6) δ ppm 12.9 (brs, 1H), 7.77 (s, 1H), 7.65 (dm, 1H), 7.65 (m, 1H), 7.6 (t, 1H), 7.47 (m, 1H).

¹³C-NMR (125 MHz dmso-d6) δ ppm 159.5, 154.6, 151.2, 140.8, 131.4, 128.5, 125.6, 23.8, 118.6, 112.6.

### Preparation R4ab: tert-butyl 7-[(5-amino-6-oxo-1H-pyrimidin-4-yl)oxy]-3,4-dihydro-1H-isoquinoline-2-carboxylate

Using General Procedure 4 starting from Preparation R3ah as reagent, 5-amino-4-(1,2,3,4-tetrahydroisoquinolin-7-yloxy)-1H-pyrimidin-6-one, hydrochloride was formed. The resulted crude product was reacted using General Procedure 6 to give

### Preparation R4ab. HRMS calculated for C₁₈H₂₂N₄O₄: 358.1641; found 359.1717 ((M+H)+ form).

¹H-NMR (500 MHz, dmso-d6) δ ppm 12.43 (s, 1H), 7.49 (s, 1H) 7.12 (d, 1H), 6.85 (d, 1H), 6.84 (dd, 1H), 4.55 (s, 2H), 4.45 (s, 2H), 3.54 (t, 2H), 2.73 (t, 2H), 1.42 (s, 9H)

¹³C-NMR (125 MHz, dmso-d6) δ ppm 159.3, 154.4, 153.4, 147.7, 135.8, 135.2, 130, 130, 121.4, 118.3, 117.4, 79.7, 45.5, 41.7, 28.6, 28

### Preparation R4ac: methyl [(5-amino-6-oxo-1H-pyrimidin-4-yl)oxy]benzoate

Using General Procedure 4 starting from Preparation R3ac as reagent. Preparation R4ac was obtained. HRMS calculated for C₁₂H₁₁N₃O₄: 261.075; found 262.0825 ((M+H)⁺ form).

¹H-NMR (500 MHz, dmso-d6) δ ppm 12.51 (brs, 1H), 7.71 (dm, 1H), 7.53 (s, 1H), 7.52 (m, 1H), 7.51 (t, 1H), 7.35 (dm, 1H), 4.73 (s, 2H), 3.84 (s, 3H).

¹³C-NMR (125 MHz, dmso-d6) δ ppm 166.2, 159.4, 155.5, 146.6, 135.7, 131.3, 130.6, 124.6, 124.5, 122.2, 119.7, 52.8.

### Preparation R4ad: 5-amino-4-[3-(hydroxymethyl)phenoxy]-1H-pyrimidin-6-one

Using General Procedure 4 starting from Preparation R3ad as reagent, Preparation R4ad was obtained. HRMS calculated for C₁₁H₁₁N₃O₃: 233.08; found 234.0875 ((M+H)⁺ form).

¹H-NMR (500 MHz, dmso-d6) δ ppm 12.14 (brs, 1H), 7.5 (s, 1H), 7.28 (t, 1H), 7.04 (dm, 6.96 (t, 1H), 6.88 (dm, 1H), 5.25 (brt, 1H), 4.58 (s, 2H), 4.47 (brd, 2H).

¹³ C-NMR (125 MHz, dmso-d6) δ ppm 159.3, 147.4, 135.7, 129.5, 121.7, 121.7, 117.9, 117.4, 62.9.

### Preparation R4ae: 5-amino-4-[3-(hydroxypropyl)phenoxy]-1H-pyrimidin-6-one hydrochloride

Using General Procedure 4 starting from Preparation R3ae as reagent. Preparation R4ae was obtained. HRMS calculated for C₁₃H₁₅N₃O₃: 261.1113; found 262.1184 ((M+H)⁺ form).

¹H-NMR (500 MHz, dmso-d6) δ ppm 7.9 (s, 1H), 7.21 (d, 2H), 7.02 (d, 2H), 3.4 (t, 2H), 2.6 (m, 2H), 1.7 (m, 2H).

¹³ C-NMR (125 MHz, dmso-d6) δ ppm 151.6, 143.1, 139.1, 129.7, 121, 60.6, 34.8, 31.4.

### Preparation R4af: 5-amino-4-phenylsulfanyl-1H-pyrimidin-6-one hydrochloride

Using General Procedure 4 starting from Preparation R3af as reagent. Preparation R4af was obtained. HRMS calculated for C₁₀H₉N₃OS: 219.0466; found 220.0537 ((M+H)⁺ form).

¹H-NMR (500 MHz, dmso-d6) δ ppm 7.96 (s, 1H), 7.36-7.23 (m, 5H).

¹³C-NMR (125 MHz, dmso-d6) δ ppm 156.3, 137.7, 133.5, 123.8,

### Preparation R4ag: tert-butyl 6-[(5-amino-6-oxo-1H-pyrimidin-4-yl)oxy]-3,4-dihydro-1H-isoquinoline-2-carboxylate

Using General Procedure 4 starting from Preparation R3ag as reagent. 5-amino-4-(1,2,3,4-tetrahydroisoquinolin-6-yloxy)-1H-pyrimidin-6-one hydrochloride salt was formed. The resulted crude product was reacted using General Procedure 6 to give Preparation R4ag. HRMS calculated for C₁₈H₂₂N₄O₄: 358.1641. found 359.1713 ((M+H)⁺ form).

¹H-NMR (400 MHz, dmso-d6) δ ppm 12.46 (brs, 1H), 7.48 (s, 1H), 7.13 (d, 1H), 6.85 (dd, 1H), 6.82 (d, 1H), 4.55 (brs, 2H), 4.45 (s, 2H), 3.52 (t, 2H), 2.74 (t, 2H), 1.42 (s, 9H).

¹³C-NMR (125 MHz, dmso-d6) δ ppm 159.4, 147.6, 135.8, 127.6, 119.5, 118, 45.2, 41.3, 28.7, 28.6.

### Preparation R4ah: tert-butyl 5-[(5-amino-6-oxo-1H-pyrimidin-4-yl)oxy]isoindoline-2-carboxylate

Using General Procedure 4 starting from Preparation R3ah as reagent, 5-amino-4-isoindolin-5-yloxy-1H-pyrimidin-6-one hydrochloride was formed. The resulted crude product was reacted using General Procedure 6 to give Preparation R4ah. HRMS calculated for C₁₇H₂₀N₄O₄: 344.1485; found 345.1555 ((M+H)⁺ form).

¹H-NMR (400 MHz, dmso-d6) δ ppm 12.45 (brs, 1H), 7.49 (d, 1H), 7.28/7.27 (d, 1H), 7 (d, 1H), 6.95/6.93 (dd, 1H), 4.6-4.5 (brs, 4H), 4.57 (brs, 2H), 1.45 (s, 9H).

¹³C-NMR (125 MHz, dmso-d6) δ ppm 159.4, 147.7, 135.9, 124, 119.3, 114.4, 28.6.

### Preparation R4ai: 5-amino-4-[4-(2-hydroxyethoxy)phenoxy]-1H-pyrimidin-6-one hydrochloride

Using General Procedure 4 starting from Preparation R3ai as reagent. Preparation R4ai was obtained. HRMS calculated for C₁₂H₁₃N₃O₄: 263.0906; found 264.0975 ((M+H)⁺ form).

¹H-NMR (500 MHz, dmso-d6) δ ppm 13.12 (brs, 1H), 7.94 (s, 1H), 7.07 (m, 2H), 6.95 (m, 2H), 3.97 (t, 2H), 3.7 (t, 2H).

¹³C-NMR (125 MHz, dmso-d6) δ ppm 159.4, 157.6, 156.3, 146.6, 145.3, 122.5, 115.5, 70.4, 60.

### Preparation R4aj: tert-butyl 2-[4-[(5-amino-6-oxo-1H-pyrimidin-4-yl)oxy]phenyl]pyrrolidine-1-carboxylate

Using General Procedure 4 starting from Preparation R3aj as reagent. 5-amino-4-(4-pyrrolidin-2-ylphenoxy)-1H-pyrimidin-6-one hydrochloride was formed. The resulted crude product was reacted using General Procedure 6 to give Preparation R4aj. HRMS calculated for C₂₀H₂₆N₄O₄: 372.1797; found 373.1872 ((M+H)⁺ form).

¹H-NMR (400 MHz, dmso-d6) δ ppm 12.45 (s, 1H), 7.5 (s, 1H), 7.14 (dm, 2H), 6.98 (dm, 2H), 4.82/4.71 (m, 1H), 4.58 (s, 2H), 3.55-3.41 (m, 2H), 2.27/1.7 (m+m, 2H), 1.82 (m, 2H), 1.39/1.15 (s, 9H).

¹³C-NMR (100 MHz, dmso-d6) δ ppm 159.5, 154, 147.3, 140,5, 135.7, 126.9, 119.6, 60.7/60.2, 47.4/47.2, 36.1/34.9, 28.7/28.3, 23.4/23.1

### Preparation R4 al: 5-amino-4-(1H-indazol-6-yloxy)-1H-pyrimidin-6-one hydrochloride

Using General Procedure 4 starting from Preparation R3al as reagent. Preparation R4al was obtained. HRMS calculated for C₁₁H₉N₅O₂; 243.0756; found 244.0833 ((M+H)⁺ form).

¹H-NMR (500 MHz dmso-d6) δ ppm 13.2 (brs, 1H), 9.36 (brs, 2H), 8.1 (d, 1H), 8.02 (d, 1H), 7.79 (d, 1H) 7.32 (d, 1H), 6.95 (dd, 1H).

¹³C-NMR (125 MHz, dmso-d6) δ ppm 146.7, 133.9, 121.8, 115.8, 102.1.

### Preparation R4am: 5-amino-4-[4-(2-hydroxyethyl)phenoxy]-1H-pyrimidin-6-one hydrochloride

Using General Procedure 4 smiling from Preparation R3am as reagent, Preparation R4am was obtained. HRMS calculated for C₁₂H₁₃N₃O₄: 247.0957; found 248.103 ((M+H)⁺ form).

¹H-NMR (500 MHz, dmso-d6) δ ppm 12.97 (brs, 1H), 7.86 (s, 1H), 7.23 (m, 2H), 7.01 (m, 2H), 3.59 (t, 2H), 2.71 (t, 2H).

¹³ C-NMR (125 MHz, dmso-d6) δ ppm 143.4, 1303, 120.8, 62.6, 38.7.

### Preparation R4an: 5-amino-[4-(2,2,2-trifluoroethyl)phenoxy]-1H -pyrimidin-6-one hydrochloride

Using General Procedure 4 starting from Preparation R3an as reagent. Preparation R4an was obtained. HRMS calculated for C₁₂H₁₀F₃N₃O₂: 285.0725; found 286.0800 ((M+H)⁺ form).

¹H-NMR(400 MHz, dmso-d6) δ ppm 12.8 (br., 1H), 7.73 (s, 1H), 7.36 (dm, 2H), 7.09 (dm, 2H), 3.64 (q, 2H)

¹³ C-NMR (125 MHz, dmso-d6) δ ppm 159.4, 154.1, 151.9, 140.4, 11.9, 127.3, 126.6, 120.5, 38.2

### Preparation R4ao: 5-amino-4-[4-(2,2-difluoroethyl)phenoxy]-1H-pyrimidin-6-one hydrochloride

Using General Procedure 4 starting from Preparation R3ao as reagent, Preparation R4ao was obtained. HRMS calculated for C₁₂H₁₁F₂N₃O₂: 267.0819; found 268.0895 ((M+H)⁺ form).

¹H-NMR (500 MHz, dmso-d6) δ ppm 12.9 (brs, 1H), 7.8 (s, 1H), 7.31 (m, 2H), 7.07 (m, 2H), 6.24 (tt, 1H), 3.17 (td, 2H)

¹³C-NMR (125 MHz, dmso-d6) δ ppm 142.1, 131.5, 120.8, 117.5, 39.3

### Preparation R4an: 5-amino-4-[4-(2-fluoroethyl)phenoxy]-1H-pyrimidin-6-one hydrochloride

Using General Procedure 4 starting from Preparation R3ap as reagent. Preparation R4ap was obtained. HRMS calculated for C₁₂H₁₁F₂N₃O₂: 249.0914; found 250.0988 ((M+H)⁺ form).

¹H-NMR (500 MHz, dmso-d6) δ ppm 13.03 (br., 1H), 7.89 (s, 1H), 7.29 (dm, 2H), 7.06 (dm, 2H), 4.64 (dt, 2H), 2.97 (dt, 2H).

¹³ C-NMR (125 MHz, dmso-d6) δ ppm 159.4, 155.21, 152.3, 143.9, 134.4, 130.5, 121.1, 84.4, 35.8

### Preparation R4aq: 5-amin0-4-[4-fluoro-3-(trifluoromethoxy)phenoxy]-1H-pyrimidin-6-one hydrochloride

Using General Procedure 4 starting from Preparation R3aq as reagent. Preparation R4nq was obtained. HRMS calculated for C₁₁H₇F₄N₃O₃: 305.0424; found 306.0501 ((M+H)⁺ form).

¹H-NMR (400 MHz, dmso-d6) δ ppm 12.84 (br., 1H), 7.73 (s, 1H), 7.54 (dd, 1H), 7.41 (dm, 1H), 7.22 (ddd, 1H)

¹³C-NMR (125 MHz, dmso-d6) δ ppm 159.4, 150.8, 150.6, 140, 135.5, 121.3, 118.3, 116.6

### Preparation R4ar: 5-amino-4-(4-fluoro-3-hydroxy-phenoxy)-1H-pyrimidin-6-one

Preparation R3p was dissolved in abs. DCM (5 mL) and cooled to 0° C., then 1M boron tribromide (2.0 eq.) was added. It was stirred at 0° C. for 1 hour, then it was allowed warm to r.t. After 40 hours, the solid compound was filtered off (180 mg), it was purified by Hanbon preparative HPLC (C18 Silica, Gemini NX 5 μm, 0.02% HCOOH-MeCN, gradient method 5-90%) to give Preparation R4ar. HRMS calculated for C₁₀H₈FN₃O₃: 237.055; found 238.0618 ((M+H)⁺ form).

¹H-NMR (500 MHz, dmso-d6) δ ppm 12.45/10 (brs, 2H), 7.51 (s, 1H), 7.08 (dd, 1H), 6.6 (dd, 1H), 6.44 (m, 1H), 4.59 (brs, 2H)

¹³ C-NMR (125 MHz, dmso-d6) δ ppm 159.3, 151.2, 148, 147.4, 145.7, 135.7, 121.5, 116.4, 110.1, 109.3

### Preparation R4as: 5-amino-4-(4-chloro-3-ethyl-phenoxy)-1H-pyrimidin-6-one

Using General Procedure 4 starting from Preparation R3as as reagent. Preparation R4as was obtained. HRMS calculated for C₁₂H₁₂ClN₃O₂: 265.0618; found 266.0691 ((M+H)⁺ form).

¹H-NMR (500 MHz, dmso-d6) δ ppm 12.46 (brs, 1H), 7.51 (s, 1H) 7.36 (d, 1H), 7.04 (d, 1H), 6.88 (dd, 1H), 4.64 (s, 2H), 2.67 (q, 2H), 1.15 (t, 3H).

¹H-NMR (125 MHz, dmso-d6) δ ppm 135.7, 130.2, 121, 118.9, 26.6, 14.4

### Preparation R4at: 5-amino-4-(3-benzyloxyphenoxy)-1H-pyrimidin-6-one hydrochloride

Using General Procedure 4 starting from Preparation R3at as reagent. Preparation R4at was obtained. HRMS calculated for C₁₇H₁₅N₃O₃: 309.1113; found 310.1182 ((M+H)⁺ form).

¹H-NMR (400 MHz, dmso-d6) δ ppm 12.94 (brs, 1H), 7.83 (s, 1H), 7.44 (dm, 2H), 7.4 (tm, 2H), 7.34 (tm, 1H), 7.29 (t, 1H), 6.85 (dd, 1H), 6.78 (t, 1H), 6.69 (dd, 1H),5.1 (s, 2H).

¹³C-NMR (125 MHz, dmso-d6) δ ppm 159.8, 159.5, 155.1, 142.3, 137.3, 130.4, 128.9, 128.4, 128.3, 113, 111.3. 107.7, 69.9.

### Preparation R4au: 4-[(5-amino-6-oxo-1H-pyrimidin-4-yl)oxy]benzaldehyde

Using General Procedure 4 starting from Preparation R3au as reagent. Preparation R4au was obtained. HRMS calculated for C₁₁H₉N₃O₃: 231.0644; found 232.0714 ((M+H)⁺ form).

¹H-NMR (400 MHz, dmso-d6) δ ppm 12.55 (brs, 1H), 9.94 (s, 1H), 7.92 (d, 2H), 7.57 (s, 1H), 7.21 (d, 2H), 4.82 (brs, 2H).

¹³C-NMR (125 MHz, dmso-d6) δ ppm 192.1, 159.6, 131.9, 119.2.

### Preparation R4az: tert-butyl 2-[3-[[5-(tert-butoxycarbonylamino)-6-oxo-1H-pyrimidin-4-yl]oxy]phenyl]piperidine-1-carboxylate

Using General Procedure 4 starting from Preparation R3az as reagent, 5-amino-4-[3-(2-piperidyl)phenoxy]-1H-pyrimidin-6-one hydrochloride was formed. The resulted crude product was reacted using General Procedure 6 to give Preparation R4az. HRMS calculated for C₂₅H₃₄N₄O₆: 486.2478; found 487.2547 ((M+H)⁺ form).

¹H-NMR (500 MHz, dmso-d6) δ ppm 12.72 (brs, 1H), 8.04 (s, 1H), 7.97 (s, 1H), 7.38 (t, 1H), 7.03 (dd, 1H), 6.95 (dd, 1H), 6.85 (dd, 1H), 5.26 (d, 1H), 3.91/2.68 (d+t, 2H), 2.26/1.76 (d+t, 2H), 1.54/1.24 (d=dd, 2H), 1.53/1.38 (d+t, 2H), 1.38 (s, 9H), 1.37 (s, 9H).

¹³C-NMR (125 MHz, dmso-d6) δ ppm 163.2, 161.6, 155, 154.3, 154.1, 147.8, 142.5, 130.1, 123, 120.4, 119.7, 119.4, 79.4, 78.9, 53, 40.2, 28.5, 28.5, 28.3, 25.3, 19.4

### Preparation R4bc: 5-amino-4-(2-hydroxyindan-5-yl)oxy-1H-pyrimidin-6-one

Using General Procedure 4 starting from Preparation R3bc as reagent. Preparation R4bc was obtained HRMS calculated for C₁₃H₁₃N₃O₃: 259.0957; found 260.1027 ((M+H)⁺ form).

¹H-NMR (500 MHz, dmso-d6) δ ppm 13.02 (br., 1H), 7.89 (s, 1H), 7.2 (d, 1H), 6.95 (d, 1H), 6.87 (dd, 1H), 4.51 (m, 1H)

¹³ C-NMR (125 MHz, dmso-d6) δ ppm 159.4, 156.3, 152.3, 144.3, 143.7, 138.6, 125.6, 119.2, 117.7, 72, 42.7, 41.9

### Preparation R4bf: 5-amino-4-[3-(1-hydroxy-1-methyl-ethyl)phenoxy]-1H-pyrimidin-6-one

Using General Procedure 4 starting from Preparation R3bf as reagent. Preparation R4bf was obtained. HRMS calculated for C₁₃H₁₅N₃O₃: 261.1113; found 262.1186 ((M+H)⁺ form).

¹H-NMR (500 MHz, dmso-d6) δ ppm 12.44 (brs, 1H), 7.5 (s, 1H), 7.25 (t, 1H), 7.17 (dm, 1H), 7.14 (t, 1H), 6.82 (dm, 1H), 5.04 (s, 1H), 4.57 (s, 2H), 1.4 (s, 6H)

¹³C-NMR (125 MHz, dmso-d6) δ ppm 159.5, 155, 153, 147.8, 135.8, 129, 121.6, 120.2, 117.3, 116.1, 71, 32.3

### Preparation R4bg: 4-[(5-amino-6-oxo-1H-pyrimidin-4yl)oxy]-2-fluoro-benzonitrile hydrochloride

Using General Procedure 4 starting from Preparation R3bg as reagent, Preparation R4bg was obtained. HRMS calculated for C₁₁H₇FN₄O₂: 246.0553; found 247.0629 ((M+H)⁺ form).

¹H-NMR (500 MHz, dmso-d6) δ ppm 12.81 (brs, 1H), 7.92 (s, 1H), 7.7 (s, 1H), 7.39 (brs, 2H), 7.32 (dd, 1H), 7.1 (dd, 1H).

¹³C-NMR (125 MHz, dmso-d6) δ ppm 163.9, 160.5, 159.7, 147.4, 138.4, 135.2, 116.4, 114.5, 107.8, 95.

### Preparation R4bh: 4-[(5-amino-6-oxo-1H-pyrimidin-4-yl)oxy]-2-chloro-benzonitrile hydrochloride

Using General Procedure 4 starting from Preparation R3bh as reagent, Preparation R4bh was obtained. HRMS calculated for C₁₁H₇ClN₄O₂: 262.0258; found 263.0335 ((M+H)⁺ form).

¹H-NMR (500 MHz, dmso-d6) δ ppm 12.83 (brs, 1H), 7.98 (d, 1H”), 7.72 (s, 1H), 7.51 (d, 1H). 7.49 (br., 2H), 7.24 (dd, 1H).

¹³C-NMR (125 MHz, dmso-d6) δ ppm 159.6, 159.2, 147.9, 138.9, 137, 136.3, 120.8, 119, 116.5, 107.1.

### Preparation R4bj: 3-[4-[(5-amino-6-oxo-1H-pyrimidin-4-yl)oxy]phenyl]propanenitrile hydrochloride

Using General Procedure 4 starting from Preparation R3bj as reagent. Preparation R4bj was obtained. HRMS calculated for C₁₃H₁₂N₄O₂: 256.096; found 257.103 ((M+H)⁺ form).

¹H-NMR (500 MHz, dmso-d6) δ ppm 12.89 (brs, 1H ), 7.8 (s, 1H), 7.3 (d, 2H), 7.06 (d, 2H), 2.87 (t, 2H), 2.81 (t, 2H).

¹³C-NMR (125 MHz, dmso-d6) δ ppm 159.4, 152.8, 141.7, 135.4, 130, 120.7, 30.3, 18.8.

### Preparation R4bk: 4-[(5-amino-6-oxo-1H-pyrimidin-4-yl)oxy]-3-chloro-benzonitrile

Using General Procedure 4 starting from Preparation R3bk as reagent. Preparation R4bk was obtained. HRMS calculated for C₁₁H₇ClN₄O₂: 262.0258; found 263.0330 ((M+H)⁺ form).

¹H-NMR (400 MHz, dmso-d6) δ ppm 12.57 (br., 1H), 8.18 (d, 1H), 7.8 (dd, 1H), 7.51 (s, 1H), 7.25 (d, 1H), 4.84 (s, 2H)

¹³ C-NMR (125 MHz, dmso-d6) δ ppm 159.4, 154.9, 145.5, 135.7, 134.6, 133.1, 125.7, 122.4, 121.9, 118, 107.7

### Preparation R4bm: tert-butyl N-[1-[4-[(5-amino-6-oxo-1H-pyrimidin-4-yl)oxy]phenyl]ethyl]carbamate

Using General Procedure 4 starting from Preparation R3bm as reagent, 5-amino-4-[4-(1-aminoethyl)phenoxy]-1H-pyrimidin-6-one hydrochloride was formed. The resulted crude product was reacted using General Procedure 6 to give Preparation R4bm. HRMS calculated for C₁₇H₂₂H₄O₄: 346.1641; found 347.1716 ((M+H)⁺ form).

¹H-NMR (500 MHz, dmso-d6) δ ppm 12.16 (brs, 1H), 7.48 (s, 1H), 7.37 (d, 1H), 7.24 (d, 2H), 6.94 (d, 2H), 4.59 (qn, 1H), 4.49 (s, 2H), 1.36 (s, 9H), 1.28 (d, 3H)

¹³C-NMR (125 MHz, dmso-d6) δ ppm 160, 155.3, 153.9, 147.9, 141, 136.6, 127.2, 119.6, 78.1, 49.5, 28.7, 23.5

### Preparation R4bn: tert-butyl N-[1-[4-[(5-amino-6-oxo-1H-pyrimidin-4-yl)oxy]phenyl]propyl]carbamate

Using General Procedure 4 starting from Preparation R3bn as reagent. 5-amino-4-[4-(1-aminopropyl)phenoxy]-1H-pyrimidin-6-one hydrochloride was formed. The resulted crude product was reacted using General Procedure 6 to give Preparation R4bn. HRMS calculated for C₁₈H₂₄N₄O₄: 360.1797; found 361.186 ((M+H)⁺ form).

¹H-NMR (500 MHz, dmso-d6) δ ppm 12.32 (br., 1H), 7.49 (s, 1H), 7.32 (d, 1H), 7.23 (d, 2H), 6.95 (d, 2H), 4.57 (s, 2H), 4.33 (m, 1H), 1.6 (m, 2H), 1.36 (s, 9H), 0.81 (t, 3H)

¹³C-NMR (125 MHz, dmso-d6) δ ppm 159.3, 155.6, 153.7, 147.6, 140, 135.7, 127.8, 121.4, 119.5, 78, 55.9, 30, 28.7, 11.6

### Preparation R4bq: 5-amino4-(3-pyridyloxy)-1H-pyrimidin-6-one

Using General Procedure 4 starting from Preparation R3bq as reagent, Preparation R4bq was obtained. HRMS calculated for C₉H₈N₄O₂: 204.0647; found 205.07216 ((M+H) form).

### Preparation R4bss: 5-amino-4-(1H-pyrrolo[3,2-b]pyridin-6-yloxy)-1H-pyrimidin-6-one, hydrochloride

Using General Procedure 4 starting from Preparation R3bs as reagent. Preparation R4bs was obtained. HRMS calculated for C₁₁H₉N₅O₂: 243.0756; found 244.083 ((M+H)⁺ form).

¹H-NMR (500 MHz, dmso-d6) δ ppm 12.82 (br., 1H), 12.75 (s, 1H), 8.76 (d, 1H), 8.44 (dd, 1H), 8.19 (t, 1H), 7.68 (s, 1H), 6.85 (m, 1H)

¹³C-NMR (125 MHz, dmso-d6) δ ppm 159.4, 45.7, 138.5, 137.4, 134.6, 132.4, 129.1, 120.5, 97

### Preparation R4bt: 5-amino-4-(3-benzyloxy-4-chloro-phenoxy)-1H-pyrimidin-6-one

Using General Procedure 4 starting from Preparation R3bt as reagent. Preparation R4bt was obtained. HRMS calculated for C₁₇H₁₄ClN₃O₃: 343.0724; found 344.0792 ((M+H)⁺ form).

¹H-NMR (500 MHz, dmso-d6) δ ppm 13.03 (brs, 1H), 7.84 (s, 1H), 7.49-7.31 (m, 5H), 7.44 (d, 1H), 7.09 (d, 1H), 6.73 (dd, 1H), 5.18 (s, 2H)

¹³C-NMR (125 MHz, dmso-d6) δ ppm 142.8, 130.5, 113.9, 107.8, 70.7

### Preparation R4bu: 5-amino-4-(3-benzyloxy-4-methyl-phenoxy)-1H-pyrimidin-6-one

Using General Procedure 4 starting from Preparation R3bu as reagent, Preparation R4bu was obtained. HRMS calculated for C₁₈H₁₇N₃O₃: 323.127; found 324.1338 ((M+H)⁺ form).

¹H-NMR (500 MHz, dmso-d6) δ ppm 7.87 (s, 1H), 7.48-7.29 (m, 5H), 13.04 (brs, 1H), 7.15 (dm, 1H), 6.85 (d, 1H), 6.61 (dd, 1H), 5.05 (s, 2H), 2.18 (brs, 3H)

¹³C-NMR (125 MHz, dmso-d6) δ ppm 144, 130.9, 112.8, 105.8. 69.8, 16.1

### Preparation R4bv: tert-butyl 4-[2-[4-[(5-amino-6-oxo-1H-pyrimidin-4-yl)oxy]phenyl]ethyl]piperidine-1-carboxylate

Using General Procedure 4 starting from Preparation R3bv as reagent. 5-amino-4-[4-[2-(4-piperidyl)ethyl]phenoxy]-1H-pyrimidin-6-one hydrochloride was formed. The resulted crude product was reacted using General Procedure 6 to give Preparation R4bv. HRMS calculated for C₂₂H₃₀N₄O₄: 414.2267; found 415.23350 ((M+H)⁺ form).

¹H-NMR (400 MHz, dmso-d6) δ ppm 12.42 (brs, 1H), 7.48 (s, 1H), 7.16 (m, 2H), 6.92 (m, 2H), 4.54 (s, 2H), 3.92/2.66 (brd+brs, 4H), 2.57 (t, 2H), 1.68/0.99 (m+tn, 4H), 1.49 (m, 2H), 1.38 (s, 9H), 1.38 (m, 1H).

¹³C-NMR (100 MHz, dmso-d6) δ ppm 135.7, 129.5, 119.8, 43.9, 38.5, 35.2, 32.1, 31.8, 28.5

### Preparation R4bw: 5-amino-4-[4-(morpholinomethyl)phenoxy]-1H-pyrimidin-6-one

Using General Procedure 4 starting from Preparation R3bw as reagent, Preparation R4bw was obtained. HRMS calculated for C₁₅H₁₈N₄O₃: 302.1379; found 303.1450 ((M+H)⁺ form).

¹H-NMR (400 MHz, dmso-d6) δ ppm 12.88 (brs, 1H), 11.53 (brs, 1H), 7.76 (s, 1H), 7.64 (m, 2H), 7.16 (m, 2H), 4.3 (d, 2H), 3.83/3.82 (m+m, 4H), 3.19/3.06 (m+m, 4H)

¹³C-NMR (100 MHz, dmso-d6) δ ppm 140.6, 133.4, 120.4, 63.5, 58.7, 50.9

### Preparation R4bx: tert-butyl 2-[2-[4-[(5-amino-6-oxo-1H-pyrimidin-4-yl)oxy]phenyl]ethyl]piperidine-1-carboxylate

Using General Procedure 4 starting from Preparation R3bx as reagent. 5-amino-4-[4-[2-(2-piperidyl)ethyl]phenoxy]-1H-pyrimidin-6-one hydrochloride was formed. The resulted crude product was reacted using General Procedure 6 to give Preparation R4bx. HRMS calculated for C₂₂H₃₀N₄O₄: 414.22671 found 415.23386 ((M+H)⁺ form).

¹H-NMR (400 MHz, dmso-d6) δ ppm 12.46 (br., 1H), 7.48 (s, 1H), 7.18 (dm, 2H), 6.93 (dm, 2H), 4.54 (br., 2H), 4.14 (br., 1H), 3.85/2.79 (brd+brt, 2H), 2.53/2.42 (m+m, 2H), 1.91/1.7 (m+m, 2H), 1.60-1.44 (br., 2H). 1.57/1.49 (br.+br., 2H), 1.56/1.26 (br.+br., 2H), 1.38 (s, 9H).

¹³C-NMR (100 MHz,. dmso-d6) δ ppm 159.3, 154.6, 153.1, 147.8, 137.4, 135.7, 129.5, 119.8, 78.8, 50.1, 38.7, 31.8, 31.8, 28.6, 28.5, 25.8, 19

### Preparation R4by: tert-butyl 2-[3-[(5-amino-6-oxo-1H-pyrimidin-4-yl)oxy]phenyl]morpholine-4-carboxylate

Using General Procedure 4 starting from Preparation R3by as reagent, 5-amino-4-(3-morpholin-2-ylphenoxy)-1H-pyrimidin-6-one hydrochloride was formed. The resulted crude product was reacted using General Procedure 6 to give Preparation R4by. HRMS calculated for C₁₉H₂₄N₄O₅: 388.1747; found 389.1813 ((M+H)⁺ form).

¹H-NMR (500 MHz, dmso-d6) δ ppm 12.46 (brs, 1H), 7.51 (s, 1H), 7.34 (1H), 7.12 (dd, 1H), 7.02 (t, 1H), 6.97 (dd, 1H), 4.62 (brs, 2H), 4.4 (dd, 1H), 3.93/3.53 (brd+td, 2H), 3.88/2.77 (br.+br., 2H), 3.76/2.96 (br.+br., 2H), 1.41 (s, 9H).

¹³C-NMR (125 MHz, dmso-d6) δ ppm 159.4, 155.3, 154.3, 146.9, 141.6, 135.7, 129.9, 121.9, 121.7, 119.2, 117.4, 79.9, 76.9, 66.5, 49.6, 43.1, 28.5

### Preparation R4bz: tert-butyl N-[(1R)-1-[4-[(5-amino-6-oxo-1H-pyrimidin-4-yl)oxy]phenyl]-2,2,2-trifluoro-ethyl]carbamate

Using General Procedure 4 starting from Preparation R3bz as reagent, 5-amino-4-[4-[(1R)-1-amino-2,2,2-trifluoro-ethyl]phenoxy]-1H-pyrimidin-6-one was formed. The resulted crude product was reacted using General Procedure 6 to give Preparation R4bz. HRMS calculated for C₁₇H₁₉F₃N₄O₄: 400.1358; found 401.1424 ((M+H)⁺ form).

¹H-NMR (500 MHz, dmso-d6) δ ppm 12.46 (s, 1H), 8.36 (d, 1H), 7.54 (m, 2H), 7.5 (s, 1H), 7.04 (m, 2H), 5.39(m, 1H), 4.64 (s, 2H), 1.41 (s, 3H)

¹³C-NMR (125 MHz, dmso-d6) δ ppm 159.4, 146.8, 135.7, 130.1, 119.5, 55.3, 28.5

### Preparation R4ca: 2-[4-[(5-amino-6-oxo-1H-pyrimidin-4-yl)oxy]phenyl]acetonitrile

Using General Procedure 4 starting from Preparation R3ca as reagent. Preparation R4ca was obtained. HRMS calculated for C₁₂H₁₀N₄O₂: 242.0804; found 243.0878 ((M+H)⁺ form).

¹H-NMR (500 MHz, dmso-d6) δ ppm 12.45 (brs, 1H) 7.49 (s, 7.32 (d, 2H), 7.05 (d, 2H), 4.62 (s, 2H), 4 (s, 2H)

¹³ C-NMR (125 MHz, dmso-d6) δ ppm 159.4, 154.6, 147.3, 135.7, 129.6, 126.7, 121.7, 120.3, 119.9, 22.2

### Preparation R4cb: 5-amino-4-[4-fluoro-3-(hydroxymethyl)phenoxy]-1 H-pyrimidin-6-one

Using General Procedure 4 starting from Preparation R3cb as reagent. Preparation R4cb was obtained. HRMS calculated for C₁₁H₁₀FN₃O₃: 251.0706; found 252.0779 ((M+H)⁺ form).

¹H-NMR (500 MHz, dmso-d6) δ ppm 12.99 (br, 1H), 7.86 (s, 1H), 7.19 (d, 1H), 7.18 (t, 1H), 7.04 (dm, 1H), 4.54 (s, 2H)

¹³C-NMR (125 MHz, dmso-d6) δ ppm 159.4, 156.5, 154.9, 149.8, 143.1, 131, 121, 120.9, 116, 56.8

### Preparation R4cc: 5-amino-4-(3-amino-4-fluoro-phenoxy)-1H-pyrimidin-6-one

Using General Procedure 4 starting from Preparation R3cb as reagent, it was purified by Hanbon preparative HPLC,C18 Silica, Gemini NX 5 μm, 5 mM NH₄HCO₃-MeCN using gradient method 5-90%. Solvent was evaporated under reduced pressure to give Preparation R4cb. HRMS calculated for C₁₀H₉FN₄O₂: 236.071; found 237.0782 ((M+H)⁺ form).

¹H-NMR (500 MHz, dmso-d6) δ ppm 12.42 (brs, 1H), 7.5 (s, 1H), 6.91 (t, 1H), 6.39 (dd, 1H), 6.14 (dm, 1H), 5.21 (s, 2H), 4.53 (s, 2H)

¹³C-NMR (125 MHz, dmso-d6) δ ppm 159.3, 151.6, 147.8, 147.5, 137.5, 135.7, 121.4, 115.3, 107.3, 106.6

### Preparation R4cd: 5-amino-4-(3-hydroxy-4-isopropyl-phenoxy)-1H-pyrimidin-6-one

Preparation R3cd (159 mg, 0.55 mmol) was dissolved in abs. DCM (5 mL) and cooled to 0° C., then 1M boron tribromide (3.0 eq.) was added. It was stirred at 0° C. for 1 hour, then it was allowed warm to r.t. After 20 hours, the reaction mixture was cooled to 0° C. and poured to crashed ice and sat. NaHCO₃ was added. It was extracted with ethyl acetate several times. Combined organic phases were dried over magnesium sulfate and evaporated to give Preparation R4cd. HRMS calculated for C₁₃H₁₅N₃O₄: 261.1 113; found 262.1183 (M+H)+ form).

¹H-NMR (500 MHz, dmso-d6) δ ppm 12.44 (s, 1H), 9.38 (s, 1H), 7.51 (s, 1H, 7.03 (d, 1H), 6.43 (d, 1H), 6.41 (dd, 1H). 3.13 (m, 1H), 1.13 (d, 6H)

¹³C-NMR (125 MHz, dmso-d6) δ ppm 159.3, 155.4, 153.6, 147.9, 136, 130, 126.6, 110.1, 106.5, 26.4, 23.1

### Preparation R4ce: tert-butyl 2-[4-[(5-amino-6-oxo-1H-pyrimidin-4-yl)oxy]phenyl]piperidine-1-carboxylate

Using General Procedure 4 starting from Preparation R3ce as reagent, 5-amino-4-[4-(2-(piperidyl)phenoxy]-1H-pyrimidin-6-one hydrochloride was formed. The resulted crude product was reacted using General Procedure 6 to give Preparation R4ce. HRMS calculated for C₂₀H₂₆N₄O₄: 386.1954; found 387.2018 ((M +H)⁺ form).

¹H-NMR (500 MHz, dmso-d6) δ ppm 12.44 (brs, 1H), 7.5 (s, 1H), 7.14 (m, 2H), 7.02 (m, 2H), 5.26 (dd, 1H), 4.59 (s, 2H), 3.92/2.69 (m+m, 2H), 2.27/1.75 (m+m, 2H), 1.4 (s, 9H)

¹³ C-NMR (125 MHz, dmso-d6) δ ppm 135.7, 127.7, 119.8, 52.8, 40.1, 28.5, 28.4

### Preparation R5a: [(1R,2R)-4,4-difluoro-2-phenyl-cyclohexyl]-(2-oxa-6-azaspiro[2.5]octan-6-yl)methanone

### Step 1: (1R,2R)-4,4-difluoro-2-phenyl-cyclohexanecarboxylic acid

2-trimethylsilyloxy-4-phenyl-1,3-butadiene (synthesized according to Tetrahedron 2001, 57, 6311-6327; 1.0 eq.) and ethyl propiolate (1.0 eq.) were placed in a sealed tube into anhydrous toluene. The reaction mixture was heated to 150° C. and it was stirred at this temperature overnight. Then the toluene was evaporated by reduced pressure and the residue was dissolved in a mixture of THF, water, and cc. sulfuric acid (3 eq.) The mixture was stirred for 1 hour at 25° C. Reaction mixture was diluted with water (150 ml) and the product was isolated by extraction with DEE. The organic layer was dried and concentrated. Crude product was used without further purification.

The unsaturated cyclohexenone derivative was placed in a flask and dissolved in cyclohexene. The reaction mixture was refluxed overnight in the presence 0.05 eq. 10% Pd/C. After 16 hours, the Pd/C was filtered off through Celite pad. The saturated crude product was refluxed in methanol in the presence sodium ethoxide to give ethyl trans-4-oxo-2-phenyl-cyclohexanecarboxylate.

Ethyl trans-4-oxo-2-phenyl-cyclohexanecarboxylate was dissolved in DCM, then DAST was added (5.0 eq.). After 1 hour, water and DCM was added, then layers were separated. Organic layer was dried and evaporated. The residue was purified by flash chromatography (hexane:EEO). The enantiomers were separated by chiral chromatography to give ethyl (1R,2R)-4,4-fluoro-2-phenyl-cyclohexanecarboxylate and ethyl (1S,2S)-4,4-difluoro-2-phenyl-cyclohexanecarboxylate.

Ethyl (1R,2R)-4,4-difluoro-2-phenyl-cyclohexanecarboxylate was dissolved in ethanol and water (1:1) and lithium hydroxide hydrate (4.0 eq.) was added. It was heated and stirred at 80° C. for 17 hours. Then the ethanol was evaporated under reduced pressure and 1N HCl was added till solid compound was formed which was filtered of to give (1R,2R)-4,4-difluoro-2-phenyl-cyclohexanecarboxylic acid. HRMS calculated for C₁₃H₁₄F₂O₂: 240.0962; found 258.1302 [(M+NH₄)⁺ form].

¹H-NMR (500 MHz dmso-d6) δ ppm 12.06 (s, 1H), 7.34-7.16 (m, 5H), 2.95 (m, 1H), 2.73 (m, 1H), 2.24-2.00 (m, 2H), 2.20-1.93 (m, 2H), 2.05/1.68 (m+m, 2H)

¹³C-NMR (125 MHz, dmso-d6) δ ppm 47.5, 43.3, 40.2, 32.3, 26.6

### Step 2: 1-[(1R,2R)-4,4-difluoro-2-phenyl-cyclohexanecarbonyl]piperidin-4-one

4-Piperidone hydrochloride hydrate (3.14 g, 20.5 mmol). HBTU (11.66 g. 30.74 mmol), (1R,2R)-4,4-difluoro-2-phenyl-cyclohexanecarboxylic acid. (4.92 g, 20.5 mmol) and N,N-diisapropylethylamine (13.26 g, 17.8 ml, 102.5 mmol) were dissolved in MeCN (50 mL) and stirred at r.t. for 5 hours. After evaporation, the residue was dissolved in DCM and it was washed with 1N NaOH and then with 1N HCl and then with water. Organic layer was dried (MgSO₄) and evaporated. DIPO was added, solid compound was formed, which was filtered off to give the product of the title.

HRMS calculated for C₁₈H₂₁F₂NO₂: 321.154; found 322.1611 [(M+H)+ form].

¹H-NMR (500 MHz, MSM-d6) δ ppm 7.31-7.14 (m, 5H), 3.86-3.16 (m, 4H), 3.31 (m, 1H), 3.09 (m, 1H), 2.3/2.12 (m+m, 2H), 2.23-1.41 (m, 4H), 2.18-1.97 (m, 2H), 1.88/1.77 (m+m, 2H)

¹³ C-NMR (125 MHz, MSM-d6) δ ppm 43.8, 43.1, 39.3, 32.4, 26.7

### Step 3: Preparation R5a

1-[(1R,2R)-4,4-difluoro-2-phenyl-cyclohexanecarbonyl]piperidin-4-one 12.0 g, 6.2 mmol, 1.0 eq.) and trimethylsulfoxonium-iodide (3.4 g, 15.5 mmol. 2.5 eq.) was charged into a round bottom flask and dissolved/suspended in MeCN (10 ml) and MTBE (10 ml). NaOH (0.62 g, 15.5 mmol, 2.5 eq.) was dissolved in water (1.3 ml) and the obtained solution was added to the mixture and stirred at 60° C. for 6 hours. After the reaction completed, the reaction mixture was filtered through Celite, and washed with MTBE (3×4 ml). Water (15 ml) was added to the solution, layers were separated, and the aqueous layer was extracted with MTBE (2×4 ml). Combined organic layers were dried over MgSO₄ and after filtration evaporated to give Preparation R5a. HRMS calculated for C₁₉H₂₃F₂NO₃: 335.1697; found 336.1779 [(M+H)+form].

¹H-NMR (500 MHz, MSM-d6) δ ppm 7.4-7 (m, 5H), 3.85-2.9 (m, 4H), 3.28 (m, 1H), 3.07 (brm. 1H), 2.59-2.5 (m, 2H), 2.36-2.05 (m, 2H), 2.17-1.96 (m, 2H), 1.83/1.74 5 (dm+tm, 2H), 1.38-0.79 (m, 4H).

¹³C-NMR (125 MHz, MSM-d6) δ ppm 57.4/57, 53.4/53, 43.7, 42.8, 39.4, 32.4, 26.7

### Preparation R5b: (8,8-difluoro-2-oxa-6-azaspiro[2.5]octan-6-yl)-[(1R,2R)-4,4-difluoro-2-phenyl-cyclohexyl]methanone

### Step 1: (3,3-difluoro-4,4-dihydroxy-1-piperidyl)-[(1R,2R)-4,4-difluoro-2-phenyl-cyclohexyl]methanone

3,3-difluoropiperidine-4,4-diol hydrochloride (740 mg, 3.903 mmol), 3-(ethyliminomethyleneamino)-N,N-dimethyl-propan-1-amine, hydrochloride (1:1) (2992 mg, 15.613 mmol, 4.0 eq.) and (1R,2R)-4,4-difluoro-2-phenyl-cyclohexanecarboxylic acid (1031 mg, 4.2935 mmol) were dissolved in pyridine (10 mL) and stirred at r.t. for 23 hours. The mixture was purified by preparative HPLC (on C-18 Gemini-NX 5 μm column, 5 mM NH₄HCO₃ aqueous solution—MeCN, gradient). The solvents were removed under reduced pressure to give the product of the title.

### Step 2: Preparation R5b

3,3-difluoro-4,4-dihydroxy-1-piperidyl)-[(1R,2R)-4,4-difluoro-2-phenyl-cyclohexyl]methanone (200 mg, 0.5328 mmol, 1.0 eq.) and trimethylsulfaxonium-iodide (293 mg, 1.332 mmol, 2.5 eq.) was charged into a round bottom flask and dissolved/suspended in MeCN (2 ml) and MTBE (2 ml). NaOH (53 mg. 1.332 mmol, 2.5 eq.) was dissolved in water (0.4 ml) and the obtained solution was added to the mixture and stirred at 60° C. for 2 hours. After the reaction completed. the reaction mixture was cooled down to r.t., MTBE (2 ml) and water (2 ml) were added to the solution layers were separated, and the aqueous layer was extracted with MTBE (3×3 ml). Combined organic layers were dried over MgSO₄ and after filtration evaporated to give Preparation R5b. HRMS calculated for C₁₉H₂₁F₄NO₂: 371.1508; 371.15063[M⁺ form]

¹H-NMR (400/500 MHz, dmso-d6) δ ppm 7.33-7.11 (m, 5H), 4.24-3.35 (m, 2H), 3.78-3.07 (m, 2H), 2.29-1.98 (m, 2H), 2.11-1.98 (m, 2H), 1.93-1.52 (m, 2H), 1.71-1.20 (m, 2H), 3.47/3.4 (m/m, 1H), 3.07 (m, 1H).

¹³C-NMR (400/500 MHz, dmso-d6) δ ppm 172.8, 142.5, 124.1, 117.2, 57.1, 50/46.3, 49.9, 43.6/43.5, 43/39.5, 42.6/41.8, 40.1, 32.3, 30.5/29.7, 26.8

### Preparation R5c: [trans-5,5-difluoro-2-phenyl-cyclohexyl]-(2-oxa-6-azaspiro[2.5]octan-6-yl)methanone

A mixture of cinnamic acid, 1,4-hydroquinone (catalytic amount) were suspended in 1,4-butadiene (20 wt % in toluene), and the resulted mixture was heated together in a sealed tube or microwave vial at 200° C. for 2 hours. After being cooled to r.t., the sealed tube was cooled in an ice/water bath. Solid compound was formed which was littered off, and it was washed with cold toluene three times and dried in air, then in vacuum, to afford trans-6-phenylcyclohex-3-ene-1-carboxylic acid.

A part of this product was dissolved in chloroform and cooled to 0° C. Bromotrimethylsilane (1.0 eq.) in chloroform and MSM was added dropwise to the cooled solution. Then, diisopropylethylamine (1M eq.) was added dropwise at 0° C. to the mixture. It was stirred for 15 minutes at 0° C., was warmed up to r.t., then it was refluxed overnight. Reaction mixture was diluted with EEO, and washed with water, 10% HCl solution, water and finally with brine. The organic layer was dried (MgSO₄) and evaporated. The isolated product was used without further purification.

The isolated product was dissolved in methanol and freshly prepared sodium methoxide (1.0 eq.) was added and the mixture was stirred at 40° C. for 16 hours. The mixture was then treated with 0.5M HCl solution, and methanol was evaporated. The residue was dissolved in EEO and washed with water and the layers were separated. The aqueous layer was extracted with additional EEO, and the combined organic layers were washed with water, 5% Na₂C₃ and brine, and dried (Na₂SO₄) to give methyl trans-5-oxo-2-phenyl-cyclohexanecarboxylate as a crude product.

Methyl trans-5-oxo-2-phenyl-cyclohexanecarbmate was dissolved in DCM, then DAST was added (5.0 eq). After 1 hour, water and DCM was added, then layers were separated. Organic layer was dried and evaporated. The residue was purified by flash chromatography (hexane:EEO).

Then the product obtained from the previous step was dissolved with isopropyl alcohol and 10 cc. HCl (5.0) was added. It was heated and stirred at 90° C. for 4 days, then the solid compound was filtered off and it was purified by preparative HPLC (on C-18 Gemini-NX 5 μm column, 0.02% HCOOH aqueous solution—MeCN, gradient) to give trans-5,5-difluoro-2-phenyl-cyclohexanecarboxylic acid.

Using Steps 6 and 7 or General Procedure 8 and starting from trans-4,4-difluoro-2-(3-thienyl)cyclohexanecarboxylic acid. Preparation R5c was obtained. HRMS calculated for C₁₉H₂₃F₂NO₂: 335.1697; 336.1771 [(M+H)⁺ form]

¹H-NMR (500 MHz, dmso-d6) δ ppm 7.37-7.05 (m, 5H), 3.86-2.88 (m, 4H), 3.29 (m, 1H), 2.91 (m, 1H), 2.55 (m, 2H), 2.21-1.76 (m, 6H), 1.38-0.72 (m, 4H)

¹³C-NMR (125 MHz, dmso-d6) δ ppm 170.7, 143.2, 124.3, 57.3/57, 53.4/53, 45.3/45.2, 42/41.9

### Preparation R5d: [trans-4,4-difluoro-2-pyrrol-1-yl-cyclohexyl]-(2-oxa-6-azaspiro[2.5]octan-6-yl)methanone

Ethyl trans-2-(tert-butoxycarbonylamino)-4-oxo-cyclohexanecarboxlate was synthesized by literature method (Molecules 2015, 20(12), 21094-21102).

XtalFluor-E® (1.5 eq.) was suspended in DCM. Et₃N.3HF (2.0 eq.) was added in 10 minutes followed by Et₃N (1.0 eq.) in 20 minutes. The suspension was dissolved totally. Ethyl (1R,2R)-2-(tert-butoxycarbonylamino)-4-oxo-cyclohexanecarboxylate 1.0 eq.) dissolved in DCM and it was added in 40 minutes to the solution. After stirring 20 hours at r.t., the mixture was neutralized with KHCO₃ (10% in water). Phases were separated and the organic phase was extracted with brine. The combined aqueous phase was extracted with DCM. The combined organic phase was washed with brine dried with MgSO₃ and concentrated under vacuum to give ethyl trans-2-(tert-butoxycarbonylamino)-4,4-difluoro-cyclohexanecarboxylate.

Ethyl trans-2-(tert-butoxycarbonylamino)-4,4-difluoro-cyclohexanecarboxylate was dissolved in DCM and TFA (4.0 eq.) was added. It was stirred at r.t. for 4 hours. Then it was washed with sat. NaHCO₃, the organic phase was dried over MgSO₄ and concentrated under vacuum to give ethyl trans-2-amino-4,4-difluoro-cyclohexanecarboxylate.

Ethyl trans-2-amino-4,4-difluoro-cyclohexanecarboxylate (1.0 eq.) and 2,5-dimethoxytetrahydrofuran (1.0 eq.) and acetic acid (4.5 eq.) were heated at 80° C. for 2 hours, then it was evaporated under reduced pressure. It was dissolved in EtOAc and extracted with sat, NaHCO₃. The organic phase was dried over MgSO₃ and concentrated under vacuum. It was purified by flash chromatography (hexane: EtOAc=9:1) to give ethyl trans-4,4-difluoro-2-pyrrol-1-yl-cyclohexanecarboxylate.

The ethyl trans-4,4-difluoro-2-pyrrol-1-yl-cyclohexanecarboxylate was dissolved in the mixture or ethanol and water (5:1, v/v) and lithium hydroxide hydrate (5.0 eq.) was added. It was stirred at 50° C. for 3 hours. Then the ethanol was evaporated under reduced pressure. 1N HCl was added till pH 1. Solid compound was formed, which was filtered off to give trans-4,4-difluoro-2-pyrrol-1-yl-cyclohexanecarboxylic acid.

Starting from trans-4,4-difluoro-2-pyrrol-1-yl-cyclohexanecarboxylic acid using Steps 6 and 7 of General procedure 8, Preparation R5d was obtained. HRMS calculated for C₁₇H₂₂F₂N₂O₂: 324.1649; 325.1717 [(M+H)⁺ form]

¹H-NMR (500 MHz, dmso-d6) δ ppm 6.79 (m, 2H), 5.96 (m, 2H), 4.32 (m, 1H), 3.93-2.93 (m 4H), 3.5 (m, 1H), 2.65-2.28 (m, 2H), 2.59 (m, 2H), 2.17-1.93 (m, 2H), 1.84/1.64 (m+m, 2H), 1.43-1.06 (m, 4H).

¹³C-NMR (125 MHz, dmso-d6) δ ppm 170.9, 119.9/119.8, 108/107.9, 57.4/57.3, 57.1, 53.4/53.1, 43.5/43.4, 39.8/39.6, 31.9, 25

### Preparation R5e: [trans-4,4-difluoro-2-(2-pyridyl)cyclohexyl]-(2-oxa-6-azaspiro[2.5]octan-6-yl)methanone

Using General Procedure 8 and starting from pyridine-2-carbaldehyde. Preparation R5e was obtained. HRMS calculated for C₁₈H₂₂F₂N₂O₂: 336.1649; found 337.1717 ((M+H)⁺ form).

Preparation R5g: [trans-4,4-difluoro-2-(5-methyl-3-thienyl)cyclohexyl]-(2-oxa-6-azaspiro[2.5]octan-6-yl)methanone

Using General Procedure 8 and starting from 5-methylthiophene-3-carbaldehyde, Preparation R5g was obtained. HRMS calculated for C₁₈H₂₃F₂NO₂S: 355.1418; found 356.149 ((M+H)⁺ form).

### Preparation R5i: [trans-2-(1-ethylpyrazol-4-yl)-4,4-difluoro-cyclohexyl]-(2-oxa-6-azaspiro[2.5]octan-6-yl)methanone

Using Step 1 of General Procedure 8 and starting from 1-ethyl-1H-pyrazole-4-carbaldehyde, (E)-4-(1-ethyl-1H-pyrazol-4-yl)but-3-en-2-one was obtained.

It was dissolved in DCM and DBU (1.3 eq.) was added. Then, TMSCl (1.2 eq.) was added dropwise at 0° C. The solution was stirred for 2 hours at 40° C. then cooled and washed with NaHCO₃ solution 3 times. The organic layer was dried over MgSO₄, then the solvent was evaporated under reduced pressure.

(E)-1-ethyl-4-(4-(3-((trimethylsily)oxy)buta-1,3-dien-1-yl)-1H-pyrazole was used without further purification according to Steps 3 to 7 of General Procedure 8 to give Preparation R5i. HRMS calculated for C₁₈H₂₅F₂N₃O₂: 353.1915; found 354.1979 ((M+H)⁺ form).

Preparation R5k: [trans-4,4-difluoro-2-(2-furyl)cyclohexyl]-(2-oxa-6-azaspiro[2.5]octan-6-yl)methanone

Using General Procedure 8 and starting from furan-2-carbaldehyde. Preparation R5k was obtained. HRMS calculated for C₁₇H₂₁F₂NO₃: 325.149; found 326.1558 ((M+H)⁺ form).

Preparation R5l: [trans-4,4-difluoro-2-(3-thienyl)cyclohexyl]-2-oxa-6-azaspiro[2.5]octan-6-yl)methanone

Using General Procedure 8 and starling from 3-thiophenecarboxaldehyde. Preparation R5l was obtained. HRMS calculated for C₁₇H₂₁F₂NO₂S: 341.1261; found 342.1329 ((M+H)⁺ form).

### EXAMPLES

The following Examples illustrate the invention but do not limit it in any way.

### 5-amino-3-({1-[(1R,2R)-4,4-difluoro-2-phenylcyclohexane-1-carbonyl]-4-hydroxypiperidin-4-yl}methyl)-6-phenoxypyrimidin-4(3H)-one (EXAMPLE 1)

Using General Procedure 5 starting from Preparation R4a and Preparation R5a as reagents, EXAMPLE 1 was obtained. HRMS calculated for C₂₉H₃₂N₄O₄F₂: 538.2391; found 539.2465 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(2-fluorophenoxy)pyrimidin-4(3H)-one

### Example 2

Using General Procedure 5 starting from Preparation R4b and Preparation R5a as reagents, EXAMPLE 2 was obtained. HRMS calculated for C₂₉H₃₁N₄O₄F₃: 556.2297; found 557.2362 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(4-methoxyphenoxy)pyrimidin-4(3H)-one

### Example 3

Using General Procedure 5 starting from Preparation R4c and Preparation R5a as reagents, EXAMPLE 3 was obtained. HRMS calculated for C₃₀H₃₄N₄O₅F₂: 568.2498; found 569.257 ((M+H)⁺ form).

5-amino-3-({1-[(1R,2R)-4,4-difluoro-2-phenylcyclohexane-1-carbonyl]-4-hydroxypiperidin-4-yl}methyl)-6-(3-methoxyphenoxy)pyrimidin-4(3H)-one

### Example 4

Using General Procedure 5 starting from Preparation R4d and Preparation R5a as reagents, EXAMPLE 4 was obtained. HRMS calculated for C₃₀H₃₄N₄O₅F₂: 568.2498; found 569.257 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(4-fluorophenoxy)pyrimidin-4(3H)-one

### Example 5

Using General Procedure 5 starting from Preparation R4e and Preparation R5a as reagents. EXAMPLE 5 was obtained. HRMS calculated for C₂₉H₃₁N₄F₃: 556.2297; found 557.237 ((M+H)⁺ form).

### 5-amino-3-({1-[(1R,2R)-4,4-difluoro-2-phenylcyclohexane-1 -carbonyl]-4-hydroxypiperidin-4-yl}methyl)-6-(3-fluorophenoxy)pyrimidin-4(3H)-one

### Example 6

Using General Procedure 5 starting from Preparation R4f and Preparation R5a as reagents, EXAMPLE 6 was obtained. HRMS calculated for C₂₉H₃₁N₄O₄F₃: 556.2297; found 557.2361 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(3,5-dimethoxyphenoxy)pyrimidin-4(3H)-one

### Example 7

Using General Procedure 5 starting from Preparation R4g and Preparation R5a as reagents. EXAMPLE 7 was obtained. HRMS calculated for C₃₁H₃₆N₄O₆F₂: 598.2603; found 599.267 ((M+H)⁺ form).

### 5-amino-6-(3,5-difluorophenoxy)-3-({1-[(1R,2R)-4,4-difluoro-2-phenylcyclohexane-1-carbonyl]-4-hydroxypiperidin-4-yl}methyl)pyrimidin-4(3H)-one (EXAMPLE 8)

Using General Procedure 5 starting from Preparation R4h and Preparation R5a as reagents, EXAMPLE 8 was obtained. HRMS calculated for C₂₉H₃₀N₄O₄F₄: 574.2203; found 575.2277 ((M+H)⁺ form).

### 3-({1-[(1R4R)-4,4-difluoro-2-phenylcyclohexane-1-carbonyl]-4-hydroxypiperidin-4-yl}methyl)-5-(methylamino)-6-phenoxypyrimidin-4(3H)-one (EXAMPLE 9)

Using General Procedure 5 starting from Preparation R4i and Preparation R5a as reagents. EXAMPLE 9 was obtained. HRMS calculated for C₃₀H₃₄N₄O₄F₂: 552.2548; found 553.2618 ((M+H)⁺ form).

### 5-amino-3-[[(4R)-1-[(1R,2R)-4,4-difluoro-2-phenyl-cyclohexanecarbonyl]-3,3-difluoro-4-hydroxy-4-piperidyl]methyl]-6-phenoxy-pyrimidin-4-one (EXAMPLE 10)

Using General Procedure 5 starting from Preparation R4a and Preparation R5b as reagents. EXAMPLE 10 was obtained. HRMS calculated for C₂₉H₃₀N₄O₄F₄: 574,2203; found 575.2276 ((M+H)⁺ form).

### 5-amino-6-(4-chlorophenoxy)-3-({1-[(1R,2R)-4,4-difluoro-2-phenylcyclohexane-1-carbonyl]-4-hydroxypiperidin-4-yl}methyl)pyrimidin-4(3H)-one (EXAMPLE 11)

Using General Procedure 5 starting from Preparation R4j and Preparation R5a as reagents, EXAMPLE 11 was obtained. HRMS calculated for C₂₉H₃₁N₄O₄F₂Cl: 572.2002; found 573.2069 ((M+H)⁺ form).

### 5-amino-6-(4-chloro-3-methoxy-phenoxy)-3-({1-[(1R,2R)-4,4-difluoro-2-phenylcyclohexane-1-carbonyl]-4-hydroxypiperidin-4-yl}methyl)pyrimidin-4(3H)-one

### Example 12

Using General Procedure 5 starting from Preparation R4k and Preparation R5a as reagents, EXAMPLE 12 was obtained. HRMS calculated for C₃₀H₃₃N₄O₅F₂Cl: 602.2108; found 603.2183 ((M+H)⁺ form)

### 5-amino-6-(3-chlorophenyl)-3-({1-[(1R,2R)-4,4-difluoro-2-phenylcyclohexane-1-carbonyl]-4-hydroxypiperidin-4-yl}methyl)pyrimidin-4(3H)-one (EXAMPLE 13)

Using General Procedure 5 starting from Preparation R4l and Preparation R5a as reagents, EXAMPLE 13 was obtained. HRMS calculated for C₂₉H₃₁N₄O₄F₂Cl: 572.2002; found 573.2079 ((M+H)⁺ form).

### 5-amino-6-[(2H-1,3-benzedioiol-5-yl)oxy[-3-({1-[(1R,2R)-4,4-difluoro-2-phenylcyclohexane-1-carbonyl]-4-hydroxypiperidin-4-yl}methyl)pyrimidin-4(3H)-one

### Example 14

Using General Procedure 5 starting from Preparation R4n and Preparation R5a as reagents. EXAMPLE 14 was obtained. HRMS calculated for C₃₀H₃₂N₄O₆F₂: 582.229; found 583.2378 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(3-hydroxy-5-methoxyphenoxy)pyrimidin-4(3H)-one (EXAMPLE 15)

Using General Procedure 5 starting from Preparation R4o and Preparation R5a as reagents, EXAMPLE 15 was obtained. HRMS calculated for C₃₀H₃₄N₄O₆F₂: 584.2446; found 585.2524 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(4-fluoro-3-methoxyphenoxy)pyrimidin-4(3H)-one

### Example 16

Using General Procedure 5 starting from Preparation R4p and Preparation R5a as reagents, EXAMPLE 16 was obtained. HRMS calculated for C₃₀H₃₃N₄O₅F₃: 586.2403; found 587.2468 ((M+H)⁺ form)

### 3-({1-[(1R,2R)-4,4-difluoro-2-phenylcyclohexane-1-carbonyl]-4-hydroxypiperidin-4-yl}methyl)-6-phenoxy-5-[(2,2,2-trifluoroethyl)amino]pyrimidin-4(3H)-one

### Example 17

Using General Procedure 5 starting from Preparation R4q and Preparation R5a as reagents, EXAMPLE 17 was obtained. HRMS calculated for C₃₁H₃₃N₄O₄F₅: 620.2422; found 621.2493 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-(trifluoromethoxy)phenoxy]pyrimidin-4(3H)-one

### Example 18

Using General Procedure 5 starting from Preparation R4r and Preparation R5a as reagents, EXAMPLE 18 was obtained. HRMS calculated for C₃₀H₃₁N₄O₅F₅: 622.2214; found 623.2286 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(3-methylphenoxy)pyrimidin-4(3H)-one

### Example 19

Using General Procedure 5 starting from Preparation R4s and Preparation R5a as reagents, EXAMPLE 19 was obtained. HRMS calculated for C₃₀H₃₄N₄O₄F₂: 552.2548; found 553.2625 ((M+H)⁺ form).

### 5-amino-6-(3-bromophenoxy)-3-([1-{(1R,2R)-4,4-difluoro-2-phenylcyclohexane-1-carbonyl]-4-hydroxypiperidin-4-yl}methyl)pyrimidin-4(3H)-one EXANIPLE 20)

Using General Procedure 5 starting from Preparation R4t and Preparation R5a as reagents. EXAMPLE 20 was obtained. HRMS calculated for C₂₉H₃₁N₄O₄F₂Br: 616.1497; found 617.1568 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-(pentafluoro-λ⁶-sulfanyl)phenoxy]pyrimidin-4(3H)-one (EXAMPLE 21)

Using General Procedure 5 starting from Preparation R4u and Preparation R5a as reagents, EXAMPLE 21 was obtained. HRMS calculated for C₂₉H₃₁N₄O₄F₇S: 664.1954; found 665.2018 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-(trifluoromethyl)phenoxy]pyrimidin-4(3H)-one

### Example 22

Using General Procedure 5 starting from Preparation R4v and Preparation R5a as reagents, EXAMPLE 22 was obtained. HRMS calculated for C₃₀H₃₁N₄O₄F₅: 606.2266; found 607.2339 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[4-(hydroymethyl)phenoxy]pyrimidin-4(3H)-one

### Example 23

Using General Procedure 5 starting from Preparation R4w and Preparation R5a as reagents. EXAMPLE 23 was obtained. HRMS calculated for C₃₀H₃₄F₂N₄O₅: 568.2498; found 569.2559 ((M+H)⁺ form).

### 4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}benzonitrile

### Example 24

Using General Procedure 5 starting from Preparation R4z and Preparation R5a as reagents, EXAMPLE 24 was obtained. HRMS calculated for C₃₀H₃₁F₂N₅O₄: 563.2344; found 564.2421 ((M+H)⁺ form).

### 3-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}benzonitrile

### Example 25

Using General Procedure 5 starting from Preparation R₄aa and Preparation R₅a as reagents, EXAMPLE 25 was obtained. HRMS calculated for C₃₀H₃₁F₂N₅O₄: 563.2344; found 564.24 ((M+H)⁺ form)

### 5-amino-3-({1-[(1R,2R)-4,4-difluoro-2-phenylcyclohexane-1-carbonyl]-4-hydroxypiperidin-4-yl}methyl)-6-[(1,2,3,4-tetrahydroisoquinolin-7-yl)oxy]pyrimidin-4(3H)-one, hydrochloride (EXAMPLE 26)

Using General Procedure 5 starling from Preparation R₄ab and Preparation R₅a as reagents. tert-butyl 7-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxy-3,4-dihydro-1H-isoquinoline-2-carboxylate was formed. The resulted Boc-protected crude product was reacted using General Procedure 7 to give EXAMPLE 26 as HCl salt. HRMS calculated for C₃₂H₃₇F₂N₅O₄: 593.2814; found 594.2883 ((M+H)⁺ form).

### methyl 3-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}benzoate

### Example 27

Using General Procedure 5 starting from Preparation R4ac and Preparation R5a res reagents. EXAMPLE 27 was obtained. HRMS calculated for C₃₁H₃₄F₂N₄O₆: 596.2446; found 597.252 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-(hydroxymethyl)phenoxy]pyrimidin-4(3H)-one

### Example 28

Using General Procedure 5 starting from Preparation R₄ad and Preparation R₅a as reagents. EXAMPLE 28 was obtained. HRMS calculated for C₃₀H₃₄F₂N₄O₅: 568.2498; found 569.2563 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[4-(3-hydroxypropyl)phenoxy]pyrimidin-4(3H)-one

### Example 29

Using General Procedure 5 starting from Preparation R₄ae and Preparation R₅a as reagents, EXAMPLE 29 was obtained. HRMS calculated for C₃₂H₃₈F₂N₄O₅: 596.281; found 597.2878 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(phenylsulfanyl)pyrimidin-4(3H)-one (EXAMPLE 30)

Using General Procedure 5 starting from Preparation R4af and Preparation R5a as reagents, EXAMPLE 30 was obtained. HRMS calculated for C₂₉H₃₂F₂N₄O₃S: 554.2163; found 555.2232 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[(1,2,3,4-tetrahydroisoquinolin-6-yl)oxy]pyrimidin-4(3H)-one (EXAMPLE 31)

Using General Procedure 5 starting from Preparation R4ag and Preparation R5a as reagents tert-butyl 6-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxy-3,4-dihydro-1H-isoquinoline-2-carboxylate was formed. The resulted Boc-protected crude product was reacted using General Procedure 7 to give EXAMPLE 31. HRMS calculated for C₃₂H₃₇F₂N₅O₄: 593.2814; found 594.2885 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[(2,3-dihydro-1H-isoindol-5-yl)oxy]pyrimidin-4(3H)-one (EXAMPLE 32)

Using General Procedure 5 starting from Preparation R₄ah and Preparation R₅a as reagents, tert-butyl 5-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxyisoindoline-2-carboxylate was formed. The resulted Boc-protected crude product was reacted using General Procedure 7 to give EXAMPLE 32. HRMS calculated for C₃₁H₃₅F₂N₅O₄: 579.2657; found 580.2717 ((M+H)⁺ form),

### 5-amino-3-({1-[(1R,2R)-4,4-difluoro-2-phenylcyclohexane-1-carbonyl]-4-hydroxypiperidin-4-yl}methyl)-6-[4-(2-hydroxyethoxy)phenoxy]pyrimidin-4(3H)-one

### Example 33

Using General Procedure 5 starting from Preparation R4ai and Preparation R5a its reagents, EXAMPLE 33 was obtained. HRMS calculated for C₃₁H₃₆F₂N₄O₆: 598.2603; found 599.2676 ((M+H)⁺ form),

### 5-amino-3({1-[(1R,2R)-4,4-difluoro-2-phenylcyclohexane-1-carbonyl]-4-hydroxypiperidin-4-yl}methyl)-6-[4-(pyrrolidin-2-yl)phenoxy]pyrimidin-4(3H)-one

### Example 34

Using General Procedure 5 starting from Preparation R4aj and Preparation R5a as reagents. tert-butyl 2-[4-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxyphenyl]pyrrolidine-1-carboxylate was formed. The resulted Boc-protected crude product was reacted using General Procedure 7 to give EXAMPLE 34, HRMS calculated for C₃₃H₃₉F₂N₅O₄: 607.297; found 608.3026 ((M+H)⁺ form).

### 5-amino-3-({1-[(1R,2R)-4,4-difluoro-2-phenylcyclohexane-1 -carbonyl]-4-hydroypiperidin-4-yl}methyl)-6-[(1H-indazol-6-yl)oxy]pyrimidin-4(3H)-one

### Example 35

Using General Procedure 5 stoning, from Preparation R4al and Preparation R5a as reagents. EXAMPLE 35 was obtained. HRMS calculated for C₃₀H₃₂F₂N₆O₄: 578.2453; found 579.2525 ((M+H)⁺ form).

### 5-amino-3-({1-[(1R,2R)-4,4-difluoro-2-phenylcyclohexane-1-carbonyl]-4-hydroxypiperidin-4-}methyl)-6-[4-(2-hydroxyethyl)phenoxy]pyrimdin-4(3H)-one

### Example 36)

Using General Procedure 5 starting from Preparation R4am and Preparation R5a as reagents, EXAMPLE 36 was obtained. HRMS calculated for C₃₁H₃₆F₂N₄O₅: 582.2654; found 583.2725 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[4-(2,2,2-trifluoroethyl)phenoxyl]pyrimidin-4(3H)-one (EXAMPLE 37)

Using General Procedure S starting from Preparation R4an and Preparation R5a as reagents, EXAMPLE 37 was obtained. HRMS calculated for C₃₁H₃₃F₅N₄O₄: 620.2422; found 621.2493 ((M+H)⁺ form).

### 5-amino-6-[4-(2,2-difluoroethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 38

Using General Procedure 5 starting from Preparation R4ao and Preparation R5a as reagents, EXAMPLE 38 was obtained. HRMS calculated for C₃₁H₃₄F₄N₄O₄: 602.2516; found 603.2594 ((M+H)⁺ form).

### 5-amino-3-({1-[(1R,2R)-4,4-difluoro-2-phenylcyclohexane-1-carbanyl]-4-hydroxypiperidin-4-yl}methyl)-6-[4-(2-fluoroethyl)phenoxy]pyrimidin4(3H)-one

### Example 39

Using General Procedure 5 starting from Preparation R4ap and Preparation R5a as reagents. EXAMPLE 39 was obtained. HRMS calculated for C₃₁H₃₅F₃N₄O₄: 584.261; found 585.2689 ((M+H)⁺ form).

### 5-amino-3-({1-[(1R,2R)-4,4-difluoro-2-phenylcyclohexane-1 -carbonyl]-4-hydroxypiperidin-4-yl}methyl)-6-[4-fluoro-3-(trifluoromethoxy)phenoxy]pyrimidin-4(3H)-one (EXAMPLE 40)

Using General Procedure 5 starting from Preparation R4aq and Preparation R5a as reagents. EXAMPLE 40 was obtained. HRMS calculated for C₃₀H₃₀F₆N₄O₅: 640.212; found 641.2183 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(4-fluoro-3-hydroxyphenoxy)pyrimidin-4(3H)-one

### Example 41

Using General Procedure 5 starting from Preparation R4ar and Preparation R5a as reagents, EXAMPLE 41 was obtained. HRMS calculated for C₂₉H₃₁F₃N₄O₅: 572.2247; found 573.2319 ((M+H)⁺ form).

### 5-amino-6-(4-chloro-3-ethylphenoxy)-3-({1-[(1R,2R)-4,4-difluoro-2-phenylcyclohexane-1-carbonyl]-4-hydroxypiperidin-4-yl}methyl)pyrimidin-4(3H)-one

### Example 42

Using General Procedure 5 starting from Preparation R4as and Preparation R5a as reagents, EXAMPLE 42 was obtained. HRMS calculated for C₃₁H₃₅ClF₂N₄O₄: 600.2315; found 601.2385 ((M+H)⁺ form).

### 5-amino-6-[3-(benzyloxy)phenoxy]-3-([1-[(1R,2R)-4,4-difluoro-2-phenylcyclohexane-1-carbonyl]-4-hydroxypiperidin-4-yl]methyl)pyrimidin-4(3H)-one

### Example 43

Using General Procedure 5 starting from Preparation R4at and Preparation R5a as reagents, EXAMPLE 43 was obtained. HRMS calculated for C₃₆H₃₈F₂N₄O₅: 644.281; found 645.2891 ((M+H)⁺ form).

### 4-{[(5-amino-1-([1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy]benzaldehyde

### Example 44

Using General Procedure 5 starting from Preparation R4au and Preparation R5a as reagents, EXAMPLE 44 was obtained. HRMS calculated for C₃₀H₃₂F₂N₄O₅: 566.2341; found 567.2407 ((M+H)⁺ form).

### 5-amino-3-({1-[(1R,2R)-4,4-difluoro-2-phenylcyclohexane-1-carbonyl]-3,3-difluoro-4-hydroxypiperidin-4-yl}methyl)-6-(4-fluorophenoxy)pyrimidin-4(3H)-one

### Example 45

Using General Procedure 5 starting from Preparation R4e and Preparation R5b as reagents, EXAMPLE 45 was obtained. HRMS calculated for C₂₉H₂₉F₅N₄O₄: 592.2109; found 593.2177 ((M+H)⁺ form).

### 4-{[5-amino-({1-[(1R,2R)-4,4-difluoro-2-phenylcyclohexane-1-carbonyl]-3,3-difluoro-4-hydroxypiperidin-1-yl}methyl)-6-oxo-1,6-dihydropyrimidin-4-yl]oxy}benzonitrile, racemic (EXAMPLE 46)

Using General Procedure 5 starting from Preparation R4z and Preparation R5b as reagents. EXAMPLE 46 was obtained.

¹H-NMR (500 MHz, dmso-d6) δ ppm 7.85 (m, 2H), 7.7/7.66 (s/s, 1H), 7.19 (m, 2H), 6.08/6.03 (s/s, 1H), 4.97/4.93 (s/s, 2H), 4.61/4.45/3.92/3.76 (d/d+d/d, 2H), 3.42/3.37 (td/td, 1H), 3.04 (m, 1H), 1.54/1.17 (m, 2H)

¹³C-NMR (125 MHz, dmso-d₆) δ ppm 159.5/159.4, 159/158.9, 139.1/139, 134.6, 124.1, 120.1, 119.7, 119.3, 106, 47.5/47.2, 44/43.4, 42.4/41.9, 32.7/31.6, 26.5/26.4

### 5-amino-3-({1-[(1R,2R)-4,4-difluoro-2-phenylcyclohexane-1-carbonyl]-4-hydroxypiperidin-4-yl}methyl)-6-[3-(piperidin-2-yl)phenoxy]pyrimidin-4(3H)-one

### Example 47

Using General Procedure 5 starting from Preparation R4az and Preparation R5a as reagents, tert-butyl 2-[3-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxyphenyl]piperidine-1-carboxylate was formed. The resulted Boc-protected crude product was reacted using General Procedure 7 to give EXAMPLE 47. HRMS calculated for C₃₄H₄₁F₂N₅O₄: 621.3127; found 622.3198 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[(2-hydroxy-2,3-dihydro-1H-inden-5-yl)oxy]pyrimidin-4(3H)-one (EXAMPLE 48)

Using General Procedure 5 starting from Preparation R4bc and Preparation R5a as reagents, EXAMPLE 48 was obtained. HRMS calculated for C₃₂H₃₆F₂N₄O₅: 594.2654; found 595.2722 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-(2-hydroxypropan-2yl)phenoxy]pyrimidin-4(3H)-one (EXAMPLE 49)

Using General Procedure 5 starting from Preparation R4bf and Preparation R5a as reagents, EXAMPLE 49 was obtained. HRMS calculated for C₃₂H₃₈F₂N₄O₅: 596.281; found 619.2695 ((M+Na)⁺ form).

### 4-{[5-amino-1-({1-[(1R,2R)-4,4-fluoro-2-phenylcyclohexane-1-carbonyl]-4-hydroxypiperidin-4-yl}methyl)-6-oxo-1,6-dihydropyrimidin-4-yl]oxy}-2-fluorobenzonitrile (EXAMPLE 50)

Using General Procedure 5 starting from Preparation R4bg and Preparation R5a as reagents, EXAMPLE 50 was obtained. HRMS calculated for C₃₀H₃₀F₃N₅O₄: 581.225; found 582.2324 ((M+H)⁺ form).

### 4-{[5-amino-({1-[(1R,2R)-4,4difluoro-2-phenylcyclohexane-1-carbonyl]-4-hydroxypiperidin-4-yl}methyl)-6-oxo-1,6-dihydropyrimidin-4-yl]oxy}-2-chlorobenzonitrile (EXAMPLE 51)

Using General Procedure 5 starting from Preparation R4bh and Preparation R5a as reagents. EXAMPLE 51 was obtained. HRMS calculated for C₃₀H₃₀ClF₂N₅O₄: 597.1954; found 598.2019 ((M+H)⁺ form).

### 3-(4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}-2-chlorophenyl)propanenitrile (EXAMPLE 52)

Using General Procedure 5 starting from Preparation R4bj and Preparation R5a as reagents, EXAMPLE 52 was obtained. HRMS calculated for C₃₂H₃₅F₂N₅O₄: 591.2657; found 592.2728 ((M+H)⁺ form).

### 4-{[5-amino-1-([1-[(1R,2R)-4,4-difluoro-2-phenylcyclohexane-1-carbanyl]-4-hydroxypiperidin-4-yl]methyl)-6-oxo-1,6-dihydropyrimidin-4-yl]oxy}-3-chlorobenzonitrile (EXAMPLE 53)

Using General Procedure 5 starting from Preparation R4bk and Preparation R5a as reagents. EXAMPLE 53 was obtained. HRMS calculated for C₃₀H₃₀ClF₂N₅O₄: 597.1954; found 598.2031 ((M+H)⁺ form).

### 5-amino-6-[4-(1-aminoethyl)phenoxy]-3-([1-[(1R,2R)-4,4-difluoro-2-phenylcyclohexane-1-carbonyl]-4-hydroxypiperidin-4-yl]methyl)pyrimidin-4(3H)-one, diastereoisomer 1 (EXAMPLE 54) and

### 5-amino-6-[4-(1-aminoethyl)phenoxy]-3({1-[(1R,2R)-4,4-difluoro-2-phenylcyclohexane-1-carbonyl]-4-hydroxypiperidin-4-yl}methyl)pyrimidin-4(3H)-one, diastereoisomer 2 (EXAMPLE 55)

Using General Procedure 5 starting from Preparation R4bm and Preparation R5a as reagents. tert-butyl N-[1-[4-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxyphenyl]ethyl]carbamate was formed. tert-Butyl N-[1-[4-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]ethyl]carbamate, diastereoisomer 1 and tert-butyl N-[1-[4-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]ethyl]carbamate, diastereoisomer 2 were obtained separately by chiral chromatography.

tert-Butyl N-[1-[4-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]ethyl]carbamate, diastereoisomer 1 was reacted using General Procedure 7 to give EXAMPLE 54.

tert-Butyl N-[1-[4-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]ethyl]carbamate, diastereoisomer 2 was reacted using General Procedure 7 to give EXAMPLE 55. HRMS calculated for C₃₁H₃₇F₂N₅O₄: 581.2814; found 582.2883 ((M+H)⁺ form) for both diastereoisomers.

### 5-amino-6-[4-(1-aminopropyl)phenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 56

Using General Procedure 5 starling from Preparation R4bn and Preparation R5a as reagents, tert-butyl N-[1-[4-[5-amino-1-[[1-[(1R,2R)-4,4-difluoro-2-phenyl-cyclohexane carbonyl]-4-hydroxy-4-piperidyl]methyl]-6-oxo-pyrimidin-4-yl]oxyphenyl]propyl]carbamate was formed. The resulted Boc-protected crude product was reacted using General Procedure 7 to give EXAMPLE 56. HRMS calculated for C₃₂H₃₉F₂N₅O₄: 595.297; found 596.3038 ((M+H)⁺ form).

### 5-(benzylamino)-3-({1-[(1R,2R)-4,4-difluoro-2-phenylcyclohexane-1-carbonyl]-4-hydroxypiperidin-4-yl}methyl)-6-(4-fluorophenoxy)pyrimidin-4(3H)-one

### Example 57

EXAMPLE 5 (100 mg, 0.1797 mmol), benzaldehyde (2.0 eq.), sodium triacetoxyborohydride (5.0 eq.), acetic acid (2.0 eq.) were dissolved in THF and heated and stirred at 70° C. for 2 days. The reaction mixture was purified by preparative LC (on C-18 Gemini-NX 5 μm column. 5 mM aqueous NH₄HCO₃-MeCN, gradient), then it was repurified by preparative LC (on C-18 Gemini-NX 5 μm column. 0.2% aqueous formic acid solution-MeCN, gradient). Solvent was evaporated under reduced pressure to give EXAMPLE 57. HRMS calculated for C₃₆H₃₇F₃N₄O₄: 646.2767; found 647.2839 ((M+H)⁺ form).

### 4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}benzamide

### Example 58

EXAMPLE 24 (80 mg, 0.1419 mmol), (1E)-acetaldehyde oxime (83.84 mg, 0.0865 mL, 1.419 mmol, 10 eq.). Cu²⁺ on 4 Å molecular sieve (100 mg) were dissolved in methanol (3 mL) and 1,4-dioxane (2 mL). The reaction mixture was then warmed up to 60° C. and stirred at that temperature for 3 days. The mixture was filtered, the filtrate was evaporated and purified by preparative LC (on C-18 Gemini-NX 5 μm column, 5 mM aqueous NH₄HCO₃-MeCN, gradient). Solvent was evaporated under reduced pressure to give EXAMPLE 58. HRMS calculated for C₃₀H₃₃F₂N₅O₅: 581.245; found 582.2533 ((M+H)⁺ form).

### 5-amino-6-[4-(aminomethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 591

Autoclave was charged with EXAMPLE 24 (100 mg, 0.1774 mmol). Raney-nickel catalyst (100 mg) and 7N ammonia in methanol (5 mL) and 1,4-dioxane (5 mL) and then placed under a nitrogen atmosphere. After that it was filled with 10 bar H₂ gas. The reaction mixture was stirred in autoclave at r.t. for 20 hours. The reaction mixture was removed from the autoclave and filtered. The nitrate was purified by preparative LC (on C-18 Gemini-NX 5 μm column. 5 mM aqueous NH₄HCO₁-MeCN, gradient). Solvent was evaporated under reduced pressure to give EXAMPLE 59, HRMS calculated for C₃₀H₃₅F₂N₅O₄: 567.2657; found 568.2735 ((M+H)⁺ form).

### 5-amino-3-({1-((1R,2R)-4,4-difluoro-2-phenylcyclohexane-1-carbony]-4-hydroxypiperidin-4-yl}methyl)-6-{4-[(methylamino)methyl]phenoxy}pyrimidin-4(3H)-one ((EXAMPLE 60)

EXAMPLE 44 (80 mg, 0.1412 mmol), methylamine (2M in THE) (20 eq.), sodium triacetoxyborohydride (5.0 eq.), acetic acid (5.0 eq.) were dissolved in THF and stirred r.t. for 24 hours. The reaction mixture was evaporated, dissolved in DMF/methanol and it was purified by preparative LC (on C-18 Gemini-NX 5 μm column, 5 mM aqueous NE₄HCO₃-MeCN, gradient). Solvent was evaporated under reduced pressure to give EXAMPLE 60, HRMS calculated for C₃₁H₃₂F₂N₅O₄: 581.2814; found 582.2878 ((M+H)⁺ form).

### 5-amino-6-[3-(aminomethyl)phenoxy]-3-({1-[(1R,2R)-4,4-difluoro-2-phenylcyclohexane-1-carbonyl]-4-hydroxypiperidin-4-hydroxypiperidin-4-yl}methyl)pyrimidin-4(3H)-one

### Example 61

Autoclave was charged with EXAMPLE 25 (100 mg, (1.1774 mmol), Raney-nickel catalyst (150 mg) and 7N ammonia in methanol (5 mL) and 1,4-dioxane (5 mL) and then placed under a nitrogen atmosphere. After that it was filled with 10 bar H₂ gas. The reaction mixture was stirred in autoclave at r.t. for 20 hours. The reaction mixture was removed from the autoclave and filtered. The filtrate was purified by preparative LC (on C-18 Gemini-NX 5 μm column, 5 mM aqueous NH₄HCO₃-MeCN, gradient). Solvent was evaporated under reduced pressure to give EXAMPLE 61. HRMS calculated for C₃₀H₃₅F₂N₅O₄: 567.2657; found 568.2723 ((M+H)⁺ form).

### 3-{[5-amino-1-([1-[(1R,2R)-4,4-difluoro-2-phenylcyclohexane-1-carbonyl]-4-hydroxypiperidin-4-yl]methyl)-6-oxy-1,6-dihydropyrimidin-4-yl]oxy}benzamide

### Example 62

EXAMPLE 25 (100 mg, 0.1774 mmol, (1E)-acetaldehyde oxime (104.8 mg, 0.108 mL, 1.774 mmol, 10 eq.). Cu²⁺ on 4 Å molecular sieve (100 mg) were dissolved in methanol (3 mL) and 1,4-dioxane 12 mL). The reaction mixture was then warmed up to 60° C. and stirred at that temperature for 70 hours. The mixture was filtered. the filtrate was evaporated and purified by preparative LC (on C-18 Gemini-NX 5 μm column, 5 mM aqueous NH₄HCO₃-MeCN, gradient). Solvent was evaporated under reduced pressure to give EXAMPLE 62. HRMS calculated for C₃₀H₃₃F₂N₅O₅: 581.245; found 582.2528 ((M+H)⁺ form).

### 3-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-1-yl]oxy}benzoic acid

### Example 63

EXAMPLE 27 (100 mg. 0.1676 mmol), lithium-hydroxide monohydrate (28.13 mg, 0.6705 mmol, 4.0 eq.) were stirred in methanol (3 ml) and water (3 ml) at r.t. for 15 hours. The mixture was partially evaporated and the aqueous residue was acidified with 1N HCl (670 μL, aq.). The resulted precipitate was filtered off, washed with water and dried to give EXAMPLE 63. HRMS calculated for C₃₀H₃₂F₂N₄O₆: 582.229; found 583.2358 ((M±H)⁺ form).

### 5-amino-3-({1-[(1R,2R)-4,4-difluoro-2-phenylcyclohexnne-1-carbonyl]-4-hydroxypiperidin-4-yl}methyl)-6-(3-hydroxyphenoxy)pyrimidin-4(3H)-one

### Example 64

Autoclave was charged with EXAMPLE 43 (138 mg. 0.2141 mmol) 10% palladium on charcoal (30 mg) and methanol (10 mL), and then placed under a nitrogen atmosphere. After that it was filled with 10 bar H₂ gas, The reaction mixture was stirred in autoclave at r.t. for 20 hours. The catalyst was washed with methanol and tittered off. The mother liquor was purified by Hanbon prep HPLC,C 18 Silica, Gemini NX 5 μm, 5 mM NH₄HCO₃-MeCN using gradient method 5-90%. Solvent was evaporated under reduced pressure to give EXAMPLE 64. HRMS calculated for C₂₉H₃₂F₂N₄O₅: 554.2341; found 555.2405 ((M+H)⁺ form).

### 5-amino-6-[4-(aminomethyl)-3-fluorophenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 65

Autoclave was charged with EXAMPLE 50 (60.8 mg, 0.105 mmol), Raney-nickel catalyst (60 mg) and 7N ammonia in methanol (5 mL) and 1,4-dioxane (5 mL) and then placed under a nitrogen atmosphere. After that it was lulled with 10 bar H₂ gas. The reaction mixture was stirred in autoclave at r.t. for 20 hours. The reaction mixture was removed from the autoclave and filtered. The filtrate was purified by preparative LC (on C-18 Gemini-NX 5 μm column. 5 mM aqueous NH₄HCO₃-MeCN, gradient). Solvent was evaporated under reduced pressure to give EXAMPLE 65. HRMS calculated for C₃₀H₃₄F₃N₅O₄: 585.2563; found 586.2627 ((M+H)⁺ form).

### 5-amino-6-[4-(aminomethyl)-3-chlorophenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 66

Autoclave was charged with EXAMPLE 51 (64.8 mg, 0.108 mmol), Raney-nickel catalyst (60 mg) and 7N ammonia in methanol (5 mL) and 1,4-dioxane (5 mL) and then placed under a nitrogen atmosphere. After that it was filled with 10 bar H₂ gas. The reaction mixture was stirred in autoclave at r.t. for 20 hours. The reaction mixture was removed from the autoclave and filtered. The filtrate was purified by preparative LC (on C-18 Gemini-NX 5 μm column, 5 mM aqueous NH₄HCO₃-MeCN, gradient). Solvent was evaporated under reduced pressure to give EXAMPLE 66. HRMS calculated for C₃₀H₃₄ClF₂N₅O₄: 601.2267; found 602.2327 ((M+H)⁺ form).

### 3-(4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}phenyl)propanamide (EXAMPLE 67)

EXAMPLE 52 (80 mg. 0.1352 mmol), (1E)-acetaldehyde oxime (79.88 mg, 0.0824 mL, 1.352 mmol 10 eq.), Cu²⁺ on 4 Å molecular sieve (100 mg) were dissolved in methanol (3 mL) and 1,4-dioxane (2 mL). The reaction mixture was stirred at r.t. for 4 days. The mixture was filtered, the filtrate was evaporated and purified by preparative LC (on C-18 Gemini-NX 5 μm column, 5 mM aqueous NH₄HCO₃-MeCN, gradient). Solvent was evaporated under reduced pressure to give EXAMPLE 67. HRMS calculated for C₃₂H₃₇F₂N₅O₅: 609.2763; found 610.2832 ((M+H)⁺ form).

### 5-amino-6-[4-(3-aminopropyl)phenoxy]-3-([1-[(1R,2R)-4,4-difluoro-2-phenylcyclohexane-1-carbonyl]-4-hydroxypiperidin-4-yl]methyl)pyrimidin-4(3H)-one

### Example 68

Autoclave was charged with EXAMPLE 52 (80 mg, 0.1352 mmol), Raney-nickel catalyst (80 mg) and 7N ammonia in methanol (5 mL) and 1,4-dioxane (5 ml) and then placed under a nitrogen atmosphere. After that it was filled with 10 bar H₂ gas. The reaction mixture was stirred in autoclave at r.t. for 20 hours. The reaction mixture was removed from the autoclave and filtered The filtrate was purified by preparative LC (on C-18 Gemini-NX 5 μm column, 5 mM aqueous NH₄HCO₃-MeCN, gradient). Solvent was evaporated under reduced pressure to give EXAMPLE 68. HRMS calculated for C₃₂H₃₉F₂N₅O₄: 595.297; found 596.3037 ((M+H)⁺ form).

### 5-amino-6-[4-(aminomethyl)-2-chlorophenoxyl]-3-({1-[(1R,2R)-4,4-difluoro-2-phenylcyclohexane-1-carbonyl]-4-hydroxypiperidin04-yl}methyl)pyrimidin-4(3H)-one

### Example 69

Autoclave was charged with EXAMPLE 53 (1.0 eq.). Raney-nickel catalyst (10 w/w %) and 7N ammonia in methanol (5 mL) and 1,4-dioxane (5 mL) and then placed under a nitrogen atmosphere. After that it was filled with 10 bar H₂ gas. The reaction mixture was stirred in autoclave at r.t for 20 hours. The reaction mixture was removed from the autoclave and filtered. The filtrate was purified by preparative LC (on C-18 Gemini-NX 5 μm column. 5 mM aqueous NH₄HCO₃-MeCN. gradient). Solvent was evaporated under reduced pressure to give EXAMPLE 69. HRMS calculated for C₃₀H₃₄ClF₂N₅O₄: 601.2267; found 602.2335 ((M+H)⁺ form).

### 5-amino-6-[4-(anilinomethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one

### Example 70

EXAMPLE 44 (140 mg, 0.1412 mmol), aniline (65.7486 mg, 11.706 mmol, 5.0 eq.), sodium triacetoxyborohydride (149.63 mg, 0.706 mmol, 5.0 eq,). acetic acid (42.40 mg, 0.04041 mL, 0.706 mmol. 5.0 eq.) were dissolved in THF and stirred at r.t. for 24 hours. The reaction mixture was evaporated, dissolved in DMF/methanol and it was purified by preparative LC (on C-18 Gemini-NX 5 μm column. 5 mM aqueous NH₄HCO₃-MeCN, gradient). Solvent was evaporated under reduced pressure to give EXAMPLE 70. HRMS calculated for C₃₆H₃₉F₂N₅O₄: 643.297; found 644.3038 ((M+H)⁺ form).

### 4-{[5-amino-1-({(4S)-1-[(1R,2R)-4,4-difluoro-2-phenylcyclohexane-1-carbonyl]-3,3-difluoro-4-hydroxypiperidin-4-yl}methyl)-6-oxo-1,6-dihydropyrimidin-4-yl]oxy}benzonitrile (EXAMPLE 71) and

### 4-{[5-amino-1-({(4R)-1-[(1R,2R)-4,4-difluoro-2-phenylcyclohexane-1-carbonyl]-3,3-difluoro-4-hydroxypiperidin-4-yl}methyl)-6-oxo-1,6-dihydropyrimidin-4-yl]oxy}benzonitrile (EXAMPLE 72)

Starting from EXAMPLE 46, EXAMPLE 71 and EXAMPLE 72 were obtained separately by chiral chromatouraphy.

### Example 71

¹H-NMR (500 MHz, dmso-d6) δ ppm 7.85 (m, 2H), 7.7/7.66 (s/s, 1H), 7.33-7.14 (m, 5H), 7.19 (m 2H), 6.08/6.03 (s/s, 1H), 4.97/4.93 (s/s, 2H), 4.61/4.45/3.92/3.76 (d/d+d/d, 2H), 4.30-2.33 (m, 4H), 3.42/3.37 (td/td, 1H), 3.04 (m, 1H), 2.24-1.95 (m, 4H), 1.90-1.55 (m, 2H), 1.54/1.17 (m, 2H).

¹³C-NMR (125 MHz, dmso-d6) δ ppm 159.5/159.4, 159/158.9, 139.1/139, 134.6, 124.1, 120.1, 119.7, 119.3 106, 47.5/47.2, 44/43.4, 42.4/41.9, 32.7/31.6, 26.5/26.4

### Example 72

¹H-NMR (500 MHz, dmso-d6) δ ppm 7.85 (m, 2H), 7.71/7.7 (s/s, 1M, 7.29-7.15 (m, 5H), 7.19 (m, 2H), 6.08/6.06 (s/s, 1H), 4.95/4.91 (s/s, 2H), 4.49/4.41/3.89/3.86 (d/d+d/d, 2H), 4.38-2.68 (m, 4H) 3.45/3.35 (td/td, 1H). 3.05 (m, 1H) 1.96-1.44 (m, 4H), 1.90-1.55 (m, 2H), 1.75-1.14 (m, 2H).

¹³C-NMR (125 MHz, dmso-d6) δ ppm 159.5/159.4, 159, 139, 134.6, 124.1, 120.5, 119.7, 119.3, 106, 47.3/47.2,43.5, 42.4/41.6, 32.9/31.7, 26.8/26.4

### 5-amino-3-({1-[(1R,2R)-4,4-difluoro-2-phenylcyclohexane-1-carbonyl]-4-hydroxy piperidin-4-yl}methyl)-6-(4-[[(2,2,2-trifluoromethyl)amino]methyl]phenoxy)pyrimidin-4(3H)-one (EXAMPLE 73) and

### N-[(4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}phenyl)methyl]-N-(2,2,2-trifluoroethyl)formamide (EXAMPLE 74)

EXAMPLE 44 (0.158 mmol; 1.0 eq.), 2,2,2-trifluoroethanamine (5.0 eq.), sodium triacetoxyborohydride (5.0 eq.), acetic acid (5.0 eq.) were dissolved in THF and stirred r.t. for 3.5 hours. The reaction mixture was evaporated, dissolved in DMF and it was purified by preparative LC (on C-18 Gemini-NX 5 μm column, 5 mM aqueous NH₄HCO₃-MeCN, gradient) and compounds were isolated separately. Solvent was evaporated under reduced pressure to give EXAMPLE 73 and EXAMPLE 74.

EXAMPLE 73: HRMS calculated for C₃₂H₃₆F₅N₅O₄: 649.2687; found 650.2753 ((M+H)⁺ form).

EXAMPLE 74: HRMS calculated for C₃₃H₃₆F₅N₅O₅: 677.2637; found 678.2707 ((M+H)⁺ form).

### 5-amino-6-{4-[(tert-butylamino)methyl]phenoxy}-3-({1-[(1R,2R)-4,4-difluoro-2-phenylcyclohexane-1-carbonyl]-4-hydroxypiperidin-4-yl}methyl)pyrimidin-4(3H)-one

### Example 75

EXAMPLE 44 (0.158 mmol; 1.0 eq.), 2-methylpropan-2-amine (5.0 eq.), sodium triacetoxyborohydride (5.0 eq.). acetic acid (5.0 eq.) were dissolved in THF and stirred r.t. for 3.5 hours. The reaction mixture was evaporated. dissolved in DMF and it was purified by preparative LC (on C-18 Gemini-NX 5 μm column, 5 mM aqueous NH₄HCO₃-MeCN, gradient). Solvent was evaporated under reduced pressure to give EXAMPLE 75. HRMS calculated for C₃₄H₄₃F₂N₅O₄: 623.3283; found 624.3346 ((M+H)⁺ form).

### 5-amino-6-{4-[(benzylamino)methyl]phenoxy}-3-({1-[(1R,2R)-4,4-difluoro-2-phenylcyclohexane-1-carbonyl]-4-hydroxypiperidin-4-yl}methyl)pyrimidin-4(3H)-one

### Example 76

EXAMPLE 44 (0.123 mmol; 1.0 eq.), benzylamine (5.0 eq.), sodium triacetoxyborohydride (5.0 eq.), acetic acid (5.0 eq.) were dissolved in THF and stirred r.t. for 70 hours. The reaction mixture was evaporated, dissolved in DMF/methanol and it was purified by preparative LC (on C-18 Gemini-NX 5 μm column. 5 mM aqueous NH₄HCO₃-MeCN, gradient). Solvent was evaporated under reduced pressure to give EXAMPLE 76. HRMS calculated for C₃₇H₄₁F₂N₅O₄: 657.3127; found 658.3202 ((M+H)⁺ form).

### 5-amino-6-[4-](cyclopropylamino)methyl]phenoxy}-3-({1-[(1R,2R)-4,4-difluoro-2-phenylcyclohexane-1-carbonyl]-4-hydroxypiperidin-4-yl}methyl)pyrimidin-4(3H)-one

### Example 77

EXAMPLE 44 (0.1521 mmol; 1.0 eq.), cyclopropylamine (5 0 eq.), sodium triacetoxyborohydride (5.0 eq.), acetic acid (5.0 eq.) were dissolved in THF and stirred r.t. for 24 hours. The reaction mixture was evaporated, dissolved in DMF/methanol and it was purified by preparative LC (an C-18 Gemini-NX 5 μm column, 5 mM aqueous NH₄HCO₃-MeCN, gradient). Solvent was evaporated under reduced pressure to give EXAMPLE 77. HRMS calculated for C₃₃H₃₉F₂N₅O₄: 607.297; found 608.3039 ((M+H)⁺ form).

### 5-amino-3-({(4S)-1-[(1R,2R)-4,4-difluoro-2-phenylcyclohexane-1-carbonyl]-3,3-difluoro-4-hydroxypiperidin-4-yl}methyl)-6-(4-fluorophenoxy)pyrimidin-4(3H)-one

### Example 78

and

### 5-amino-3-({(4R)-1-[(1R,2R)-4,4-difluoro-2-phenylcyclohexane-1-carbonyl]-3,3-difluoro-4-hydroxypiperidin-4-yl}methyl)-6-(4-fluorophenoxy)pyrimidin-4(3H)-one

### Example 79

Starting from EXAMPLE 45, EXAMPLE 78 and EXAMPLE 79 were obtained separately by chiral chromatography.

EXAMPLE 78: HRMS calculated for C₂₉H₂₉F₅N₄O₄: 592.2109; found 593.2182 ((M+H)⁺ form).

EXAMPLE 79: HRMS calculated for C₂₉H₂₉F₅N₄O₄: 5912109; found 593.2177 ((M+H)⁺ form).

### 4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}-2-chlorobentamide (EXAMPLE 80)

EXAMPLE 51 (65 mg, 0.108 mmol), (1E)-acetaldehyde oxime (1.08 mmol, 10 eq.), Cu²⁺ on 4 Å molecular sieve (80 mg) were dissolved in methanol (3 mL) and 1.4-dioxane (2 mL). The reaction mixture was stirred at r.t. for 4 days. The mixture was filtered, the filtrate was evaporated and purified by preparative LC (on C-18 Gemini-NX 5 μm column, 5 mM aqueous NH₄HCO₃-MeCN, gradient). Solvent was evaporated under reduced pressure to give EXAMPLE 80. HRMS calculated for C₃₀H₃₂ClF₂N₅O₅: 615.206; found 616.2129 ((M+H)⁺ form).

### 4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}-2-fluorobenzamide (EXAMPLE 81)

EXAMPLE 50 (65 mg, 0.111 mmol), (1E)-acetaldehyde oxime (1.11 mmol, 10 eq.), Cu²⁺ on 4 Å molecular sieve (80 mg) were dissolved in methanol (3 mL) and 1,4-dioxane (2 mL). The reaction mixture was stirred at r.t. for 4 days. The mixture was filtered, the filtrate was evaporated and purified by preparative LC (on C-18 Gemini-NX 5 μm column 5 mM aqueous NH₄HCO₃-MeCN gradient). Solvent was evaporated under reduced pressure to give EXAMPLE 81. HRMS calculated for C₃₀H₃₂F₃N₅O₅: 599.2355; 600.2428 ((M+H)⁺ form).

### 5-amino-3-({1-[(1R,2R)-5,5-difluoro-2-phenylcyclohexane-1-carbonyl]-4-hydroxypiperidin-4-yl}methyl)-6-phenoxypyrimidin-4(3H)-one (EXAMPLE 82)

Using General Procedure 5 starting from Preparation R4a and Preparation R5c as reagents, EXAMPLE 82 was obtained. HRMS calculated for C₂₉H₃₂F₂N₄O₄: 538.2391; found 539.2468 ((M+H)⁺ form).

### 5-amino-3-({1-[(1R,2R)-4,4-difluoro-2-(1H-pyrrol-1-yl)cyclohexane-1-carbonyl]-4-hydroxypiperidin-4-yl}methyl)-6-phenoxypyrimidin-4(3H)-one (EXAMPLE 83)

Using General Procedure 5 starting from Preparation R4a and Preparation R5d as reagents, EXAMPLE 83 was obtained. HRMS calculated for C₂₇H₃₁F₂N₅O₄: 527.2344; found 528.2416 ((M+H)⁺ form).

### 5-amino-3-({1-[(1,2-trans)-4,4-difluoro-2-phenylcyclohexane-1-carbonyl]-4-hydroxypiperidin-4-yl}methyl)-6-[(pyridin-3-yl)oxy]pyrimidin-4(3H)-one

### Example 84

Using General Procedure 5 starting from Preparation R4bq and Preparation R5a as reagents, EXAMPLE 84 was obtained. HRMS calculated for C₂₈H₃₁F₂N₅O₄: 539.2344; found 540.2425 ((M+H)⁺ form).

### 5-amino-3-({1-[(1,2-trans)-4,4-difluoro-2-(pyridin-2-yl)cyclohexane-1-carbonyl]-4-hydroxypiperidin-4-yl}methyl)-6-(4-fluorophenoxy)pyrimidin-4(3H)-one

### Example 85

Using General Procedure 5 starting from Preparation R4e and Preparation R5e as reagents, EXAMPLE 85 was obtained. HRMS calculated for C₂₈H₃₀F₃N₅O₄: 557125; found 558.2321 ((M+H)⁺ form).

### 5-amino-3-({1-[(1,2-trans)-4,4-difluoro-2-(5-methylthiophen-3-yl)cyclohexane-1-carbonyl]-4-hydroxypiperidin-4-yl}methyl)-6-(4-fluorophenoxy)pyrimidin-4(3H)-one

### Example 86

Using General Procedure 5 starting, from Preparation R4e and Preparation R5g as reagents. EXAMPLE 86 was obtained. HRMS calculated for C₂₈H₃₁F₃N₄O₄S: 576.2018; found 577.2091 ((M+H)⁺ form).

### 5-amino-3-({1-[(1,2-trans)-2-(1-ethyl-1H-pyrazol-4-yl)-4,4-difluorocyclohexane-1-carbonyl]-4-hydroxypiperidin-4-yl}methyl)-6-(4-fluorophenoxy)pyrimidin-4(3H)-one

### Example 87

Using General Procedure 5 starting from Preparation R4e and Preparation R5i as reagents. EXAMPLE 87 was obtained. HRMS calculated for C₂₈H₃₃F₃N₆O₄: 574.2515; found 575.2586 ((M+H)⁺ form).

### 5-amino-3-({1-[(1,2-trans)-4,4-difluoro-2-(furan-2-yl)cyclohexane-1-carbonyl]-4-hydroxypiperidin-4-yl}methyl)-6-(4-fluorophenoxy)pyrimidin-4(3H)-one

### Example 88

Using General Procedure 5 starting from Preparation R4e and Preparation R5k as reagents, EXAMPLE 88 was obtained. HRMS calculated for C₂₇H₂₉F₃N₄O₅: 546.209; found 547.2164 ((M+H)⁺ form).

### 5-amino-3-({1-[(1R,2R)-4,4-difluoro-2-(thiophen-3-yl)cyclohexane-1-carbonyl]-4-hydroxypiperidin-4-yl}methyl)-6-(4-fluorophenoxy)pyrimidin-4(3H)-one

### Example 89

Using General Procedure S starting from Preparation R4e and Preparation R5l as reagents, EXAMPLE 89 was obtained. HRMS calculated for C₂₇H₂₉F₂N₄O₄S: 562.1862; found 563.1932 ((M+H)⁺ form).

### 5-amino-6-[4-(aminomethyl)phenoxy]-3-({(4S)-1-[(1R,2R)-4,4-difluoro-2-phenylcyclohexane-1-carbonyl]-3,3-difluoro-4-hydroxypiperidin-4-yl}methyl)pyrimidin-4(3H)-one (EXAMPLE 90)

Autoclave was charged with EXAMPLE 71 (50 mg, 0.083 mmol), Raney-nickel catalyst (100 mg) and 7N ammonia in methanol (5 mL) and 1,4-dioxane (5 mL) and then placed under a nitrogen atmosphere. After that it was filled with 10 bar H₂ gas. The reaction mixture was stirred in autoclave at r.t. for 20 hours. The reaction mixture was removed from the autoclave and filtered. The filtrate was purified by preparative LC (on C-18 Gemini-NX 5 μm column. 0.2% aqueous HCOOH-MeCN. gradient). Solvent was evaporated under reduced pressure to give EXAMPLE 90. HRMS calculated for C₃₀H₃₃F₄N₅O₄: 603.2469; found 604.2530 ((M+H)⁺ form).

### General Procedures for Examples 91 to 103 and 122 to 177

All reagents obtained from commercial sources were used without further purification. Anhydrous solvents were obtained from commercial sources and used without further drying.

Flash chromatography was performed with pre-packed silica gel cartridges (Strata S1-1; 61 Å. Phenomenex, Cheshire UK or 1ST Flash II, 54 Å, Argonaut, Hengoed, UK) or by automated flash chromatography using a Combi flash R_{f} apparatus (Teledyne Isco Inc.) using RediSep R₁ prepacked silica columns (Teledyne Isco Inc.) or SilaSep pre-packed columns (Silicycle Inc.). Thin layer chromatography was conducted with 5×10 cm plates coated with Merck Type 60 F₂₅₄ silica gel.

The compounds of the present invention were characterized by high performance liquid chromatography-mass spectroscopy (HPLC-MS) on either an Agnew HP1200 Rapid Resolution Mass detector 6140 multimode source M/z range 150 to 1000 amu or an Agilent HP100 Mass detector 1946D ESI source M/z range 150 to 1000 amu. The conditions and methods listed below are identical for both machines.
Column for 7.5 min run: GeminiNX, 5 μm, C18, 30×2.1 mm (Phenomenex) or Zorbax Eclipse Plus, 3.5 μm, C18, 30×2.1 mm (Agilent). Temperature: 35° C.
Column for 3.75 min run: GeminiNX, 5 μm, C18, 30×2.1 mm (Phenomenex) or Zorbax Eclipse Plus, 3.5 μm, C18, 30×2.1 mm (Agilent). Temperature: 35° C.
Column for 1.9 min run: Kinetex, 2.5 μm, C18, 50×2.1 mm (Phenomenex) or Accticore, 2.6 μm, C18, 50×2.1 mm. Temperature: 55 ° C.
Mobile Phase: A—H₂O—10 mmol/ammonium formate—0.0 8% (v/v) formic acid at pH ca 3.5.
B—95% Acetonitrile+5% A+0.08% (v/v) formic acid.
Injection Volume: 1 μL

Method A “Short” method gradient, table, either positive (pos) or positive and negative (pos/neg) ionisation

| Time (min) | Solvent A (%) | Solvent B (%) | Flow (mL/min) |
|---|---|---|---|
| 0 | 95 | 5 | 1 |
| 0.25 | 95 | 5 | 1 |
| 2.50 | 5 | 95 | 1 |
| 2.55 | 5 | 95 | 1.7 |
| 3.60 | 5 | 95 | 1.7 |
| 3.65 | 5 | 95 | 1 |
| 3.70 | 95 | 5 | 1 |
| 3.75 | 95 | 5 | 1 |

Method B “Super Short” method gradient table, either positive (pos) or positive and negative (pos neg) ionisation

| Time (min) | Solvent A (%) | Solvent B (VG) | Flow (mL/min) |
|---|---|---|---|
| 0 | 95 | 5 | 1.3 |
| 0.12 | 95 | 5 | 1.3 |
| 1.30 | 5 | 95 | 1.3 |
| 1.35 | 5 | 95 | 1.6 |
| 1.85 | 5 | 95 | 1.6 |
| 1.90 | 5 | 95 | 1.3 |
| 1.95 | 95 | 5 | 1.3 |

Detection: UV detection at 230, 254 and 270 nm.

The compounds of the present invention were also characterized by Nuclear Magnetic Resonance (NMR). Analysis was performed with a Bruker DPX-400 spectrometer and proton NMR spectra were measured at 400 MHz, The spectral reference was the known chemical shill of the solvent. Proton NMR data is reported as follows: chemical shin (δ) in ppm, followed by the multiplicity, where s=singlet, d=doublet, t=triplet, q=quartet, m=multiplet, dd=doublet of doublets, dt=doublet of triplets, dm=doublet of multiplets. ddd=doublet of double doublets, td=triplet of doublets, qd=quartet of doublets and br=broad, and finally the integration.

Some compounds of the invention were purified by preparative HPLC. These were performed on a Waters FractionLynx MS autopurification system, with a Gemini® 5 μm C18(2), 100 mm×20 mm i.d. column from Phenomenex, running at a flow rate of 20 cm³.min⁻¹ with UV diode array detection (210-400 nm) and mass-directed collection. At pH 4: solvent A=10 mM ammonium acetate in HPLC grade water+0.08% v/v formic acid. Solvent B=95% v/v HPLC grade acetonitrile+5% v/v solvent A+0.08% v/v formic acid.

At pH 9: solvent A=10 mM ammonium acetate in HPLC grade water+4.08% v/v ammonia solution. Solvent B=95% v/v HPLC grade acetonitrile+5% v/v solvent A+0.08% v/v ammonia solution.

Some compounds of the invention were purified by preparative HPLC using a Teledyne ISCO ACCQPrep HP125, with a Gemini® 5 μm C18(2), 150 mm×21 mm i.d. column from Phenomenex, running at a flow rate of 21 cm³.min⁻¹ with UV diode array detection (210-400 nm) and collection.

The mass spectrometer was a Waters Micromass ZQ2000 spectrometer, operating in positive or negative ion electrospray ionisation modes, with a molecular weight scan range of 150 to 1000.

Some compounds of the present invention were characterised using an Agilent 1290 infinity II series instrument connected to an Agilent TOF 6234) single quadrupole with an ESI source. High resolution mass spectra were recorded in positive-negative switching mode ionization unless otherwise stated, UV detection was by diode array detector at 230, 254 and 270 nm. Column: Thermo Accueore 2.6 μM C18, 50×2 mm, at 55° C. column temperature. Buffer A: Water/10 mM ammonium formate/0.04% (v/v) formic acid pH=3.5. Buffer B: Acetonitrile/5.3% (v/v) A/0.04% (v/v) formic. (Injection volume: 1 μL).

IUPAC chemical names were generated using AutoNom Standard or using ChemAxon's ‘Structure to Name’ (s2n) functionality within MarvinSketch or JChem for Excel (JChem versions 16.6.13-18.22.3).

### rel-5-amino-3-([1-[(1R,2R,4R)-4-ethynyl-4-hydroxy-2-phenyl-cyclohexanecarbonyl]-4-hydroxypiperidin-4-yl]methyl)-6-(4-fluorophenoxy)-3,4-dihydropyrimidin-4-one

### Example 9

### Step 1: rel-ethyl (1R,2R,4R)-4-hydroxy-2-phenyl-4 -[2-(trimethylsilyl)ethynyl]cyclohexane-1-carboxyate and rel-ethyl (1R,2R,4S)-4-hydroxy-2-phenyl-4-[2-(trimethylsilyl)ethylnyl]cyclohexane-1 -carboxylate

Following procedure described in Step 1 of EXAMPLE 96 and sinning from rel-ethyl (1R,2R)-4-oxo-2-phenylcyclohexane-1-carboxylate (500 mg, 2.03 mmol) and ethynyltrimethylsilane (219 mg, 2.23 mmol) instead of 2-picoline, the obtained residue was purified via flash chromatography using Heptane-20% EtOAc/Heptane (gradient) as eluent to afford:

First elute: rel-ethyl (1R,2R,4S)-4-hydroxy2-phenyl-4-[2-trimethylsilyl)ethynyl]cyclohexane-1-carboxylate as a colourless oil.

¹H NMR (399 MHz, Chloroform-d) δ 7.22-6.95 (m, 5H), 3.73 (q, J=7.1 Hz, 2H), 3.10 (ddd, J=13.0. 11.5, 3.6 Hz, 1H), 2.44 (td, J=11.8, 3.4 Hz, 1H), 2.07-1.55 (m, 5H), 1.24-0.96 (m, 2H), 0.80 (t, J=7.1 Hz, 3H), 0.00 (s, 9H).

Second elute: rel-ethyl (1R,2R,4R)-4-hydroxy-2-phenyl-4-[2-(trimethylsilyl)ethynyl]cyclohexane-1-carboxylate as a colourless oil.

¹H NMR (399 MHz, Chloroform-d) δ 7.28-6,90 (m, 5H), 3.74 (qd, J=7.1, 3.5 Hz, 2H), 3.04 (ddd, J=13.0, 11.4, 3.2 Hz, 1H), 2.38 (td, J=11.4, 4.6 Hz, 1H), 2.08-1.79 (m, 5H), 1.63 (t, J=12.8 Hz, 1H), 1.51 (td, J=12.5, 4.8 Hz,, 1H), 0.82 (t, J=7.1 H, 3H), 0.10 (s, 9H).

### Step 2: rel-ethyl (1R,2R,4R)-4-ethynyl-4-hydroxy-2-phenylcyclohexane-1-carboxylate

To a solution of rel-ethyl (1R,2R,4R)-4-hydroxy-2-phenyl-4-[2-(trimethylsilyl)ethynyl]cyclohexane-1-carboxylate (100 mg, 0.29 mmol) in THF (2 mL), TBAF (1M in THF, 1 mL, 1 mmol) was added dropwise at 0° C. under N₂. The reaction mixture was stirred at the same temperature for 10 minutes before allowed to warm to r.t. overnight. The mixture was diluted with EtOAc (20 mL) and brine (15 mL). The organic layer was separated, dried (MgSO₄) and evaporated in vacua. The residue was purified via flash chromatography using Heptane-30% EtOAc/Heptane (gradient) as eluent to afford the desired product.

rel-ethyl (1R,2R,4R)-4-ethynyl-4-hydroxy-2-phenylcyclohexane-1-carboxylate as a colourless oil.

¹H NMR (399 MHz Chloroform-d) δ 7.41-7.08 (m, 5H). 3.90 (q, J=7.2 Hz, 2H), 3.21 (ddd, J=12.9, 11.4, 3.3 Hz, 1H), 2.67 (s, 1H), 2.56 (td, J=11.5, 4.3 Hz, 1H), 2.25 (s, 1H), 2.23-2.14 (m 2H), 2.13-1.96 (m, 2H), 1.82 (t, J=12.9 Hz, 1H), 1.70 (td, J=12.7, 4.6 Hz, 1H), 0.96 (t, J=7.1 Hz, 3H).

### Step 3: rel-(1R,2R,4R)-4-ethynyl-4 -hydroxy-2-phenylcyclohexane-1-carboxylic acid

Starting from rel-ethyl (1R,2R,4R)-4-ethynyl-4-hydroxy-2-phenyl-cyclohexane-1-carboxylate ((62 mg, 0.23 mmol) following procedure described in Step 2 of EXAMPLE 94, rel-(1R,2R,4R)-4-ethynyl-4-hydroxy-2-phenylcyclohexane-1-carboxylic acid was obtained as a yellow solid. The compound was used without further purification.

¹H NMR (399 MHz, DMSO-d₆) δ 11.92 (s, 1H), 7.44-7.00 (m, 5H), 5.67 (s, 1H), 3.48 (s, 1H), 3.01 (td, J=12.4, 3.5 Hz, 1H), 2.04-1.42 (m, 7H).

### Step 4: EXAMPLE 91

Following procedure described in Step 3 of EXAMPLE 94 and starting from rel-(1R,2R,4R)-4-ethynyl-4-hydroxy-2-phenyl-cyclohexane-1-carboxylic acid (60 mg) and 5-amino-6-(4-fluorophenoxy)-3[(4-hydroxypiperidin-4-yl)methyl]pyrimidin-4-one (185 mg, 0.22 mmol), EXAMPLE 91 was obtained as a white solid.

LC/MS (Method B): RT=1.01; m/z=561 [M+H]⁺

¹H NMR (399 MHz, DMSO-d₆) δ 7.59 (d, J=24.3 Hz, 1H), 7.33-6.99 (m, 9H), 5.64 (d, J=3.4 Hz, 1H), 4.81 (d, J=10.9 Hz, 1H), 4.68 (d, J=12.5 Hz, 2H), 3.99-3.78 (m, 2H), 3.74-3.58 (m, 2H), 3.45 (d, J=2.4 Hz, 1H), 3.20-2.80 (m, 3H), 2.70-2.58 (m, 1H, 1.97-1.56 (m, 5H), 1.47-1.04 (m, 3H), 0.76 (td, J=12.8, 4.5 Hz, 1H), 0.62-0.49 (m, 1H), HRMS (TOF, ESI) m/z: Calculated for C₃₁H₃₃FN₄O₅ 560.2435, Found; 561.2538 [M+H]⁺

### rel-5-amino-3-[(1-{[(1R,2R,4S)-4-ethynyl-4-hydroxy-2-phenyl-cyclohexyl]carbonyl}-4-hydroxypiperidin-4-yl)methyl]-6-(4-fluorophenoxy)pyrimidin-4-one (EXAMPLE 92)

### Step 1: rel-ethyl (1R,2R,4S)-4-ethynyl-4-hydroxy-2-phenyl-cyclohexane-1-carboxylate

Starting from rel-ethyl (1R,2R,4S)-4-hydroxy-2-phenyl-4-[2-(trimethylsilyl)ethynyl]cyclohexane-1-carboxylate (60 mg, 0.2 mmol) following procedure described in Step 2 of EXAMPLE 91. rel-ethyl (1R,2R,4S)-4-ethynyl-4-hydroxy-2-phenylcyclohexane-1-carboxylate was obtained as a colourless oil.

¹H NMR (399 MHz, Chloroform-d) δ 739-7.12 (m, 5H), 3.90 (q, J=7.1 Hz, 2H), 3.28 (ddd, J=13.0, 11.5, 3.6 Hz, 1H) 2.64-2.57 (m, 1H), 2.49 (s, 1H), 2.26-2.01 (m, 3H), 1.99-1.76 (m, 3H), 1.30-1.24 (m, 1H), 0.96 (t, J=7.1 Hz, 2H), 0.93-0.88 (m, 1H).

### Step 2: rel-(1R,2R,4S)-4-ethynyl-4-hydroxy-2-phenyl-cyclohexane-1-carboxylic acid

Starting from rel-ethyl (1R,2R,4S)-4-ethynyl-4-hydroxy-2-phenylcyclohexane-1-carboxylate (35 mg, 0.13 mmol) following procedure described in Step 2 of EXAMPLE 94, rel-(1R,2R,4S)-4-ethynyl-4-hydroxy-2-phenylcyclohexane-1-carboxylic acid was obtained as a yellow solid.

¹H NMR (399 MHz, DMSO-d₆) δ 11.80 (s, 1H), 7.29-7.15 (m, 5H), 5.43 (s, 1H), 3.38 (m, 1H),3.08 (td, J=12.4, 3.5 Hz, 1H), 2.60 (m, 1H), 1.96-1.70 (m, 6H).

Step 3: EXAMPLE 92

Starting from rel-(1R,2R,4S)-4-ethynyl-4-hydroxy-2-phenyl-cyclohexane-1-carboxylic acid (27 mg, (1.11 mmol) and 5-amino-6-(4-fluorophenoxy)-3-[(4-hydroxypiperidin-4-yl)methyl]pyrimidin-4-one (37 mg, 0.11 mmol) following procedure described in Step 3 of EXAMPLE 94, EXAMPLE 92 was obtained as a white solid.

LC/MS (Method B): RT=1.07; m/z=561 [M+H]⁺

¹H NMR (399 MHz, DMSO-4) δ 7.60 (d, J=23.3 Hz, 1H), 7.33-7.03 (m, 9H), 5.37 (s, 1H), 4.94-4.57 (m, 3H), 3.99-3.57 (m, 4H), 3.28-2.82 (m, 3H), 2.71-2.57 (m, 1H), 2.00-1.69 (m, 5H), 1.54-1.04 (m, 4H), 0.82-0.56 (m, 2H).

HRMS (TOF, ESI) m/z: Calculated for C₁₁H₃₃FN₄O₅ 560.2435, Found: 561.2576 [M+H]⁺

### rel-5-amino-3-({1-[(1R,2R)-4-ethynyl-4-fluoro-2-phenyl-cyclohexanecarbonyl]-4-hydroxypiperidin-4-yl}methyI)-6-(4-fluorophenoxy)-3,4-dihydropyrimidin-4-one

### Example 93

Step 1: rel-ethyl (1R,2R)-4-ethynyl-4-fluoro-2-phenylcyclohexane-1-carboxylate

To an ice cooled solution of rel-ethyl (1R,2R,4R)-4-ethynyl-4-hydroxy-2-phenylcyclohexane-1-carboxylate (67 mg, 0.25 mmol) in DCM (2 mL), bis(2-methoxyethyl)aminosulphur trifluoride. (50% solution in THF, 0.21 mL, 0.49 mmol) was added dropwise under N₂. After 5 minutes, the ice bath was removed and the reaction mixture was allowed to warm to r.t. over 2 hours. Cooled to 0° C. and quenched with sat. NaHCO₃ solution (10 mL). The reaction mixture was extracted with EtOAc (2×15 mL). The combined organic layer were dried (MgSO₄) and evaporated in vacua. The residue was purified via flash chromatography using Heptane-10% EtOAc/Heptane (gradient) as eluent to afford rel-ethyl (1R,2R)-4-ethynyl-4-fluoro-2-phenylcyclohexane-1-carboxylate as a white solid.

¹H NMR (399 MHz, Chloroform-d) δ 7.46-7.12 (m, 5H), 3.90 (qd, 7.1, 1.7 Hz, 2H), 3.30-3.08 (m, 1H), 2.71-2.52 (m, 2.49-2.27 (m, 2H), 2.21-1.64 (m, 4H), 0.96 (td, J=7.1, 2.7 Hz, 3H).

### Step 2: rel-(1R,2R)-4-ethynyl-4-fluoro-2-phenylcyclohexane-1-carboxylic acid

Starting from rel-ethyl (1R,2R)-4-ethynyl-4-fluoro-2-phenylcyclohexane-1-carboxylate (30 mg, 0.11 mmol) following procedure described in Step 2 of EXAMPLE 94, rel-(1R,2R)-4-ethynyl-4-fluoro-2-phenylcyclohexane-1 -carboxylic acid was obtained as a colourless oil.

¹H NMR (399 MHz, DMSO-d₆) δ 11.97 (s, 1H), 7.43-7.01 (m, 5H), 3.57-3.14 (m, 4H), 3.12-2.56 (m, 2H), 2.30-1.41 (m, 3H)

Step 3: EXAMPLE 93

Starting from rel-(1R,2R)-4-ethynyl-4-fluoro-2-phenyl-cyclohexane-1-carboxylic acid (18 mg, 0.07 mmol) and 5-amino-6-(4-fluorophenoxy)-3-[(4-hydroxypiperidin-4-yl) methyl]pyrimidin-4-one (24 mg, 0.07 mmol) following procedure described in Step 3 of EXAMPLE 94, the obtained residue was purified via prep HPLC (Prep HPLC Column: Gemini pH 4 Dimensions: 21.1 mm×150 mm 5 μm) to give EXAMPLE 93 as a white solid. LC/MS (Method B): RT=1.177; m/z=563 [M+H]⁺

¹H NMR (399 MHz, DMSO-d₆) δ 7.60 (d, J=214 Hz, 1H), 7.43-6.99 (m, 9H), 4.86 (d, J=7.0 Hz, 1H), 4.68 (d, J=14.0 Hz, 2H), 4.00-3.52 (m, 4H), 3.30-3.01 (m, 3H), 2.97-2.82 (m, 1H), 2.65 (td, J=12.6, 2.5 Hz, 1H), 2.29-1.88 (m, 3H), 1.86-1.55 (m, 2H), 1.46-1.04 (m, 3H), 0.68 (m, 2H).

HRMS (TOF, ESI) m/z: Calculated for C₃₁H₃₂F₂N₄O₄ 562.2392, Found: 563.2528 [M+H]⁺

### rel-5-amino-3-({1-[(1R,2R,4R)-4-benzyl-4-hydroxy-2-phenyl-cyclohexanecarbonyl]-4-hydroxypiperidin-4-yl}methyl)-6-(4-fluorophenoxy)-3,4-dihydropyrimidin-4-one

### Example 94

### Step 1: rel-ethyl (1R,2R,4R)-4-benzyl-4-hydroxy-2-phenyl-cyclohexane-1-carboxylate and rel-(1R,5R,6S)-1-benzyl-6-phenyl-2-oxabicyclo[3.2.2]nonan-3-one

To a solution of benzylmagnesium chloride (0.89 mL, 1M solution in DEE, 0.89 mmol) in THF (2 mL) at 0° C. under N₂, was added dropwise a solution of rel-ethyl (1R,2R)-4-oxo-2-phenylcyclohexane-1-carboxylate (200 mg, 0.81 mmol) in THF (4 mL). The mixture was stirred at the same temperature for 10 minutes before allowed to warm to r.t. over 1 hour. Saturated ammonium chloride solution (15 mL) was added and the aqueous layer was extracted with EtOAc (25 mL). The organic layer was separated, dried (MgSO₄) and evaporated in vacuo. The residue was pun lied via flash chromatography using Heptane-80% EtOAc/Heptane (gradient) as eluent to afford:

First elute: rel-(1R,5R,6S)-1-benzyl-6-phenyl-2-oxabicyclo[3.2.2]nonan-3-one as a colourless oil.

¹H NMR (399 MHz, DMSO-d₆) δ 7.45-7.18 (m, 10H). 3.45-3.37 (m, 1H), 3.05 (s, 2H), 2.20-2.02 (m, 3H), 1.96-1.84 (m, 1H), 1.73-1.48 (m, 3H)

LC/MS (Method B): RT=1.36; m/z=293 [M+H]⁺

Second elute: rel-ethyl (1R,2R,4R)-4-benzyl-4-hydroxy-2-phenyl-cyclohexane-1-carboxylate as a colourless oil.

¹H NMR (399 MHz, DMSO-d₆) δ 7.40-7.17 (m, 10H), 4.57 (s, 1H), 3.88 (q, J=7.1 Hz, 2H), 3.03 (td, J=11.9. 4.5 Hz, 1H), 2.98-2.87 (m, 2H), 2.68 (td, J=11.7, 3.8 Hz, 1H), 1.95 (m, 1H), 1.86-1.68 (m, 2H), 1.65-1.47 (m, 3H), 0.92 (t, J=7.1 Hz, 3H).

### Step 2: rel R) 4 -benzyl 4 -hydroxy-2-phenyl-cyclohexane-1 -carboxylic acid

To a solution of rel-ethyl (1R,2R,4R)-4-benzyl-4-hydroxy-2-phenyl-cyclohexane-1-carboxylate (55 mg, 0.16 mmol) in methanol (0.5 mL) and THF (0.5 mL), sodium hydroxide (1M aq. solution, 0.49 mL, 0.49 mmol) was added and the reaction mixture stirred at 65° C. overnight. Allowed to cool to r.t., concentrated in vacua, the residue was dissolved in water (10 mL). acidified to pH 4 with 1M HCl and extracted with EtOAc (2×15 mL). The combined organic layer were dried (MgSO₄), evaporated in vacua to afford rel-(1R,2R,4R)-4-benzyl-4-hydroxy-2-phenylcyclohexane-1-carboxylic acid. The compound was used without further purification.

Step 3: EXAMPLE 94

To a solution of 5-amino-6-(4-fluorophenoxy)-3-[(4-hydroxypiperidin-4-yl)methyl]pyrimidin-4-one (54 mg, 0.16 mmol) and rel-(1R,2R,4R)-4-benzyl-4-hydroxy-2-phenylcyclohexane-1-carboxylic acid (50 mg) in acetonitrile (3 mL) was added triethylamine (0.04 mL, 0.32 mmol) followed by HATU (61 mg, 0.16 mmol) at r.t. under N₂. The reaction mixture was stirred for 2 hours and then concentrated in vacuo. The resultant residue was partitioned between EtOAc (25 mL) and sat NaHCO₃ solution (15 mL). The organic layer was separated, dried (MgSO₄) and concentrated in vacuo. The residue was purified via flash chromatography using DCM-4% MeOH/DCM (gradient) as eluent to afford EXAMPLE 94 as a white solid.

LC/MS (Method B): RT=1.15; m/z=627 [M+H]⁺

1H NMR. (399 MHz, DMSO-d6) δ 7.60 (d, 24.2 Hz, 1H), 7.33-7.06 (m, 14H), 4.82 (d, J=12.0 Hz, 1H), 4.68 (d, J=11.9 Hz, 2H), 4.46 (d, J=2.6 Hz, 1H), 4.02-3.56 (m, 5H), 3.22-2.80 (m, 4H), 2.74-2.62 (m, 1H), 1.83-1.07 (m, 8H), 0.87-0.74 (m, 1H), 0.68-0.55 (m, 1H). HRMS (TOF, ESI) m/z: Calculated for C₃₆H₃₉FN₄O₅ 626.2904. Found: 627.3001 [M+H]⁺

### rel-5-amino-3-({1-[(1R,2R,4S)-4-benzyl-4-hydroxy-2-phenyl-cyclohexanecarbonyl]-4-hydroxypiperidin-4-yl}methyl)-6-(4-fluorophenoxy)-3,4-dihydropyrimidin-4-one

### Example 95

### Step 1: rel-(1R,2R,4S)-4-benzyl-4 -hydroxy-2-phenylcyclohexane-1-carboxylic acid

Starting from rel-1R,5R,6S)-1-benzyl-6-phenyl-2-oxabicyclo[3.2.2]nonan-3-one (105 mg, 0.36 mmol) following procedure described in Step 2 of EXAMPLE 94. rel-(1R,2R,4S)-4-benzyl-4-hydroxy-2-phenylcyclohexane-1 -carboxylic acid was obtained as a yellow solid. The compound was used without further purification.

LC/MS (Method B) RT=1.20; m/z=309 [M+H]⁺

Step 2: EXAMPLE 95

Starting from 5-amino-6-(4-fluorophenoxy)-3-[(4-hydroxypiperidin-4-yl)methyl]pyrimidin-4-one (120 mg, 0.36 mmol) and rel-(1R,2R,4S)-4-benzyl-4-hydroxy-2-phenylcyclohexane-1-carboxylic acid (185 mg) following procedure described in Step 3 of EXAMPLE 94, the obtained residue was purified via prep HPLC (Prep HPLC Column: Gemini pH 4 Dimensions: 21.1 mm×150 mm 5 μm) to give EXAMPLE 95 as a white solid.

LC/MS (Method B): RT=1.21, m/z=627 [M+H]⁺

¹H NMR (399 MHz, DMSO-d₆) δ 7.60 (d, J=22.3 Hz, 1H), 7.29-7.05 (m, 14H), 4.81 (d, J=11.7 Hz, 1H), 4.67 (d, J=9.7 Hz, 2H), 4.30 (d, J=9.8 Hz, 1H), 3.99-3.60 (m, 4H), 3.27-2.81 (m, 3H), 2.73-2.56 (m, 3H), 1.97-1.78 (m, 1H), 1.66-0.98 (m, 8H), 0.82-0.65 (m, 1H). HMIS (TOF, ESI) m/z: Calculated for C36H39FN4O5 626.2904, Found: 627.3002 [M+H]⁺

### rel-5-amino-6-(4-fluorophenoxy)-3-({4-hydroxy-1-[(1R,2R,4R)-4-hydroxy-2-phenyl-4-](pyridin-2-yl)methyl]cyclohexanecarbonyl]piperidin-4-yl}methyl)-3,4-dihydropyrimidin-4-one (EXAMPLE 96)

### Step 1: rel-ethyl (1R,2R,4R)-4-hydroxy-2phenyl-4-[(pyridin-2-yl)methyl]cyclohexane-1-carboxylate and rel-ethyl (1R,2R,4S)-4-hydroxy-2-phenyl-4-[(pyridin-2-yl)methyl]cyclohexane-1-carboxylate

To a solution of 2-picoline (0.07 mL, 0.67 mmol) in THF (4 mL), n-butyl lithium (2.5M solution in hexanes, 0.26 mL, 0.64 mmol) was added dropwise under N₂ at −78° C. After 20 minutes, a solution of rel-ethyl (1R,2R)-4-oxo-2-phenylcyclohexane-1-carboxylate (150 mg, 0.61 mmol) in THF (2 mL) was added dropwise and stirring continued at −78° C. for 1 hour. The reaction mixture was allowed to warm to r.t. and quenched with aq. NH₄Cl solution (10 mL). The aqueous layer was extracted with EtOAc (20 mL), dried (MgSO₄) and concentrated in vacua. The residue was purified via flash chromatography using heptane-25% EtOAc/Heptane (gradient) as eluent to afford:

First elute: rel-ethyl (1R,2R,4R)-4-hydroxy-2-phenyl-4-[(pyridin-2-yl)methyl]cyclohexane-1-carboxylate as a colourless oil.

LC/MS (Method B): RT=1.18; m/z=340 [M+H]⁺

Second elute: rel-ethyl (1R,2R,4S)-4-hydroxy-2-phenyl-4-[(pyridin-2-yl)methyl]cyclohexane-1-carboxylate as a colourless oil.

LC/MS (Method B): RT=1.18; m/z=340 [M+H]⁺

### Step 2: rel-(1R,2R,4R)-4-hydroxy-2-phenyl-4-[(pyridin-2-yl)methyl]cyclohexane-1-carboxylic acid

Starting from rel-ethyl (1R,2R,4R)-4-hydroxy-2-phenyl-4-[(pyridin-2-yl)methyl]cyclohexane-1-carboxylate (25 mg, 0.07 mmol) following procedure described in Step 2 of EXAMPLE 94, the reaction mixture was concentrated in vacuo to give rel-(1R2R,4R)-4-hydroxy-2-phenyl-4-[(pyridin-2-yltmethyl]cyclohexane-1-carboxylic acid. The compound was used without further purification.

LC/MS (Method B): RT=0.70; m/z=312 [M+H]⁺

Step 3: EXAMPLE 96

Starting from 5-amino-6-(4-fluorophenoxy)-3-[(4-hydroxypiperidin-4-yl)methyl]pyrimidin-4-one (23 mg, 0.07 mmol) and rel-(1R,2R,4R)-4-hydroxy-2-phenyl-4-[(pyridin-2-yl)methyl]cyclohexane-1-carboxylic acid (45 mg) using Step 3 of EXAMPLE 94. EXAMPLE 96 was obtained as a white solid.

LC/MS (Method B): RT=1.07; m/z=628 [M+H]⁺

¹H NMR (399 MHz, DMSO-d₆) δ 8.57-8.44 (m, 1H), 7.72 (tt, J=7.7, 1.9 Hz, 1H), 7.60 (d, J=23.8 Hz, 1H), 7.40 (ddt, J=7.9. 2.1, 1.1 Hz, 1H), 7.31-7.02 (m, 10H), 5.08 (d, J=16.2 Hz, 1H), 4.83 (d, J=13.1 Hz, 1H), 4.68 (d, J=11.4 Hz, 2H), 4.03-157 (m, 4H), 3.28-2.83 (m, 6H), 2.76-2.60 (m, 1H), 1.86-1.04 (m, 7H), 0.72 (ddt, J=66.2, 12.8, 6.5 Hz, 2H). HRMS (TOF, ESI) m/z: Calculated for C₃₅H₃₈FN₅O₅ 627.2857, Found; 628.2951 [M+H]

### rel-5-amino-6-(4-fluorophenoxy)-3-[(4-hydroxy-1-{[(1R,2R,4S)-4-hydroxy-2-phenyl-4-(pyridin-2-ylmethyl)cyclohexyl]carbonyl}piperidin-4-yl)methyl]pyrimidin-4-one

### Example 97

### Step 1: rel-(1R,2R,4S)-4-hydroxy-2-phenyl-4-[(pyridin-2-yl)methyl]cyclohexane-1-carboxylic acid

Starting from rel-ethyl (1R,2R,4S)-4-hydroxy-2-phenyl-4-[(pyridin-2-yl)methyl]cyclohexane-1-carboxylate (35 mg, 0.10 mmol) following procedure described in Step 2 of EXAMPLE 94, the reaction mixture was concentrated in vacuo to give rel-(1R,2R,4S)-4-hydroxy-2-phenyl-4-[(pyridin-2-yl)methyl]cyclohexane-1-carboxylic acid. The compound was used without further purification.

LC/MS (Method B): RT=0.74; m/z=312 [M+H]⁺

Step 2: EXAMPLE 97

Starting from 5-amino-6-(4-fluorophenoxy)-3-[(4-hydroxypiperidin-4-yl)methyl]pyrimidin-4-one (33 mg, 0.10 mmol) and rel-(1R,2R,4S)-4-hydroxy-2-phenyl-4-[(pyridin-2-yl)methyl]cyclohexane-1 -carboxylic acid (60 mg) using Step 3 of EXAMPLE 94, EXAMPLE 97 was obtained as a white solid.

LC/MS (Method B): RT=1.08; m/z=628 [M+H]⁺

¹H NMR (399 MHz, DMSO-₆) δ 8.46 (dddd, J=4.2, 3.1, 1.8, 0.8 Hz, 1H), 7.74-7.65 (m, 1H), 7.60 (d, J=22.6 Hz, 1H), 7.32-7.05 (m, 11H), 4.84-4.74 (m, 2H), 4.68 (d, J=10.5 Hz, 2H), 3.98-3.58 (m, 4H), 3.28-2.78 (m, 6H), 2.71-2.58 (m, 1H), 1.98-1.76 (m, 1H), 1.72-1.03 (m, 7H), 0.85-0.61 (m, 1H).

HRMS (TOF, ESI) m/z: Calculated for C₃₅H₃₈FN₅O₅ 627.2857, Found; 628.2952 [M+H]⁺

### rel-5-amino-6-(4-fluorophenoxy)-3-[(4-hydroxy-1-{([(1R,2R,4R)-4-hydroxy-2-phenyl-4-(pyrazin-2-ylmethyl)cyclohexyl]carbonyl}piperidin-4-yl)methyl]pyrimidin-4-one (EXAMPLE 98)

### Step 1: rel-ethyl (1R,2R,4R)-4-hydroxy-2-phenyl-4-[(pyrazin-2-yl)methyl]cyclohexane-1-carboxylate

Starting from rel-ethyl (1R,2R,4R)-4-oxo-2-phenylcyclohexane-1-carboxylate (700 mg, 2.84 mmol) and 2-methylpyrazine (0.24 mL, 2.58 mmol) following procedure described in Step 1 of EXAMPLE 96, the obtained residue was purified via flash chromatography using Heptane-10% EtOAc/Heptane (gradient) as eluent to afford rel-ethyl (1R,2R,4R)-4-hydroxy-2-phenyl-4 -[(pyrazin-2-yl)methyl]cyclohexane-1-carboxylate. The compound was used without further purification.

LC/MS (Method B): RT=1.09; m/z=341 [M+H]⁺

Step 2: rel-(1R,2R,4R)-4-hydroxy-2-phenyl-4-(pyrazin-2-ylmethyl)cyclohexane-1-carboxylic acid

Starting from rel-ethyl (1R,2R,4R)-4-hydroxy-2-phenyl-4-[(pyrazin-2-yl)methyl]cyclohexane-1-carboxylate (136 mg, 0.4 mmol) following procedure described in Step 2 of EXAMPLE 94, the reaction mixture was concentrated in vacuo to give rel-(1R,2R,4R)-4-hydroxy-2-phenyl-4-(pyrazin-2-ylmethyl)cyclohexane-1-carboxylic acid. The compound was used without further purification.

LC/MS (Method B): RT=0.84: m/z=313 [M+H]⁺

Step 3: EXAMPLE 98

Starting from 5-amino-6(4-fluorophenoxy)-3-[(4-hydroxypiperidin-4-yl)methyl]pyrimidin-4-one (107 mg, 0.32 mmol) and rel-(1R,2R,4R)-4-hydroxy-2-phenyl-4-(pyrazin-2-ylmethyl)cyclohexane-1-carboxylic acid (100 mg) following procedure described in Step 3 or EXAMPLE 94, the obtained residue was purified via flash chromatography using DCM-2% MeOH/DCM (gradient) as eluent to give EXAMPLE 98 as a white solid.

LC/MS (Method B): RT=1.00; m/z=629 [M+H]⁺

¹H NMR (399 MHz, DMSO-d₆) δ 8.63-8.52 (m, 2H), 8.46 (dd, J=4.3, 2.6 Hz, 1H), 7.61 (d, J=24.2 Hz, 1H), 7.34-7.04 (m, 9H), 4.88-4.75 (m, 2H), 4.68 (d, J=11.7 Hz, 2H), 4.01-3.57 (m, 4H), 3.29-2.84 (m, 5H), 2.68 (t, J=11.8 Hz, 1H), 1.90-1.07 (m, 8H), 0.82 (tt, J=12.5, 4.3 Hz, 1H), 0.63 (t, J=11.5 Hz, 1H).

HRMS (TOF, ESI) m/z: Calculated for C₃₄H₃₇FN₆O₅ 628.2809, Found: 629.2979 [M+H]⁻

### rel-5-amino-6-(4-fluorophenoxy)-3-({1-[(1R,2R,4R)-4-[(4-fluorophenyl))methyl]-4-hydroxy-2-phenylcyclohexanecarbonyl]-4-hydroxypiperidin-4-yl}methyl)-3,4-dihydropyrimidin-4-one (EXAMPLE 99)

### Step 1: rel-ethyl (1R,2R,4R)-4-{(4-fluorophenyl)methyl}-4-hydroxy-2-phenylcyclohexane 1-carboxylate and rel-(1R,5R,6S)-1-[(4-fluorophenyl)methyl]-6-phenyl-2-oxabicyclo[3.2.2]nonan-3-one

Following procedure described in Step 1 of EXAMPLE 94 and starting from rel-ethyl (1R,2R)-4-oxo-2-phenylcyclohexane-1-carboxylate (500 mg. 2.03 mmol) and bromo[(4-fluorophenyl)methyl]magnesium (1.94 mL, 1.15 M, 2.23 mmol) (prepared following procedure described in WO 2011/026349), the obtained residue was purified via flash chromatography using Heptane-50% EtOAc/Heptane (gradient) as eluent to afford:

First elute: rel-(1R,5R,6S)-1-[(4-fluorophenyl)methyl]-6-phenyl-2-oxabicyclo[12.2]nonan-3-one as a colourless oil.

¹H NMR (399 MHz, DMSO-d₆) δ 7.40-7.24 (m, 7H), 7.19-7.13 (m, 2H), 3.43-3.37 (m, 1H), 3.04 (s, 2H), 2.50-2.47 (m, 1H), 2.16-2.03 (m, 2H), 1.93-1.85 (m, 1H), 1.71-1.48 (m, 3H)

Second elute: rel-ethyl (1R,4R)-4-[(4-fluorophenyl)methyl]-4-hydroxy-2-phenyl cyclohexane-1-carboxylate as a colourless oil.

¹H NMR (399 MHz, DMSO-d₆) δ 7.25-7.14 (m, 7H), 7.11-7.05 (m, 2H), 4.53 (s, 1H), 3.81 (q, J=7.1 Hz, 2H), 2.99-2.81 (m, 3H), 2.65-2.57 (m, 1H), 1.01-1.84 (m, 1H), 1.79-1.60 (m, 2H), 1.55-1.42 (m, 3H), 0.85 (t, J=7.1 Hz, 3H).

### Step 2: rel-(1R,2R,4R)-4-[(4-fluorophenyl)methyl]-4-hydroxy-2-phenylcyclohexane-1-carboxylic acid

Starting from rel-ethyl (1R,2R,4R)-4-[(4-fluorophenyl)methyl]-4-hydroxy-2-phenyl cyclohexane-1-carboxylate (105 mg, 0.29 mmol) following procedure described in Step 2 of EXAMPLE 94. rel-(1R,2R,4R)-4-[(4-fluorophenyl)methyl]-4-hydroxy-2-phenyl cyclohexane-1-carboxylic acid was obtained as a yellow oil. The compound was used without further purification.

LC/MS (Method B): RT=1.07; m/z=327 [M−H]⁻

Step 3: EXAMPLE 99

Starting from 5-amino-6-(4-fluorophenoxyl-3-[(4-hydroxypiperidin-4-yl)methyl]pyrimidin-4-one (97 mg, 0.29 mmol) and rel-(1R,2R,4R)-4-[(4-fluorophenyl)methyl]-4-hydroxy-2-phenylcyclohexane-1 -carboxylic acid (95 mg) following procedure described in Step 3 of EXAMPLE 94, the residue was purified via flash chromatography using DCM-6% MeOH/DCM (gradient) as eluent to afford crude EXAMPLE 99. The residue was purified again via flash chromatography using EtOAc-2% MeOH/EtOAc (gradient) as eluent to give EXAMPLE 99 as a white solid.

LC/MS (Method B): RT=1.16; m/z=645 [M+H]⁺

¹H NMR (399 MHz, DMSO-d₆) δ 7.60 (d, J=24.1 Hz, 1H), 7.37-7.03 (m, 13H), 4.83 (d, J=12.4 Hz, 1H), 4.68 (d, J=11.9 Hz, 2H), 4.50 (d, J=2.7 Hz, 1H), 4.00-3.82 (m, 2H), 3.79-3.59 (m, 2H), 3.19-2.79 (m, 5H), 2.73-2.61 (m, 1H), 1.79-1.07 (m, 8H), 0.90-0.72 (m, 1H), 0.67-0.54 (m, 1H).

HRMS (TOF, ESI) m/z: Calculated for C₃₆H₃₈F₂N₄O₅ 644.2810, Found; 645.2910 [M+H]

### rel-5-amino-6-(4-fluorophenoxy)-3-[(1-{[(1R,2R,4S)-4-[(4-fluorophenyl)methyl]-4-hydroxy-2-phenylcyclohexyl]carbonyl}-4-hydroxypiperidin-4-yl)methyl]pyrimidin-4-one (EXAMPLE 100)

Step 1: rel-(1R,2R,4S)-4-[(4-fluorophenyl)methyl]-4-hydroxy-2-phenylcyclohexane-1-carboxylic acid

Starting from rel-(1R,5R,6S)-1-[(4-fluorophenyl)methyl]-6-phenyl-2-oxabicyclo[3.2.2]nonan-3-one (70 mg, 0.23 mmol) following procedure described in Step 2 of EXAMPLE 94. rel-(1R,2R,4S)-4-[(4-fluorophenyl)methyl]-4-hydroxy-2-phenylcyclohexane-1-carboxylic acid was obtained as a yellow oil. The compound was used without further purification.

LC/MS (Method B): RT=1.15; m/z=327 [M−H]⁻

Step 2: EXAMPLE 100

Starting from 5-amino-6-(4-fluorophenoxy)-3-[(4-hydroxypiperidin-4-yl)methyl]pyrimidin-4-one (74 mg, 0.22 mmol) and rel-(1R,2R,4S)-4-[(4-fluorophenyl)methyl]-4-hydroxy-2-phenylcyclohexane-1-carboxylic acid (182 mg) following procedure described in Step 3 of EXAMPLE 94, the obtained residue was purified via flash chromatography using EtOAc-2% MeOH/EtOAc (gradient) as eluent to give crude EXAMPLE 100. The residue was purified via prep HPLC (Prep HPLC Column; Gemini pH 4 Dimensions: 21.1 mm×150 mm 5 μm) to give EXAMPLE 100 as a white solid.

LC/MS (Method B): RT=1.22: m/z=645 [M+H]⁺

¹H NMR (399 MHz, DMSO-d₆) δ 7.60 (d, J=22.3 Hz, 1H), 7.30-7.02 (m, 13H), 4.81 (d, J=11.7 Hz, 1H), 4.67 (d, J=9.8 Hz, 2H), 4.31 (d, J=9.2 Hz, 1H), 3.98-3.78 (m, 2H), 3.75-3.60 (m, 2H), 3.26-2.81 (m, 3H), 2.66 (d, J=4.3 Hz, 3H), 1.87 (dt, J=12.2, 5.8 Hz, 1H), 1.65-1.05 (m, 8H), 0.82-0.65 (m, 1H).

HRMS (TOF, ESI) m/z: Calculated for C₃₆H₃₈F₂N₄O₅, Found; 645.2983 [M+H]⁺

### rel-5-amino-6-(4-fluorophenoxy)-3-({4-hydroxy-1-[(1R,2R,4R)-4-hydroxy-4-[(1-methyl-1H-imidazol-2-yl)methyl]-2-phenyl-cyclohexanecarbonyl]piperidin-4-yl)}methyl)-3,4-dihydropyrimidin-4-one (EXAMPLE 101)

### Step 1: rel-ethyl (1R,2R,4R)-4-hydroxy-4-[(1-methyl-1H-imidazol-2-yl)methyl]-2-phenyl-cyclohexane-1-carboxylate

Using Step 1 of EXAMPLE 96 and starting from rel-ethyl (1R,2R)-4-oxo-2-phenylcyclonexime-1-carboxylate (200 mg, 0.81 mmol, 1.0 eq.) and 1,2-dimethyl-1H-imidazole (156 mg, 1.62 mmol) instead of 2-picoline, rel-ethyl (1R,2R,4R)-4-hydroxy-4-[(1-methyl-1H-imidazol-2-yl)methyl]-2-phenyl-cyclohexane-1-carboxylate was obtained as a yellow oil.

LC/MS (Method B): RT=1.10; m/z=343 [M+H]⁺

### Step 2: rel-(1R,2R,4R)-4-hydroxy-4-[(1-methyl-1H-imidazol-2-yl)methyl]-2-phenyl-cyclohexane-1-carboxylic acid

Starting from rel-ethyl (1R,2R,4R)-4-hydroxy-4-[(1-methyl-1H-imidazol-2-yl) methyl]-2-phenylcyclohexane-1-carboxylate (40 mg, 0.012 mmol) following procedure described in Step 2 of EXAMPLE 94, (rel-(1R,2R,4R)-4-hydroxy-4-[(1-methyl-1H-imidazol-2-yl)methyl]-2-phenylcyclohexane-1-carboxylic acid was obtained. The compound was used without further purification.

LC/MS (Method B): RT=0.66; m/z=315 [M+H]⁺

Step 3: EXAMPLE 101

Starting from (rel-(1R,2R,4R)-4-hydroxy-4-[(1-methyl-1H-imidazol-2-yl)methyl]-2-phenylcyclohexane-1-carboxylic acid (85 mg) and 5-amino-6-(4-fluorophenoxy)-3-[(4-hydroxypiperidin-4-yl)methyl]pyrimidin-4-one (39 mg, 0.12 mmol) following procedure described in Step 3 of EXAMPLE 94. EXAMPLE 101 was obtained as a white solid.

LC/MS (Method B): RT=0.80; m/z=631 [M+H]⁺

¹H NMR (399 MHz, DMSO-d₆) δ 7.60 (d, J=24.5 Hz, 1H), 7.46 (d, J=2.3 Hz, 1H), 7.37 (d, J=4.4 Hz, 1H), 7.34-7.03 (m, 9H), 4.84 (d, J=14.0 Hz, 1H), 4.68 (d, J=11.8 Hz, 2H). 4.05-3.57 (m, 7H), 3.23-2.86 (m, 5H), 2.68 (m, J=12.9, 3.1 Hz, 1H), 1.88-1.07 (m, 9H), 0.82 (qd, J=14.3, 13.1, 4.8 Hz, 1H), 0.57 (td, J=13.1, 4.3 Hz, 1H).

HRMS (TOF, ESI) m/z: Calculated for C₃₄H₃₉FN₆O₅ 630.296, Found: 631.3098 [M+H]⁻

### rel-5-amino-6-(4-fluorophenoxy)-3-[(4-hydroxy-1-{[(1R,2R,4R)-4-hydroxy-2,4-diphenylcyclohexyl]carbonyl}piperidin-4-yl)methyl]pyrimidin-4-one (EXAMPLE 102)

### Step 1: rel -ethyl (1R,2R,4R)-4-hydroxy-2,4-diphenylcyclohexane-1-carboxylate and rel-(1R,5R,6S)-1,6-diphenyl-2-oxabicyclo[3.2.2]nonan-3-one

Starting from rel-ethyl (1R,2R)-4-oxo-2-phenylcyclohexane-1-carboxylate (500 mg, 2.03 mmol) and phenylmagnesium bromide (2.23 mL, 1M, 2.23 mmol) following procedure described in Step 1 of EXAMPLE 94, the obtained residue was purified via flash chromatography using Heptane-50% EtOAc/Heptane (gradient) as eluent to afford:

First elute: rel-(1R,5R,6S)-1,6-diphenyl-2-oxabicyclo[3.2.2]nonan-3-one as a white solid.

LC/MS (Method RT=1.30; m/z=279 [M+H]⁺

Second elute: rel-ethyl (1R,2R,4R)-4-hydroxy-2,4-diphenylcyclohexane-1-carboxylate as a colourless oil.

¹H NMR (399 MHz, DMSO-d₆) δ 7.60-7.52 (m, 2H), 7.43 (dd, J=8.4, 6.9 Hz, 2H), 7.36-7.24 (m, 3H), 7.27-7.14 (m, 3H), 5.08 (s, 1H), 3.73 (qd, J=7.1. 1.7 Hz, 2H), 2.79-2.52 (m, 3H), 2.46 (dt, J=13.2, 2.8 Hz, 1H), 1.99-1.87 (m, 2H), 1.81 (td, J=13.6, 3.8 Hz, 1H), 1.47-1.32 (m, 1H), 0.79 (t, J=7.1 Hz, 3H).

### Step 2: rel-(1R,2R,4R)-4-hydroxy-2,4-diphenylcyclohexane-1-carboxylic acid

Starting from rel-ethyl (1R,2R,4R)-4 -hydroxy-2,4-diphenylcyclohexane-1 -carboxylate (70 mg, 0.22 mmol) following procedure described in Step 2 of EXAMPLE 94, rel-(1R,2R,4R)-4-hydroxy-2,4-diphenylcyclohexane-1-carboxylic acid was obtained. The compound was used without further purification.

¹H NMR (399 MHz, DMSO-d₆) δ 11.85 (s, 1H, 7.60-7.53 (m, 2), 7.47-7.38 (m, 2H), 7.35-724 (m, 1H), 7.19 (ddd, J=6.9, 1.1 Hz, 3H),5.05 (s, 1H), 2.76-2.62 (m, 2H), 2.62-2.44 (m, 2H), 2.03-1.73 (m, 3H), 1.39 (m, 1H).

Step 3: EXAMPLE 102

Starting from 5-amino-6-(4-fluorophenoxy)-3-[(4-yl)methyl]pyrimidin-4-one (68 mg, 0.20 mmol) and rel-(1R,2R,4R)-4-hydroxy-2,4-diphenylcyclohexane-1-carboxylic acid (60 mg) following procedure described in Step 3 of EXAMPLE 94, the obtained residue was purified via flash chromatography using DCM-5% MeOH/DCM (gradient) as eluent to give EXAMPLE 102 as a white solid.

LC/MS (Method B): RT=1.11; m/z=613 [M+H]⁺

¹H NMR (399 MHz, DMSO-d₆) δ 7.68-7.49 (m, 3H), 7.42 (td, J=7.6, 3.3 Hz, 2H), 7.34-7.05(m, 10H), 5.02 (d, J=2.0 Hz, 1H), 4.80 (d, J=13.4 Hz, 1H), 4.67 (d, J=11.4 Hz, 2H), 3.97-3.58 (m, 4H), 3.23-2.85 (m, 2H), 2.83-2.71 (m, 1H), 2.66-2.56 (m, 1H), 2.49-2.39 (m, 1H), 2.11-1.78 (m, 3H), 1.75-1.61 (m, 1H), 1.41 (q, J=13.9, 13.4 Hz, 1H), 1.31-1.02 (m, 2H), 0.76 (td, J=12.8, 4.5 Hz, 1H), 0.59 (td, J=12.9, 4.3 Hz, 1H). HRMS (TOF, ESI) m/z: Calculated for C₃₅H₃₇FN₄O₅ 612.2748, Found; 613.2908 [M+H]⁺

### rel-5-amino-6-(4-fluorophenoxy)-3-[(4-hydroxy-1-{[(1R,2R,4S)-4-hydroxy-2,4-diphenylcyclohexyl]carbonyl}piperidin-4-yl)methyl]pyrimidin-4-one (EXAMPLE 103)

### Step 1: rel-(1R,2R,4S)-4-hydroxy-2,4-diphenylcyclohexane-1-carboxylic acid

Starting from rel-(1R,5R,6S)-1,6-diphenyl-2-oxabicyclo[3.2.2]nonan-3-one (360 mg, 1.29 mmol) following procedure described in Step 2 of EXAMPLE 94, rel-(1R,2R,4S)-4-hydroxy-2,4-diphenylcyclohexane-1-carboxylic acid was obtained. The compound was used without further purification.

LC/MS (Method B): RT=1.11; m/z=295 [M−H]⁻

Step 2: EXAMPLE 103

Starting from rel-(1R,2R,4S)-4-hydroxy-2,4-diphenylcyclohexane-1-carboxylic acid (100 mg, 0.34 mmol) and 5-amino-6-(4-fluorophenoxy)-3-[(4-hydroxypiperidin-4-yl)methyl]pyrimidin-4-one (113 mg, 0.34 mmol) following procedure described in Step 3 of EXAMPLE 94, the obtained residue was purified via flash chromatography using EtOAc-1.6% MeOH/EtOAc (gradient) as eluent to give EXAMPLE 103 as a white solid.

LC/MS (Method B): RT=1.19; m/z=595 [other]⁺

¹H NMR (399 MHz, DMSO-d₆) δ 7.67-7.51 (m, 3H), 7.36-7.05 (m, 12H), 4.97 (d, J=9.2 Hz, 1H), 4.83 (d, J=10.7 Hz, 1H), 4.68 (d, J=12.6 Hz, 2H), 4.01-3.64 (m, 4H), 3.42 (td, J=12.7, 11.9, 3.4 Hz, 1H), 3.29-2.93 (m, 2H), 2.70 (m, 1H), 2.19-1.92 (m, 3H), 1.79-1.06 (m, 6H), 0.86-0.69 (m, 1H).

HRMS (TOF, ESI) m/z: Calculated for C₃₅H₃₇FN₄O₅ 612.2748, Found; 613.2925 [M+H]

### 5-amino-3-({(4S)-1-[(1R,2R)-4,4-difluoro-2-phenylcyclohexane-1-carbonyl]-3,3-difluoro-4-hydroxypiperidin-4-yl}methyl)-6-(3-hydroxyphenoxy)pyrimidin-4(3H)-one

### Example 104

and

### 5-amino-3-([(4R)-1-[(1R,2R)-4,4-difluoro-2-phenylcyclohexane-1-carbonyl]-3,3-difluoro-4-hydroxypiperidin-4-yl]methyl)-6-(3-hydroxyphenoxy)pyrimidin-4(3H)-one

### Example 105

Using General Procedure 5 starting from Preparation R4at and Preparation R5b. 5-ammo-6-[3-(benzyloxy)phenoxy]-3-(|1-[(1R,2R)-4,4-difluoro-2-phenylcyclohexane-1-carbonyl]-3,3-difluoro-4-hydroxypiperidin-4-yl) methyl)pyrimidin-4(3H)-one was obtained. It was separated by chiral chromatography to give 5-amino-6-[3-(benzyloxy)phenoxy]-3-([(4S)-1-[(1R,2R)-4,4-difluoro-2-phenylcyclohexane-1-carbonyl]-3,3-difluoro-4-hydroxypiperidin-4-yl]methyl)pyrimidin-4(3H)-one and 5-amino-6-[3-(benzyloxy)phenoxy]-3-([(4S)-1-[(1R,2R)-4,4-difluoro-2-phenylcyclohexane-1-carbonyl]-3,3-difluoro-4-hydroxypiperidin-4-yl]methyl)pyrimidin-4(3H)-one. Autoclave was charged with 5-amino-6-[3-(benzyloxy)phenoxy]-3-([(4S)-1-[(1R,2R)-4,4-difluoro-2-phenylcyclohexane-1-carbonyl]-3,3-difluoro-4-hydroxypiperidin-4-yl]methyl)pyrimidin-4(3H)-one (83 mg, 0.1219 mmol) 10% palladium on charcoal (17 mg) and methanol (5 mL), and then placed under a nitrogen atmosphere. After that, it was filled with 10 bar H₂ gas. The reaction mixture was stirred in autoclave at r.t. for 19 hours. The catalyst was washed with methanol and filtered off. The mother liquor was purified by Hanbon prep HPLC, C18 Silica, Gemini NX 5 μm, 5 mM NH₄HCO₃-MeCN using gradient method 5-90%. Solvent was evaporated under reduced pressure to give EXAMPLE 104. HRMS calculated for C₂₉H₃₀F₄N₄O₅: 590.2152; found 591.2228 ((M+H)⁺ form).

Autoclave was charged with 5-amino-6-[3-(benzyloxy)phenoxy]-3-({(4R)-1-[(1R,2R)-4,4-difluoro-2-phenylcyclohexane-1-carbanyl[-3,3-difluoro-4-hydroxypiperidin-4-yl}methyl)pyrimidin-4(3H)-one (55 mg, 0.081 mmol) 10% palladium on charcoal (12 mg) and methanol (5 mL), and then placed under a nitrogen atmosphere. After that it was filled with 10 bar H₂ gas. The reaction mixture was stirred in autoclave at r.t. for 19 hours. The catalyst was washed with methanol and filtered off. The mother liquor was purified by Hanbon prep HPLC, C18 Silica, Gemini NX 5 μm. 5 mM NH₄HCO₃-MeCN using gradient method 5-90%. Solvent was evaporated under reduced pressure to give EXAMPLE 105. HRMS calculated for C₂₉H₃₀F₄N₄O₅: 590.21.52; found 591.2234 ((M+H)⁺form).

### 5-amino-3-([1-[(1R,2R)-4,4-difluoro-2-phenylcyclohexane-1-carbonyl]-4-hydroxypiperidin-4-yl]methyl)-6-[(1H-pyrrolo[3,2-b]pyridin-6-yl)oxy]pyrimidin-4(3H)-one (EXAMPLE 106)

Using General Procedure 5 starting from Preparation R4bs and Preparation R5a as reagents. EXAMPLE 106 was obtained. HRMS calculated for C₃₀H₃₂F₂N₆O₄: 578.2453; found 579.252 ((M+H)⁺ form).

### 4-{[5-amino-1-({1-[(1R,2R)-4,4-difluoro-2-phenylcyclohexane-1-carbonyl]-4-hydroxypiperidin-4-yl}methyl)6-oxo-1,6-dihydropyrimidin-4-yl]oxy}-3-chlorobenzamide (EXAMPLE 107)

EXAMPLE 53 (100 mg, 0.1672 mmol), (1E)-acetaldehyde oxime (98.77 mg, 0.102 mL, 1.352 mmol 10 eq.), Cu²⁺ on 4 Å molecular sieve (100 mg) were dissolved in methanol (3 mL) and 1,4-dioxane (2 mL). The reaction mixture was stirred at 60° C. for 18 hours. The mixture was filtered, the filtrate was evaporated and purified by preparative LC (on C-18 Gemini-NX 5 μm column, 5 mM aqueous NH₄HCO₃-MeCN gradient). Solvent was evaporated under reduced pressure to give EXAMPLE 107. HRMS calculated for C₃₀H₃₂ClF₂N₅O₅: 615.206; found 616.2129 ((M+H)⁺ form).

### 5-amino-3-({1-[(1R,2R)-4,4-difluoro-2-phenylcyclohexane-1-carbonyl]-4-hydroxypiperidin-4-yl}methyl)-6-{4-[(dimethylamino)methyl]phenoxy}pyrimidin-4(3H)-one (EXAMPLE 108)

EXAMPLE 44 (100 mg, 0.1765 mmol), dimethylamine (2M in THF) (5.0 eq.), sodium triacetoxyborohydride (5.0 eq.), acetic acid (5.0 eq.) were dissolved in THF and stirred r.t. for 24 hours. The reaction mixture was diluted with water and it was purified by preparative LC (on C-18 Gemini-NX 5 μm column, 5 mM aqueous NH₄HCO₃-MeCN, gradient).

Solvent was evaporated under reduced pressure to give EXAMPLE 108. HRMS calculated for C₃₂H₃₉F₂N₅O₄: 595.297; found 596.3035 ((M+H)⁺ form).

### 5-amino-3-({(4S)-1-[(1R,2R)-4,4-fluoro-2-phenylcyclohexane-1-carbonyl]-3,3-difluoro-4-hydroxypiperidin-4-yl}methyl)-6-(4-fluoro-3-hydroxyphenoxy)pyrimidin-4(3H)-one (EXAMPLE 109)

Using General Procedure 5 starting from Preparation R4ar and Preparation R5b as reagents, after chiral separation. EXAMPLE 109 was obtained. HRMS calculated for C₂₉H₂₉F₅N₄O₅: 608.2058; found 609.2125 ((M+H)⁺ form).

### 5-amino-6-(4-chloro-3-hydroxyphenoxy)-3({1-[(1R,2R)-4,4-difluoro-2-phenylcyclohexane-1-carbonyl]-4-hydroxypiperidin-4-yl}methyl)pyrimidin-4(3H)-one

### Example 110

Using General Procedure 5 starting from Preparation R4bt and Preparation R5a as reagents, to give 5-amino-6-(3-benzyloxy-4-chloro-phenoxy)-3-[[1-[(1R,2R)-4,4-difluoro-2-phenyl-cyclohexanecarbonyl]-4-hydroxy-4-piperidyl]methyl]pyrimidin-4-one as a crude product. Autoclave was charged with 5-amino-6-(3-benzyloxy-4-chloro-phenoxy)-3-[[1-[(1R,2R)-4,4-difluoro-2-phenyl-cyclohexanecarbonyl]-4-hydroxy-4-piperidyl]methyl]pyrimidin-4-one (93 mg, 0.137 mmol) 10% palladium on charcoal (15 mg) and 1,4-dioxane (3 mL), and then placed under a nitrogen atmosphere. After that it was filled with 10 bar H₂ gas. The reaction mixture was stirred in autoclave at r.t. for 21 hours. The catalyst was washed with methanol and filtered off. The mother liquor was purified by Hanbon preparative HPLC, C18 Silica, Gemini NX 5 μm, 5 mM NH₄HCO₃-MeCN using gradient method 5-90%. Solvent was evaporated under reduced pressure to give EXAMPLE 110. HRMS calculated for C₂₉H₃₁ClF₂N₄O₅: 588.1951; found 589.2011 ((M+H)⁺ form).

### 5-amino-3-({1-[(1R,2R)-4,4-difluoro-2-phenylcyclohexane-1-carbonyl]-4-hydroxypiperidin-4-yl}methyl)-6-(3-hydroxy-4-methylphenoxy)pyrimidin-4(3H)-one

### Example 111

Using General Procedure 5 starting from Preparation R4bu and Preparation R5a as reagents, to give 5-amino-6-(3-benzyloxy-4-methyl-phenoxy)-3-[[1-[(1R,2R)-4,4-difluoro-2-phenyl-cyclohexanecarbonyl]-4-hydroxy-4-piperidyl]methyl]pyrimidin-4-one as a crude product. Autoclave was charged with 5-amino-6-(3-benzyloxy-4-methyl-phenoxy)-3-[[1-[(1R,2R)-4,4-difluoro-2-phenyl-cyclohexanecarbonyl]-4-hydroxy-4-piperidyl]methyl]pyrimidin-4-one (85 mg, 0.129 mmol) 10% palladium on charcoal (14 mg) and 1,4-dioxane (3 mL), and then placed under a nitrogen atmosphere. After that it was filled with 10 bar H₂ gas. The reaction mixture was stirred in autoclave at r.t. for 21 hours. The catalyst was washed with methanol and filtered off. The mother liquor was purified by Hanbon HPLC, C18 Silica, Gemini NX 5 μm, 5 mM NH₄HCO₃-MeCN using gradient method 5-90%. Solvent was evaporated under reduced pressure to give EXAMPLE 111. HRMS calculated for C₃₀H₃₄F₂N₄O₅: 568.2498; found 569.2569 (M+H)⁺ form).

### 5-amino-3-([1-[(1R,2R)-4,4-difluoro-2-phenylcyclohexane-1-carbonyl]-4-hydroxypiperidin-4-yl]methyl)-6-[4-[2-(piperidin-4-yl)ethyl]phenoxy]pyrimidin-4(3H)-one (EXAMPLE 112)

Using General Procedure 5 starting from Preparation R4bv and Preparation R5a as reagents. tert-butyl 4-[2-[4-[5-amino-1-][1-[(1R,2R)-4,4-difluoro-2-phenyl-cyclohexanecarbonyl]-4-hydroxy-4-piperidyl]methyl]-6-oxo-pyrimidin-4-yl]oxyphenyl]ethyl]piperidine-1-carboxylate was formed. The resulted Boc-protected crude product was reacted using General Procedure 7. It was purified by Hanbon preparative HPLC, C18 Silica, Gemini NX 5 μm, 5 mM NH₄HCO₃-MeCN using gradient method 5-90%. Solvent was evaporated under reduced pressure to give to EXAMPLE 112. HRMS calculated for C₃₆H₄₅F₂N₅O₄: 649.3439; found 650.3496 ((M+H)⁺ form).

### 5-amino-3-({1-[(1R,2R)-4,4-difluoro-2-phenylcyclohexane-1-carbonyl]-4-hydroxypiperidin-4-yl}methyl)-6-{4-[(morpholin-4-yl)methyl]phenoxyl}pyrimidin-4(3H)-one (EXAMPLE 113)

Using General Procedure 5 starting from Preparation R5bw and Preparation R5a as reagents. EXAMPLE 113 was obtained. HRMS calculated for C₃₄H₄₁F₂N₅O: 637.3076; found 638.31482 ((M+H)⁺ form).

### 5-amino-3-({1-[(1R,2R)-4,4-difluoro-2-phenylcyclohexane-1-carbonyl]-4-hydroxypiperidin-4-yl}methyl)-6-{4-[2-(piperidin-2-yl)ethyl]phenoxy}pyrimidin-4(3H)-one (EXAMPLE 114)

Using General Procedure 5 starting from Preparation R4bx and Preparation R5a as reagents. tert-butyl 2-[3-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]morpholine-4-carboxylate was formed. The resulted Boc-protected crude product was reacted using General Procedure 7. It was purified by Hanbon preparative HPLC, C18 Silica, Gemini NX 5 μm, 5 mM NH₄HCO₃-MeCN using gradient method 5-90%. Solvent was evaporated under reduced pressure to give to EXAMPLE 114. HRMS calculated for C₃₆H₄₅F₂N₅O₄: 649.3439; found 650.3505 ((M+H)⁺ form).

### 5-amino-3-([1-[(1R,2R)-4,4-difluoro-2-phenylcyclohexane-1-carbonyl]-4-hydroxypiperidin-4-yl]methyl)-6-[3-(morpholin-2-yl)phenoxy]pyrimidin-4(3H)-one

### Example 115

Using General Procedure 5 starting from Preparation R4by and Preparation R5a as reagents, tert-butyl 2-[3-[5-amino-1-[[1-[(1R,2 R)-4,4-fluoro-2-phenyl-cyclohexanecarbonyl]-4-hydroxy-4-piperidyl]methyl]-6-oxo-pyrimidin-4-yl]oxyphenyl]morpholine-4-carboxylate was formed. The resulted Boc-protected crude product was reacted using General Procedure 7. It was purified by Hanbon preparative HPLC, C18 Silica, Gemini NX 5 μm, 5 mM NH₄HCO₃-MeCN using gradient method 5-90%. Solvent was evaporated under reduced pressure to give to EXAMPLE 115. HRMS calculated for C₃₃H₃₉F₂N₅O₅: 623.2919; found 624.2990 ((M+H)⁺ form).

### 5-amino-6-{4-[(1R)-1-amino-2,2,2-trifluoroethyl]phenoxy}-3-({1-[(1R,2R)-4,4-difluoro-2-phenylcyclohexane-1-carbonyl]-4-hydroypiperidin-4-yl}methyl)pyrimidin-4(3H)-one (EXAMPLE 116)

Using General Procedure 5 starting from Preparation R4bz and Preparation R5a as reagents, tert-butyl N-[(1R)-1-[4-[5-amino-1-[[1-[(1R,2R)-4,4-difluoro-2-phenyl-cyclohexanecarbonyl]-4-hydroxy-4-piperidyl]methyl]-6-oxo-pyrimidin-4-yl]oxyphenyl]-2,2,2-trifluoro-ethyl]carbamate was formed. The resulted Boc-protected crude product was reacted using General Procedure 7. It was purified by Hanbon preparative HPLC. C18 Silica. Gemini NX 5 μm, 5 mM NH₄HCO₃-MeCN using gradient method 5-90%. Solvent was evaporated under reduced pressure to give to EXAMPLE 116. HRMS calculated for C₃₁H₃₄F₅N₅O₄: 635.2531; found 636.26 ((M+H)⁺ form).

### (4-{[5-amino-1-({1-[(1R,2R)-4,4-difluoro-2-phenylcyclohexane-1-carbonyl]-4-hydroxypiperidin-4-yl}methyl)-6-oxo-1,6-dihydropyrimidin-4-yl]oxy}phenyl)acetonitrile (EXAMPLE 117)

Using General Procedure 5 starting from Preparation R5ca and Preparation R5a as reagents, EXAMPLE 117 was obtained. HRMS calculated for C₃₁H₃₃F₂N₅O₄: 577.2501; found 578.2567 ((M+H⁺ form).

### 5-amino-3-({1-[(1R,2R)-4,4-difluoro-2-phenylcyclohexane-1-carbonyl]-4-hydroxypiperidin-4-yl}methyl)-6-[4-fluoro-3-(hydroxymethyl)phenoxy]pyrimidin-4(3H)-one (EXAMPLE 118)

Using General Procedure 5 starting from Preparation R4cb and Preparation R5a as reagents. EXAMPLE 118 was obtained. HRMS calculated for C₃₀H₃₃F₃N₄O₅: 586.2403; found 587.2473 ((M+H)⁺ form).

### 5-amino-6-(3-amino-4-fluorophenoxy)-3-({1-[(1R,2R)-4,4-difluoro-2-phenylcyclohexane-1-carbonyl]-4-hydroxypiperidin-4-yl}methyl)pyrimidin-4(3H)-one

### Example 119

Using General Procedure S starting from Preparation R4cc and Preparation R5a as reagents. EXAMPLE 119 was obtained. HRMS calculated for C₂₉H₃₂F₃N₅O₄: 571.2407; found 572.2477 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[3-hydroxy-4-(propan-2-yl)phenoxy]pyrimidin-4(3H)-one (EXAMPLE 120)

Using General Procedure 5 starting from Preparation R4cd and Preparation R5a as reagents. EXAMPLE 120 was obtained. HRMS calculated for C₃₂H₃₈F₂N₄O₅: 596.281; found 597.2883 ((M+H)⁺ form).

### 5-amino-3-({1-[(1R,2R)-4,4-difluoro-2-phenylcyclohexane-1-carbonyl]-4-hydroxypiperidin-4-yl}methyl)-6-[4-(piperidin-2-yl)phenoxy]pyrimidin-4(3H)-one

### Example 121

Using General Procedure 5 starting from Preparation R4ce and Preparation R5a as reagents, tert-butyl 2-[4-[5-amino-1-[[1-[(1 R,2R)-4,4-difluoro-2-phenyl-cyclohexane carbonyl]-4-hydroxy-4-piperidyl]methyl]-6-oxo-pyrimidin-4-yl]oxyphenyl]piperidine-1-carboxylate was formed. The resulted Boc-protected crude product was reacted using General Procedure 7 and the crude was purified by Hanbon preparative HPLC. C18 Silica, Gemini NX 5 μM, 5 mM NH₄HCO₃-MeCN using gradient method 5-90%. Solvent was evaporated under reduced pressure to give to EXAMPLE 121. HRMS calculated for C₁₄H₄₁F₂N₅O₄: 621.3127; found 622.3203 ((M+H)⁺ form).

### 5-amino-3-({1-[(1R,2R,4S)-4-ethynyl-4-fluoro-2-phenylcyclohexanecarbonyl]-4-hydroxypiperidin-4-yl}methyl)-6-(4-fluorophenoxy)-3,4-dihydropyrimidin-4-one

### Example 122

and

### 5-amino-3-({1-[(1R,2R,4R)-4-ethynyl-4-fluoro-2-phenylcyclohexanecarbonyl]-4-hydroxypiperidin-4-yl}methyl)-6-(4-fluorophenoxy)-3,4-dihydropyrimidin-4-one

### Example 123

### Step 1: Ethyl (1R,2R)-4-oxo-2-phenylcyclohexane-1-carboxylate (E1) and ethyl (1S,2S)-4-oxo-2-phenylcyclohexane-1-carboxylate (E2)

The enantiomers of rel-ethyl (1R,2R)-4-oxo-2-phenylcyclohexane-1-carboxylate were separated via chiral chromatography (Column: 1A, Eluents: heptane/DCM). The enantiomer eluting earlier was collected as E1 with 99.8% ee and the enantiomer eluting later was collected as E2 with 99.9% ee.

### Step 2: Ethyl (1R,2R)-4-hydroxy-2-phenyl-4-[2-(trimethylsilyl)ethynyl]cyclohexane-1-carboxylate

To a solution of diisopropylamine (0.4 mL, 2.84 mmol, 1.35 eq.) in THF(10 mL), was added n-butyl lithium (2.5M in hexane, I.15 mL, 2.74 mmol, 1.35 eq.) dropwise at -78° C. under nitrogen. The reaction mixture was stirred for 10 minutes before adding (trimethylsilyl)acetylene (037 mL, 2.64 mmol, 1.3 eq.) in THF (2 mL) dropwise. After 5 minutes. a solution of E1 (500 mg, 2.03 mmol) in THF (3 mL) was added dropwise and stirring continued at −78° C. for 4 hours, The reaction mixture was quenched with aq. NH₄Cl solution (10 mL). The aqueous layer was extracted with EtOAc (20 mL). dried (MgSO₄) and concentrated in vacuo. The residue was purified via flash chromatography using Heptane-15% EtOAc/Heptane (gradient) as eluent to afford ethyl (1R,2R,4R)-4-hydroxy-2-phenyl-4-[2-(trimethylsilyl)ethynyl]cyclohexane-1-carboxylate as a colourless oil. The compound was used without further purification.

¹H NMR (399 MHz, Chloroform-d) δ 7.21-7.01 (m, 5H), 3.83-3.66 (m, 2H), 104 (ddd, J=12.9, 11.4, 3.2 Hz, 1H), 2.42-2.32 (m, 1H), 2.02-1.78 (m, 4H), 1.63 (t, J=12.8 Hz, 1H), 1.51 (td, J=12.5, 4.9 Hz, 1H), 0.82 (td, J=7.1, 2.3 Hz, 3H), 0.10 (s, 9H).

### Step 3: Ethyl (1R,2R,4R)-4-ethynyl-4-hydroxy-2-phenylcyclohexane-1-carboxylate

Starting from ethyl (1R,2R,4R)-4-hydroxy-2-phenyl-4-[2-(trimethylsilyl)ethynyl]cyclohexane-1-carboxylate (970 mg, 2.82 mmol) following procedure described in Step 2 or EXAMPLE 91, the obtained residue was purified via flash chromatography using Heptane-40 % EtOAc/Heptane (gradient) as eluent to afford ethyl (1R,2R,4R)-4-ethynyl-4-hydroxy-2-phenylcyclohexane-1-carboxylate as a colourless oil.

¹H NMR (399 MHz, Chloroform-d) δ 7.37-7.13 (m, 5H), 3.90 (q, J=7.1 Hz, 2H), 3.21 (ddd, J=13.0, 11.5, 3.3 Hz, 1H), 2.67 (s, 1H), 2.56 (td, J=11.5, 4.4 Hz, 1H), 2.28-2.13 (m, 2H), 2.12-1.96 (m, 2H), 1.82 (t, J=12.9 Hz, 1H), 1.70 (td, J=12.7, 4.6 Hz, 1H), 0.96 (t, J=7.1 Hz, 3H).

### Step 4: Ethyl (1R,2R)-4-ethynyl-4-fluoro-2-phenylcyclohexane-1-carboxylate

To a solution of triethylamine trihydrofluoride (0.74 mL, 4.55 mmol, 2.0 eq.) in DCM (12 mL). was added triethylamine (0.32 ml, 2.28 mmol, 1.0 eq.) at −78° C. under nitrogen. XtalFluor-M® (830 mg, 3.41 mmol, 1.5 eq.1 and a solution of ethyl (1R,2R,4R)-4-ethynyl-4-hydroxy-2-phenylcyclohexane-1-carboxylate (620 mg, 2.28 mmol, 1.0 eq.) in DCM (12 mL) were then added sequentially The reaction mixture was stirred at the same temperature for 1 hour before being allowed to warm to r.t. overnight. The mixture was quenched with 5% aq. NaHCO) solution (40 mL) and stirred for 15 minutes. DCM (50 mL) was added and the organic layer was separated, dried (MgSO₄) and evaporated in vacuo. The residue was purified via flash chromatography using Heptane-5% EtOAc/Heptane (gradient) as eluent to afford the ethyl (1R,2R)-4-ethynyl-4-fluoro-2-phenylcyclohexane-1-carboxylate as a colourless oil.

¹H NMR (399 MHz, Chloroform-d) δ 7.38-7.11 (m, 5H), 3.90 (qd, J=7.1, 1.6 Hz, 2H), 3.21 (tdd, J=16.9,12.4, 3.3 Hz, 1H), 2.90-2.52 (m, 2H), 2.50-2.30 (m, 2H), 2.25-1.69 (m, 4H), 0.96 (td, J=7.1, 2.8 Hz, 3H).

### Step 5: (1R,2R¹⁻⁴ -ethynyl-4-fluoro-2-phenylcyclohexane-1-carboxylic acid

To a solution of ethyl (1R,2R)-4-ethynyl-4-fluoro-2-phenylcyclohexane-1-carboxylate (385 mg, 1.4 mmol) in THF (4 mL), methanol (2 mL) and water (1 mL), was added lithium hydroxide monohydrate (353 mg, 8.42 mmol, 6.0 eq.) and the mixture was stirred at r.t. for 72 hours, The reaction mixture was concentrated in vacua, water (10 mL) was added and the mixture was acidified to pH 3 using 1.2N HCl (7 mL) The aqueous layer was extracted with EtOAc (50 mL), dried (MgSO₃) and evaporated in vacuo to afford (1R,2R)-4-ethynyl-4-fluoro-2-phenylcyclohexane-1-carboxylic acid as a colourless oil. The compound was used without further purification.

¹H NMR (399 MHz, DMSO-d₆) δ 12.01 (s, 1H), 7.38-7.07 (m, 5H), 4.13-3.84 (m, 1H), 2.97 (ddd, J=11.6, 8.9, 4.7 Hz, 1H), 2.84-2.59 (m, 1H), 2.27-1.82 (m, 5H), 1.82-1.63 (m, 1H),

### Step 6: EXAMPLE 122 and EXAMPLE 123

Starting from (1R,2R)-4-ethynyl-4-fluoro-2-phenylcyclohexane-1-carboxylic acid (350 mg, 1.42 mmol) and 5-amino-6-(4-fluorophenoxy)-3-[(4-hydroxypipendin-4-yl) methyl]pyrimidin-4-one (475 mg, 1.42 mmol) following procedure described in Step 3 of EXAMPLE 94, the obtained residue was purified via flash chromatography using DCM-5% MeOH/DCM (gradient) as eluent to afford the products a mixture of diastereoisomers. Final purification via prep HPLC (Prep HPLC Column: Gemini pH 4 Dimensions: 21.1 mm×150 mm 5 μm) gave:

First elute: EXAMPLE 122 as a white solid.

LC/MS (Method B): RT=1.18; m/z: 563 [M+H]⁺

¹H NMR (399 MHz, DMSO-d₆) δ 7.59 (m, 1H), 7.35-7.13 (m, 7H), 7.09 (ddd, J=9.1, 4.6, 2.3 Hz, 2H), 4.82 (d, J=7.3 Hz, 1H). 4.69 (d, J=14.1 Hz, 2H), 3.99-3.58 (m, 5H). 3.33-3.02 (m, 3H), 2.97-2.57 (m, 1H), 2.28-1.88 (m, 4H), 1.85-1.53 (m, 2H), 1.49-1.06 (m, 3H), 0.68 (dtd, J=44.2, 12.9, 4.4 Hz, 1H),

HRMS (TOF, ESI) m/z: Calculated for C₃₁H₃₂F₂N₄O₄ 562.2392, Found: 563.2490 [M+H]⁺

Second elute: EXAMPLE 123 as a white solid.

LC/MS (Method B): RT=1.19; m/z=563 [M+H]⁺

¹H NMR (399 MHz, DMSO-d₆) δ 7.59 (m, 1H), 7.39-6.99 (m, 9H), 4.81 (d, J=7.2 Hz, 1H), 4.69 (d, J=14.6 Hz, 2H), 4.09-3.77 (m, 3H), 3.75-3.57 (m, 2H), 3.26-3.01 (m, 3H), 2.92-2.56 (m, 1H), 2.35-1.89 (m, 4H), 1.80 (d, J=14.3 Hz, 2H), 1.50-1.05 (m, 3H), 0.64 (dtd, J=81.7, 13.0, 4.4 Hz, 1H).

HRMS (TOF, ESI) m/z: Calculated for C₃₁H₃₂F₂N₄O₄ 562.2392, Found: 563.2507 [M+H]⁺

### rel-5-amino-3-[(1 -{[(1R,2 R)-4-(2-cyclopropylethynyl)-4-fluoro-2-phenylcyclohexyl]carbonyl}-4-hydroxypiperidin-4-yl)methyl]-6-(4-fluorophenoxy)pyrimidin-1-one (EXAMPLE 124)

### Step 1: rel-ethyl (1R,2R,4R)-4-(2-cyclopropylethynyl)-4-hydroxy-2-phenylcyclohexane-1-carboxylate

Starting from rel-ethyl (1R,2R)-4-oxo-2-phenylcyclohexane-1-carboxylate (500 mg, 2.03 mmol) and ethynylcyclopropane (0.22 ml, 2.64 mmol, 1.3 eq.) following procedure described in Step 2 of EXAMPLES 122 and 123, the obtained residue was purified via flash chromatography using Heptane-25% EtOAc/Heptane (gradient) as eluent to afford rel-ethyl (1R,2R,4R)-4-(2-cyclopropylethynyl)-4-hydroxy-2-phenylcyclohexane-1-carboxylate as a colourless oil.

¹H NMR (399 MHz, Chloroform-d) δ 7.34-7.25 (m, 2H), 7.25-7.17 (m, 3H), 3.90 (q, J=7.1 Hz, 2H), 3.16 (ddd, .J=13.0, 11.5, 3.3 Hz 1H), 2.53 (td, J=11.5, 4.4 Hz, 1H), 2.16-1.87 (m, 4H), 1.78(t, J=12.8 Hz, 1H), 1.71-1.61 (m, 1H), 1.41-1.24 (m, 1H), 0.96 (t, J=7.1 H), 3H), 0.91-0.72 (m, 4H).

### Step 2: rel-ethyl (1R,2R)-4-(2-cyclopropylethynyl)-4-hydroxy-2-phenylcyclohexane-1-carboxylate

Starting from rel-ethyl (1R,2R,4R)-4-(2-cyclopropylethynyl)-4-hydroxy-2-phenyl cyclohexane-1-carboxylate (233 mg, 0.96 mmol) following procedure described in Step 4 of EXAMPLES 122 and 123, rel-ethyl (1R,2R)-4-(2-cyclopropylethynyl)-4-fluoro-2-phenylcyclohexane-1-carboxylate (157 mg, 0.5 mmol, 78%) was obtained as a colourless oil.

¹H NMR (399 MHz, Chloroform-d) δ 7.35-7,25 (m, 2H), 7.27-7.17 (m, 3H), 3.90 (qd, J=7.1, 3.8 Hz, 2H), 3.26-3.09 (m, 1H), 2.68-2.51 (m, 1H), 2.43-2.21 (m, 2H), 2.15-1.68 (m, 4H), 1.42-1.27 (m, 1H), 0.96 (J=7.1 Hz, 3H), 0.92-0.69 (m, 5H).

### Step 3: rel-(1R,2R)-4-(2-cyclopropylethynyl-4-fluoro-2-phenylcyclohexane-1-carboxylic acid

Starting from rel-ethyl (1R,2R,4R)-4-(2-cyclopropylethynyI)-4-hydroxy-2-phenyl cyclohexane-1-carboxylate (255 mg, 0.81 mmol) following procedure described in Step 5 or EXAMPLES 122 and 123, rel-(1R,2R)-4-(2-cyclopropylethynyl)-4-fluoro-2-phenylcyclohexane-1-carboxylic acid was obtained as a colourless oil. The compound was used without further purification.

LC/MS (Method B): RT=1.29; m/z 285 [M−H]⁺

Step 4: EXAMPLE 124

Starting from 5-amino-6-(4-fluorophenoxy)-3-[(4-hydroxypiperidin-4-yl)methyl]pyrimidin-4-one (140 mg, 0.42 mmol) and rel-(1R,2R)-4-(2-cyclopropylethyny))-4-fluoro-2-phenylcyclohexane-1-carboxylic acid (274 mg) following procedure described in Step 3 of EXAMPLE 94, the obtained residue was punned via flash chromatography using Hexane-100% EtOAc/Hexane (gradient) as eluent to give the crude product as a white solid. Final purification via prep HPLC (Prep HPLC Column: Gemini pH 4 Dimensions: 21.1 mm×150 mm 5 μm) afforded the desired product as a white solid.

LC/MS (Method B): RT=1.28; m/z=603 [M+H]⁺

¹H NMR (399 MHz, DMSO-d₆) δ 7.65-7.53 (m, 1H), 7.34-7.13 (m, 7H), 7.09 (ddd, J=9.0, 4.6, 2.1 Hz, 2H), 4.82 (d, J=8.0 Hz, 1H), 4.69 (d, J=13.9 Hz, 2H), 4.01-3.77 (m, 2H), 3.76-3.56 (m, 2H), 3.33-2.98 (m, 3H), 2.95-2.57 (m, 1H), 2.22-1.82 (m, 4H), 1.81-1.53 (m, 2H), 1.52-1.03 (m, 4H), 0.94-0.47 (m, 5H).

HRMS (TOF, ESI) m/z: Calculated for C₃₄H₃₀F₂N₄O₄ 602.2705 Found: 603.2729 [M+H]⁺

### rel-5-amino-3-[(1-{[(1R,2R)-4-fluoro-2-phenyl-4-(prop-1-yn-1-yl)cyclohexyl]carbonyl}-4-hydroxypiperidin-4-yl)methyl]-6-(4-fluorophenoxy)pyrimidin-4-one (EXAMPLE 125)

### Step 1: rel-ethyl (1R,2R,4R)-4-hydroxy-2-phenyl-4-(prop-1-yn-1-yl)cyclohexane-1-carboxylate

Starting from rel-ethyl (1R,2R)-4-oxo-2-phenylcyclohexane-1-carboxylate (500 mg, 2.03 mmol) and propyne (2.64 mL, 1M in THF, 2.64 mmol, 13 eq.), following procedure described in Step 2 of EXAMPLES 122 and 123, the obtained residue was purified via flash chromatography using Heptane-30% EtOAc/Heptane (gradient) as eluent to afford rel-ethyl (1R,2R,4R)-4-hydroxy-2-phenyl-4-(prop-1-yn-1-yl)cyclohexane-1-carboxylate as a colourless oil.

¹H NMR (399 MHz, Chloroform-d) δ 7.35-7.14 (m, 5H), 3.90 (t, 2H), 3.17 (ddd, J=12.9, 11.4, 3.3 Hz, 1H), 2.54 (td, J=11.4, 4.6 Hz, 1H), 2.25-1.96 (m, 4H), 1.95 (s, 1H), 1.78 (t, J=12.8 Hz, 1H), 1.72-1.60 (m, 3H), 0.95 (t, J=7.1 Hz, 3H).

### Step 2: rel-ethyl (1R,2R)-4-hydroxy-2-phenyl-4-(prop-1-yn-1-yl)cyclohexane-1-carboxylate

Starting from rel-ethyl (1R,2R,4R)-4-hydroxy-2-phenyl-4-(prop-1-yn-1-yl)cyclohexane-1-carboxylate (360 mg, 1.26 mmol) following procedure described in Step 4 of EXAMPLES 122 and 123, re-ethyl (1R,2R)-4-fluoro-2-phenyl-4-(prop-1-yn-1-yl)cyclohexane-1-carboxylate was obtained as a colourless oil.

¹H NMR (399 MHz, Chloroform-d) δ 7.36-7.15 (m, 5H), 3.90 (qd, J=7.1, 2.5 Hz, 2H), 128-3.08 (m, 1H), 2.68-2.51 (m, 1H), 2.44-2.23 (m, 2H), 2.17-1.69 (m 7H), 0.96 (td, J=7.1, 3.8 Hz, 3H).

### Step 3 rel-(1R,2R)-4-fluoro-2-phenyl-4-(prop-1-yn-1-yl)cyclohexane-1-carboxylic acid

Starting from rel-ethyl (1R,2R)-4-fluoro-2-phenyl-4-(prop-1-yn-1-yl)cyclohexane-1-carboxylate (280 mg, 0.97 mmol) following procedure described in Step 5 of EXAMPLES 122 and 123, rel-(1R,2R)-4-fluoro-2-phenyl-4-(prop-1-yn-1-yl)cyclohexane-1-carboxylic acid was obtained as a colourless oil. The compound was used without further purification.

LC/MS (Method B): RT=1.20; m/z=259 [M−H]⁺

¹H NMR (399 MHz, DMSO-d₆) δ 11.97 (s, 1H), 7.34-7.15 (m, 5H), 3.03-2.92 (m, 1H), 2.80-2.60(m, 1H), 2.13-1.83 (m, 8H), 1.76 (m, 1H)

### Step 4: EXAMPLE 125

Starting from 5-amino-6-(4-fluorophenoxyl-3-[(4-hydroxypiperidin-4-yl)methyl]pyrimidin-4-one (154 mg, 0.46 mmol) and rel-(1R,2R)-4-fluoro-2-phenyl-4-(prop-1-yn-1-yl)cyclohexane-1-carboxylic acid (120 mg) following procedure described in Step 3 of EXAMPLE 94, the obtained residue was purified via flash chromatography using DCM-5% MeOH/DCM (gradient) as eluent to give the crude product as a white solid. Final purification via prep HPLC (Prep HPLC Column: Gemini pH 4 Dimensions: 21.1 mm×150 mm 5 μm afforded the desired product as a white solid.

LC/MS (Method B): RT=1.22; m/z=577 [M+H]⁺

¹H NMR (399 MHz, DMSO-d₆) δ 7.64-7.54 (m, 1H), 7.35-7.13 (m, 7H), 7.09 (ddd, J=9.1, 4.6, 2.3 Hz, 2H), 4.82 (d, J=8.2 Hz, 1H), 4.69 (d, J=14.0 Hz, 2H), 3.99-3.56 (m, 4H), 3.30-3.00 (m, 3H), 2.95-2.55 (m, 1H), 2.25-2.01) (m, 3H), 290-1.91 (m, 2H), 1.87 (dd, J=6.0, 2.6 Hz, 1H), 1.60 (t, J=14.9 Hz, 2H), 1.48-1.24 (m, 1H), 1.12 (d, J=13.0 Hz, 2H), 0.73 (dq, J=12 1. 5.8, 4.9 Hz, 1H), 0.57 (q, J=12.1, 11.3 Hz, 1H).

HRMS (TOF, ESI) m/z: Calculated for C₃₂H₃₄F₂N₄O₄ 576.2548, Found: 577.206 [M+H]⁺

### rel-5-amino-3-[(1-{[(1R,2R,4S)-4-fluoro-2-phenyl-4-(pyridin-2-ylmethyl)cyclohexyl]carbonyl)}-4-hydroxypiperidin-4-yl)methyl]-6-(4-fluorophenoxy)pyrimidin-4-one (EXAMPLE 126)

### Step 1: rel-ethyl (1R,2R,4S)-4-fluoro-2-phenyl-4-[(pyridin-2-yl)methyl]cyclohexane-1-carboxylate

Starting from re-ethyl (1R,2R)-4-hydroxy-2-phenyl-4-[(pyridin-2-yhmethyl]cyclohexane-1-carboxylate (obtained according to Step 1 or EXAMPLE 96: 230 mg, 0.68 mmol) following procedure described in Step 4 of EXAMPLES 122 and 123, rel-ethyl (1R,2R,4S)-4-fluoro-2-phenyl-4-[(pyridin-2-yl)methyl]cyclohexane-1-carboxylate was obtained as a colourless oil,

¹H NMR (399 MHz, Chloroform-d) δ 8.53 (ddd, J=4.9. 1.9, 0.9 Hz, 1H), 7.64 (td, J=7.7, 1.8 Hz, 1H), 7.35-7.11 (m, 7H), 3.87 (q, J=7.1 Hz, 2H), 3.29-3.05 (m, 2H), 2.64-2.48 (m, 1H), 2.15-1.87 (m, 4H), 1.82-1.53 (m, 2H), 0.94 (t, J=7.1 Hz, 3H).

### Step 2: rel-(1R,7R,4S)-4-fluoro-2-phenyl-4-(pyridin-2-)ylmethyl)cyclohexane-1-carboxylic acid

Starting from rel-ethyl (1R,2R)-4-fluoro-2-phenyl-4-(prop-1-yn-1-yl)cyclohexane-1-carboxylate (41 mg, 0.12 mmol) following procedure described in Step 5 of EXAMPLES 122 and 123, rel-(1R,2R,4S)-4-fluoro-2-phenyl-4-(pyridin-2-ylmethyl) cyclohexane-1-carboxylic acid was obtained as an off-white solid. The compound was used without further purification.

LC/MS (Method B). RT=0.92;=314 [M+H]⁺

### Step 3: EXAMPLE 126

Starting from 5-amino-6-(4-fluorophenoxy)-3-[(4-hydroxypiperidin-4-yl)methyl]pyrimidin-4-one (37 mg, 0.11 mmol) and rel-(1R,2R,4S)-4-fluoro-2-phenyl-4-(pyridin-2-ylmethyl)cyclohexane-1-carboxylic acid (35 mg) following procedure described in Step 3 of EXAMPLE 94, the obtained residue was purified via flash chromatography using EtOAc-10%) MeOH/EtOAc (gradient) as eluent to give the crude product as a colourless oil. Final purification via prep HPLC (Prep HPLC Column: Gemini pH 4 Dimensions: 21.1 mm×150 mm 5 μm) afforded the desired product as a yellow solid.

LC/MS (Method 8): RT=m/z=630 [M+H]⁺

¹H NMR (399 MHz, DMSO-₆) δ 8.49 (ddd, J=4.9, 1.9, 1.0 Hz, 1H), 7.72 (tt, J=7.7, 1.6 Hz, 1H), 7.59 (m, 7.35-7.04 (m, 1H), 4.81 (d, J=8.4 Hz, 1H), 4.68. (d, J=11.7 Hz, 2H), 3.98-3.60 (m, 4H), 3.23-2.82 (m, 4H), 2.73-2.56 (m, 1H), 1.97-1.50 (m, 6H), 1.48-1.05 (m, 4H), 0,80-0.59 (m, 1H).

HRMS (TOF, ESI) m/z: Calculated for C₃₅H₃₇F₂N₅O₄ 629.2814, Found: 630.287 [M+H]⁺

### rel-5-amino-3-[(1-{[(1R,2R)-4-fluoro-4-methyl-2-phenylcyclohexyl]carbonyl}-4-hydroxypiperidin-4-yl)methyl]-6-(4-fluorophenyl)pyrimidin-4-one (EXAMPLE 127)

### Step 1: rel-ethyl (1R,2R,4S)-4-hydroxy-4-methyl-2-phenylcyclohexane-1-carboxylate and rel-ethyl (1R,2R,4R)-1-hydroxy-4-methyl-2-phenylcyclohexane-1-carboxylate

To a N₂ flushed flask containing, cerium(III) chloride (840.59 mg, 3.41 mmol, 2.8 eq.), anhydrous THF (6 mL) was added and the suspension cooled to 0° C. Methylmagnesium bromide solution (1.14 mL, 3M in THF, 3.41 mmol, 2.8 eq.) was then added slowly dropwise. After addition was complete. the mixture was stirred at 0° C. for 90 minutes. rel-ethyl (1R,2R)-4-oxo-2-phenylcyclohexane-1-carboxylate (300 mg, 1.22 mmol, 1.0 eq.) dissolved in THF (2 mL) was then added dropwise and stirred for 2 hours. The reaction mixture was poured onto ice/water and acetic acid was added (0.2 mL). The mixture was extracted EtOAc (30 mL), dried (MgSO₄) and evaporated in vacuo to afford a crude oil. The residue was purified via flash chromatography using heptane-69% EtOAc/Heptane (gradient) as eluent to afford:

First ethyl: rel-ethyl (1R,2R,4S)-4-hydroxy-1-methyl-2-phenylcyclohexane-1-carboxylate

¹H NMR (399 MHz, DMSO-d₆) δ 7.33-7.06 (m, 5H), 4.28 (s, 1H), 3.78 (q, J=7.1 Hz, 2H), 3.12 (td, J=12.0, 3.9 Hz, 1H), 2.51 (d, J=1.9 Hz, 1H), 1.97-1.79 (m, 1H), 1.73-1.33 (m, 5H), 1.13 (s, 3H), 0.85 (t, J=7.1 Hz, 3H).

Second elute: rel-ethyl (1R,2R,4R)-4-hydroxy-4-methyl-2-phenylcyclohexane-1-carboxylate

¹H NMR (399 MHz, DMSO-d₆) δ 7.33-7.10 (m, 5H), 4.54 (s, 1H), 3.79 (q, J=7.1 Hz, 2H), 2.78 (ddd, J=11.5, 9.6, 7.0 Hz, 1H), 2.51 (d, J=1.9 Hz, 1H), 1.95-1.83 (m,1H), 1.68-1.37 (m, 5H), 127 1.24 (s, 3H), 0.84 (t, J=7.1 Hz, 3H).

### Step 2: rel-ethyl (1R,2R)-4-fluoro-4-methyl-2-phenylcyclohexane-1-carboxylate

Starting from rel-ethyl (1R,2R)-4-hydroxy-4-methyl-2-phenylcyclohexane-1-carboxylate (192 mg, 0.73 mmol) following the procedure described in Step 4 of EXAMPLES 122 and 123, rel-ethyl (1R,2R)-4-fluoro-4-methyl-2-phenylcyclohexane-1-carboxylate was obtained as a colourless oil.

¹H NMR (399 MHz, Chloroform-d) δ 7.35-7.26 (m, 3H), 7.26-7.17 (m, 2H), 3.95-3.84 (m, 2H), 3.33-2.83 (m, 1H), 2.67-2.49 (m, 1H), 2.20-1.62 (m, 5H), 1.57-1.46 (m, 1H), 1.40 (d, J=21.1 Hz, 3H), 0.96 (dt, J=7.8, 7.1 Hz, 3H).

### Step 3: rel-(1R,2R)-4-fluoro-4-methyl-2-phenylcyclohexane-1-carboxylic acid

Starting from rel-ethyl (1R,2R)-4-fluoro-4-methyl-2-phenylcyclohexane-1-carboxylate (100 mg, 0.38 mmol) following procedure described in Step 5 of EXAMPLES 122 and 123, rel-(1R,2R)-4-fluoro-4-methyl-2-phenylcyclohexane-1-carboxylic acid was obtained as a colourless oil, The compound was used without further purification.

¹H NMR (399 MHz, DMSO-d₆) δ 11.97 (s, 1H), 7.91 (m, 5H) 3.09-2.52 (m, 2H), 2.04-1.56 (m, 6H), 1.55-1.27 (m, 3H)

### Step 3: EXAMPLE 127

Starting from 5-amino-6-(4-fluorophenoxy)-3-[(4-hydroxypiperidin-4-yl)methyl]pyrimidin-4-one (67 mg, 0.2 mmol) and rel-(1R,2R)-4-fluoro-4-methyl-2-phenylcyclohexane-1-carboxylic acid (95 mg) following procedure described in Step 3 of EXAMPLE 94. the obtained residue was purified via flash chromatography using DCM-5% MeOH/DCM (gradient) as eluent to give the desired product as a white solid.

LC(MS (Method B): RT=1.19: m/z=553 [M+H]⁺

¹H NMR (399 MHz, DMSO-₆) δ 7.59 (m, 1H), 7.34-7.05 (m, 9H), 4.81 (dd, J=9.0, 2.5 Hz, 1H), 4.68 (d, J=12.7 Hz, 2H), 4.00-3.78 (m, 2H), 3.77-3.56 (m, 2H), 3.15-3.02 (m, 2H), 2.92 (t, J=13.6 Hz, 1H), 2.72-2.58 (m, 1H), 2.18-1.01 (m, 12H), 0.82-0.46 (m, 1H) HRMS (TOF, ESI) m/z: Calculated for C₃₀H₃₄F₂N₄O₄ 552.2548, Found: 553.2574 [M+H]⁺

### 5-amino-3-[(1-{[(1S,2S,4R)-4-ethynyl-4-fluoro-2-phenylcyclohexyl]carbonyl}-4-hydroxypiperidin-4-yl)methyl]-6-(4-(fluorophenoxy)pyrimidin-1-one (EXAMPLE 128)

and

### 5-amino-3-[(1-{[(1S,2S,4S)-4-ethynyl-4-fluoro-2-phenylcyclohexyl]carbonyl}-4-hydroxypiperidin-4-yl)methyl]-6-(4-fluorophenoxy)pyrimidin-4-one (EXAMPLE 129)

### Step 1: ethyl (1S,2S,4R)-4-hydroxy-2-phenyl-4-[2-trimethylsilyl)ethynyl]cyclohexane-1-carboxylate and ethyl (1S,2S,4R)-4-hydroxy-2-phenyl-4-[2-(trimethylsilyl)ethynyl]cyclohexane-1-carboxylate

Starting from ethyl (1S,2S)-4-oxo-2-phenylcyclohexane-1-carboxylate (2.0 g, 8.12 mmol) and ethynyltrimethylsilane (1.24 ml. 8.93 mmol) following procedure described in Step 2 of EXAMPLES 122 and 123, the obtained residue was purified via flash chromatography using Heptane-14% EtOAc/Heptane (gradient) as eluent to afford:

First elute: ethyl (1S,2S,4R)-4-hydroxy-2-phenyl-4-[2-(trimethylsilyl)ethynyl]cyclohexane-1-carboxylate obtained as a colourless oil.

¹H NMR (399 MHz, Chloroform-d) δ 7.31-6.92 (m, 5H), 3.73 (q, J=7.1 Hz, 2H), 3.10 (ddd, J=13.0, 11.6, 3.7 Hz, 1H), 2.44 (td, J=11.7, 3.4 Hz, 1H), 2.09-1.56 (m, 6H), 1.12 (t, J=7.1 Hz, 1H), 0.90-0.65 (m, 3H), 0.00 (s, 9H).

Second elute: ethyl (1S,2S,4S)-4-hydroxy-2-phenyl-4-[2-(trimethylsilyl)ethynyl]cyclohexane-1-carboxylate obtained as a colourless oil. The compound was used without further purification.

¹H NMR (399 MHz, Chloroform-d) δ 7.23-6.96 (m, 5H), 3.86-3.63 (m, H), 3.04 (ddd, J=13.0. 11.5, 3.2 Hz, 1H), 2.50-2.26 (m, 1H), 2.09-1.77 (m, 4H), 1.63 (t, J=12.8 H, 1H), 1.12 (t, J=7.1 Hz, 1H), 0.82 (td, J=7.1, 2.3 Hz, 3H), 0.10 (s, 9H).

### Step 2: ethyl (1S,2S)-4-ethynyl-4-hydroxy-2-phenylcyclohexane-1-carboxylate

Starting from ethyl (1S,2S)-4-hydroxy-2-phenyl-4-[2-(trimethylsilyl)ethynyl]cyclohexane-1-carboxylate (2.05 g, 5.95 mmol) following procedure described in Step 2 of EXAMPLE 91, the obtained residue was purified via flash chromatography using Heptane-30% EtOAc/Heptane (gradient) as eluent to afford ethyl (1S,2S)-4-ethynyl-4-hydroxy-2-phenylcyclohexane-1-carboxylate as a pale yellow oil. The compound was used without further purification.

¹H NMR (399 MHz, Chloroform-d) δ 7.44-7.14 (m, 5H), 4.02-3.80 (m, 2H), 3.21 (ddd, J=13.0, 11.5, 3.3 Hz, 1H), 2.69-2.43 (m 2H), 2.37-1.76 (m, 4H), 1.70 (td, J=2,7, 4.5 Hz, 1H), 1.28 (t, J=7.1 Hz, 1H), 1.04-0.80 (m, 3H).

### Step 3: ethyl 1S,2S)-4-ethynyl-4-hydroxy-2-phenylcyclohexane-1-carboxylate

Starting from ethyl (1S,2S)-4-ethynyl-4-hydroxy-2-phenylcyclohexane-1-carboxylate (890 mg) following procedure described in Step 4 of EXAMPLES 122 and 123, ethyl (1S,2S)-4-ethynyl-4-fluoro-2-phenylcyclohexane-1-carboxylate was obtained as a colourless oil.

LC/MS (Method B): RT=1.22: m/z=275 [M+H]⁺

### Step 4: (1S,2S)-4-ethynyl-4-fluoro-2-phenylcyclohexane-1-carboxylate acid

Starting from ethyl (1S,2S)-4-ethynyl-4-fluoro-2-phenylcyclohexane-1-carboxylate (300 mg, 1.09 mmol) following procedure described in Step 5 of EXAMPLES 122 and 123, (1S,2S)-4-ethynyl-4-fluoro-2-phenylcyclohexane-1-carboxylic acid was obtained as a colourless oil. The compound was used without further purification.

LC/MS (Method B): RT=1.15; m/z=245 [M−H]⁻

### Step 5: EXAMPLE 128 and EXAMPLE 129

Starting from (1S,2S)-4-ethynyl-4-fluoro-2-phenylcyclohexane-1-carboxylic acid (110 mg) and 5-amino-6-(4-fluorophenoxy)-3-[(4-hydroxypiperidin-4-yl)methyl]pyrimidin-4-one (136 mg, 0.41 mmol) following procedure described in Step 3 or EXAMPLE 94, the obtained residue was purified via flash chromatography using DCM-5% MeOH/DCM (gradient) as eluent to afford the products a mixture of diastereoisomers. Final purification via prep HPLC (Prep HPLC Column: Gemini pH 4 Dimensions: 21.1 mm×150 mm 5 μm) gave:

First elute: EXAMPLE 128 as a white solid.

LC/MS (Method B): RT=1.18; m/z=563 [M+H]⁺

¹H NMR (399 MHz, DMSO-d₆) δ 7.60 (m, 1H), 7.35-7.13 (m, 7H), 7.09 (ddd, J=9.1, 4.6, 2.3 Hz, 2H), 4.83 (d, J=7.3 Hz, 1H), 4.69 (d, J=14.1 Hz), 2H), 3.99-3.78 (m, 3H), 3.77-3.55 (m, 2H), 3.30-3.01 (m, 3H), 2.96-2.57 (m, 1H), 2.35-1.87 (m, 4H), 1.85-1.54 (m, 2H), 1.49-1.03 (m, 3H), 0.67 (dtd, J=44.5, 13.0, 4.4 Hz, 1H).

HRMS (TOF, ESI) Calculated for C₃₁H₃₂F₂N₄O₄ 562.2392, Found: 563.2404 [M+H]⁺

Second elute: EXAMPLE 129 as a white solid.

LC/MS (Method B): RT=1.19; m/z=563 [M+H]⁺

¹H NMR (399 MHz, DMSO-d₆) δ 7.59 (m, 1H), 7.37-7.14 (m, 7H), 7.09 (ddd, J=9.1, 4.6, 2.2 Hz, 2H), 4.84 (d, J=7.1 Hz, 1H), 4.69 (d, J=14.7 Hz, 2H), 4.06 (dd, J=5.3, 3.3 Hz, 1H), 3.99-3.77 (m, 2H), 3.75-3.56 (m, 2H), 3.27-3.03 (m, 3H), 2.91-2.57 (m, 1H), 2.28-1.90 (m, 4H), 1.79-1.59 (m, 2H), 1.49-1.03 (m, 3H), 0.63 (dtd, J=81.5, 12,7. 4.2 Hz, 1H).

HRMS (TOF, ESI) m/z: Calculated for C₃₁H₃₂F₂N₄O₄ 562.2392, Found: 563.2402 [M+H]⁺

### 5-amino-3-[(1-{[(1R,2R,4R)-4-fluoro-2-phenyl-4-[2-(pyridin-3-yl)ethynyl]cyclohexyl]carbonyl}-4-hydroxypiperidin-4-yl)methyl]-6-(4-fluorophenoxy)pyrimidin-4-one (EXAMPLE 130)

### Step 1: ethyl (1R,2R,4R)-4-hydroxy-2-phenyl-4-[2-(pyridin-3-yl)ethynyl]cyclohexane-1-carboxylate

Starting from ethyl (1R,2R)-4-oxo-2-phenylcyclohexane-1-carboxylate (400 mg, 1.62 mmol) and 3-ethynylpyridine, (218 mg, 2.11 mmol, 1.3 eq.), following procedure described in Step 2 of EXAMPLES 122 and 123, the obtained residue was purified via flash chromatography using Heptane-78% EtOAc/Heptane (gradient) as eluent to afford ethyl (1R,2R,4R)-4-hydroxy-2-phenyl-4-[2-(pyridin-3-y)ethynyl]cyclohexane-1 -carboxylate as a white foam.

LC/MS (Method B): RT=1.15; m/z=350 [M+H]⁺

### Step 2: ethyl (1R,2R)-4-fluoro-2-phenyl-4-[2-(pyridin-3-yl)ethynyl]cyclohexane-1-carboxylate

Starting from ethyl (1R,2R,4R)-4-hydroxy-2-phenyl-4-[2-(pyridin-3-yl)ethynyl]cyclohexane-1-carboxylate (420 mg, 1.2 mmol) following procedure described in Step 4 of EXAMPLES 122 and 123, ethyl (1R,2R)-4-fluoro-2-phenyl-4-[2-(pyridin-3-yl)ethynyl]cyclohexane-1-carboxylate was obtained as a colourless oil.

LC/MS (Method B): RT=1.35: m/z=352 [M+H]⁺

Step 3: (1 R,2R)-4-fluoro-2-phenyl-4-[2-(pyridin-3-yl)ethynyl]cyclohexane-1-carboxylic acid

Starting from ethyl (1R,2 R)-4-fluoro-2-phenyl-4-[2-(pyridin-3-yl)ethynyl]cyclohexane-1-carboxylate (330 mg, 0.94 mmol) following procedure described in Step 5 of EXAMPLES 122 and 123, (1R ,2R)-4-fluoro-2-phenyl-4-[2-(pyridin-3-yl)ethynyl]cyclohexane-1-carboxylic acid was obtained as a white solid. The compound was used without further purification.

LC/MS (Method B): RT=1.01; m/z=342 [M+H]⁺

### Step 4: EXAMPLE 130

Starting from 5-amino-6-(4-fluorophenoxy)-3-[(4-hydroxypiperidin-4-yl)methyl]pyrimidin-4-one (110 mg, 0.33 mmol) and (1R,2R)-4-fluoro-2-phenyl-4-[2-(pyridin-3-yl)ethynyl]cyclohexane-1-carboxylic acid (152 mg) following procedure described in Step 3 or EXAMPLE 94, the obtained residue was purified via flash chromatography using DCM-5% MeOH/DCM (gradient) as eluent to give the crude product as a white solid. Final purification via prep HPLC (Prep HPLC Column: Gemini pH 4 Dimensions: 21.1 mm×150 mm 5 μm) afforded the desired product as a white solid.

LC/MS (Method B): RT=1.08; m/z=640 [M+H]⁺

¹H NMR (399 MHz, DMSO-₆) δ 8.80-874 (m, 1H), 8.66 (dt, J=4.9, 1.5 Hz, 1H), 8.01 (ddd, J=7.8, 3,5, 1.7 Hz, 1H), 7.59 (m, 1H), 7.54-7.48 (m, 1H), 7.37-7.14 (m, 7H), 7.09 (ddd, J=9.2, 4.6, 2.4 Hz, 2H), 4.82 (d, J=7.5 Hz, 1H), 4.69 (d, J=14.3 Hz, 2H),4.02-3.78 s, 2H), 3.77-3.58 (m, 3H), 3.31-3.08 (m, 2H), 2.98-2.56 (m, 1H), 2.40-2.01 (m, 4H), 1.87 (s, 2H), 1.54-1.03 (m, 2H), 0.76 (td, J=12.5, 4.2 Hz, 1H), 0.57 (td, J=13.0, 4.3 H, 1H), HRMS (TOF, ESI) m/z: Calculated for C₃₆H₃₅F₂N₅O₄ 639.2657, Found: 640.2765 [M÷H]⁺

### 5-amino-3-[(1-{[(1R,2R,4S)-4-fluoro-2-phenyl-4-[2-(pyridin-2-yl)ethynyl]cyclohexyl]carbonyl}-4-hydroxypiperidin-4-yl)methyl]-6-(4-fluorophenoxy)pyrimidin-4-one (EXAMPLE 131)

and

### 5-amino-3-[(1-{[(1R,2R4R)-4-fluoro-2-phenyl-4-[2-(pyridin-2-yl)ethynyl]cyclohexyl]carbonyl}-4-hydroxypiperidin-4-yl)methyl]-6-(4-fluorophenoxy)pyrimidin-4-one (EXAMPLE 132)

### Step 1: ethyl (1R,2R)-4-hydroxy-2-phenyl-4-[2-(pyridin-2-yl)ethynyl]cyclohexane-1-carboxylate

Starting from ethyl (1R,2R)-4-oxo-2-phenylcyclohexane-1-carboxylate (400 mg. 1.62 mmol) and 2-ethynylpyridine (0.21 ml, 2.11 mmol, 1.3 eq.) following procedure described in Step 2 of EXAMPLES 122 and 123, the obtained residue was purified via flash chromatography using Heptane-63% EtOAc/Heptane (gradient) as eluent to afford ethyl (1R,2R)-4-hydroxy-2-phenyl-4-[2-(pyridin-2-yl)ethynyl]cyclohexane-1-carboxylate as a brown foam.

LC/MS (Method B); RT=1.15; m/z=350 [M+H]⁺

### Step 2: ethyl (1R,2R)-4-fluoro-2-phenyl-4-[2-(pyridin-2-yl)ethynyl]cyclohexane-1-carboxylate

Starting from ethyl (1R,2R)-4-hydroxy-2-phenyl-4-[2-(pyridin-2-yl)ethynyl]cyclohexane-1-carboxylate (480 mg, 1.37 mmol) following procedure described in Step 4 or EXAMPLES 122 and 123, ethyl (1R,2R)-4-fluoro-2-phenyl-4-[2-(pyridin-2-yl)ethynyl]cyclohexane-1-carboxylate was obtained as a colourless oil. The compound was used without further purification.

LC/MS (Method B): RT=1.34; m/z=352 [M+H]⁺

Step 3: (1R,2R)-4-fluoro-2-phenyl-4-[2-pyridin-2-yl)ethynyl]cyclohexane-1-carboxylic acid

Starting from ethyl (1R,2R)-4-fluoro-2-phenyl-4-[2-(pyridin-2-yl)ethynyl]cyclohexane-1-carboxylate (360 mg) following procedure described in Step 5 of EXAMPLES 122 and 123, 5 (1R,2R)-4-fluoro-2-phenyl-4-[2-(pyridin-2-yl)ethynyl]cyclohexane-1-carboxylic acid was obtained as a yellow oil. The compound was used without further purification.

LC/MS (Method B): RT=1.13 and 1.14; m/z=324 [M+H]⁺

### Step 4: EXAMPLE 131 and EXAMPLE 132

To a mixture of (1R,2R)-4-fluoro-2-phenyl-4-[2-(pyridin-2-yl)ethynyl]cyclohexane-1-carboxylic acid (180 mg) and 5-ammo-6-(4-fluorophenoxyl-3-[(4-hydroxypiperidin-4-yl)methyl]pyrimidin-4-one (112 mg, 0.33 mmol) in acetonitrile (4 mL), triethylamine (0.14 mL, 1 mmol, 3.0 eq.) and 1-hydroxybenzotriazole hydrate (56.26 mg, 0.37 mmol, 1.1 eq.) were added. After 1 minute, 1-(3-dimethylaminopropyl)-3-ethyl-carbodiimide hydrochloride (70.43 mg, 0.37 mmol, 1.1 eq.) was added and the resulting yellow solution was stirred overnight. The reaction mixture was concentrated in vacua and the residue was partitioned between EtOAc (25 mL) and sat. NaHCO₃ solution (20 mL). The organic layer was separated, dried (MgSO₄) and evaporated in vacua to afford a crude oil. The residue was purified via flash chromatography using DCM-5% MeOH/DCM (gradient) as eluent to afford the products a mixture of diastereoisomers. Final purification via prep HPLC (Prep HPLC Column: Gemini pH4 Dimensions: 21.1 mm×150 mm 5 μm) gave:

First elute: EXAMPLE 131 as a white solid.

LC/MS (Method B): RT=1.19; m/z=640 [M+H]⁺

¹H NMR (399 MHz, DMSO-d₆) δ 8.59 (ddd, J=4.9, 1.7, 1.0 Hz, 1H), 7.89-7.75 (m, 1H), 7.66-7.48 (m, 2H), 7.44 (ddt, J=7.5, 4.9, 1.2 Hz, 1H), 7.40-7.14 (m, 7H), 7.10 (ddq, J=6.9, 4.6, 2.2 Hz, 2H), 4.83 (d, J=6.9 Hz, 1H), 4.69 (d, J=14.3 Hz, 2H) 4.03-3.49 (m, 4H), 3.33-2.55 (m, 4H), 2.45-2.01 (m, 3H), 1.92-1.59 (m, 2H), 1.51-0.97 (m, 3H), 0.68 (dtd, J=50.8. 12.7, 4.3 Hz, 2H).

HRMS (TOF, ESI) m/z: Calculated for C₃₆H₃₅F₂N₅O₄ 639.2657, Found: 640.2683 [M+H]⁺

Second elute: EXAMPLE 132 as a white solid.

LC/MS (Method B): RT=1.22; m/z=640 [M+H]⁺

¹H NMR (399 MHz, DMSO-d₆) δ 8.64 (ddd, J=4.7, 1.8, 1.0 Hz, 1H), 7.96-7.81 (m, 1H), 7.67 (ddt, J=7.9, 2.3, 1.1 Hz, 1H), 7.59 (m, 7.49 (ddt, J=7.6, 4.9, 1.3 Hz, 1H), 7.37-7.15 (m, 7H), 7.09 (ddd, J=9.1, 4.7, 2.4 Hz, 2H), 4.82 (d, J=6.9 Hz, 1H), 4.69 (d, J=14.8 Hz, 2H), 4.01-3.78 (m, 2H), 3.77-3.56 (m, 2H), 3.31-3.06 (m, 3H), 2.95-2.59 (m, 1H, 2.40-1.99 (m, 4H), 1.88 (d, J=14.6 Hz, 2H), 1.52-1.03 (m, 2H), 0.76 (td, J=13.0, 4.4 Hz, 1H), 0.56 (dd, J=13.0, 9.1 Hz, 1H).

HRMS (TOF, ESI) m/z: Calculated for C₃₆H₃₅F₂N₅O₄ 639.2657, Found: 640.2672 [M+H]⁺

### 5-amino-3-[(1-{[(1R,2R4R)-4-fluoro-4-[2-(5-fluoropyridin-2-yl)ethynyl]-2-phenylcyclohexyl]carbonyl}-4-hydroxypiperidin-4-yl)methyl]-6-(4-fluorophenoxy)pyrimidin-4-one (EXAMPLE 133)

### Step 1: (1R,2R)-4-oxo-2-phenylcyclohexane-1-carboxylic acid

Starting from ethyl (1R,2R)-4-oxo-2-phenylcyclohexane-1-carboxylate (2.27 g, 9.22 mmol, 1.0 eq.) following procedure described in Step 5 of EXAMPLES 122 and 123, (1R,2R)-4-oxo-2-phenylcyclohexane-1-carboxylic add was obtained as a yellow solid.

LC/MS (Method B): RT=0.84; m/z=217 [M−H]⁺

### Step 2: tert-butyl (1R,2R)-4-oxo-2-phenylcyclohexane-1-carboxylate

To a solution of (1R,2R)-4-oxo-2-phenylcyclohexane-1-carboxylic acid (1.43 g, 6.55 mmol, 1.0 eq.) and tert-butanol (1.84 mL, 19.66 mmol, 3 eq.) in DCM (40 mL), was added a solution of dicyclohexylcarbodiimide (1.57 ml, 7.21 mmol, 1.0 eq.) in DCM (16 mL) at 0° C. dropwise followed by DMAP (800 mg, 6.55 mmol, 1.0 eq.). The reaction mixture was stirred at the same temperature for 30 minutes before being allowed to warm to r.t. overnight. Diethyl ether (50 mL) was added and the suspension was filtered through a pad of celite. washed with diethyl ether. The filtrate was concentrated in vacuo to afford a crude solid. Purification via flash chromatography using Heptane-33% EtOAc/Heptane (gradient) as eluent afforded the desired product as a white solid.

¹H NMR (399 MHz, Chloroform-d) δ 7.34 (ddd, J=8.9, 6.5, 0.9 Hz, 2H), 7.29-7.18 (m, 3H), 3.23 (ddd, J=12.0, 10.9, 5.2 Hz, 1H), 2.98-2.87 (m, 1H), 2.68-2.42 (m, 4H), 2.32 (ddt, J=13.1, 5.9, 3.4 Hz, 1H), 2.03 (tdd, J=13.3, 11.7, 5.0 Hz, 1H), 1.18 (s, 9H).

### Step 3: tert-butyl (1R,2R)-4-[2-(5-fluoropyridin-2-yl)ethynyl]-4-hydroxy-2-phenylcyclohexane-1-carboxylate

Starting from tert-butyl (1R,2R)-4-oxo-2-phenylcyclohexane-1-carboxylate (300 mg, 1.09 mmol) and 2-ethynyl-5-fluoropyridine (172 mg, 1.42 mmol, 1.3 eq.) following procedure described in Step 2 of EXAMPLES 122 and 123, the obtained residue was purified via flash chromatography using Heptane-30% EtOAc/Heptane (gradient) as eluent to afford the desired product as a colourless oil.

LC/MS (Method B): RT=1.32; m/z=396 [M+H]⁺

### Step 4: tert-butyl (1R,2R,4R)-4-fluoro-4-[2-(5-fluoropyridin-2-yl)ethynyl]-2-phenylcyclohexane-1-carboxylate and tert-butyl (1R,2R,4S)-4-fluoro-4-[2-(5-fluoropyridin-2-yl)ethynyl]-2-phenylcyclohexane-1-carboxylate

Starting from tert-butyl (1R,2R)-4-[2-(5-fluoropyridin-2-yl)ethynyl]-4-hydroxy-2-phenylcyclohexane-1-carboxylate (272 mg, 0.69 mmol) following procedure described in Step 4 of EXAMPLES 122 and 123, the obtained residue was purified via flash chromatography using Heptane-10% EtOAc/Heptane (gradient) as eluent to afford:

First elute: tert-butyl (1R,2R,4R)-4-fluoro-4-[2-(5-fluoropyridin-2-yl)ethynyl]-2-phenyl cyclohexane-1 -carboxylate obtained as a colourless oil. The compound was used without further purification

LC/MS (Method B): RT=1.46; m/z=398 [M+H]⁺

Second elute: tert-butyl (1R,2R,4S)-4-fluoro-4-[2-(5-fluoropyridin-2-yl)ethynyl]-2-phenylcyclohexane-1-carboxylate obtained as a colourless oil.

LC/MS (Method B): RT=1.47; m/z=398 [M+H]⁺

### Step 5: (1R,2R,4R)-4-fluoro-4-[2-(5-fluoropyridin-2-yl)ethynyl]-2-phenylcyclohexane-1-carboxylic acid

To a solution of tert-butyl (1R,2R,4R)-4-fluoro-4-[2-(5-fluoropyridin-2-yl)ethynyl]-2-phenylcyclohexane-1 -carboxylate (100 mg, 0.25 mmol) in DCM (2 mL), TFA (2 mL) was added slowly at 0° C. under nitrogen. The mixture was allowed to warm to r.t. over 2 hours. The reaction mixture was concentrated in vacua. The residue was partitioned between DCM (25 mL) and water (25 mL). The organic layer was separated. dried (MgSO₄) and evaporated in vacuo to afford (1R,2R,4R)-4-fluoro-4-[2-(5-fluoropyridin-2-yl)ethynyl]-2-phenylcyclohexane-1-carboxylic acid as an off-white solid. The compound was used without further purification.

LC/MS (Method B): RT=1.18; m/z=342 [M+H]⁺

### Step 6: EXAMPLE 133

Starting from 5-amino-6-(4-fluonophenoxy)-3-[(4-hydroxypiperidin-4-yl)methyl]pyrimidin-4-one (75 mg, 0.23 mmol) and (1R2R,4R)-4-fluoro-4-[2-(5-fluoropyridin-2-yl)ethynyl]-2-phenylcyclohexane-1-carboxylic acid (70 mg) following procedure described in Step 3 or EXAMPLE 94, the obtained residue was purified via flash chromatography using Heptane-100% EtOAc (gradient) as eluent to give the crude product as a colourless oil. Final purification via prep HPLC (Prep HPLC Column: Gemini pH 4 Dimensions 21.1 mm×150 mm 5 μm) afforded the desired product as a beige solid.

LC/MS (Method B): RT=1.25; m/z=658 [M+H]⁺

¹H NMR (399 MHz, DMSO₆) δ 8.66 (dd, J=2.8, 1.8 Hz, 1H), 7.86 (tdd, J=8.6, 3.0, 1.5 Hz, 1H), 7.82-7.74 (m, 1H), 7.60 (m, 1H), 7.40-7.14 (m, 7H), 7.09 (ddd, J=9.2. 4.6, 2.4 Hz, 2H), 4.82 (s, 3H), 4.00-3.56 (m, 4H), 3.30-2.96 (m, 3H), 2.95-2.58 (m, 1 H), 2.40-2.02 (m, 4H), 1.96-1.70 (m, 2H), 1.51-0.97 (m, 2H), 0.76 (td, J=12.8, 4.5 Hz, 1H), 0.56 (td, J=13.4, 4.7 Hz, 1H).

HRMS (TOF, ESI) m/z: Calculated for C₃₆H₃₄F₃N₅O₄ 657.2563, Found: 658.2665 [M+H]⁺

### 5-amino-3-[(1-{[(1R,2R,4S)-4-fluoro-4-[2-(5-fluoropyridin-2-yl)ethynyl]-2-phenylcyclohexyl]carbonyl}-4-hydroxypiperidin-4-yl)methyl]-6-(4-fluorophenoxy)pyrimidin-4-one (EXAMPLE 134)

### Step 1: (1R,2R,4S)-4-fluoro-4-[2-(5-fluoropyridin-2-yl)ethynyl]-2-phenylcyclohexane-1-carboxylic acid

Starting from tert-butyl (1R,2R,4S)-4-fluoro-4-[2-(5-fluoropyridin-2-yl)ethynyl]-2-phenyl cyclohexane-1-carboxylate (98 mg, 0.25 mmol, 1.0 eq.) following procedure described in Step 5 of EXAMPLE 133, (1R,2R,4S)-4-fluoro-4-[2-(5-fluoropyridin-2-yl)ethynyl]-2-phenylcyclohexane-1-carboxylic acid (84 mg) was obtained as an off-white solid. The compound was used without further purification.

LC/MS (Method RT=1.073; m/z=342 [M+H]⁺

Step 2: EXAMPLE 134

Starting from 5-amino-6-(4-fluorophenoxy)-3-[(4-hydroxypiperidin-4-yl)methyl]pyrimidin-4-one (83 mg, 0.25 mmol) and (1R,2R,4S)-4-fluoro-4-[2-(5-fluoropyridin-2-yl)ethynyl]-2-phenylcyclohexane-1-carboxylic acid (84 mg) following procedure described in Step 3 of EXAMPLE 94, the obtained residue was purified of via flash chromatography (C18) using 10% to 100% acetonitrile/water (gradient) as eluent to afford EXAMPLE 134 as a white solid.

LC/MS (Method B): RT=1.23; m/z=638 [M+HF]⁺

¹H NMR (399 MHz, DMSO-₆) δ 8.60 (m, 1H), 7.81 (tdd, J=8.6, 3.0, 1.6 Hz, 1H), 7.69 (ddd, J=8.8. 4.6, 2.7 Hz), 7.61 (m, 1H), 7.36-7.18 (m, 7H), 7.11-7.07 (m, 2H), 4.83 (m, 1H), 4.70 (m, 2H), 3.97-3.62 (m, 4H), 3.33-3.11 (m, 3H), 2.94-2.63 (m, 1H, 2.41-2.07 (m, 4H), 1.84-1.65 (m, 2H), 1.47-1.11 (m, 3H), 0.78-0.58 (m, 1H).

HRMS (TOF, ESL) m/z: Calculated for C₃₆H₃₄F₃N₅)₄ 657.2563, Found: 658.2652 [M+H]⁺

### 5-amino-3-({1-[(1R,2R,4R)-4-fluoro-2-phenyl-4-(2-phenylethynyl)cyclohexanecarbonyl]-4-hydroxypiperidin-4-yl}methyl)-6-(4-fluorophenoxy)-3,4-dihydropyrimidin-4-one (EXAMPLE 135)

and

### 5-amino-3-({1-[(1R,2R,4S)-4-fluoro-2-phenyl-4-(2-phenylethynyl)cyclobexanecarbonyl]-4-hydroxypiperidin-4-yl}methyl)-6-(4-fluorophenoxy)-3,4-dihydropyrimidin-4-one (EXAMPLE 136)

To a solution of 5-amino-3-({1-[(1R,2R)-4-ethynyl-4-fluoro-2-phenylcyclohexane carbonyl]-4-hydroxypiperidin-4-yl}methyl)-6-(4-fluorophenoxy)-3,4-dihydropyrimidin-4-one (110 mg, 0.2 mmol, 1.0 eq.) in THF (2 mL) was added copper(I) iodide (33 mg, 0.02 mmol, 0.08 eq.) and bis(triphenylphosphine)palladium(II) dichloride (5.5 mg, 0.01 mmol, 0.04 eq.). The reaction mixture was degassed under nitrogen for 5 minutes before adding triethylamine (0.04 mL, 0.29 mmol, 1.5 eq.) followed by iodobenzene (0.03 mL 0.29 mmol, 1.5 eq.). The reaction mixture was stirred at r.t. for 1 hour. The mixture was partitioned between EtOAc (20 mL) and brine (20 mL). The organic layer was separated, dried (MgSO₄) and evaporated In vacua to afford a crude brown oil. The residue was purified via flash chromatography using DCM-4.5% MeOH/DCM (gradient) as eluent to give:

First elute: EXAMPLE 135 obtained as a yellow oil.

LC/MS (Method B): RT=1.22; m/z=639 [M+H]⁺

¹H NMR (399 MHz, DMSO-₆) δ 7.66-7.41 (m, 6H, 7.34-7.18 (m, 7H), 7.11-7.07 (m, 2H) 4.94-4.49 (m, 3H), 4.05-3.53 (m, 4H), 3.17 (m, 2H), 2.70 (s, 1H), . 39-1.99 (m, 4H), 1.86 (d, J=9.1 Hz, 2H), 1.52-1.02 (m, 3H), 0.77 (dt, J=12.7, 6.2 Hz, 1H), 0.66-0.41 (m, 1H).

Second elute: EXAMPLE 136 obtained as a brown oil.

LC/MS (Method RT=1.12; m/z=619 [M+H]⁺

¹H NMR (399 MHz, DMSO-₆) 7.66-7.40 (m, 6H), 7.33-7.17 (m, 7H), 7.12-7.08 (m, 2H), 4.77 (dd, J=49.4, 5.7 Hz, 3H), 4.11-3.53 (m, 4H), 3.18 (dt, J=27.8, 11.8 Hz, 2H), 2.99-2.59 (m, 1H), 2.41-1.93 (m, 4H), 1.74 (dt, J=51.7, 12.9 Hz, 2H), 1.50-1.01 (m, 3H), 0.92-0.48 (m, 2H).

### 5-amino-3-({1-[(1R42R,4S)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4-one (EXAMPLE 137)

and

### 5-amino-3-({1-[(1R,2R,4R)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4-one (EXAMPLE 138)

### Step 1: ethyl (1R,2R)-4-hydroxy-2-phenyl-4-[2-(pyrazin-2-yl)ethynyl]cyclohexane-1-carboxylate

Starting from ethyl (1R,2R)-4-oxo-2-phenylcyclohexane-1-carboxylate (400 mg, 1.62 mmol) and 2-ethynylpyrazine (220 mg, 2.11 mmol) following procedure described in Step 2 of EXAMPLES 122 and 123, the obtained residue was purified via flash chromatography using Heptane-20% EtOAc/Heptane (gradient) as eluent to afford ethyl (1R,2R)-4-hydroxy-2-phenyl-4-[2-(pyrazin-2-yl)ethynyl]cyclohexane-1-carboxylate as a colourless oil.

LC/MS (Method B): RT=1.096; m/z=351 [M+H]⁺

### Step 2: ethyl (1R,2R)-4-fluoro-2phenyl-4-[2-(pyrazin-2-yl)ethynyl]cyclohexane-1-carboxylate

Starting from ethyl (1R,2R)-4-hydroxy-2-phenyl-4-[2-(pyrazin-2-yl)ethynyl]cyclohexane-1-carboxylate (476 mg, 1.36 mmol) following procedure described in Step 4 or EXAMPLES 122 and 123, the obtained residue was purified via flash chromatography using Heptane-20% EtOAc/Heptane (gradient) as eluent to afford ethyl (1R,2R)-4-fluoro-2-phenyl-4-[2-(pyrazin-2-yl)ethynyl]cyclohexane-1-carboxylate as colourless oil.

LC/MS (Method B): RT=1.317; m/z=353 [M+H]⁺

### Step 3: (1R,2R)-4-fluoro-2-phenyl-4-[2-(pyrazin-2-yl)ethynyl]cyclohexane-1-carboxylic acid

Starting from ethyl (1R,2R)-4-fluoro-2-phenyl-4-[2-(pyrazin-2-yl)ethynyl]cyclohexane-1-carboxylate (129 mg, 0.37 mmol) following procedure described in Step 5 of EXAMPLES 122 and 123, (1R,2R)-4-fluoro-2-phenyl-4-[2-(pyrazin-2-yl)ethynyl]cyclohexane-1-carboxylic acid was obtained as a colourless oil. The compound was used without further purification.

LC/MS (Method B): RT=1.069; m/z=323 [M−H]⁺

### Step 4 EXAMPLE 137 and EXAMPLE 138

Starting from (1R,2R)-4-fluoro-2-phenyl-4-[2-(pyrazin-2-yl)ethynyl]cyclohexane-1-carboxylic acid (115 mg) and 5-amino-6-(4-fluorophenoxy)-3-[(4-hydroxypiperidin-4-yl)methyl]pyrimidin-4-one (79 mg, 0.24 mmol) following procedure described in Step 3 of EXAMPLE 94, the obtained residue was purified via prep HPLC (Prep HPLC Column: Gemini pH 7.4 Dimensions: 21.1 mm×150 mm 5 μm) to give:

First elute: EXAMPLE 137 as a white solid.

LC/MS (Method B): RT=1.155; m/z=621 [M−H]⁺

¹H NMR (399 MHz, DMSO-d₆) δ 8.82 (m, 1H), 8.77-8.66 (m, 2H), 7.60 (m, 1H, 7.34-7.18 (m, 7H), 7.10-7.07 (m, 2H), 4.84 (s, 1H), 4.68 (m, 2H), 3.97-3.82 (m, 2H), 3.76-3.63 (m, 2H), 3.31-2.88 (m, 3H), 2.72-2.61 (m, 1H), 2.45-2.10 (m, 4H), 1.85-1.67 (m, 2H), 1.47-1.12 (m, 3H), 0.78-0.58 (m, 1H).

Second elute; EXAMPLE 138 as a white solid.

LC/MS (Method B): RT=1.176; m/z=641 [M−H]⁺

¹H NMR (399 MHz, DMSO-d₆) δ 8.91 (m, 1H), 8.75-8.72 (m, 2H), 7.59 (m, 1H), 7.35-7.18 (m, 7H), 7.11-7.07 (m, 2H), 4.82 (m, 1H), 4.69 (m, 2H), 3.96-3.82 (m, 2H), 3.74-3.61 (m, 2H), 3.32-2.86 (m, 3H), 2.66 (m, 1H). 2.36-2.11 (m, 4H), 1.90-1.82 (m, 2H), 1.47-1.11 (m, 3H), 0.79-053 (m, 1H).

### 5-amino-6-(4-fluorophenoxy)-3-[(4-hydroxy-1{[(1R, 2R,4R)-4-methoxy-2-phenyl-4-[2-(pyrimidin-5-yl)ethynyl]cyclohexyl]carbonyl}piperidin-4-yl)methyl]pyrimidin-4-one (EXAMPLE 139)

### Step 1: Ethyl (1R,2R)-4-hydroxy-2-phenyl-4-[2-(pyrimidin-5-yl)ethynyl]cyclohexane-1-carboxylate

Starting from E1 (410 mg, 1.62 mmol) and 5-ethynylpyrimidine (220 mg, 2.11 mmol), following procedure described in Step 2 of EXAMPLES 122 and 123, the obtained residue was purified via flash chromatography using Heptane-100% EtOAc (gradient) as eluent to afford ethyl (1R,2R)-4-hydroxy-2-phenyl-4-[2-(pyrimidin-5-yl)ethynyl]cyclohexane-1-carboxylate as an white oil.

LC/MS (Method B): RT=1.1 min=351 [M+H]⁺

### Step 2: Ethyl (1R, 2R)-4-fluoro-2-phenyl-4-[2-(pyrimidin-5-yl)ethynyl]cyclohexane-1-carboxylate

Starting from ethyl (1R,2R)-4-hydroxy-2-phenyl-4-[2-(pyrimidin-5-yl)ethynyl]cyclohexane-1-carboxylate (410 mg, 1.17 mmol) following procedure described in Step 4 of EXAMPLES 122 and 123, the residue was purified via flash chromatography using Heptane-30% EtOAc/Heptane (gradient) as eluent to afford ethyl (1R,2R)-4-fluoro-2-phenyl-4-[2-(pyrimidin-5-yl)ethynyl]cyclohexane-1-carboxylate as a colourless oil.

LC/MS (Method B): RT=131; m/z=353 [M+H]⁺

### Step 3: (1R,2R)-4-methoxy-2-phenyl-4-[2-(pyrimidin-5-yl)ethynyl]cyclohexane-1-carboxylic acid

To a solution of ethyl (1R,2R)-4-fluoro-2-phenyl-4-[2-(pyrimidin-5-yl)ethynyl]cyclohexane-1-carboxylate (100 mg. 0.28 mmol) in methanol (3.75 mL) and water (1.25 mL) was added at r.t. 50 (w/w) sodium hydroxide (0.25 mL). The reaction mixture was stirred at r.t. for 71 hours. The reaction mixture was concentrated in vacua, diluted with water (30 mL) and extracted with diethyl ether (2×30 mL). The aqueous layer was acidified to pH 2 with 2N HCl and extracted with EtOAc (3×50 mL). The organic layers were combined, dried (MgSO₄) and evaporated to afford (1R,2R)-4-methoxy-2-phenyl-4-[2-(pyrimidin-5-yl)ethynyl]cyclohexane-1-carboxylic acid as a yellow solid. The compound was used without further purification.

LC/MS (Method B): RT=1.05; m/z=337 [M+H]⁺

### Step 4: EXAMPLE 139

Starting from 5-amino-6-(4-fluorophenoxyl-3-[(4-hydroxypiperidin-4-yl)methyl]pyrimidin-4-one (56 mg. 0.17 mmol) and (1R,2R)-4-methoxy-2-phenyl-4-[2-(pyrimidin-5-yl)ethynyl]cyclohexane-1-carboxylic acid (56 mg, 0.17 mmol) following procedure described in Step 3 of EXAMPLE 94, the obtained residue was purified via flash chromatography using DCM-5% MeOH/DCM (gradient) as eluent to give the crude product as a white solid. Final purification via prep HPLC (Prep HPLC Column: Gemini pH 4 Dimensions: 21.1 mm×150 mm 5 μm) afforded the desired product as a white solid.

LC/MS (Method B): RT=1.14; m/z=653 [M+H]⁺

1H NMR (399 MHz, DMSO-d6) δ 9.23 (d, J=1.3 Hz, 1H), 9.02 (d, J=3.1 Hz, 2H), 7.60 (m, 1H), 7.40-7.14 (m, 7H), 7.13-7.04 (m, 2H), 4.82 (d, J=7.5 Hz, 1H), 4.69 (d, J=14.3 Hz, 2H), 4.01-3.59 (m, 4H), 3.38 (d, J=2.5 Hz, 3H), 3.27-3.05 (m, 3H), 2.76-2.58 (m, 1H), 2.29-2,09 (m, 1H), 1.97-1.64 (m, 4H), 1.51-1.03 (m, 3H), 0.88-0.49 (m, 1H).

### 5-amino-3-({1-[(1R,2R,4S)-4-fluoro-2-phenyl-4-[2-(pyrimidin-5-yl)ethynyl]cyclohexanecarbonyl]-4-hydroxypiperidin-4-yl}methyl)-6-(4-fluorophenoxy)-3,4-dihydropyrimidin-1-one (EXAMPLE 140)

### Step 1: tert-butyl (1R,2R)-4 -hydroxy-2phenyl-4 -[2-(pyrimdin-5yl)ethynyl]cyclohexane-1-carboxylate

Starting from tert-butyl (1R,2R)-4-oxo-2-phenylcyclohexane-1-carboxylate (300 mg, 1.09 mmol) and 5-ethynylpyrimidine (148 mg, 1.42 mmol) following procedure described in Step 2 of EXAMPLES 122 and 123, the obtained residue was purified via flash chromatography using Heptane-40% EtOAc/Heptane (gradient) as eluent to afford tert-butyl (1R,2R)-4-hydroxy-2-phenyl-4-[2-(pyrimidin-5-yl)ethynyl]cyclohexane-1-carboxylate as a white solid.

LC/MS (Method B): RT=1.217; m/z=379 [M+H]⁺

### Step 2: tert -butyl (1R,2R,4R)-4-fluoro-2-phenyl-4-[2-(pyrimidin-5 -yl)ethynyl]cyclohexane-1-carboxylate

Starting from tert-butyl (1R,2R)-4-hydroxy-2-phenyl-4-[2-(pyrimidin-5-yl)ethynyl]cyclohexane-1-carboxylate (233 mg, 0.62 mmol) following procedure described in Step 4 of EXAMPLES 122 and 123, the obtained residue was purified via flash chromatography using Heptane-50% EtOAc/Heptane (gradient) as eluent to afford:

First elute: tert-butyl (1R,2R,4R)-4-fluoro-2-phenyl-4-[2-(pyrimidin-5-yl)ethynyl]cyclohexane-1-carboxylate as a white solid.

LC/MS (Method B): RT=1.404; m/z=381 [M+H]⁺

Second elute: tert-butyl (1R,2R,4S)-4-fluoro-2-phenyl-4-[2-(pyrimidin-5-yl)ethynyl]cyclohexane-1-carboxylate as a white solid.

LC/MS (Method B): RT=1.411, m/z=381 [M+H]⁺

### Step 3: (1R,2R,4S)-4 -fluoro-2 phenyl-4-[2-(pyrimidin-5 -yl)ethynyl]cyclohexane-1-carboxylic acid

Starting from tert-butyl (1R,2R,4S)-4-fluoro-2-phenyl-4-[2-(pyrimidin-5-yl)ethynyl]cyclohexane-1-carboxylate (85 mg, 0.22 mmol) following procedure described in Step 5 of EXAMPLE 133, (1R,2R,4S)-4-fluoro-2-phenyl-4-[2-(pyrimidin-5-yl)ethynyl]cyclohexane-1-carboxylic acid was obtained as a white solid.

LC/MS (Method RT=1.068; m/z=323 [M+H]⁺

### Step 4: EXAMPLE 140

Starting from (1R,2R,4S)-4-fluoro-2-phenyl-4-[2-(pyrimidin-5-yl)ethynyl]cyclohexane-1-carboxylic acid (67 mg, 0.21 mmol) and 5-amino-6-(4-fluorophenoxy)-3-[(4-hydroxypiperidin-4-yl)methyl]pyrimidin-4-one (69 mg, 0.21 mmol) following procedure described in Step 3 of EXAMPLE 94, the obtained residue was purified via flash chromatography using water-100% MeCN/water (gradient) as eluent to afford EXAMPLE 144) as a white solid.

LC/MS (Method B): RT=1.151; m/z=621 [M−HF+H]⁺

¹H NMR (399 MHz, DMSO-d₆) 9.22 (s, 1H), 8.96 (m 2H), 7.65-7.57 (m, 1H). 7.34-7.16 (m, 7H), 7.12-7.08 (m, 2H), 4.83 (m, 1H), 4.69 (m, 2H), 3.97-3.63 (m 4H), 3.27-2.90 (m, 3H), 2.70-2.64 (m, 1H), 2.42-2.09 (m, 4H), 1.85-1.67 (m, 2H), 1.47-1.12 (m, 3H), 0.76-0.64 (m, 1H).

HRMS (TOF, ESI) m/z: Calculated for C₃₅H₃₄F₂N₆O₄ 640.2610, Found: 641.2699 [M+H]⁺

### 5-amino-3-[(1-{[(1R,2R,4R)-4-fluoro-2-phenyl-4-[2-(pyrimidin-2-yl)ethynyl]cyclohexyl]carbonyl}-4-hydroxypiperidin-4-yl)methyl]-6-(4-fluorophenoxy)pyrimidin-4-one (EXAMPLE 141).

### 5-amino-3-[(1-{[(1R,2R,4S)-4-fluoro-2-phenyl-4-[2-(pyrimidin-2-yl)ethynyl]cyclohexyl]carbonyl}-4-hydroxypiperidin-4-yl)methyl]-6-(4-fluorophenoxy)pyrimidin-4-one (EXAMPLE 142)

and

### 5-amino-6-(4-fluorophenoxy)-3-[(4-hydroxy-1-{[(1R,6R)-6-phenyl-4-[2-(pyrimidin-2-yl)ethynyl]cyclohex-3-en-1-yl]carbonyl}piperidin-4-yl)methyl]pyrimidin-4-one (EXAMPLE 143)

### Step 1: tert-butyl (1R,2R)-4-[2-(5-fluoropyridin-2-yl)ethynyl]-4-hydroxy-2-phenylcyclohexane-1-carboxylate

Starting from tert-butyl (1R,2R)-4-oxo-2-phenylcyclohexane-1-carboxylate (600 mg, 2.19 mmol) and 2-ethynylpyrimidine (296 mg, 2.84 mmol, 1.3 eq.) following procedure described in Step 2 of EXAMPLES 122 and 123, the obtained residue was purified via flash chromatography using Heptane-65% EtOAc/Heptane (gradient) as eluent to give:

First elute: tri-butyl (1R,2R,4S)-4-hydroxy-2-phenyl-4-[2-(pyrimidin-2-yl)ethynyl]cyclohexane-1 -carboxylate obtained as a white solid.

LC/MS (Method B): RT=1.233; m/z=379 [M+H]⁺

Second elute: tert-butyl (1R,2R,4R)-4-hydroxy-2-phenyl-4-[2-(pyrimidin-2-yl)ethynyl]cyclohexane-1-carboxylate obtained as a colourless oil.

LC/MS (Method B): RT=1.205; m/z=379 [M+H]⁺

### Step 2: tert-butyl (1R,2R)-4-fluoro-2-phenyl-4-[2-(pyrimidin-2-yl)ethynyl]cyclohexane-1-carboxylate

Starting from tert-butyl (1R,2R,4R)-4-hydroxy-2-phenyl-4-[2-(pyrimidin-2-yl)ethynyl]cyclohexane-1-carboxylate (220 mg, 0.58 mmol) following procedure described in Step 4 of EXAMPLES 122 and 123. the obtained residue was purified via flash chromatography using Heptane-35% EtOAc/Heptane (gradient) as eluent to afford tert-butyl (1R,2R)-4-fluoro-2-phenyl-4-[2-(pyrimidin-2-yl)ethynyl]cyclohexane-1-carboxylate as a yellow oil.

LC/MS (Method 13), RT=1.376; m/z=381 [M+H]⁺

### Step 3: (1R,2R)-4-fluoro-2-phenyl-4-[2-(pyrimidin-2-yl)ethynyl]cyclohexane-1-carboxylic acid

Starting from tert-butyl (1R,2R)-4-fluoro-2-phenyl-4-[2-(pyrimidin-2-yl)ethynyl]cyclohexane-1-carboxylate (150 mg, 0.39 mmol) following procedure described in Step 5 of EXAMPLE 133, (1R,2R)-4-fluoro-2-phenyl-4-[2-(pyrimidin-2-yl)ethynyl](cyclohexane-1-carboxylic acid was obtained as a yellow oil. The compound was used without further purification.

LC/MS (Method B): RT=1.02; m/z=325 [M+H]⁺

### Step 4: EXAMPLES 141, 142 and 144

Starting from 5-amino-6-(4-fluorophenoxy)-3-[(4-hydroxypiperidin-4-yl)methyl]pyrimidin-4-one (127 mg, 0.38 mmol) and (1R,2R)-4-fluoro-2-phenyl-4-[2-(pyrimidin-2-yl)ethynyl]cyclohexane-1-carboxylic acid (190 mg) following procedure described in Step 3 of EXAMPLE 94, the obtained residue was purified via flash chromatography using DCM-5% MeOH (gradient) as eluent to give the still impure product as colourless oil. Final purification was performed via prep HPLC Prep (HPLC Column: Gemini pH4 Dimensions: 21.1 mm×150 mm 5 μm) to afford the desired products.

First elute: EXAMPLE 143 as a white solid.

LC/MS (Method 1290): RT=0.93; m/z=621 [M+H]⁺

¹H NMR (399 MHz, DMSO-₆) 8.79 (m, 2H), 7.63-7.59 (m, 1H), 7.47 (t, 1H), 7.33-7.18 (m, 7H), 7.12-7.07 (m, 2H), 6.49 (m, 1H), 4.86 (m, 1H), 4.69 (m, 2H), 3.94-3.68 (m, 4H), 3.47-3.35 (m, 1H), 3.18-2.93 (m, 2H), 2.7-2.54 (m, 2H), 2.40 (m, 3H) 1.46-1.13 (m, 3H), 0.93-0.64 (m, 1H).

HRMS (TOF, ESI) m/z: Calculated for C₃₅H₃₃FN₆O₄ 620.2547, Found: 621.2659 [M+H]⁺

Second elute: EXAMPLE 142 as a white solid.

LC/MS (Method 1290): RT=0.94; m/z=641 [M+H]⁺

¹H NMR (399 MHz, DMSO-d₆) δ 8.83 (m, 2H), 7.63-7.54 (m, 2H), 7.37-7.15 (m, 7H), 7.11-7.07 (m, 2H), 4.86 (m, 1H), 4.69 (m, 2H), 3.97-3.62 (m, 4H), 3.27-2.87 (m, 3H), 2.66 (m, 1H), 2.43-2.10 (m, 4H), 1.84-1.66 (m, 2H), 1.45-1.11 (m, 3H), 0.78-0.57 (m, 1H) HRMS (TOF, ESI) m/z: Calculated for C₃₅H₃₄F₂N₆O₄ 640.261, Found: 641.2705 [M+H]⁺

Third elute: EXAMPLE 141 as a white solid.

LC/MS (Method 1290): RT=0.96; m/z=641 [M+H]⁺

¹H NMR (399 MHz, DMSO-d₆) δ 8.87 (m, 2H), 7.62-7.56 (m, 2H), 7.35-7.15 (m, 7H), 7.11-7.07 (m, 2H), 4.82 (m, 1H), 4.69 (m, 2H), 3.96-3.61 (m, 4H), 3.31-2.85 (m, 3H), 2.65 (m, 1H), 2.36-2.10 (m, 4H), 1.92-1.80 (m, 2H), 1.46-1.11 (m, 3H), 0.78-0.53 (m, 1H) HRMS (TOF, ESI) m/z: Calculated for C₃₅H₃₄F₂N₆O₄ 640.261, Found: 641.2613 [M+H]⁺

### 5-amino-3-({1-[(1R,2R,4R)-4-fluoro-2-phenyl-4-[2-(pyrimidin-5-yl)ethynyl]cyclohexanecarbonyl]-4-hydroxypiperidin-4-yl}methyl)-6-(4-fluorophenyoxy)-3,4-dihydropyrimidin-4-one (EXAMPLE 144)

### Step 1: (1R,2R,4R)-4-fluoro-2-phenyl-4-[2-(pyrimidin-5-yl)ethlynyl]cyclohexane-1-carboxylic acid

Starting from tert-butyl (1R,2R,4R)-4-fluoro-2-phenyl-4-[2-(pyrimidin-5-yl)ethynyl]cyclohexane-1-carboxylate (68 mg, 0.18 mmol) following procedure described in Step 5 of EXAMPLE 133, (1R,2R,4R)-4-fluoro-2-phenyl-4-[2-(pyrimidin-5-yl)ethynyl]cyclohexane-1-carboxylic acid was obtained as a white solid.

LC/MS (Method B): RT=1.099; m/z=323 [M−H]⁺

### Step 2: EXAMPLE 144

Starting from (1R,2R,4R)-4-fluoro-2-phenyl-4-[2-(pyrimidin-5-yl)ethynyl]cyclohexane-1-carboxylic acid (46 mg, 0.14 mmol) and 5-amino-6-(4-fluorophenoxy)-3-[(4-hydroxypiperidin-4-yl)methyl]pyrimidin-4-one (47 mg, 0.14 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 144 as a white solid.

LC/MS (Method B): RT=1.17 m/z=641 [M+H]⁺

¹H NMR (399 MHz, DMSO-d₆) 9.27 (s, 1H), 9.06 (m, 2H), 7.65-7.57 (m, 1H), 7.34-7.17 (m, 7H), 7.12-7.07 (m, 2H), 4.82 (m, 1H), 4.69 (m, 2H), 3.97-3.61 (m, 4H), 3.32-2.87 (m, 3H), 168-2.52 (m, 1H), 2.33-2.09 (m, 4H), 1.87 (m, 2H), 1.47-1.11 (m, 3H), 0.79-0.55 (m, 1H),

HRMS (TOF, ESI) m/z: Calculated for C₃₅H₃₄F₂N₆O₄ 640.2610, Found 641.2627 [M+H]⁺

### 5-amino-3-[(1-{[(1R,2R,4R)-4-fluoro-2-phenyl-4-[2-(pyridazin-3-yl)ethynyl]cyclohexyl]carbonyl}-4-hydroxypiperidin-4-yl)methyl]-6-(4-fluorophenoxy)pyrimidin-4-one (EXAMPLE 145)

### Step 1: tert-butyl (1R,2R)-4-hydroxy-2-phenyl-4-[2-(pyridazin-3-yl)ethyl]cyclohexane-1-carboxylate

Starting from tert-butyl (1R,2R)-4-oxo-2-phenylcyclohexane-1-carboxylate (400 mg, 1.46 mmol) and 3-ethynylpyridazine, (197 mg, 1.9 mmol, 1.3 eq.) following procedure described in Step 2 of EXAMPLES 122 and 123, the obtained residue was purified via flash chromatography using Heptane-88% EtOAc/Heptane (gradient) as eluent to give tert-butyl (1R,2R)-4-hydroxy-2-phenyl-4-[2-(pyridazin-3-yl)ethynyl](cyclohexane-1-carboxylate as a yellow solid.

LC/MS (Method B): RT=118; m/z=379 [M+H]⁺

### Step 2: tert-butyl (1R,2R)-4fluoro-2-phenyl-4-[2-(pyridazin-3-yl)ethynyl]cyclohexane-1-carboxylate

Starting from tert-butyl (1R,2R)-4-hydroxy-2-phenyl-4-[2-(pyridazin-3-yl)ethynyl]cyclohexane-1-carboxylate (260 g, 0.69 mmol) following procedure described in Step 4 of EXAMPLES 122 and 123, the obtained residue was purified via flash chromatography using Heptane-93% EtOAc/Heptane (gradient) as eluent to afford tert-butyl (1R,2R)-4-fluoro-2-phenyl-4-[2-(pyridazin-3-yl)ethynyl]cyclohexane-1-carboxylate as a yellow foam. LC/MS (Method 8): RT=1.15; m/z=381 [M+H]⁺

### Step 3: (1R,2R)-4-fluoro-2-phenyl-4-[2-(pyridazin-3-yl)ethynyl]cyclohexane-1-carboxylic acid

Starting from tert-butyl (1R,2R)-4-fluoro-2-phenyl-4-[2-(pyridazin-3-yl)ethynyl]cyclohexane-1-carboxylate (90 mg, 0.24 mmol) following procedure described in Step 5 of EXAMPLE 133, (1R,2R)-4-fluoro-2-phenyl-4-[2-(pyridazin-3-yl)ethynyl]cyclohexane-1-carboxylic acid was obtained as a yellow oil. The compound was used without further purification.

LC/MS (Method B): RT=0.987; m/z=325 [M+H]⁺

### Step 4: EXAMPLE 145

Starting from 5-amino-6-(4-fluorophenoxy)-3-[(4-hydroxypiperidin-4-yl)methyl]pyrimidin-4-one (123 mg, 0.37 mmol) and (1R,2R)-4-fluoro-2-phenyl-4-[2-(pyridazin-3-yl)ethynyl]cyclohexane-1-carboxylic acid (170 mg) following procedure described in Step 3 of EXAMPLE 94, the obtained residue was purified via flash chromatography using DCM-6% MeOH (gradient) as eluent to give the still impure product as colourless oil. Final purification was performed via prep HPLC Prep (HPLC Column: Gemini pH4 Dimensions: 211 mm×mm 5 μm) to afford EXAMPLE 145 as a white solid.

LC/MS (Method B): RT=1.13; m/z=641 [M+H]⁺

¹H NMR (399 MHz, DMSO-d₆) δ 9.31 (dt, J=5.1, 1.6 Hz, 1H), 7.99 (ddd, J=8.5, 3.2, 1.7 Hz, 1H), 7.82 (ddd, J=8.5. 5.1, 1.5 Hz, 1H), 7.62-7.57 (m, 1H), 7.35-7.17 (m, 7H), 7.12-7.07 (m, 2H), 4.82 (m, 1H), 4.68 (m, 2H), 3.96-3.61 (m, 4H), 3.31-2.87 (m, 3H), 2.65 (m, 1H), 2.40-2.12 (m, 4H), 1.89 (m, 2H),1.47-1.11 (m, 3H), 0.79-0.55 (m, 1H), HRMS (TOF, ESI) m/z: Calculated for C₃₅H₃₄F₂N₆O₄ 640.2610, Found: 641.2684 [M+H]⁺

### 5-amino-6-(4-fluorophenoxy)-3-[(4-hydroxy-{[(1R,2R,4R)-4-methoxy-2-phenyl-4-[2-(pyridazin-3-yl)ethynyl]cyclohexyl]carbonyl}piperidin-4-yl)methyl]pyrimidin-4-one (EXAMPLE 146)

and

### 5-amino-6-(4-fluorophenoxy)-3-[(4-hydroxy-1-{[(1R,2R,4S)-4-methoxy-2-phenyl-4-[2-(pyridazin-3-yl)ethynyl]cyclohexyl]carbonyl}piperidin-4-yl)methyl]pyrimidin-4-one (EXAMPLE 147)

### Step 1: tert-butyl (1R,2R)-4-methoxy-2-phenyl-4-[2-(pyridazin-3-yl)ethynyl]cyclohexane-1-carboxylate

To a solution of tert-butyl (1R,2R)-4-hydroxy-2-phenyl-4-[2-(pyridazin-3-yl)ethynyl]cyclohexane-1-carboxylate (100 mg, 0.26 mmol, 1 eq.) in DMF (3 mL) at 0° C. under nitrogen, sodium hydride (13 mg, 0.53 mmol, 2 eq.) was added. The mixture stirred for 10 minutes before adding iodomethane (0.02 mL, 0.26 mmol, 1 eq.). After 10 minutes the reaction mixture was allowed to warm to r.t. over 1 hour, Aq. NH₄Cl solution was added (15 mL) and extracted with EtOAc (2×20 mL). The combined organic layer were washed with brine (3×20 mL), dried over MgSO₄ and evaporated in vacuo to afford a crude oil (200 mg), This residue was purified via flash chromatography using Heptane-60% EtOAc (gradient) as eluent to give tert-butyl (1R,2R)-4-methoxy-2-phenyl-4-[2-(pyridazin-3-yl)ethynyl]cyclohexane-1-carboxylate as a brown oil.

LC/MS (Method B): RT=1.11; m=393 [M+H]⁺

### Step 2: (1R, 2R)-4-methoxy-2-phenyl-4-[2-pyridazin-3-yl)ethynyl]cyclohexane-1-carboxylic acid

Starting from tert-butyl (1R,2R)-4-methoxy-2-phenyl-4-[2-(pyridazin-3-yl)ethynyl]cyclohexane-1-carboxylate (80 mg, 0.2 mmol) following procedure described in Step 5 of EXAMPLE 133, (1R,2R)-4-methoxy-2-phenyl-4-[2-(pyridazin-3-yl)ethynyl]cyclohexane-1-carboxylic acid was obtained as a yellow oil, The compound was used without further purification.

LC/MS (Method B): RT=0.80; m/z=337 [M+H]⁺

### Step 3: EXAMPLES 146 and 147

Starting from 5-amino-6-(4-fluorophenoxy)-3-[(4-hydroxypiperidin-4-yl)methyl]pyrimidin-4-one (60 mg, 0.18 mmol) and (1R,2R)-4-methoxy-2-phenyl-4-[2-(pyridazin-3-yl)ethynyl]cyclohexane-1-carboxylic acid (60 mg, 1 eq.) following procedure described in Step 3 of EXAMPLE 94, the obtained residue was purified via flash chromatography using DCM-5% MeOH (gradient) as eluent to give the still impure product as colourless oil. Final purification was performed via prep HPLC Prep (HPLC Column: Gemini pH4 Dimensions: 21.1 mm×150 min 5 μm) to afford:

First elute: EXAMPLE 147 as a white solid.

LC/MS (Method RT=1.08, m/z=653 [M+H]⁺

¹H NMR (399 MHz, DMSO-₆) δ 9.28 (dt, J=5.1. 1.6 Hz, 1H), 7.94 (ddd, J=8.5. 2.8, 1.7 Hz, 1H), 7.79 (ddd, J=8.5, 5.0, 1.3 Hz, 1H), 7.63-7.57 (m, 1H), 7.33-7.16 (m, 7H), 7.11-7.07 (m, 2H), 4.82 (m, 1H), 4.68 (m, 2H), 3.96-3.61 (m, 4H), 3.41 (m, 3H), 3.25-2.89 (m, 3H), 2.67 (m, 1H), 2.22 (m, 2H), 1.97-1.74 (m, 4H), 1.46-1.11 (m, 3H), 0.80-0.55 (m, 1H)

HRMS (TOF, ESI) m/z: Calculated for C₃₆H₃₇FN₆O₅ 652.809, Found: 653.2886 [M+H]⁺

Second elute: EXAMPLE 146 as a white solid

LC/MS (Method B): RT=1.11, m/z=653 [M+H]⁺

¹H NMR (399 MHz, DMSO-d₆) 9.15 (dt, J=5.0, 1.6 Hz, 1H), 7.76 (ddd, J=8.5, 3.2, 1.7 Hz, 1H), 7.66 (ddd, J=8.5, 5.0, 1.8 Hz, 1H) 7.57-7.51 (m, 1H), 7.25-7.09 (m, 7H), 7.05-7.00 (m, 2H), 4.80 (m, 2H), 3.89-3.53 (m, 4H), 3.41 (m, 3H), 3.17-2.83 (m, 4H), 2.58 (m, 1H), 2.22-1.86 (m, 5H), 1.68 (m, 1H), 1.54-1.06 (m, 3H), 0.73-0.50 (m, 1H)

HRMS (TOE, ESI) m/z: Calculated for C₃₆H₃₇FN₄O₅ 652.2809, Found: 653.2889 [M+H]⁺

### 5-amino-3-({1-[(1R,2R,4R)-4-fluoro-4-[2-(3-methylpyrazin-2yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 148)

### Step 1: tert-butyl (1R,2R)-4-hydroxy-4-[2-(3-methylpyrazin-2-yl)ethynyl]-2-phenylcyclohexane-1-carboxylate

Starting from tert-butyl (1R,2R)-4-oxo-2-phenylcyclohexane-1-carboxylate (300 mg, 1.09 mmol) and 2-ethynyl-3-methylpyrazine (167.93 mg, 1.42 mmol) following procedure described in Step 2 of EXAMPLES 122 and 123, the obtained residue was purified via flash chromatography using Heptane-60% EtOAc/Hemline (gradient) as eluent to afford tert-butyl (1R,2R)-4-hydroxy-4-[2-(3-methylpyrazin-2-yl)ethynyl]-2-phenylcyclohexane-1-carboxylate as a yellow oil.

LC/MS (Method B); RT=1.247; m/z=393 [M+H]⁺

### Step 2: tert-butyl (1R,2R)-4-fluoro-4-[2-(3-methylpyrazin-2-yl)ethynyl]-2-phenylcyclohexane-1-carboxyate

Starting from tert-butyl (1R,2R)-4-hydroxy-4-[2-(3-methylpyrazin-2-yl)ethynyl]-2-phenylcyclohexane-1-carboxylate (252 mg, 0.64 mmol) following procedure described in Step 4 of EXAMPLES 122 and 123, the obtained residue was purified via flash chromatography using Heptane-30% EtOAc/Heptane (gradient) as eluent to afford tert-butyl (1R,2R)-4-fluoro-4-[2-(3-methylpyrazin-2-yl)ethynyl]-2-phenylcyclohexane-1-carboxylate as an oil.

LC/MS (Method B): RT=1.427; m/z=395 [M+H]⁺

### Step 3: (1R,2R)-4-fluoro-4-[2-(3-methylpyrazin-2-yl)ethynyl]-2-phenylcyclohexane-1-carboxylic acid

Starting from tert-butyl (1R,2R)-4-fluoro-4-[2-(3-methylpyrazin-2-yl)ethynyl]-2-phenylcyclohexane-1-carboxylate (47 mg, 0.12 mmol) following procedure described in Step 5 of EXAMPLE 133, (1R,2R)-4-fluoro-4-[2-(3-methylpyrazin-2-yl)ethynyl]-2-phenylcyclohexane-1-carboxylic acid was obtained as a yellow oil. The compound was used without further purification.

LC/MS (Method B): RT=1.12; m/z=339 [M−H]⁺

### Step 4: EXAMPLE 148

Starting from (1R,2R)-4-fluoro-4-[2-(3-methylpyrazin-2-yl)ethynyl]-2-phenylcyclohexane-1-carboxylic acid (150 mg) and 5-amino-6-(4-fluorophenoxy)-3-[(4-hydroxypiperidin-4-yl)methyl]pyrimidin-4-one (148 mg, 0.44 mmol) following procedure described in Step 3 of EXAMPLE 94, the obtained residue was purified via prep HPLC Prep (HPLC Column: Gemini pH4 Dimensions: 21.1 min×150 mm 5 μm) to afford EXAMPLE 148 as a white solid.

LC/MS (Method B). RT=1.203, m/z=655 [M+H]⁺

¹H NMR (399 MHz, DMSO-d₆) 8.59 (m, 1H), 8.56 (m, 1H), 7.62-7.56 (m, 1H), 7.35-7.18 (m, 7H), 7.11-7.09 (m, 2H), 4.82 (m, 1H), 4.67 (m, 2H), 3.96-3.59 (m, 4H), 3.27-2.85 (m, 3H), 2.74 (s, 3H), 2.66 (m, 1H), 2.35-2.10 (m, 4H), 1.89 (m, 2H), 1.47-1.11 (m, 3H), 0.74-0.56 (m, 1H).

HRMS (TOF, ESI) m/z: Calculated for C₃₆H₃₆F₂N₆O₄ 654.2766, Found: 655.2849 [M+H]⁺

### 5-amino-3-[(1-{[(1R,2R,4R)-4-fluoro-4-[2-(1-methylimidazol-2-yl)ethynyl]-2-phenylcyclohexyl]carbonyl}-4-hydroxypiperidin-4-yl)methyl]-6-(4-fluorophenoxy)pyrimidin-4-one (EXAMPLE 149)

### Step 1: tert-butyl (1R,2R)-4-hydroxy-4-[2-(1-methylimidazol-2-yl)ethynyl]-2-phenylcyclohexane-1-carboxylate

Starting from tert-butyl (1R,2R)-1-oxo-2-phenylcyclohexane-1-carboxylate (400 mg, 1.46 mmol) and 2-ethynyl-1-methyl-1H-imidazole (201 mg, 1.0 mmol, 1.3 eq.) following procedure described in Step 2 of EXAMPLES 122 and 123, the obtained residue was purified via flash chromatography using DCM-5% MeOH/DCM (gradient) as eluent to give tert-butyl (1R,2R)-4-hydroxy-4-[2-(1-methylimidazole-2-yl)ethynyl]-2-phenylcyclohexane-1-carboxylate as a white solid.

LC/MS (Method B): RT=1.122; m/z=381 [M+H]⁺

### Step 2: tert-butyl (1R,2R,4S)-4-fluoro-1-[2-(1-methylimidazol-2-yl)ethynyl]-2-phenylcyclohexane-1-carboxylate (XX) and tert-butyl (1R,2R,4R)-4-fluoro-4-[2-(1-methylimidazol-2yl)ethynyl]-2-phenylcyclohexane-1-carboxylate

Starting from tert-butyl (1R,2R)-4-hydroxy-4-[2-(1-methylimidazol-2-yl)ethynyl]-2-phenylcyclohexane-1-carboxylate (380 mg, 1.00 mmol) following procedure described in Step 4 of EXAMPLES 122 and 123, the obtained residue was purified via flash chromatography using Heptane-58% EtOAc/Heptane (gradient) as eluent to afford:

First elute: tert-butyl (1R,2R,4R)-4-fluoro-4-[2-(1-methylimidazol-2-yl)ethynyl]-2-phenylcyclohexane-1-carboxylate

LC/MS (Method B): RT=132; m/z=383 [M+H]⁺

Second elute: tert-butyl (1R,2R,4S)-4-fluoro-4-[2-(1-methylimidazol-2-yl)ethynyl]-2-phenylcyclohexane-1-carboxylate

LC/MS (Method RT=1.33; m/z=383 [M+H]⁺

### Step 3: (1R,2R,4R)-4-fluoro-4-[2-(1-methylimidazol-2-yl)ethynyl]-phenylcyclohexane-1-carboxylic acid

Starting from tert-butyl (1R,2R,4R)-4-fluoro-4-[2-(1-methylimidazol-2-yl)ethynyl]-2-phenylcyclohexane-1-carboxylate (65 mg, 0.17 mmol) following procedure described in Step 5 of EXAMPLE 133, (1R,2R,4R)-4-fluoro-4-[2-(1-methylimidazol-2-yl)ethynyl]-2-phenylcyclohexane-1-carboxylic acid was obtained as a yellow oil. The compound was used without further purification.

LC/MS (Method B): RT=1.003; m/z=327 [M+H]⁺

### Step 4: EXAMPLE 149

Starting from 5-amino-6-(4-fluorophenoxy)-3-[(4-hydroxypiperidin-4-yl) methyl]pyrimidin-4-one (92 mg, 0.28 mmol) and (1R,2R,4R)-4-fluoro-4-[2-(1-methylimidazol-2-yl)ethynyl]-2-phenylcyclohexane-1-carboxylic acid (90 mg) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 149 as a white solid.

LC/MS (Method B): RT=1.11; m/z=643 [M+H]⁺

¹H NMR (399 MHz, DMSO-d₆) δ 7.55-7.50 (m, 1H), 7.33 (s, 1H), 7.27-6.94 (m, 10H), 4.75 (m, 3H), 3.89-3.77 (m, 2H), 3.72 (s, 3H), 3.65-3.52 (m, 2H), 3.24-2.77 (m, 3H), 2.62-2.54 (m, 1H), 2.30-2.00 (m, 4H), 1.84-1.73 (m, 2H), 1.39-1.03 (m, 3H), 0.71-0.42 (m, 1H),

HRMS (TOF, ESI) m/z: Calculated for C₃₅H₃₆F₂N₆O₄ 642.2766, Found: 643.2808 [M+H]⁺

### 5-amino-3-({1-[1R,2R,4S)-4-fluoro-4-[2-(5-methylpyrazin-2-yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 150)

and

### 5-amino-3-({1-[(1R,2R,4S)-4-fluoro-4-[2-(5-methylpyrazin-2-yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 151)

### Step 1: tert-butyl (1R,2R) -4-hydroxy-4-[2-(5-methylpyrazin-2-yl)ethynyl]-2-phenylcyclohexane-1-carboxylate

Starting from tert-butyl (1R,2R)-4-oxo-2-phenylcyclohexane-1-carboxylate (300 mg, 1.09 mmol) and 2-ethynyl-5-methylpyrazine (168 mg, 1.42 mmol, 1.3 eq.) following procedure described in Step 2 of EXAMPLES 122 and 123, the obtained residue was purified via flash chromatography using Heptane-60% EtOAc/Heptane (gradient) as eluent to afford tert-butyl (1R,2R)-4-hydroxy-4-[2-(5-methylpyrazin-2-yl)ethynyl]-2-phenylcyclohexane-1-carboxylate as an off-white solid.

LC/MS (Method B): RT=1.26; m/z=393 [M+H]⁺

### Step 2: tert-butyl (1R,2R)-4-fluoro-4-[2-(5-methylpyrazin-2-yl)ethynyl]-2 -phenylcyclohexane-1-carboxylate

Starting from tert-butyl (1R,2R)-4-hydroxy-4-[2-(5-methylpyrazin-2-yl)ethynyl]-2-phenylcyclohexane-1-carboxylate (185 mg, 0.47 mmol) following procedure described in Step 4 of EXAMPLES 122 and 123, the obtained residue was purified via flash chromatography using Heptane-60% EtOAc/Heptane (gradient) as eluent to afford tert-butyl (1R,2R)-4-fluoro-4-[2-(5-methylpyrazin-2-yl)ethynyl]-2-phenylcyclohexane-1-carboxylate as an oil.

LC/MS (Method B): RT=1.437; m/z=395 [M+H]⁺

### Step 3: (1R,2R)-4-fluoro-4-[2-(5-methylpyrazin-2-yl)ethynyl]-2-phenylcyclohexane-1-carboxylic acid

Starting from tert-butyl (1R,2R)-4-fluoro-4-[2-(5-methylpyrazin-2-yl)ethynyl]-2-phenylcyclohexane-1-carboxylate (147 mg, 0.37 mmol) following procedure described in Step 5 of EXAMPLE 133, (1R,2R)-4-fluoro-4-[2-(5-methylpyrazin-2-yl)ethynyl]-2-phenylcyclohexane-1-carboxylic acid was obtained as a yellow oil. The compound was used without further purification.

LC/MS (Method B): RT=1.136; m/z=339 [M+H]⁺

### Step 4: 5 EXAMPLES 150 and 151

Starting from (1R,2R)-4-fluoro-4-[2-(5-methylpyrazin-2-yl)ethynyl]-2-phenylcyclohexane-1-carboxylic acid (144 mg) and 5-amino-6-(4-fluorophenoxy)-3-[(4-hydroxypiperidin-4-yl)methyl]pyrimidin4-one (142 mg, 0.44 mmol) following procedure described in Step 3 of EXAMPLE 94, the obtained residue was purified via prep HPLC Prep (HPLC Column: Gemini pH4 Dimensions: 21.1 mm×150 mm 5 μm to give:

First elute:. EXAMPLE 159 as a white solid.

LC/MS (Method B): RT=1.198; m/z=635 [M+H]⁺

¹H NMR (399 MHz, DMSO-d₆) 8.68 (m, 1H), 8.59 (m, 1H), 7.63-7.57 (m, 1H), 7.33-7.17 (m, 7H), 7.12-7.07 (m, 2H), 4.82 (m, 1H), 4.68 (m, 2H), 3.97-3.62 (m, 4H), 3.28-2.88 (m, 3H), 2.67 (m, 1H), 2.53 (s, 3H), 2.46-2.08 (m, 4H), 1.87-1.65 (m, 2H), 1.47-1.11 (m, 3H), 0.79-0.57 (m, 1H).

HRMS (TOF, ESI) m/z: Calculated for C₃₆H₃₆F₂N₆O₄ 654.2766, Found: 655.2818 [M+H]⁺

Second elute: EXAMPLE 151 as a white solid.

LC/MS (Method B): RT=1.216; m/z=655 [M+H]⁺

¹H NMR (399 MHz, DMSO-d₆) 8.76 (m, 1H), 8.64 (m, 1H), 7.62-7.56 (m, 1H), 7.35-7.17 (m, 7H), 7.12-7.07 (m, 2H), 4.82 (m, 1H), 4.68 (m, 2H), 3.96-3.61 (m, 4H), 3.29-2.85 (m, 3H), 2.66 (m, 1H), 2.57 (s, 3H), 2.34-2.08 (m, 4H), 1.86 (m, 2H), 1.47-1.11 (m, 3H), 0.78-0.53(m, 1H).

HRMS (TOF, ESI) m/z: Calculated for C₃₆H₃₆F₂N₆O₄ 654.2766, Found: 655.2809 [M+H]⁺

### 5-amino-3-[(1-{[(1R,2R4S)-4-fluoro-1-[2-(1-methylimidazol-2-yl)ethynyl]-2-phenylcyclohexyl]carbonyl}-4-hydroxypiperidin-4-yl)methyl]-6-(4-fluorophenoxy)pyrimidin-4-one (EXAMPLE 152)

### Step 1: (1R,2R,4S)-4-fluoro-4-[2-(1-methylimidazol-2-yl)ethynyl]-2-phenylcyclohexane-1-carboxylic acid

Starting from tert-butyl (1R,2R,4S)-4-fluoro-4-[2-(1-methylimidazol-2-yl)ethynyl]-2-phenylcyclohexane-1 -carboxylate (190 mg, 0.50 mmol) following procedure described in Step 5 of EXAMPLE 133, (1R,2R,4S)-4-fluoro-4-[2-(1-methylimidazol-2-yl)ethynyl]-2-phenylcyclohexane-1-carboxylic acid was obtained as a yellow oil. The compound was used without further purification.

LC/MS (Method B): RT=0.77; m/z=327 [M+H]⁺

### Step 2: EXAMPLE 152

Starting from 5-amino-6-(4-fluorophenoxy)-3-[(4-hydroxypiperidin-4-yl)methyl]pyrimidin-4-one (195 mg, 0.98 mmol) and (1R,2R,4S)-4-fluoro-4-[2-(1-methylimidazol-2-yl)ethynyl]-2-phenylcyclohexane-1-carboxylic acid (190 mg) following procedure described in Step 3 of EXAMPLE 94, the obtained residue was purified via flash chromatography using DCM-3% MeOH (gradient) as eluent to give the still impure product as colourless oil. Final purification was performed via prep HPLC Prep (HPLC Column: Gemini pH-4 Dimensions: 21.1 mm×150 mm 5 μm) to afford EXAMPLE 152 as a white solid.

LC/MS (Method B): RT=1.088; m/z=643 [M+H]⁺

¹H NMR (399 MHz, DMSO-d₆) δ 7.55-7.50 (m, 1H), 7.26-7.08 (m, 1H), 7.04-7.00 (m, 2H), 6.91 (m, 1H), 4.76 (m, 1H) 4.60 (m, 2H), 3.90-3.55 (m, 5H), 3.41-2.82 (m, 5H), 2.62-2.51 (m, 1H), 2.20-1.98 (m, 4H), 1.84-1.53 (m, 2H), 1.43-0.97 (m, 3H), 0.70-0.55 (m, 1H),

HRMS (TOF, ESI) m/z: Calculated for C₃₅H₃₆F₂N₆O₄ 642.2766, Found: 643.281 [M+H]⁺

### 5-amino-3-({1-[(1R,2R,4R)-4-[2-(3-chloropyrazin-2-yl)ethynyl]-4-fluoro-2-phenylcyclohexanecarbonyl]-4-hydroxypiperidin-4-yl}methyl)-6-(4-fluorophenoxy)-3,4-dihydropyrimidin-4-one (EXAMPLE 153)

### Step 1: tert-butyl (1R,2R)-4-[2-(3-chloropyrazin-2-yl)ethynyl]-4-hydroxy-2-phenylcyclohexane-1-carboxylate

Starting from tert-butyl (1R2R)-4-oxo-2-phenylcyclohexane-1-carboxylate (300 mg, 1.09 mmol) and 2-chloro-3-ethynylpyrazine (196.96 mg, 1.42 mmol) following procedure described in Step 2 of EXAMPLES 122 and 123, the obtained residue was purified via flash chromatography using Heptane-40% EtOAc/Heptane (gradient) as eluent to afford tert-butyl (1R,2R)-4-[2-(3-chloropyrazin-2-yl)ethynyl]-4-hydroxy-2-phenylcyclohexane-1-carboxylate as a yellow foam.

¹H NMR (399 MHz, Chloroform-d) 8.44 (d, J=2.5 Hz, 1H), 8.27 (d, J=2.5 Hz, 1H), 7.26-7.09 (m, 5H), 3.22 (ddd, J 13.1, 11.4, 3.3 Hz, 1H), 2.49-2.42 (m, 1H), 2.38 (s, 1H), 2.28-2.23 (m, 2H), 2.07-2.01 (m, 2H), 1.91-1.67 (m, 2H), 1.25-1.15 (m, 1H), 1.07 (s, 9H).

### Step 2: tert-butyl (1R,2 R)-4-[2-chloropyrazin-2yl)ethynyl]-4-fluoro-2-phenylcyclohexane-1-carboxylate

Starting from tert-butyl (1R,2R)-4-[2-(3-chloropyrazin-2-yl)ethynyl]-4-hydroxy-2-phenylcyclohexane-1-carboxylate (196 mg, (1.47 mmol) following procedure described in Step 4 of EXAMPLES 122 and 123, the obtained residue was purified via flash chromatography using Heptane-60% EtOAc/Heptane (gradient) as eluent to afford tert-butyl (1R,2R)-4-[2-(3-chloropyrazin-2-yl)ethynyl]-4-fluoro-2-phenylcyclohexane-1-carboxylate as an oil.

LC/MS (Method B): RT=1.471; m/z=394 Others

### Step 3: (1R,2R)-4-[2-(3-chloropyrazin-2yl)ethynyl]-4-fluoro-2-phenylcyclohexane-1-carboxylic acid

Starting from tert-butyl (1R,2R)-4-[2-(3-chloropyrazin-2-yl)ethynyl]-4-fluoro-2-phenylcyclohexane-1-carboxylate (121 mg, 0.29 mmol) following procedure described in Step 5 of EXAMPLE 133, (1R,2R)-4-fluoro-4 -[2-(3-methylpyrazin-2-yl)ethynyl]-2-phenylcyclohexane-1-carboxylic acid was obtained as a yellow oil. The compound was used without further purification.

LC/MS (Method B): RT=1.185; m/z=357 [M+H]⁺

### Step 4: EXAMPLE 153

Starting from (1R,2R)-4-fluoro-4-[2-(3-methylpyrazin-2-yl)ethynyl]-2-phenylcyclohexane-1-carboxylic acid (123 mg) and 5-amino-6-(4-fluorophenoxy)-3-[(4-hydroxypiperidin-4-yl)methyl]pyrimidin-4-one (14 mg, 0.34 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 153 as a white solid.

LC/MS (Method B): RT=1.258 m/z=675 [M+H]⁺

¹H NMR (399 MHz, DMSO-₆) 8.75 (t, J=2.3 Hz, 1H), 8.61 (t, J=2.1 Hz, 1H), 7.62-7.57 (m, 1H), 7.35-7.17 (m, 7H), 7.12-7.07 (m, 2H), 4.82 (m, 1H), 4.69 (m, 2H), 3.95-3.61 (m, 4H), 3.31-2.86 (m, 3H), 2.66 (m, 1H), 2.42-2.13 (m, 4H), 1.89 (m, 2H), 1.47-1.10 (m, 3H), (1.78-11.46 (m, 1H).

HRMS (TOF, ESI) m/z: Calculated for C₃₅H₃₃ClF₂N₆O₄ 674.222, Found: 675.2265 [M+H]⁺

### 5-amino-3-({1-[(1R,2R,4R)-4-fluoro-4-[2-(3-methoxypyrazin-2-yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 154)

### Step 1: tert-butyl (1R,2R) -4-hydroxy-4-[2-(3-methoxypyrazin-2 -yl)ethynyl]-2-phenylcyclohexane-1-carboxylate

Starting from tert-butyl (1R,2R)-4-oxo-2-phenylcyclohexane-1-carboxylate (300 mg, 1.09 mmol) and 2-ethynyl-3-methoxypyrazine (191 mg, 1.42 mmol) following procedure described in Step 2 of EXAMPLES 122 and 123, the obtained residue was purified via flash chromatography using Heptane-60% EtOAc/Heptane (gradient) as eluent to afford tert-butyl (1R,2R)-4-hydroxy-4-[2-(3-methoxypyrazin-2-yl)ethynyl]-2-phenylcyclohexane-1-carboxylate as an off-white solid.

LC/MS (Method B): RT=1.318; m/z=409 [M+H]⁺

### Step 2: tert-butyl (1R,2R)-4-fluoro-4[3-(3-methoxypyrazin-2-yl)ethynyl]-2-phenylcyclohexane-1-carboxylate

Starting from tert-butyl (1R,2R)-4-hydroxy-4-[2-(3-methoxypyrazin-2-yl)ethynyl]-2-phenylcyclohexane-1-carboxylate (230 mg, 0.56 mmol) following procedure described in Step 4 of EXAMPLES 122 and 123, the obtained residue was purified via flash chromatography using Heptane-30% EtOAc/Heptane (gradient) as eluent to afford tert-butyl (1R,2R)-4-fluoro-4-[2-(3-methoxypyrazin-2-yl)ethynyl]-2-phenylcyclohexane-1-carboxylate as an oil.

LC/MS (Method B): RT=1.488; m/z=411 [M+H]⁺

### Step 3: (1R,2R)-4-fluoro-4-[2-(3-methoxypyrazin-2-yl)ethynyl]-2-phenylcyclohexame-1-carboxylic acid

Starting from tert-butyl (1R,2R)-4-fluoro-4-[2-(3-methoxypyrazin-2-yl)ethynyl]-2-phenylcyclohexane-1-carboxylate (113 mg, 0.28 mmol) following procedure described in Step 5 of EXAMPLE 133, (1R,2R)-4-fluoro-4-[2-(3-methoxypyrazin-2-yl)ethynyl]-2-phenylcyclohexane-1-carboxylic acid was obtained as a yellow oil. The compound was used without further purification.

LC/MS (Method B): RT=1.17; m/z=355 [M+H]⁺

### Step 4: EXAMPLE 134

Starting from (1R,2R)-4-fluoro-4-[2-(3-methoxypyrazin-2-yl)ethynyl]-2-phenylcyclohexane-1-carboxylic acid (110 mg) and 5-amino-6-(4-fluorophenoxy)-3-[(4-hydroxypiperidin-4-yl)methyl]pyrimidin-4-one (106 mg, 0.32 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 154 as a white solid.

LC/MS (Method B): RT=1.06; m/z=671 [M+H]⁺

¹H NMR (399 MHz, DMSO-d₆) 8.32 (dd, J=2.7, 1.7 Hz, 1H), 8.29 (dd, J=2.7, 1.9 Hz, 1H), 7.62-7.57 (m, 1H), 7.35-7.16 (m, 7H), 7.12-7.07 (m, 2H), 4.83 (s, 1H), 4.68 (m, 2H), 4.04 (s, 3H), 3.96-3.82 (m, 2H), 3.75-3.61 (m, 2H), 3.26-2.87 (m, 3H), 2.65 (m, 1H), 2.36-2.10 (m, 4H), 1.88 (m, 2H), 1.47-1.11 (m, 3H), 0.78-0.56 (m, 1H).

HRMS (TOF, ESI) m/z: Calculated for C₃₆H₃₆F₂N₆O₅ 670.2715, Found: 671.2759 [M+H]⁺

### 5-amino-3-[(1-{[(1R,2R,4R)-4-ethynyl-4-methoxy-2-phenylcyclohexyl]carbonyl}-4-hydroxypiperidin-4-yl)methyl]-6-(4-fluorophenoxy)pyrimidin-4-one (EXAMPLE 155)

### Step 1: tert-butyl (1R,2R)-4-hydroxy-2-phenyl-4-[2-(trimethylsilyl)ethynyl]cyclohexane-1-carboxylate

Starting from tert-butyl (1R,2R)-4-oxo-2-phenylcyclohexane-1-carboxylate (500 mg, 1.82 mmol) and ethynyltrimethylsilane (233 mg, 2.37 mmol) following procedure described in Step 2 of EXAMPLES 122 and 123, the obtained residue was purified via flash chromatography using Heptane-20% EtOAc/Heptane (gradient) as eluent to afford tert-butyl (1R,2R)-4-hydroxy-2-phenyl-4-[2-(trimethylsilyl)ethynyl]cyclohexane-1-carboxylate as an off-white solid.

¹H NMR (399 MHz, DMSO-d₆) δ 7.11-6.94 (m, 5H), 5.42 (s, 1H), 2.87-2.74 (m, 1H), 2.57-2.34 (m, 1H), 2.23 (td, J=11.6, 3.8 H), 1H), 2.10-1.92 (m, 1H), 1.69-1.51 (m, 3H), 1.47-1.29 (m, 1H), 0.87 (s, 9H), 0.00 (s, 9H).

### Step 2: tert-butyl (1R,2R)-4-ethynyl-4-methoxy-2-phenylcyclohexane-1-carboxylate

Starting from tert-butyl (1R,2R)-4-hydroxy-2-phenyl-4-[2-(trimethylsilyl)ethynyl]cyclohexane-1-carboxylate (525 mg, 0.78 mmol) following procedure described in Step 1 of EXAMPLES 146 and 147, tert-butyl (1R,2R)-4-ethynyl-4-methoxy-2-phenylcyclohexane-1-carboxylate was obtained as a white solid.

¹H NMR (399 MHz, DMSO-d₆) δ 7.30-7.18 (m, 5H), 3.71 (s, 1H) 3.28 (s, 3H), 2.94 (ddd, J=13.0, 11.5, 3.4 Hz, 1H), 2.56 (dd, J=11.8, 3.6 Hz, 1H), 2.10-1.88 (m, 3H), 1.82-1.48 (m, 3H), 1.08 (s, 9H).

### Step 3:(1R,2R)-1-ethynyl-4-methoxy-2-phenylcyclohexane-1-carboxylic acid

Starting from ten-butyl (1R,2R)-4-ethynyl-4-methoxy-2-phenylcyclohexane-1-carboxylate (110 mg, 0.35 mmol) following procedure described in Step 5 of EXAMPLE 133, (1R,2R)-4-ethynyl-4-methoxy-2-phenylcyclohexane-1-carboxylic acid was obtained as a colourless oil. The compound was used without further purification.

LC/MS (Method B): RT=1.08; m/z=257 [M−H]⁺

### Step 4: EXAMPLE 155

Starting from 5-amino6-(4-fluorophenoxy)-3-[(4-hydroxypiperidin-4-yl)methyl]pyrimidin-4-one (131 mg, 0.39 mmol) and (1R,2R)-4-ethynyl-4-methoxy-2-phenylcyclohexane-1-carboxylic acid (92 mg) following, procedure described in Step 3 of EXAMPLE 94, the obtained residue was purified via flash chromatography using DCM-5% MeOH (gradient) as eluent to give the still impure product as colourless oil. Final purification was performed via flash chromatography using Heptane-100% EtOAc (gradient) as eluent to afford EXAMPLE 155 as a white solid.

LC/MS (Method B): RT=m/z=575 [M+H]⁺

¹H NMR (399 MHz, DMSO-d₆) δ 7.62-7.56 (m, 1H), 7.34-7.05 (m, 9H), 4.81 (m, 1H), 4.68 (d, 2H), 3.96-3.81 (m, 2H), 3.71-3.60 (m, 3H), 3.29 (s, 3H), 3.17-2.85 (m, 3H), 2.68-2.62 (m, 1H), 2.11-1.95 (m, 2H), 1.90-1.54 (m, 4H), 1.48-1.05 (m, 3H), 0.76-0.54 (m, 1H).

HRMS (TOF, ESI) m/z: Calculated for C₃₂H₃₅FN₄O₅ 574.2591, Found: 575.2646 [M+H]⁺

### 5-amino-6-(4-fluorophenoxy)-3-({4-hydroxy-1-[(1R,2R,4R)-4-methoxy-2-phenyl-4-[2-(pyrazin-2-yl)ethynyl]cyclohexanecarbonyl]piperidin-4-yl}methyl)-3,4-dihydropyrimidin-4-one (EXAMPLE 156)

### Step 1: tert -butyl (1R,2R)-4-hydroxy-2-phenyl-4-[2-(pyrazin-2-yl)ethynyl]cyclohexane-1-carboxylate

Starting from tert-butyl (1R,2R)-4-oxo-2-phenylcyclohexane-1-carboxylate (300 mg, 1.09 mmol) and 2-ethynylpyrazine (148 mg, 1.42 mmol) following procedure described in Step 2 of EXAMPLES 122 and 123, the obtained residue was purified via flash chromatography using Heptane-60% EtOAc/Heptane (gradient) as eluent to afford tert-butyl (1R,2R)-4-hydroxy-2-phenyl-4-[2-(pyrazin-2-yl)ethynyl]cyclohexane-1-carboxylate as a white solid.

LC/MS (Method B): RT=1.230; m/z=379 [M+H]⁺

### Step 2: tert-butyl (1R,2R)-4-methoxy-2-phenyl-4-[2-(pyrazin-2-yl)rthynyl]cyclohexane-1-carboxylate

Starting from tert-butyl (1R,2R)-4-hydroxy-2-phenyl-4-[2-(pyrazin-2-yl)ethynyl]cyclohexane-1-carboxylate (293 mg, 0.77 mmol) following procedure described in Step 1 of EXAMPLES 146 and 147, the obtained residue was purified via flash chromatography using Heptane-30% EtOAc/Heptane (gradient) as eluent to afford tert-butyl (1R,2R)-4-methoxy-2-phenyl-4-[2-(pyrazin-2-yl)ethynyl]cyclohexane-1-carboxylate as an oil.

LC/MS (Method B): RT=1.39; m/z=393 [M+H]⁺

### Step 3: (1R,2R)-4-methoxy-2-phenyl-4-[2-pyrazin-2-yl)ethynyl]cyclohexane-1-carboxylic acid

Starting from tert-butyl (1R,2R)-4-methoxy-2-phenyl-4-[2-(pyrazin-2-yl)ethynyl]cyclohexane-1-carboxylate (223 mg, 0.57 mmol) following procedure described in Step 5 of EXAMPLE 133, (1R,2R)-4-methoxy-2-phenyl-4-[2-(pyrazin-2-yl)ethynyl]cyclohexane-1-carboxylic acid was obtained as a yellow oil. The compound was used without further purification.

LC/MS (Method B): RT=1.061; m/z=337 [M+H]⁺

### Step 4: EXAMPLE 156

Starting from (1R,2R)-4-methoxy-2-phenyl-4-[2-(pyrazin-2-yl)ethynyl]cyclohexane-1-carboxylic acid (193 mg) and 5-amino-6-(4-fluoro)phenoxy)-3-[(4-hydroxypiperidin-4-yl)methyl]pyrimidin-4-one (192 mg, 0.57 mmol) following procedure described in Step 3 of EXAMPLE 94, the obtained residue was purified via flash chromatography using water-100 % MeCN/water (gradient) as eluent to afford EXAMPLE 156 as a white solid.

LC/MS (Method B): RT=1.162; m/z=653 [M+H]⁺

¹H NMR (399 MHz, DMSO-d₆) 8.87 (m, 1H), 8.71 (m, 1H), 8.69 (m, 1H), 7.62-7.56 (m, 1H), 7.33-7.14 (m, 7H), 7.12-7.07 (m, 2H), 4.82 (m, 1H), 4.68 (m, 2H), 3.96-3.62 (m, 4H), 3.40 (s, 3H), 323-2.88 (m, 3H), 2.66 (m, 1H), 2.21 (m, 2H), 1.95-1.72 (m, 4H), 1.45-1.11 (m, 3H), 0.81-0.56 (m, 1H).

HRMS (TOF, ESI) m/z: Calculated for C₃₆H₃₇FN₆O₅ 652.280, Found: 653.2857 [M+H]⁺

### 5-amino-3-({1-[(1R,2R,4R)-4-fluoro-4-[2-(4-fluoropyridin-2-yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 157)

### Step 1: tert-butyl (1R,2R)-4-[2-(4-fluoropyridin-2-yl)ethynyl]-4-hydroxy-2-phenylcyclohexane-1-carboxylate

Starting from tert-butyl (1R,2R)-4-oxo-2-phenylcyclohexane-1-carboxylate (348 mg, 1.27 mmol) and 2-ethynyl-4-fluoropyridine (148 mg, 1.42 mmol) following procedure described in Step 2 of EXAMPLES 122 and 123, the obtained residue was purified via flash chromatography using Heptane-50% EtOAc/Heptane (gradient) as eluent to afford tert-butyl (1R,2R)-4-[2-(4-fluoropyridin-2-yl)ethynyl]-4-hydroxy-2-phenylcyclohexane-1-carboxylate as a white solid.

LC/MS (Method B): RT=1.310; m/z=396 [M+H]⁺

### Step 2: tert-butyl (1R,2R,4R)-4-fluoro-4-[2-(4-fluoropyridin-2-yl)ethynyl]-2-phenylcyclohexane-1-carboxylate and tert-butyl (1R,2R,4S)-4-fluoro-[2-(4-fluoropyridin-2-yl)ethyttyl]-2-phenylcyclohexane-1-carboxylate

Starting from tert-butyl (1R,2R)-4-[2-(4-fluoropyridin-2-yl)ethynyl]-4-hydroxy-2-phenylcyclohexane-1-carboxylate (200 mg, 0.51 mmol) following procedure described in Step 4 of EXAMPLES 122 and 123, the obtained residue was purified via flash chromatography using Heptane-25% EtOAc/Heptane (gradient) as eluent to afford:

First elute: tert-butyl (1R,2R,4R)-4-fluoro-4-[2-(4-fluoropyridin-2-yl)ethynyl]-2-phenylcyclohexane-1-carboxylate as a gum.

LC/MS (Method B): RT=1.470; m/z=398 [M+H]⁺

Second elute: tort-butyl (1R,2R,4S)-4-fluoro-4-[2-(4-fluoropyridin-2-yl)ethynyl]-2-phenylcyclohexane-1-carboxylate as a gum.

LC/MS (Method B): RT=1.480 m/z=398 [M+H]⁺

### Step 3: (1R,2R,4R)-4-fluoro-4-[2-(4-fluoropyridin-2-yl)ethynyl]-2-phenylcyclohexane-1-carboxylic acid

Starting from tert-butyl (1R,2R,4R)-4-fluoro-4-[2-(4-fluoropyridin-2-yl)ethynyl]-2-phenylcyclohexane-1-carboxylate (73 mg, 0.19 mmol) following procedure described in Step 5 of EXAMPLE 133, (1R,2R,4R)-4-fluoro-4-[2-(4-fluoropyridin-2-yl)ethynyl]-2-phenylcyclohexane-1-carboxylic acid was obtained as a white solid.

LC/MS (Method B): RT=1.190; m/z=342 [M−H]⁺

### Step 4: EXAMPLE 157

Starting from ((1R,2R,4R)-4-fluoro-4-[2-(4-fluoropyridin-2-yl)ethynyl]-2-phenylcyclohexane-1-carboxylic acid (55 mg, 0.161 mmol) and 5-amino-6-(4-fluorophenoxy)-3-[(4-hydroxypiperidin-4-yl)methyl]pyrimidin-4-one (54 mg, 0.161 mmol) following procedure described in Step 3 of EXAMPLE 94, the obtained residue was purified via flash chromatography using DCM-10% MeOH/DCM (gradient) as eluent to afford EXAMPLE 157 as a white solid.

LC/MS (Method B): RT=1.07; m/z=658 [M+H]⁺

¹H NMR (399 MHz, DMSO-d₆) 8.68 (m, 1H), 7.70 (m, 1H), 7.62-7.57 (m, 1H), 7.47 (m, 1H), 7.34-7.18 (m, 7H), 7.11-7.08 (m, 2H), 4.82 (m, 1H), 4.70 (m, 2H), 3.96-3.60 (m, 4H), 3.28-2.86 (m, 3H), 2.66 (m, 1H), 2.37-2.06 (m, 4H), 1.85 (m, 2H), 1.46-1.12 (m, 0.78-1.52 (m, 1H).

### 5-amino-3-({1-[(1R,2R,4S)-4-fluoro-4-[2-(4-fluoropyridin-2-yl)ethynyl]-2-phenylcyclohexanecarbonyl]-4-hydroxypiperidin-4-yl}methyl)-6-(4-fluorophenoxy)-3,4-dihydropyrimidin-4-one (EXAMPLE 158)

### Step 1: (1R,2R,4S)-4 -fluoro-4-[2-(4-fluoropyridin-2-yl)ethynyl]-2-phenylcyclohexane-1-carboxylic acid

Starting from tert-butyl (1R,2R,4S)-4-fluoro-4-[2-(4-fluoropyridin-2-yl)ethynyl]-2-phenylcyclohexane-1-carboxylate (69 mg, 0.17 mmol) following procedure described in Step 5 of EXAMPLE 133, (1R,2R,4S)-4-fluoro-4-[2-(4-fluoropyridin-2-yl)ethynyl]-2-phenylcyclohexane-1-carboxylic acid was obtained as a white solid.

LC/MS (Method B): RT=1.180; m/z=342 [M−H]⁻

### Step 2: EXAMPLE 158

Starting from ((1R,2R,4S)-4-fluoro-4-[2-(4-fluoropyridin-2-yl)ethynyl]-2-phenylcyclohexane-1-carboxylic acid (55 mg, 0.161 mmol) and 5-amino-6-(4-fluorophenoxy)-3-[(4-hydroxypiperidin-4-yl)methyl]pyrimidin-4-one (54 mg, 0.161 mmol) following procedure described in Step 3 of EXAMPLE 94, the obtained residue was purified via flash chromatography using DCM-10% MeOH/DCM (gradient) as eluent to afford. EXAMPLE 158 as a white solid.

LC/MS (Method B): RT=1.04; m/z=638 [M−H]⁺

¹H NMR (399 MHz, DMSO-d₆) 8.62 (m, 1H), 7.62-7.57 (m, 2H), 7.43 (m, 1H), 7.34-7.17 (m, 7H), 7.12-7.07 (m, 2H), 4.82 (m, 1H), 4.69 (m, 2H), 3.96-3.61 (m, 4H), 3.29-2.86 (m, 3H), 2.67 (m, 1H), 2.41-2.05 (m, 4H), 1.84-1.66 (m, 2H), 1.46-1.10 (m, 3H), 0.78-0.58 (m, 1H).

HRMS (TOF, ESI) m/z: Calculated for C₃₀H₃₄F₃N₅O₄ 657.2563,Found: 658.2644 [M+H]⁺

### 5-amino-6-(4-fluorophenoxy)-3-[(4-hydroxy-1-{[(1R,2R,4R)-4-methoxy-4-[2-(1-methylimidazol-2-yl)ethynyl]-2-phenytcyclohexyl]carbonyl}piperidin-4-yl)methyl]pyrimidin-4-one (EXAMPLE 159)

and

### 5-amino-6-(4-fluorophenoxy)-3-[(4-hydroxy-1-{[(1R,2R,4S)-1-methoxy-4-[2-(1-methylimidazol-2-yl)ethynyl]-2-phenylcyclohexyl]carbonyl}piperidin-4-yl)methyl]pyrimidin-4-one (EXAMPLE 160)

### Step 1: tert-butyl (1R,2R)-4-methoxy-4-[2-(1-methylimidazol-2-yl)ethynyl]-2-phenylcyclohexane-1-carboxylate

Starting from tert-butyl (1R,2R)-4-hydroxy-4-[2-(1-methyl-1H-imidazol-2-yl)ethynyl]-2-phenylcyclohexane-1-carboxylate (100 mg, 0.26 mmol) following procedure described in Step 1 of EXAMPLES 146 and 147, tert-butyl (1R,2R)-4-methoxy-4-[2-(1-methylimidazol-2-yl)ethynyl]-2-phenylcyclohexane-1-carboxylate was obtained as a brown oil.

LC/MS (Method B): RT=1.28; m/z=395 [M+H]⁺

### Step 2 (1R,2)-4-methoxy-4-[2-(1-methylimidazol-2-yl)ethynyl]-2-phenylcyclohexane-1-carboxylic acid

Starting from tert-butyl (1R,2R)-4-methoxy-4-[2-(1-methylimidazol-2-yl)ethynyl]-2-phenylcyclohexane-1-carboxylate (95 mg, 0.24 mmol) following procedure described in Step 5 of EXAMPLE 133, (1R,2R)-4-methoxy-4 -[2-(1-methylimidazol-2-yl)ethynyl]-2-phenylcyclohexane-1-carboxylic acid was obtained as a yellow oil. The compound was used without further purification.

LC/MS (Method B): RT=0.919; m/z=339 [M+H]⁺

### Step 3: EXAMPLES 159 and 160

Starting from 5-amino-6-(4-fluorophenoxy)-3-[(4-hydroxypiperidin-4-yl)methyl]pyrimidin-4-one (145 mg, 0.43 mmol) and (1R,2R)-4-methoxy-4-[2-(1-methylimidazol-2-yl)ethynyl]-2-phenylcyclohexane-1-carboxylic acid (98 mg) following procedure described in Step 3 of EXAMPLE 94, the obtained residue was purified via flash chromatography using DCM-10% MeOH (gradient) as eluent to give the still impure product as colourless oil. Final purification was performed via prep HPLC Prep (HPLC Column: Gemini pH9 Dimensions: 21.1 mm×150 mm 5 μm) to afford:

First elute: EXAMPLE 160 as a white solid.

LC/MS (Method B): RT=0.850; 655 [M+H]⁺

1H NMR (399 MHz, DMSO-d6) δ 7.56-7.50 (m,1H), 7.29-7.07 (m, 8H), 7.02 (m, 2H), 6.87 (m, 1H), 4.78 (m, 1H), 4.61 (m, 2H), 3.89-3.55 (m, 7H), 3.30 (s, 3H), 3.21-2.79 (m, 3H), 2.53 (m, 1H), 2.18-1.82 (m, 4H), 1.78-1.44 (m, 2H), 1.35-1.04 (m, 3H), 0.68-0.53 (m, 1H),

HRMS (TOF, ESI) m/z: Calculated for C₃₆H₃₉FN₆O₅ 654.2966, Found: 655.3045 [M+H]⁺

Second elute: EXAMPLE 159 as a white solid.

LC/MS (Method B): RT=0.866; m/z=655 [M+H]⁺

1H NMR (399 MHz, DMSO-d6) δ 7.55-7.49 (m, 1H), 7.26-7.06 (m, 8H), 7.03-6.99 (m, 2H), 6.91 (m, 1H), 4.74 (m, 1H), 4.59 (m, 2H), 3.88-3.52 (m, 7H), 3.30 (s, 3H), 3.16-2.78 (m, 3H), 2.57 (m, 1H), 2.10 (m, 2H), 1.88-1.62 (m, 4H), 1.38-1.02 (m, 3H), 0.73-0.44 (m, 1H),

HRMS (TOF, ESI) m/z: Calculated for C₃₆H₃₉FN₆O₅ 654.2966, Found: 655.3048 [M+H]⁺

### 5-amino-3-[(1-{[(1R,2R,4R)-4-[2-(6-aminopyridin-2-yl)ethynyl]-4-fluoro-2-phenylcyclohexyl]carbonyl}-4-hydroxypiperidin-4-yl)methyl]-6-(4-fluurophenoxy)pyrimidin-4-one (EXAMPLE 161)

### Step 1: tert-butyl (1R,2R)-4-(2-{6-[bis(tert-butoxycarbonyl)amino]pyridin-2-yl}ethynyl)-4-hydroxy-2-phenylcyclohexane-1-carboxylate

Starting from tert-butyl (1R,2R)-4-oxo-2-phenylcyclohexane-1-carboxylate (500 mg, 1.82 mmol) and tert-butyl N-[(tert-butoxy)carbonyl]-N-(6-ethynylpyridin-2-yl)carbamate (754 mg, 2.37 mmol, 1.3 eq.) following procedure described in Step 2 of EXAMPLES 122 and 123, the obtained residue was purified via flash chromatography using Heptane 34% EtOAc/Heptane (gradient) as eluent to give tert-butyl (1R,2R)-4-(2-{6-[bis(tert-butoxycarbonyl)amino]pyridin-2-yl}ethynyl)-4-hydroxy-2-phenylcyclohexane-1-carboxylate as a white solid.

LC/MS (Method B): RT=1.35; m/z=593 [M+H]⁺

### Step 2 tert-butyl (1R,2R)-4-(2-{6-[bis(tert-butoxycarbonyl)amino]pyridin-2-yl}ethynyl)-4-fluoro-2-phenylcyclohexane-1-carboxylate

Starting from tert-butyl (1R,2R)-4(2-{6-[bis(tert-butoxycarbonyl)amino]pyridin-2-yl}ethynyl)-4-hydroxy-2-phenylcyclohexane-1-carboxylate (545 mg, 0.92 mmol) following procedure described in Step 4 or EXAMPLES 122 and 123. the obtained residue was purified via flash chromatography using Heptane-18% EtOAc/Heptane (gradient) as eluent to afford tert-butyl (1R,2R)-4-(2-{6-[bis(tert-butoxycarbonyl)amino]pyridin-2-yl}ethynyl)-4-fluoro-2-phenylcyclohexane-1-carboxylate as a colourless oil. The compound was used without further purification.

LC/MS (Method B): RT=1.44; m/z=595 [M+H]⁺

### Step 3: (1R,2R)-4-[2-(6-aminopyridin-2-yl)ethynyl]-4-fluoro-2-phenylcyclohexane-1-carboxylic acid

Starting from tert-butyl (1R,2R)-4-(2-{6-[bis(tert-butoxycarbonyl)amino]pyridin-2-yl}ethynyl)-4-fluoro-2-phenylcyclohexane-1-carboxylate (520 mg) following procedure described in Step 5 of EXAMPLE 133, (1R,2R)-4-[2-(6-aminopyridin-2-yl)ethynyl]-4-fluoro-2-phenylcyclohexane-1-carboxylic acid (350 mg) was obtained as a white foam. The compound was used without further purification.

LC/MS (Method B): RT=0.818; m/z=339 [M+H]⁺

### Step 4: EXAMPLE 161

Starting from 5-amino-6-(4-fluorophenoxy)-3-[(4-hydroxypiperidin-4-yl)methyl]pyrimidin-4-one (166 mg. 0.50 mmol) and (1R,2R)-4-[2-(6-aminopyridin-2-yl)ethynyl]-4-fluoro-2-phenylcyclohexane-1-carboxylic acid (175 mg) following procedure described in Step 3 of EXAMPLE 94, the obtained residue was purified via flash chromatography using DCM-9% MeOH (gradient) as eluent to give the still impure product as colourless oil. Final purification was performed prep HPLC Prep (HPLC Column: Gemini pH9 Dimensions: 21.1 mm×150 mm 5 μm) to afford EXAMPLE 161 as a white solid.

LC/MS (Method B): RT=1.15; m/z=655 [M+H]⁺

¹H NMR (399 MHz, DMSO-d₆) δ 7.54-7.49 (m, 1H), 7.34 (ddd, J=8.5, 7.2, 1.6 Hz, 1H), 7.28-7.07 (m, 7H), 7.03-6.99 (m, 2H), 6.67 (dd, J=7.0, 2.5 Hz, 1H), 6.43 (dt, J=8.5, 1.2 Hz, 1H), 6.18 (s, 2H), 4.76 (s, 1H), 4.61 (m, 2H), 3.88-3.53 (m, 4H), 3.18-2.77 (m, 3H), 2.57 (m, 1H), 2.26-1.94 (m, 4H), 1.75 (m, 2H), 1.36-0.93 (m, 3H), 0.71-0.45 (m, 1H),

HRMS (TOF, ESI) m/z: Calculated for C₃₆H₃₆F₂N₆O₄ 654.2766, Found: 655.281 [M+H]⁺

### 5-amino-3-[[(4S)-3,3-difluoro-1-[(1R,2R,4R)-4-fluoro-4-[2-(6-methylpyridazin-3-yl)ethynyl]-2-phenylcyclohexanecarbonyl]-4-hydroxypiperidin-4-yl]methyl]-6-(4-fluorophenoxy)-3,4-dihydropyrimidin-4-one (EXAMPLE 162)

### Step 1: tert-butyl (1R,2R)-4-hydroxy-4-[2-(6-methylpyridazin-3-yl)ethynyl]-2-phenylcyclohexane-1 -carboxylate

Starting from tert-butyl (1R,2R)-4-oxo-2-phenylcyclohexane-1-carboxylate (400 mg, 1.46 mmol) and 3-ethynyl-6-methylpyridazine (224 mg, 1.90 mmol) following procedure described in Step 2 of EXAMPLES 122 and 123, the obtained residue was purified via flash chromatography using Heptane-100% EtOAc/Heptane (gradient) its eluent to afford tert-butyl (1R,2R)-4-hydroxy-4-[2-(6-methylpyridazin-3-yl)ethynyl]-2-phenylcyclohexane-1-carboxylate as a yellow solid.

LC/MS (Method B): RT=1.206; m/z=393 [M+H]⁺

### Step 2: tert-butyl (1R,2R)-4-fluoro-4-[2-(6-methylpyridazin-3-yl)ethynyl]-2-phenylcyclohexane-1-carboxylate

Starting from tert-butyl (1R,2R)-4-hydroxy-4-[2-(6-methylpyridazin-3-yl)ethynyl]-2-phenylcyclohexane-1-carboxylate (293 mg, 0.75 mmol) following procedure described in Step 4 of EXAMPLES 122 and 123, the obtained residue was purified via flash chromatography using Heptane-30% EtOAc/Heptane (gradient) as eluent to afford tert-butyl (1R,2R)-4-fluoro-4-[2-(6-methylpyridazin-3-yl)ethynyl]-2-phenylcyclohexane-1-carboxylate as an off-white solid.

LC/MS (Method B): RT=1.19; m/z=395 [M+H]⁺

### Step 3: (1R,2R)-4-fluoro-4-[2-(6-methylpyridazin-3-yl)ethynyl]-2-phenylcyclohexane-1-carboxylic acid

Starting from tert-butyl (1R,2R)-4-fluoro-4-[2-(6-methylpyridazin-3-yl)ethynyl]-2-phenylcyclohexane-1-carboxylate (98 mg, 0.25 mmol) following procedure described in Step 5 of EXAMPLE 133, (1R,2R)-4-fluoro-4-[2-(6-methylpyridazin-3-yl)ethynyl]-2-phenylcyclohexane-1-carboxylic acid was obtained us a red oil. The compound was used without further purification.

LC/MS (Method B): RT=1.032; m/z=339 [M−H]⁺

### Step 4: EXAMPLE 162

Starting from ((1R,2R)-4-fluoro-4-[2-(6-methylpyridazin-3-yl)ethynyl]-2-phenylcyclohexane-1-carboxylic acid (150 mg) and 5-amino-3-{[(4S)-3,3-difluoro-4-hydroxypiperidin-4-yl]methyl}-6-(4-fluorophenoxy)-3,4-dihydropyrimidin-4-one (98.5 mg, 0.27 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 162 as a white solid.

LC/MS (Method B): RT=1.225; m/z=691 [M+H]⁺

¹H NMR (399 MHz, DMSO-d₆) 7.87 (dd, J=8.6, 3.1 Hz, 1H), 7.70-7.62 (m, 2H), 7.32-7.17 (m, 7H), 7.13-7.07 (m, 2H), 6.05 (s, 1H), 4.77-4.72 (m, 2H), 4.47-4.20 (m, 2H), 4.02-3.74 (m, 2H), 3.45-3.08 (m, 4H), 2.69 (s, 3H), 2.45-2.12 (m, 4H), 1.96-1.75 (m, 2H), 1.51-1.20 (m, 2H)

HRMS (TOF, ESI) m/z: Calculated for C₃₆H₃₄F₄N₄O₄ 690.2578, Found: 691.2685 [M+H]⁺

### 5-amino-3-{[(4S)-1-[(1R,2R,4R)-4-[2-(6-aminopyridin-2-yl)ethynyl]-4-fluoro-2-phenylcyclohexanecarbonyl]-3,3-difluoro-4-hydroxypiperidin-4-yl]methyl}-6-(4-fluorophenoxy)-3,4-dihydropyrimidin-4-one (EXAMPLE 163)

Starting from 5-amino-3-{[(4S)-3,3-difluoro-4-hydroxypiperidin-4-yl]methyl}-6-(4-fluorophenoxy)-3,4-dihydropyrimidin-4-one (100 mg, 0.13 mmol) and (1R,2R)-4-[2-(6-aminopyridin-2-yl)ethynyl]-4-fluoro-2-phenylcyclohexane-1-carboxylic acid (91 mg) following procedure described in Step 3 of EXAMPLE 94, the obtained residue was purified via flash chromatography using Heptane-100% EtOAc (gradient) as eluent to give the still impure product as colourless oil. Final purification was performed via prep HPLC Prep (HPLC Column: Gemini pH4 Dimensions: 30 mm×250 mm 5 μm) to afford EXAMPLE 163 as a white solid.

LC/MS (Method B): RT=1.210; m/z=691 [M+H]⁺

1H NMR (399 MHz, DMSO-d6) δ 7.67-7.62 (m, 1H), 7.41 (dd, J=8.4, 7.2 Hz, 1H), 7.31-7.17 (m, 7H), 7.13-7.07 (m, 2H), 6.75 (dd, J=7.2, 2.6 Hz, 1H), 6.50 (d, J=8.4, 1H), 6.25 (s, 2H), 6.07 (s, 1H), 4.76-4.72 (m, 2H), 4.47-4.20 (m, 2H), 4.01-3.74 (m, 2H), 3.20-3.07 (m, 4H), 2.43-2.07 (m, 4H), 1.91-1.67 (m, 2H), 1.48-1.21 (m, 2H).

HRMS (TOF, ESI) m/z: Calculated for C₃₆H₃₄F₄N₆O₄ 690.2578. Found: 691.2663 [M+H]⁺

### 5-amino-3-{[(4S)-1-[(1R,2R,4R)-4-[2-(6-aminopyridin-2-yl)ethynyl]-4-methoxy-2-phenylcyclohexanecarbonyl]-3,3-difluoro-4-hydroxypiperidin-4-yl]methyl}-6-(4-fluorophenoxy)-3,4-dihydropyrimidin-4-one (EXAMPLE 164)

### Step 1: tert-butyl (1R,2R)-4-(2-{6-[bis(tert-butoxycarbonyl)amino]pyridin-2-yl}ethynyl)-4-hydroxy-2-phenylcyclohexane-1-carboxylate

Starting from tert-butyl (1R,2R)-4-[2-(6-{bis[(tert-butoxy)carbonyl]amino}pyridin-2-yl)ethynyl]-4-hydroxy-2-phenylcyclohexane-1-carboxylate (90 mg, 0.15 mmol) following procedure described in Step 1 of EXAMPLES 146 and 147, tert-butyl (1R,2R)-4-(2-{6-[bis(tert-butoxycarbonyl)amino]pyridin-2-yl}ethynyl)-4-hydroxy-2-phenylcyclohexane-1-carboxylate was obtained as a colourless oil

LC/MS (Method B): RT=1.61; m/z=507 [M-BOC+H]⁺

### Step 2: (1R,2R)-4-[2-(6-aminopyridin-2-yl)ethynyl]-4-methoxy-2-phenylcyclohexane-1-carboxylic acid

Starting from tert-butyl (1R,2R)-4-(2-{6-[bis(tert-butoxycarbonyl)amino]pyridin-2-yl}ethynyl)-4-hydroxy-2-phenylcyclohexane-1-carboxylate (50 mg, 0.08 mmol) following procedure described in Step 5 of EXAMPLE 133, (1R,2R)-4-[2-(6-aminopyridin-2-yl)ethynyl]-4-methoxy-2-phenylcyclohexane-1-carboxylic acid was obtained 85 a yellow solid. The compound was used without further purification.

LC/MS (Method B): RT=0.98; m/z=351 [M+H]⁺

### Step 3: EXAMPLE 164

Starting from 5-amino-3-{[(4S)-3,3-difluoro-4-hydroxypiperidin-4-yl]methyl}-6-(4-fluorophenoxy)-3,4-dihydropyrimidin-4-one (48 mg, 0.13 mmol) and (1R,2R)-4-[2-(6-aminopyridin-2-yl)ethynyl]-4-methoxy-2-phenylcyclohexane-1-carboxylic acid (45 mg) following procedure described in Step 3 of EXAMPLE 94, the obtained residue was purified via flash chromatography using. Heptane-100% EtOAc (gradient) us eluent to give the still impure product as colourless oil. Final purification was performed via prep HPLC Prep (HPLC Column: Gemini pH7 Dimensions: 30 mm×250 mm 5 μm) to afford EXAMPLE 164 as a white solid.

LC/MS (Method B): RT=1.16; m/z=703 [M+H]⁺

1H NMR (399 MHz, DMSO-d6) δ 7.60-7.55 (m, 1H), 7.32 (dd, J=8.4, 7.2 Hz, 1H), 7.24-7.09 (m, 7H), 7.04-6.99 (m, 2H), 6.65 (ddd, J=7.2, 2.9, 0.9 Hz, 1H), 6.39 (dd, J=8.4, 0.9 Hz, 1H), 6.13 (s, 2H), 5.97 (m, 1H), 4.66 (m, 2H), 4.40-4.12 (m, 2H), 3.99-3.67 (m, 2H), 3.27-3.00 (m, 7H), 2.35-2.01 (m, 2H), 1.81-1.58 (m, 4H), 1.41-1.15 (m, 2H).

HRMS (TOF, ESI) m/z: Calculated for C₃₇H₃₇F₃N₆O₅ 702.2778, Found: 703.288 [M+H]⁺

### 5-amino-3-{[(4S)-3,3-difluoro-4-hydroxy-1-{[(1R,2R,4R)-4-methoxy-2-phenyl-4-[2-(pyridazin-3-yl)ethynyl]cyclohexyl]carbonyl}piperidin-4-yl]methyl}-6-(4-fluorophenoxy)pyrimidin-4-one (EXAMPLE 165)

Starting from 5-amino-3-{[(4S)-3,3-difluoro-4-hydroxypiperidin-4-yl]methyl}-6-(4-fluorophenoxy)-3,4-dihydropyrimidin-4-one (83 mg, 0.22 mmol) and (1R,2R)-4-methoxy-2-phenyl-4-[2-(pyridazin-3-yl)ethynyl]cyclohexane-1-carboxylic acid (75 mg, 0.22 mmol) following procedure described in Step 3 of EXAMPLE 94, the obtained residue was purified via flash chromatography using Heptane-100% EtOAc/Heptane (gradient) as eluent to give the still impure product as colourless oil. Final purification was performed via prep HPLC Prep (HPLC Column: Gemini pH4 Dimensions: 21.1 mm×150 mm 5 μm) to afford EXAMPLE 165 as a white foam.

LC/MS (Method B): RT=1.18; m/z=689 [M+H]⁺

¹H NMR (399 MHz, DMSO-d₆) δ 9.20 (dd, J=5.0, 1.7 Hz, 1H), 7.86 (ddd, J=8.5, 3.4, 1.7 Hz, 1H), 7.72 (dd, J=8.5. 5.0 Hz, 1H), 7.60-7.55 (m, 1H), 7.22-6.99 (m, 9H), 6.03-5.97 (m, 1H), 4.67 (m, 2H), 4.40-4.13 (m, 2H), 3.95-3.67 (m, 2H), 3.33 (s, 3H), 3.25-3.00 (m, 4H), 2.37-2.05 (m, 2H), 1.85-1.66 (m, 4H), 1.44-1.16 (m, 2H)

HRMS (TOF, ESI) m/z: Calculated for C₃₆H₃₅F₃N₆O₅ 688.2621, Found: 689.2724 [M+H]⁺

### 5-amino-3-{[(4S)-3,3-difluoro-4-hydroxy-1-[(1R,2R,4R)-4-methoxy-2-phenyl-4-[2-(pyridin-2-yl)ethynyl]cyclohexanecarbonyl]piperidin-4-yl]methyl}-6-(4-fluorophenoxy)-3,4-dihydropyrimidin-4-one (EXAMPLE 166)

### Step 1: tert-butyl (1R,2R)-4-hydroxy-2-phenyl-4-[2-(pyridin-2 -yl)ethynyl]cyclohexane-1-carboxylate

Starting from tert-butyl (1R,2R)-4-oxo-2-phenylcyclohexane-1-carboxylate (300 mg, 1.09 mmol) and 2-ethynylpyridine (150 mg, 1.42 mmol) following procedure described in Step 2 of EXAMPLES 122 and 123, the obtained residue was purified via flash chromatography using Heptane-100% EtOAc/Heptane (gradient) as eluent to afford tert-butyl (1R, 2R)-4-hydroxy-2-phenyl-4-[2-(pyridin-2-yl)ethynyl]cyclohexane-1-carboxylate as a white solid.

LC/MS (Method B): RT=1 267; m/z=378 [M+H]⁺

### Step 2: tert-butyl (1R,2R)-4-methoxy-2-phenyl-4-[2-(pyridin-2-yl)ethynyl]cyclohexane-1-carboxylate

Starting from tert-butyl (1R,2R)-4-hydroxy-4-phenyl-4-[2-(pyridin-2-yl)ethynyl]cyclohexane-1-carboxylate (47 mg, 0.12 mmol) following procedure described in Step 1 of EXAMPLES 146 and 147, the obtained residue was purified via flash chromatography using Heptane-88% EtOAc/Heptane (gradient) as eluent to afford tert-butyl (1R,2R)-4-methoxy-2-phenyl-4-[2-(pyridin-2-yl)ethynyl]cyclohexane-1-carboxylate as an yellow solid.

LC/MS (Method B): RT=1.424; m/z=392 [M+H]⁺

### Step 3: (1R,2R)-4-methoxy-2-phenyl-4-[2-(pyridin-2-yl)ethynyl]cyclohexane-1-carboxylic acid

Starting from tert-butyl (1R,2R)-4-methoxy-2-phenyl-4-[2-(pyridin-2-yl)ethynyl]cyclohexane-1-carboxylate (41 mg, 0.1 mmol) following procedure described in Step 5 of EXAMPLE 133, (1R,2R)-4-methoxy-2-phenyl-4-[2-(pyridin-2-yl)ethynyl]cyclohexane-1-carboxylic acid was obtained as a red oil. The compound was used without further purification.

LC/MS (Method B): RT=1.098; m/z=336 [M−H]⁺

### Step 4: EXAMPLE 166

Starting from (1R,2R)-4-methoxy-2-phenyl-4-[2-(pyridin-2-yl)ethynyl]cyclohexane-1-carboxylic acid (35 mg) and 5-amino-3-{[(4S)-3,3-difluoro-4-hydroxypiperidin-4-yl]methyl}-6-(4-fluorophenoxy)-3,4-dihydropyrimidin-4-one (39 mg, 0.1 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 166 as a white solid.

LC/MS (Method B): RT=1.256; m/z=688 [M+H]⁺

¹H NMR (399 MHz, DMSO-d₆) 8.62 (ddt, J=4.8, 1.8, 0.9 Hz, 1H), 7.87 (td, J=7.7, 1.8 Hz, 1H), 7.67-7.61 (m, 2H), 7.45 (ddd, J=7.7, 4.9, 1.2 Hz, 1H), 7.30-7.07 (m, 9H), 6.06-6.01 (m, 1H), 4.77-4.72 (m, 2H), 4.48-4.21 (m, 2H), 4.03-3.74 (m, 2H), 3.38 (s, 3H), 3.22-3.08 (m, 3H), 2.44 -2.14 (m, 4H), 1.89-1.73 (m, 3H), 1.51-1.26 (m, 2H)

HRMS (TOF, ESI) m/z: Calculated for C₃₇H₃₆F₃N₅O₅ 687.2669, Found: 688.2769 [M+H]⁺

### 5-amino-3-{[(4S)-3,3-difluoro-1-[(1R,2R,4R)-4-fluoro-2-phenyl-4-[2-(pyrimidin-2-yl)ethynyl]cyclohexanecarbonyl]-4-hydroxypiperldin-4-yl]methyl}-6-(4-fluorophenoxy)-3,4-dihydropyrimidin-4-one (EXAMPLE 167)

Starting from (1R,2R)-4-fluoro-2-phenyl-4-[2-(pyrimidin-2-yl)ethynyl]cyclohexane-1-carboxylic acid (87 mg) and 5-amino-3-{[(4S)-3,3-difluoro-4-hydroxypiperidin-4-yl]methyl}-6-(4-fluorophenoxy)-3,4-dihydropyrimidin-4-one (99 mg, 0.27 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 167 as a white solid.

LC/MS (Method B): RT=1.23; m/z=677 [M+H]⁺

¹H NMR (399 MHz, DMSO-d₆) 8.89 (dd, J=5.0, 0.8 Hz, 2H), 7.67-7.59 (m, 2H), 7.32-7.17 (m, 7H), 7.13-7.07 (m, 2H), 6.08-6.02 (m, 1H), 4.77-4.72 (m, 2H), 4.47-4.20 (m, 2H), 4.02-3.74 (m, 2H), 3.44-3.08 (m, 4H), 2.45-2.12 (m, 4H), 1.96-1.72 (m, 2H), 1.50-1.20 (m, 2H).

HRMS (TOF, ESI) m/z: Calculated for C₃₅H₃₂F₄N₆O₄ 676.2421, Found: 677.2498 [M+H]⁺

### 5-amino-3-{[(4S)-3,3-difluoro-1-{[(1R,2R,4R)-4-fluoro-2-phenyl-4-[2-(pyridazin-3-yl)ethynyl]cyclohexyl]carbonyl}-4-hydroxypiperidin-4-yl]methyl}-6-(4-fluorophenoxy)pyrimidin-4-one (EXAMPLE 168)

Starting from 5-amino-3-{[(4S)-3,3-difluoro-4-hydroxypiperidin-4-yl]methyl}-6-(4-fluorophenoxy)-3,4-dihydropyrimidin-4-one (80 mg, 0.12 mmol, 1 eq.) and (1R,2R)-4-fluoro-2-phenyl-4-[2-(pyridazin-3-yl)ethynyl]cyclohexane-1-carboxylic acid (70 mg, 0.22 mmol, 1 eq.) following procedure described in Step 3 of EXAMPLE 94, the obtained residue was purified via flash chromatography using DCM-1% MeOH/DCM (gradient) as eluent to give the still impure product as colourless oil. Final purification was performed via prep HPLC Prep (HPLC Column; Gemini pH4 Dimensions: 21.1 mm×150 mm 5 μm) to afford EXAMPLE 168 as a pale yellow solid.

LC/MS (Method B): RT=1.20; m/z=677 [M+H]⁺

1H NMR (399 MHz, DMSO-d₆) δ 9.31 (dd, J=5.1, 1.7 Hz, 1H), 7.99 (ddd, J=85, 3.2, 1.7 Hz, 1H), 7.82 (dd, J=8.5, 5.1 Hz, 1H), 7.68-7.63 (m, 1H), 7.32-7.07 (m, 9H), 6.09-6.03 (m, 1H), 4.76-4.71 (m 2H), 4.48-4.21 (m, 2H), 4.02-3.74 (m, 2H), 3.43-3.08 (m, 4H), 2.44-2.12 (m, 4H), 1.97-1.73 (m, 2H), 1.53-1.19 (m, 2H).

HRMS (TOF, ESI) m/z: Calculated for C₃₅H₃₂F₄N₆O₄ 676.2421, Found: 677.2533 [M+H]⁺

### 5-amino-3-{[(4S)-3,3-difluoro-4-hydroxy-1-[(1R,2R,4R)-4-methoxy-1-[2-(5-methylpyridazin-3-yl)ethynyl]-2-phenylcyclohexanecarbonyl]piperidin-4-yl]methyl}-6-(4-fluorophenoxy)-3,4-dihydropyrimidin-4-one (EXAMPLE 169)

### Step 1: 5 -methyl-3-[2-(trimethylsilyl)ethynyl]pyridazine

Starting from 3-chloro-5-methyl-pyridazine (1 g, 7.78 mmol) and ethynyltrimethylsilane (0.97 g, 9.33 mmol, 1.2 eq.) following procedure described in Step 1 of EXAMPLES 135 and 136, the obtained residue was purified via flash chromatography using Heptane-100% EtOAc/Heptane (gradient) as eluent to afford 5-methyl-3-[2-(trimethylsilyl)ethynyl]pyridazine as a brown solid.

LC/MS (Method B): RT=0.97; m/z=191 [M+H]⁺

Step 2: 3 -ethynyl-5-methlylpyridazine

To a solution of 5-methyl-3-[2-(trimethylsilyl)ethynyl]pyridazine (0.93 g, 4.89 mmol) in THF/MeOH (1:1, 20 mL) was added potassium carbonate (68 mg, 0.4) mmol, 0.1 eq.). The reaction mixture was stirred at r.t. for 1 hour and evaporated in vacuo. The residue was purified via flash chromatography using Heptane-100% EtOAc/Heptane (gradient) as eluent to afford 3-ethynyl-5-methylpyridazine as an off-white solid.

LC/MS (Method B): RT=0.426; m/z=119 [M+H]⁺

### Step 3: tert-butyl (1R,2R)-4-hydroxy-4-[2-(5-methylpyridazin-3-yl)ethynyl]-2-phenylcyclohexane-1-carboxylate

Starting from tert-butyl (1R,2R)-4-oxo-2-phenylcyclohexane-1-carboxylate (400 mg, 1.46 mmol) and 3-ethynyl-5-methylpyridazine (241 mg, 2.04 mmol) following procedure described in Step 2 of EXAMPLES 122 and 123, the obtained residue was purified via flash chromatography using Heptane-100% EtOAc/Heptane (gradient) as eluent to afford tert-butyl (1R,2R)-4-hydroxy-4-[2-(5-methylpyridazin-3-yl)ethynyl]-2-phenylcyclohexane-1-carboxylate as a white solid.

LC/MS (Method B): RT=1.028; m/z=393 [M+H]⁺

### Step 4: tert-butyl (1R,2R)-4-methoxy-4-[2-(5-methylpyridazin-3-yl)ethynyl]-2-phenylcyclohexane-1-carboxylate

Starting from tert-butyl (1R,2R)-4-hydroxy-4-[2-(5-methylpyridazin-3-yl)ethynyl]-2-phenylcyclohexane-1-carboxylate (142 mg, 0.36 mmol) following procedure described in Step 1 of EXAMPLES 146 and 147, the obtained residue was purified via flash chromatography using Heptane-100% EtOAc/Heptane (gradient) as eluent to afford tert-butyl (1R,2R)-4-methoxy-4-[2-(5-methylpyridazin-3-yl)ethynyl]-2-phenylcyclohexane-1-carboxylate as an oil.

LC/MS (Method B): RT=1.172; m/z=407 [M+H]⁺

### Step 5: (1R,2R)-4-methoxy-4-[2-(5-methylpyridazin-3-yl)ethynyl]-2-phenylcyclohexane-1-carboxylic acid

Starting from tert-butyl (1R,2R)-4-methoxy-4-[2-(5-methylpyridazin-3-yl)ethynyl]-2-phenylcyclohexane-1-carboxylate (71 mg, 0.19 mmol) following procedure described in Step 5 of EXAMPLE 133, (1R,2R)-4-methoxy-4-[2-(5-methylpyridazin-3-yl)ethynyl]-2-phenylcyclohexane-1-carboxylic acid was obtained as a yellow oil. The compound was used without further purification.

LC/MS (Method B): RT=0.85; m/z=351 [M+H]⁺

### Step 6: EXAMPLE 169

Starting from (1R,2R)-4-methoxy-4-[2-(5-methylpyridazin-3-yl)ethynyl]-2-phenylcyclohexane-1-carboxylic acid (73 mg) and 5-amino-3-[((4S)-3,3-difluoro-4-hydroxypiperidin-4-yl]methyl)-6-(4-fluorophenoxy)-3,4-dihydropyrimidin-4-one (77 mg, 0.21 mmol) following procedure described in Step 3 of EXAMPLE 94, the obtained residue was purified via flash chromatography using Heptane-100% EtOAc/Heptane (gradient) as eluent to afford EXAMPLE 169 as a white solid.

LC/MS (Method B): RT=1.025, m/z 703 [M+H]⁺

¹H NMR (399 MHz, DMSO₆) 9.15 (d, J=2.1 Hz, 1H), 7.79 (m, 1H), 7.67-7.63 (m, 1H), 7.30-7.17 (m, 7H), 7.13-7.07 (m, 2H), 6.07-6.02 (m, 1H), 4.77-4.72 (m, 2H), 4.47-4.21 (m, 2H), 4.03-3.74 (m, 2H), 3.39 (s, 3H), 3.28-3.08 (m, 4H), 2.37 (s, 3H), 2.26 -2.17 m, 2H), 1.91-1.77 (m, 4H), 1.51-1.23 (m, 2H).

HRMS (TOF, ESI) m/z: Calculated for C₃₇H₃₇F₃N₆O₅ 702.2778, Found: 703.2877 [M+H]⁺

### 5-amino-3-{[(4S)-3,3-difluoro-4-hydroxy-1-[(1R,2R,4R)-4-methoxy-4-[2-(4-methylpyridazin-3-yl)ethynyl]-2-phenylcyclohexanecarbonyl]piperidin-4-yl]methyl}-6-(4-fluorophenoxy)-3,4-dihydropyrimidin-4-one (EXAMPLE 170)

### Step 1: 4-methyl-3-[2-(trimethylsilyl)ethynyl]pyridazine

Starting from 3-chloro-4-methyl-pyridazine (525 mg, 4.08 mmol) and ethynyltrimethylsilane (481 mg, 4.90 mmol, 1.2 eq.) following procedure described in Step 1 of EXAMPLES 135 and 136, the obtained residue was purified via flash chromatography using Heptane-100% EtOAc/Heptane (gradient) as eluent to afford 4-methyl-3-[2-(trimethylsilyl)ethynyl]pyridazine as a yellow solid.

LC/MS (Method B): RT=0.963; m/z=191 [M+H]⁺

### Step 2, 3-ethynyl-4-methylpyridazine

To a solution of 4-methyl-3-[2-(trimethylsilyl)ethynyl]pyridazine (0.723 g, 3.80 mmol) in THF/MeOH (1:1, 20 mL) was added potassium carbonate (52 mg, 0.38 mmol, 0.1 eq.). The reaction mixture was stirred at r.t. for 1 hour and evaporated in vacua. The residue was purified via flash chromatography using Heptane-100% EtOAc/Heptane (gradient) as eluent to afford 3-ethynyl-4-methylpyridazine as an off-white solid.

LC/MS (Method B): RT=0.419; m/z=119 [M+H]⁺

### Step 3: tert-butyl (1R,2R)-4-hydroxy-4-[2-(4-methylpyridazin-3yl)ethynyl]-2-phenylcyclohexane-1-carboxylate

Starting from tert-butyl (1R,2R)-4-oxo-2-phenylcyclohexane-1-carboxylate (400 mg, 1.46 mmol) and 3-ethynyl-4-methylpyridazine (241 mg, 2.04 mmol) following procedure described in Step 2 of EXAMPLES 122 and 123, the obtained residue was purified via flash chromatography using Heptane-100% EtOAc/Heptane (gradient) as eluent to afford tert-butyl (1R,2R)-4-hydroxy-4-[2-(4-methylpyridazin-3-yl)ethynyl]-2-phenylcyclohexane-1-carboxylate as a off-white solid.

LC/MS (Method B): RT=1.017; m/z=393 [M+H]⁺

### Step 4: tert-butyl (1R,2R)-4-methyl-4-[2-(4-methylpyridazin-3-yl)ethynyl]-2-phenylcyclohexane-1-carboxylate

Starting from tert-butyl (1R,2R)-4-hydroxy-4-[2-(4-methylpyridazin-3-yl)ethynyl]-2-phenylcyclohexane-1-carboxylate (100 mg, 0.25 mmol) following procedure described in Step 1 of EXAMPLES 146 and 147, the obtained residue was punned yea flash chromatography using Heptane-100% EtOAc/Heptane (gradient) as eluent to afford tert-butyl (1R,2R)-4-methoxy-4-[2-(4-methylpyridazin-3-yl)ethynyl]-2-phenylcyclohexane-1-carboxylate as an yellow oil.

LC/MS (Method B): RT=1.152; m/z=407 [M+H]⁺

### Step 5: (1R,2R)-4-methoxy-4-[2-(4-methylpyridazin-3-yl)ethynyl]-2-phenylcyclohexane-1-carboxylic acid

Starting from tert-butyl (1R,2R)-4-methoxy-4-[2-(4-methylpyridazin-3-yl)ethynyl]-2-phenylcyclohexane-1 -carboxylate (73 mg, 0.18 mmol) following procedure described in Step 5 of EXAMPLE 133, (1R,2R)-4-methoxy-4-[2-(5-methylpyridazin-3-yl)ethynyl]-2-phenylcyclohexane-1-carboxylic acid was obtained as a yellow oil. The compound was used without further purification.

LC/MS (Method B): RT=0.841; m/z=351 [M−H]⁺

### Step 6: EXAMPLE 170

Starting from (1R,2R)-4-methoxy-4-[2-(4-methylpyridazin-3-yl)ethynyl]-2-phenylcyclohexane-1-carboxylic acid (56 mg) and 5-amino-3-{[(4S)-3,3-difluoro-4-hydroxypiperidin-4-yl]methyl}-6-(4-fluorophenoxy)-3,4-dihydropyrimidin-4-one (59 mg, 0.16 mmol) following procedure described in Step 3 of EXAMPLE 94, the obtained residue was purified via flash chromatography using Heptane-100% EtOAc/Heptane (gradient) as eluent to afford EXAMPLE 170 as a white solid.

LC/MS (Method B): RT=1.015; m/z=703 [M+H]

¹H NMR (399 MHz, DMSO-d₆) 9.10 (d, J=2.1 Hz, 1H), 7.72-7.62 (m, 2H), 7.30-7.07 (m, 9H), 6.07-6.01 (m, 1H), 4.77-4.71 (m, 2H), 4.47-4.20 (m, 2H), 4.06-3.74 (m, 2H), 3.43 (s, 3H), 3.30-3.07 (m, 4H), 2.53 (s, 3H), 2.29-2.20 (m, 2H), 1.92-1.77 (m, 4H), 1.51-1.23 (m, 2H).

HRMS (TOF, ESI) Calculated for C₃₇H₃₇FN₆O₅ 702.2778, Found: 703.288 [M+H]⁺

### 5-amino-3-{[(4S)-3,3-difluoro-1-[(1R,2R,4R)-4-fluoro-4-[2-(4-methylpyridazin-3-yl)ethynyl]-2-phenylcyclohexanecarbonyl]-4-hydroxypiperidin-4-yl]methyl}-6-(4-fluorophenoxy)-3,4-dihydropyrimidin-4-one (EXAMPLE 171)

### Step 1: tert-butyl (1R,2R)-4-hydroxy-4-[2-(4-methylpyridazin-3-yl)ethynyl]-2-phenylcyclohexane-1-carboxylate

Starting from tert-butyl (1R,2R)-4-hydroxy-4-[2-(4-methylpyridazin-3)ethynyl]-2-phenylcyclohexane-1-carboxylate (334 mg, 0.85 mmol) following procedure described in Step 4 of EXAMPLES 122 and 123, the obtained residue was purified via flash chromatography using Heptane-100% EtOAc/Heptane (gradient) as eluent to afford tert-butyl (1R,2R)-4-fluoro-4-[2-(4-methylpyridazin-3-yl)ethynyl]-2-phenylcyclohexane-1-carboxylate as an brown oil.

LC/MS (Method B): RT=1.16; m/z=395 [M+H]⁺

### Step 2: (1R,2R)-4-fluoro-4-[2-(4-methylpyridazin-3-yl)ethynyl]-2-phenylcyclohexane-1-carboxylic acid

Starting from tert-butyl (1R,2R)-4-fluoro-4-[2-(4-methylpyridazin-3-yl)ethynyl]-2-phenylcyclohextane-1-carboxylate (176 mg. 0.45 mmol) following procedure described in Step 5 of EXAMPLE 133, (1R,2R)-4-fluoro-4-[2-(4-methylpyridazin-3-yl)ethynyl]-2-phenylcyclohexane-1-carboxylic acid was obtained as a brown oil. The compound was used without further purification.

LC/MS (Method B): RT=0.866; m/z=339 [M+H]⁺

### Step 3: EXAMPLE 171

Starting from ((1R,2R)-4-fluoro-4-[2-(4-methylpyridazin-3-yl)ethynyl]-2-phenylcyclohexane-1-carboxylic acid (151 mg) and 5-amino-3-{[(4S)-3,3-difluoro-4-hydroxypiperidin-4-yl]methyl}-6-(4-fluorophenoxy)-3,4-dihydropyrimidin-4-one (165 mg, 0.45 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 171 as a white solid.

LC/MS (Method B): RT=1.044; m/z=691 [M+H]⁺

¹H NMR (399 MHz, DMSO-d₆) 9.07 (d, J=5.2 Hz, 1H), 7.65 (d, J=5.2 Hz, 1H), 7.60-7.55 (m, 1H), 7.25-6.99 (m, 9H), 6,07-5.99 (m, 1H), 4.69-4.64 (m, 2H), 4.39-4.15 (m, 2H). 3.94-3.66 (m, 2H), 136-2.99 (m, 4H), 2.43 (s, 3H), 2.33-2.06 (m, 4H), 1.92-1.67 (m, 2H), 1.44-1.15 (m, 2H)

HRMS (TOF, ESI) m/z: Calculated for C₃₆H₃₄F₄N₆O₄ 690.2578, Found: 691.2689 [M+H]⁺

### 5-amino-3-{[(4S)-3,3-difluoro-1-[(1R,2R,4R)-4-fluoro-4-[2-(5-methylpyridazin-3-yl)ethynyl]-2-phenylcyclohexanecarbonyl]-4-hydroxypiperidin-4-yl]methyl}-6-(4-fluorophenoxy)-3,4-dihydropyrimidin-4-one (EXAMPLE 172)

### Step 1: tert-butyl (1R,2R)-4-fluoro-4-[2-(5-methylpyridazin-3-yl)ethynyl]-2-phenylcyclohexane-1-carboxylate

Starting from tert-butyl (1R,2R)-4-hydroxy-4-[2-(5-methylpyridazin-3-yl)ethynyl]-2-phenylcyclohexane-1-carboxylate (246 mg, 0.63 mmol) following procedure described in Step 4 of EXAMPLES 122 and 123, the obtained residue was purified via flash chromatography using Heptane-100% EtOAc/Heptane (gradient) as eluent to afford tert-butyl (1R,2R)-4-fluoro-4-[2-(4-methylpyridazin-3-yl)ethynyl]-2-phenylcyclohexane-1-carboxylate as a yellow oil.

LC/MS (Method B): RT=1.2; m/z=395 [M+H]⁺

### Step 2: (1R,2R)-4 -fluoro-4-[2-(5-methylpyridazin-3-yl)ethynyl]-2 -phenylcyclohexane-1-carboxylic acid

Starting from tert-butyl (1R,2R)-4-fluoro-4-[2-(5-methylpyridazin-3-yl)ethynyl]-2-phenylcyclohexane-1 -carboxylate (207 mg, 0.52 mmol) following procedure described in Step 5 of EXAMPLE 133, (1R,2R)-4-fluoro-4-[2-(5-methylpyridazin-3-yl)ethynyl]-2-phenylcyclohexane-1-carboxylic acid was obtained as a brown oil. The compound was used without further purification.

LC/MS (Method B): RT=0898; m/z=339 [M−H]⁺

### Step 3: EXAMPLE 172

Starting from ((1R,2R)-4-fluoro-4-[2-(5-methylpyridazin-3-yl)ethynyl]-2-phenylcyclohexane-1-carboxylic acid (177 mg) and 5-amino-3-{[(4S)-3,3-difluoro-4-hydroxypiperidin-4-yl]methyl}-6-(4-fluorophenoxy)-3,4-dihydropyrimidin-4-one (194 mg, 0.52 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 172 as a while solid.

LC/MS (Method B): RT=1.053; m/z=691 [M+H]⁺

¹H NMR (399 MHz, DMSO-d₆) 9.11 (s, 1H), 7.77 (m, 1H), 7.60-7.55 (m, 1H), 7.24-7.10 (m, 7H), 7.03-6.99 (m, 2H), 5.99 (s, 1H), 4.69-4.64 (m, 2H), 4.40-4.11 (m, 2H), 3.94-3.67 (m, 2H), 3.15-3.00 (m, 4H), 2.30 (s, 3H), 2.25-2.06 (m, 4H), 1.91-1.67 (m, 2H), 1.44-1.15 (m, 2H)

HRMS (TOF, ESI) Calculated for C₃₆H₃₄F₄N₆O₄ 690.2578, Found: 691.2686 [M+H]⁺

### 5-amino-3-{[(4S)-3,3-difluoro-4-hydroxy-1-[(1R,4R)-4-methoxy-4-[2-(5-methoxypyridazin-3-yl)ethynyl]-2-phenylcyclohexanecarbonyl]piperidin-4-yl]methyl}-6-(4-fluorophenoxy)-3,4-dihydropyrimidin-4-one (EXAMPLE 173)

### Step 1: 5-methoxy-3-[2-(trimethylsilyl)ethynyl]pyridazine

Starting from 3-chloro-5-methoxypyridazine (1 g, 6.92 mmol) and ethynyltrimethylsilane (0.82 mg, 8.30 mmol, 1.2 eq.) following procedure described in Step 1 of EXAMPLES 135 and 136, the obtained residue was purified via flash chromatography using Heptane-100% EtOAc/Heptane (gradient) as eluent to afford 5-methoxy-3-[2-(trimethylsilyl)ethynyl]pyridazine as a brown solid.

LC/MS (Method B): RT=0.950; m/z=207 [M+H]⁺

### Step 2: 3 -ethynyl-5-methoxypyridazine

To a solution of 5-methoxy-3-[2-(trimethylsilyl)ethynyl]pyridazine (0.651 g, 3.16 mmol) in THF/MeOH (1:1, 20 mL), was added potassium carbonate (44 mg, 0.32 mmol, 0.1 eq.). The reaction mixture was stirred at r.t. for 1 hour and evaporated in vacuo. The obtained residue was purl lied via flash chromatography using Heptane-100% EtOAc/Heptane (gradient) as eluent to afford 3-ethynyl-5-methoxypyridazine as an off-white solid

LC/MS (Method B): RT=0.373; m/z=135 [M+H]⁺

### Step 3: tert-butyl (1R,2R)-4-oxo-hydroxy-1-[2-(5-methoxypyridazin-3-yl)ethynyl]-2-phenylcyclohexane-1-carboxylate

Starting from tert-butyl (1R,2R)-4-oxo-2-phenylcyclohexane-1-carboxylate (400 mg, 1.46 mmol) and 3-ethynyl-5-methoxypyridazine (254 mg, 1.90 mmol) following procedure described in Step 2 of EXAMPLES 122 and 123, the obtained residue was purified via flash chromatography using Heptane-85% EtOAc/Heptane (gradient) as eluent to afford tert-butyl (1R,2R)-4-hydroxy-4-[2-(5-methoxypyridazin-3-yl)ethynyl]-2-phenylcyclohexane-1-carboxylate as an off-white solid.

LC/MS (Method B): RT=1.208; m/z=409 [M+H]⁺

### Step 4: tert-butyl (1R,2R)-4-methoxy-4-[2(-5-methoxypyridazin-3-yl)ethynyl]-2-phenylcyclohexane-1-carboxylate

Starting from (1R,2R)-4-hydroxy-4-[2-(5-methoxypyridazin-3-yl)ethynyl]-2-phenylcyclohexane-1-carboxylate (109 mg, 0.27 mmol) following procedure described in Step 1 of EXAMPLES 146 and 147, the obtained residue was purified via flash chromatography using Heptane-70% EtOAc/Heptane (gradient) as eluent to afford tert-butyl (1R,2R)-4-methoxy-4-[2-(5-methoxypyridazin-3-yl)ethynyl]-2-phenylcyclohexane-1-carboxylate as a yellow oil.

LC/MS (Method B): RT=1.346; m/z=423 [M+H]⁺

### Step 5: (1R,2R)-4-methoxy-4-[2-(5-methoxypyridazin-3-yl)ethynyl]-2 -phenylcyclohexane-1-carboxylic acid

Starting from tert-butyl (1R,2R)-4-methoxy-4-[2-(5-methoxypyridazin-3-yl)ethynyl]-2-phenylcyclohexane-1-carboxylate (95 mg, 0.22 mmol) following procedure described in Step 5 of EXAMPLE 133, (1R,2R)-4-methoxy-4-[2-(5-methoxypyridazin-3-yl)ethynyl]-2-phenylcyclohexane-1-carboxylic acid was obtained as a yellow oil. The compound was used without further purification.

LC/MS (Method B): RT=1.037; m/z=367 [M−H]⁺

### Step 6: EXAMPLE 173

Starting from (1R,2R)-4-methoxy-4-[2-(5-methoxypyridazin-3-yl)ethynyl]-2-phenylcyclohexane-1-carboxylic acid (110 mg) and 5-amino-3-{[(4S)-3,3-difluoro-4-hydroxypiperidin-4-yl]methyl}-6-(4-fluorophenoxy)-3,4-dihydropyrimidin-4-one (83 mg, 0.23 mmol) following procedure described in Step 3 of EXAMPLE 94, the obtained residue was purified via prep HPLC Prep (HPLC Column: Gemini pH4 Dimensions: 21.1 mm×150 mm 5 μm) to afford EXAMPLE 173 as a white solid.

LC/MS (Method B): RT=1.203; m/z=719 [M+H]⁺

¹H NMR (399 MHz, DMSO-d₆) 9.02 (d, J=2.1 Hz, 1H), 7.64 (m, 1H), 0.51 (m, 1H), 7.30-7.17 (m, 7H), 7.13-7.07 (m, 2H), 6.07-6.02 (m, 1H), 4.76-4.71 (m, 2H) 4.47-4.21 (m, 2H), 4.04-3.74 (m, 5H), 3.41 (s, 3H), 3.28-3.09 (m, 4H), 2.27-2.19 (m, 2H), 1.90-1.77 (m, 4H), 1.55-1.24 (m, 2H).

HRMS (TOF, ESI) m/z: Calculated for C₃₇H₃₇F₃N₆O₆ 718.2727, Found: 719.2832 [M+H]⁺

### 5-amino-3-{[(4S)-3,3-difluoro-4-hydroxy-1-[(1R,2R,4R)-4-methoxy-4-[2-(5-methylpyrimidin-2-yl)ethynyl]-2-phenylcyclohexanecarbonyl]piperidin-4-yl]methyl}-6-(4-fluorophenoxy)-3,4-dihydropyrimidin-4-one (EXAMPLE 174)

### Step 1: tert-butyl (1R,2R)-4-hydroxy-4-[2-(5-methylpyrimidin-2-yl)ethynyl]-2-phenylcyclohexane-1-carboxylate

Starting from tert-butyl (1R,2R)-4-oxo-2-phenylcyclohexane-1-carboxylate (400 mg, 1.46 mmol) and 2-ethynyl-5-methylpyrimidine (224 mg, 1.90 mmol) following procedure described in Step 2 of EXAMPLES 122 and 123, the obtained residue was purified via flash chromatography using Heptane-100% EtOAc/Heptane (gradient) as eluent to afford tert-butyl (1R,2R)-4-hydroxy-4-[2-(5-methylpyrimidin-2-yl)ethynyl]-2-phenylcyclohexane-1-carboxylate as an off-white solid.

LC/MS (Method B): RT=1.248; m/z=393 [M+H]⁺

### Step 2: tert-butyl (1R,2R)-4-hydroxy-4-[2-(5-methylpyrimidin-2 -yl)ethynyl]-2-phenylcyclohexane-1-carboxylate

Starting from tert-butyl (1R,2R)-4-hydroxy-4-[2-(5-methylpyrimidin-2-yl)ethynyl]-2-phenylcyclohexane-1-carboxylate (100 mg, 0.25 mmol) following procedure described in Step 1 of EXAMPLES 146 and 147, the obtained residue was purified via flash chromatography using Heptane-100% EtOAc/Heptane (gradient) as eluent to afford tert-butyl (1R,2R)-4-methoxy-4-[2-(5-methylpyrimidin-2-yl)ethynyl]-2-phenylcyclohexane-1-carboxylate as a white solid.

LC/MS (Method B): RT=1.406; m/z=407 [M+H]⁺

### Step 3: (1R,2R)-4-methoxy-4-[2-(5-methylpyrimidin-2-yl)ethynyl]-2-phenylcyclohexane-1-carboxylic acid

Starting from tert-butyl (1R,2R)-4-methoxy-4-[2-(5-methylpyrimidin-2-yl)ethynyl]-2-phenylcyclohexane-1-carboxylate (100 mg, 0.25 mmol) following procedure described in Step 5 of EXAMPLE 133, (1R,2R)-4-methoxy-4-[2-(5-methylpyrimidin-2-yl)ethynyl]-2-phenylcyclohexane-1-carboxylic acid was obtained as a yellow oil

LC/MS (Method B): RT=1.075; m/z=351 [M−H]⁺

### Step 4: EXAMPLE 174

Starting from (1R,2R)-4-methoxy-4-[2-(5-methylpyrimidin-2-yl)ethynyl]-2-phenylcyclohexane-1-carboxylic acid (43 mg, 0.12 mmol) and 5-amino-3-{[(4S)-3,3-difluoro-4-hydroxypiperidin-4-yl]methyl}-6-(4-fluorophenoxy)-3,4-dihydropyrimidin-4-one (45 mg, 0.12 mmol) following procedure described in Step 3 of EXAMPLE 94, the obtained residue was purified via prep HPLC Prep (HPLC Column: Gemini pH7 Dimensions: 21.1 mm×150 mm 5 μm) to afford EXAMPLE 174 as a white solid.

LC/MS (Method B): RT=1.24; m/z=703 [M+H]⁺

¹H NMR (399 MHz, DMSO-d₆) 8.70 (s, 2H), 7.67-7.63 (m, 1H), 7.30-7.15 (m, 7H), 7.13-7.07 (m, 2H), 6.06-6.01 (m 1H), 4.76-4.71 (m, 2H), 4.47-4.20 (m, 2H), 4.02-3.74 (m, 2H), 3.39 (s, 3H), 3.27-3.09 (m, 4H), 2.43-2.15 (m, 5H), 1.88-1.74 (m, 4H), 1.53-1.26 (m, 2H).

HRMS (TOF, ESI) m/z: Calculated for C₃₇H₃₇F₃N₆O₅ 702.2778, Found: 703.291 [M+H]⁺

### 5-amino-3-{[(4S)-3,3-difluoro-4-hydroxy-1-[(1R,2R,4R)-4-methoxy-2-phenyl-4-[2-(pyrimidin-5-yl)ethynyl]cyclohexanecarbonyl]piperidin-4-yl]methyl}-6-(4-fluorophenoxy)-3,4-dihydropyrimidin-4-one (EXAMPLE 175)

Starting from (1R,2R)-4-methoxy-2-phenyl-4-[2-(pyrimidin-5-yl)ethynyl]cyclohexane-1-carboxylic acid (80 mg. 0.24 mmol) and 5-amino-3-{[(4S)-3,3-difluoro-4-hydroxypiperidin-4-yl]methyl}-6-(4-fluorophenoxy)-3,4-dihydropyrimidin-4-one (88 mg, 0.24 mmol) following procedure described in Step 3 of EXAMPLE 94, the obtained residue was purified via prep HPLC Prep (HPLC Column: Gemini pH7 Dimensions: 21.1 mm×150 mm 5 μm) to afford EXAMPLE 175 as a white solid.

LC/MS (Method B): RT=1.229; m/z=689 [M+H]⁺

¹H NMR (399 MHz, DMSO-d₆) 9.05 (d, J=2.1 Hz, 1H), 7.67-7.58 (m, 7.30-7.17 (m, 8H), 7.12-7.07 (m, 2H), 6.08-6.02 (m, 1H), 4.76-4.71 (m, 2H), 4.47-4.21 (m, 2H), 4.02-3.75 (m, 5H), 3.22-3.08 (m, 4H), 2.45-2.11 (m, 4H), 1.96-1.74 (m, 2H), 1.55-1.24 (m, 2H).

HRMS (TOF, ESI) m/z: Calculated for C₃₆H₃₅F₃N₆O₅ 688.2621, Found: 689.2763 [M+H]⁺

### 5-amino-3-{[(4S)-3,3-difluoro-4-hydroxy-1-[(1R,2R,4R)-4-methoxy-4-[2-(4-methylpyrimidin-2-yl)ethynyl]-2-phenylcyclohexanecarbonyl]piperidin-4-yl]methyl}-6-(4-fluorophenoxy)-3,4-dihydropyrimidin-4-one (EXAMPLE 176)

### Step 1: tert-butyl (1R,2R) -hydroxy-4-[2-(4-methylpyrimidin-2-yl)ethynyl]-2-phenylcyclohexane-1-carboxylate

Starting from tert-butyl (1R,2R)-4-oxo-2-phenylcyclohexane-1 -carboxylate (600 mg, 2.19 mmol) and 2-ethynyl-4-methylpyrimidine (336 mg, 2.54 mmol) following procedure described in Step 2 of EXAMPLES 122 and 123, the obtained residue was purified via flash chromatography using Heptane-100% EtOAc/Heptane (gradient) as eluent to afford tert-butyl (1R,2R)-4 -hydroxy-4-[2-(4-methylpyrimidin-2-yl)ethynyl]-2-phenylcyclohexane-1-carboxylate as an off-white solid.

LC/MS (Method B): RT=1.243; m/z=393 [M+H]⁺

### Step 2: tert-butyl (1R,2R)-4-methoxy-4-[2-(4-methylpyrimidin-2-yl)ethynyl]-2-phenylcyclohexane-1-carboxylate

Starting from tert-butyl (1R,2R)-4-hydroxy-4-[2-(4-methylpyrimdin-2-yl)ethynyl]-2-phenylcyclohexane-1-carboxylate (214 mg, 0.55 mmol) following procedure described in Step 1 of EXAMPLES 146 and 147, the obtained residue was purified via flash chromatography using Heptane-100% EtOAc/Heptane (gradient) as eluent to afford tert-butyl (1R,2R)-4-methoxy-4-[2-(4-methylpyrimidin-2-yl)ethynyl]-2-phenylcyclohexane-1-carboxylate as an oil.

LC/MS (Method B): RT=1.207; m/z=407 [M+H]⁺

### Step 3: (1R,2R)-4-methoxy-4-[2-(4-methylpyrimidin-2-yl)ethynyl]-2-phenylcyclohexane-1-carboxylic acid

Starting from tert-butyl (1R,2R)-4-methoxy-4-[2-(4-methylpyrimidin-2-yl)ethynyl]-2-phenylcyclohexane-1-carboxylate (121 mg, 0.30 mmol) following procedure described in Step 5 of EXAMPLE 133, (1R,2R)-4-methoxy-4-[2-(4-methylpyrimidin-2-yl)ethynyl]-2-phenylcyclohexane-1-carboxylic acid was obtained as a yellow oil.

LC/MS (Method B): RT=1.060; m/z=351 [M−H]⁺

### Step 4: EXAMPLE 176

Starting from (1R,2R)-4-methoxy-4-[2-(4-methylpyrimidin-2-yl)ethynyl]-2-phenylcyclohexane-1-carboxylic acid (52 mg 0.15 mmol) and 5-amino-3-{[(4S)-3,3-difluoro-4-hydroxypiperidin-4-yl]methyl}-6-(4-fluorophenoxyl-3,4-dihydropyrimidin-4-one (55 mg, 0.15 mmol) following procedure described in Step 3 of EXAMPLE 94, the obtained residue was purified via prep HPLC Prep (HPLC Column: Gemini pH7 Dimensions: 21.1 mm×150 mm 5 μm) to afford EXAMPLE 176 as a white solid.

LC/MS (Method B): RT=1.038; m/z=703 [M+H]⁺

¹H NMR (399 MHz, DMSO-d₆) (d, J=5.2 Hz, 1H), 7.67-7.63 (m, 1H), 7.43 (d, J=5.2 Hz, 1H), 7.30-7.15 (m, 7H), 7.12-7.07 (m, 2H), 6.06-6.01 (m, 1H), 4.75-4.71 (m, 2H), 4.47-4.20 (m, 2H), 4.02-3.74 (m, 2H), 3.38 (s, 3H), 3.30-3.09 (m, 4H), 2.50-2.15 (m, 5H), 1.88-1.74 (m, 4H), 1.52-1.24 (m, 2H).

HRMS (TOF, ESI) m/z: Calculated for C₃₇H₃₇F₃N₆O₅ 702.2778 Found: 703.2915 [M+H]⁺

### 5-amino-3-{[(4S)-3,3-difluoro-4-hydroxy-1-[(1R,2R,4R)-4-methoxy-4-[2-(2-methylpyrimidin-5-yl)ethynyl]-2-phenylcyclohexanecarbonyl]piperidin-4-yl]methyl}-6-(4-fluorophenoxy)-3,4-dihydropyrimidin-4-one (EXAMPLE 177)

### Step 1: tert-butyl (1R,2R)-4-hydroxy-4-[2-(2-methylpyrimidin-5-yl)ethynyl]-2-phenylcyclohexane-1-carboxylate

Starting from tert-butyl (1R,2R)-4-oxo-2-phenylcyclohexane-1-carboxylate (600 mg, 2.19 mmol) and 5-ethynyl-2-methylpyrimidine (362 mg, 3.06 mmol) following procedure described in Step 2 of EXAMPLES 122 and 123, the obtained residue was purified via flash chromatography using Heptane-65% EtOAc/Heptane (gradient) as eluent to afford tert-butyl (1R,2R)-4-hydroxy-4-[2-(2-methylpyrimidin-5-yl)ethynyl]-phenylcyclohexane-1-carboxylate as an oil.

LC/MS (Method B): RT=1.032; m/z=393 [M+H]⁺

### Step 2: tert-butyl (1R,2R)-4-methoxy-4-[2-(2-methylpyrimidin-5-yl)ethynyl]-2-phenylcyclohexane-1-carboxylate

Starting from tert-butyl (1R,2R)-4-hydroxy-4-[2-(2-methylpyrimidin-5-yl)ethynyl]-2-phenylcyclohexane-1-carboxylate (326 mg, 0.83 mmol) following procedure described in Step 1 of EXAMPLES 146 and 147, the obtained residue was purified via flash chromatography using Heptane-70% EtOAc/Heptane (gradient) as eluent to afford tert-butyl (1R,2R)-4-methoxy-4-[2-(2-methylpyrimidin-5-yl)ethynyl]-2-phenylcyclohexane-1-carboxylate as an off-white solid.

LC/MS (Method B): RT=1.217; m/z=407 [M+H]⁺

### Step 3: (1R,2R)-4-methoxy-4-[2-(2-methylpyrimidin-5 -yl)ethynyl]-2-phenylcyclohexane-1-carboxylic acid

Starting from tert-butyl (1R,2R)-4-methoxy-4-[2-(2-methylpyrimidin-5-yl)ethynyl]-2-phenylcyclohexane-1-carboxylate (170 mg, 0.42 mmol) following procedure described in Step 5 of EXAMPLE 133, (1R,2R)-4-methoxy-4-[2-(2-methylpyrimidin-5-yl)ethynyl]-2-phenylcyclohexane-1-carboxylic acid was obtained as a yellow oil. The compound was used without further purification.

LC/MS (Method B): RT=0.898, m/z=351 [M−H]⁺

### Step 4: EXAMPLE 177

Starting from (1R,2R)-4-methoxy-4-[2-(2-methylpyrimidin-5-yl)ethynyl]-2-phenylcyclohexane-1-carboxylic acid (78 mg) and 5-amino-3-{[(4S)-3,3-difluoro-4-hydroxypiperidin-4-yl]methyl}-6-(4-fluorophenoxy)-3,4-dihydropyrimidin-4-one (82 mg, 0.22 mmol) following procedure described in Step 3 of EXAMPLE 94, the obtained residue was purified via prep HPLC Prep (HPLC Column: Gemini pH7 Dimensions: 21.1 mm×150 mm 5 μm) to afford EXAMPLE 177 as a white solid.

LC/MS (Method B): RT=1.054; m/z=703 [M+H]⁺

¹H NMR (399 MHz, DMSO-d₆) 8.88 (m, 2H), 7.67-7.63 (m, 1H), 7.29-7.15 (m, 7H), 7.13-7.07 (m, 2H), 6.08-6.02 (m, 1H), 4.77-4.72 (m, 2H), 4.47-4.21 (m, 2H), 4.02-3.74 (m, 2H), 3.38 (s, 3H), 3.27-3.07 (m, 4H), 2.67 (s, 3H), 2.43-2.14 (m, 2H), 1.87-1.74 (m, 4H), 1.53-1.26 (m, 2H).

HRMS (TOF, ESI) m/z: Calculated for C₃₇H₃₇F₃N₆O₅ 702.2778, Found: 703.2907 [M+H]⁺

### Pharmacological Study

### EXAMPLE A

### Evaluation of the Inhibition of USP7 by the Fluorescence Intensity (FLINT) Readings

USP7 activity was measured using Rhodamine-110 c-terminal labelled Ubiquitin as a substrate (UbiQ Bio). Incubation with USP7 results in the release of Rhodamine-110 leading to an increase in fluorescence which can be used in the continuous measurement of USP7 activity.

The USP7 reactions were performed in a 50 μL volume, in 384 well black solid low binding plates (Corning #3575). The reaction buffer consisted of 100 mM Bicine pH 8.0, 0.01% TritonX100, 1 mM TCEP, and 10% DMSO.

0.25 nM His-His-USP7 (aa208-560, |C315A|) was incubated with compound (final concentration 10% DMSO) for 60 minutes at 30° C. The reaction was then initiated by the addition of 500 nM Ubiquitin-Rhodamme-110 substrate and the plate read every 3 minutes for 21 minutes to measure the release of Rhodamine-110. Fluorescence Intensity (FLINT) readings were measured using a Biomek Neo plate reader (Ex.485 nm, Em.535 nm).

The inhibition of increasing doses of compound was expressed as a percentage reduction in kinetic rate compared to the kinetic rates established between ‘DMSO only’ and ‘total inhibition’ controls (no USP7). The inhibitory concentrations that gave a 50% reduction in kinetic rate (IC₅₀) were determined, from 11-point dose response cures, in XL-Fit using a 4-Parameter Logistic Model 205 (Sigmoidal Dose-Response Model).

The results presented in Table 1 below show that compounds of the invention inhibit interaction between USP7 protein and the fluorescent peptide described hereinbefore.

### EXAMPLE B

### In Vitro Cytotoxicity

The cytotoxicity studies were evaluated by MTT [3-(4,5-dimethylthiazol-2-yl)-2,5-diphenyltetrazolium bromide] assay and carried out on Z138 mantle cell lymphoma tumour cell lines. The cells are distributed onto microplates and exposed to the test compounds for 96 hours. MTT is then added for 4 hours and converted by NAD(P)H-dependent cellular oxidoreductase enzymes in formazan, which has a purple color. The viable cell number is proportional to the production of formuan salts and cell viability can be quantified by the absorbance of the solution at 540 nm with a speetrophotometer (Carmichael et al., Cancer Res. 1987, 47, 936-942). The results are expressed in IC₅₀ (the concentration of compound that inhibits cell viability by 50% compared to DMSO treated cells only) and are presented in Table 1 below.

The results show that the compounds of the invention are cytotoxic.

| Example | IC₅₀ (M) USP7 FLINT | IC₅₀ (μM) MTT Z138 |
|---|---|---|
| 1 | 4.37E−08 | 8.66E−02 |
| 2 | 3.04E−07 | NT |
| 3 | 8.02E−08 | 5.95E−02 |
| 4 | 9.38E−08 | NT |
| 5 | 6.34E−08 | 4.35E−02 |
| 6 | 1.83E−07 | NT |
| 7 | 1.81E−07 | NT |
| 8 | 4.72E−07 | NT |
| 9 | 8.58E−08 | 2.87E−01 |
| 10 | 4.32E−06 | NT |
| 11 | 5.87E−08 | 1.97E−01 |
| 12 | 5.71E−08 | 9.09E−02 |
| 13 | 9.75E−08 | 1.78E−01 |
| 14 | 7.18E−08 | 4.13E−02 |
| 15 | 2.35E−08 | 1.95E−02 |
| 16 | 5.16E−08 | 4.24E−02 |
| 17 | 1.25E−06 | NT |
| 18 | 2.47E−07 | NT |
| 19 | 8.31E−08 | 7.16E−02 |
| 20 | 1.05E−07 | 1.46E−01 |
| 21 | 7.16E−07 | NT |
| 22 | 2.64E−07 | NT |
| 23 | 4.0E−08 | 2.2E−01 |
| 24 | 3.0E−08 | 1.65E−01 |
| 25 | 2.03E−07 | NT |
| 26 | 2.0E−08 | 4.06E−01 |
| 27 | 8.5E−08 | NT |
| 28 | 5.3E−08 | 5.2E−02 |
| 29 | 1.75E−07 | NT |
| 30 | 1.51E−07 | NT |
| 31 | 4.7E−08 | 6.48E−01 |
| 32 | 3.2E−08 | 8.96E−01 |
| 33 | 4.59E−08 | 6.46E−02 |
| 34 | 1.08E−08 | 5.2E−01 |
| 35 | 3.42E−08 | 8.1E−02 |
| 36 | 2.9E−08 | 3.5E−02 |
| 37 | 1.58E−07 | NT |
| 38 | 6.39E−08 | 1.28E−01 |
| 39 | 1.16E−07 | NT |
| 40 | 1.32E−07 | NT |
| 41 | 4.2E−08 | 9.6E−03 |
| 42 | 8.97E−08 | 1.69E−01 |
| 44 | 3.87E−8 | 7.36E−02 |
| 45 | 5.7E−08 | 1.15E−01 |
| 47 | 8.81E−09 | 3.11E−0I |
| 48 | 6.2E−08 | 7.2E−02 |
| 49 | 1.38E−07 | NT |
| 50 | 1.16E−07 | NT |
| 51 | 1.2E−07 | NT |
| 53 | 4.39E−07 | NT |
| 54 | 7.4 E−09 | 2.73E−01 |
| 55 | 8.69E−09 | 2.64E−01 |
| 56 | 1.5E−08 | 5.39E−01 |
| 57 | 6.11E−07 | NT |
| 58 | 6.7E−08 | NT |
| 59 | 4.2E−09 | 6.4E−02 |
| 60 | 6.5E−09 | NT |
| 61 | 9.8E−09 | 2.8E−02 |
| 62 | 1.0E−07 | NT |
| 63 | 1.62E−06 | NT |
| 64 | 2.1E−08 | 1.4E−02 |
| 65 | 1.0E−08 | 3.86E−01 |
| 66 | 1.1E−08 | 3.01 E−01 |
| 67 | 7.5E−08 | 6.68E−01 |
| 68 | 3.9E−08 | 4.54E−01 |
| 69 | 1.19E−07 | NT |
| 70 | 1.34E−07 | NT |
| 73 | 1.13E−07 | 5.64E−01 |
| 74 | 2.27E−07 | NT |
| 75 | 1.0E−08 | 1.84E−01 |
| 76 | 1.5E−08 | 4.0E−01 |
| 77 | 2.5E−08 | 1.56E−01 |
| 78 | 5.75E−08 | 1.15E−01 |
| 79 | 2.28E−07 | NT |
| 80 | 3.75E−07 | NT |
| 81 | 4.03E−07 | NT |
| 82 | 9.03E−08 | 1.80E−01 |
| 83 | 2.2E−07 | 3.27E−01 |
| 84 | 2.12E−07 | NT |
| 88 | 1.11E−07 | NT |
| 89 | 9.2E−08 | 5.5E−02 |
| 90 | 3.9E−09 | 1.6E−01 |
| 91 | 2.89E−07 | NT |
| 92 | 2.30E−07 | 1.89E−01 |
| 93 | 6.63E−08 | 4.11E−02 |
| 94 | 6.34E−08 | 4.94E−02 |
| 95 | 1.72E−07 | 2.53E−01 |
| 96 | 1.09E−07 | 1.16E−01 |
| 97 | 3.82E−08 | 4.97E−01 |
| 98 | 2.23E−07 | NT |
| 99 | 1.0.2E−07 | 1.28E−02 |
| 100 | 2.44E−07 | NT |
| 101 | 3.63E−07 | NT |
| 102 | 4.61E−07 | NT |
| 103 | 3.12E−07 | NT |
| 104 | 4.1E−08 | 1.4E−02 |
| 106 | 1.52E−07 | NT |
| 107 | 4.79E−07 | NT |
| 108 | 6.39E−08 | 6.54E−01 |
| 109 | 4.03E−08 | 1.55E−02 |
| 110 | 2.8E−08 | 2.54E−02 |
| 111 | 2.21E−08 | 5.88E−02 |
| 112 | 7.08E−08 | NT |
| 113 | 1.4E−07 | NT |
| 114 | 4.87E−08 | NT |
| 115 | 3.72E−08 | 8.5E−01 |
| 116 | 8.62E−08 | NT |
| 117 | 8.22E−08 | 6.17E−01 |
| 118 | 3.31E−08 | 4.13E−02 |
| 119 | 3.8E−08 | 6.13E−02 |
| 120 | 1.63E−08 | 1.03E−01 |
| 121 | 3.08E−09 | 5.46E−01 |
| 122 | 2.63E−08 | 3.74E−02 |
| 123 | 3.54E−08 | 4.66E−02 |
| 124 | 8.96E−08 | 2.36E−01 |
| 125 | 8.95E−08 | 6.03E−02 |
| 126 | 7.95E−08 | 1.70E−01 |
| 127 | 4.66E−08 | NT |
| 130 | 1.70E−08 | 1.44E−02 |
| 131 | 2.18E−08 | NT |
| 132 | 3.29E−09 | 2.14E−03 |
| 133 | 3.15E−08 | 1.87E−02 |
| 134 | 8.07E−08 | 3.09E−02 |
| 135 | 3.64E−08 | NT |
| 136 | 1.97E−07 | NT |
| 137 | 6.27E−08 | 2.46E−02 |
| 138 | 2.03E−08 | 6.57E−03 |
| 139 | 4.97E−08 | 2.91E−02 |
| 140 | 6.91E−08 | 4.98E−02 |
| 141 | 4.10E−08 | 1.17E−02 |
| 142 | 5.88E−08 | 1.76E−02 |
| 143 | 5.65E−08 | 7.87E−02 |
| 144 | 2.10E−08 | 1.22E−02 |
| 145 | 8.66E−09 | 2.31E−03 |
| 146 | 1.85E−08 | 5.97E−03 |
| 147 | 1.24E−07 | 1.99E−01 |
| 148 | 2.66E−08 | 1.01E−02 |
| 149 | 8.55E−09 | 2.59E−01 |
| 150 | 3.77E−08 | 2.58E−02 |
| 151 | 1.14E−08 | 1.91E−02 |
| 152 | 3.03E−08 | 2.68E−01 |
| 153 | 3.71E−08 | 1.75E−02 |
| 154 | 2.47E−08 | 1.60E−02 |
| 155 | 1.79E−07 | NT |
| 156 | 2.96E−08 | 6.53E−03 |
| 157 | 6.77E−09 | 7.16E−03 |
| 158 | 6.77E−08 | NT |
| 159 | 2.82E−08 | 1.68E−02 |
| 160 | 1.38E−07 | NT |
| 161 | 2.94E−08 | 1.26E−02 |
| 162 | 1.00E−08 | 6.47E−03 |
| 163 | 5.24E−08 | 2.77E−02 |
| 164 | 2.46E−08 | 1.65E−02 |
| 165 | 5.57E−09 | 4.15E−03 |
| 166 | 1.12E−08 | 9.43E−03 |
| 167 | 2.72E−08 | 1.24E−02 |
| 168 | 3.48E−09 | 1.57E−03 |
| 169 | 8.80E−09 | 2.18E−03 |
| 170 | 3.98E−08 | 2.19E−02 |
| 171 | 2.38E−08 | 1.68E−02 |
| 175 | 5.40E−08 | 9.98E−03 |
| 176 | 7.72E−08 | 3.7E−02 |
| 177 | 3.33E−08 | 2.18E−02 |
| 172 | 5.50E−09 | 5.01E−03 |
| 173 | 1.46E−08 | 5.21E−03 |
| 174 | 6.77E−08 | 1.74E−02 |

NT: not tested

### EXAMPLE C

### Pharmaceutical Composition: Tablets

| | |
|---|---|
| 1000 tablets containing a dose of 5 mg of a compound selected | 5 g |
| from Examples 1 to 177 | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1-32. (canceled)

33. A compound of formula (I): 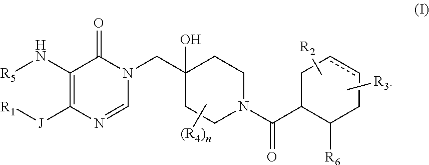
wherein:
J represents an oxygen atom or a sulphur atom,
R₁ represents a cycloalkyl group, a heterocycloalkyl group, an aryl group, or a heteroaryl group,
R₂ represents a hydrogen atom, a halogen atom, a hydroxy group, or a linear or branched (C₁-C₆)alkoxy group,
R₃ represents a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₂-C₆)alkynyl-R₇ group, a cycloalkyl group, an aryl group, a heteroaryl group, an aryl(C₁-C₆)alkyl group, or a heteroaryl(C₁-C₆)alkyl group,
R₄ represents a hydrogen atom or a halogen atom,
R₅ represents a hydrogen atom, a linear or branched (C₁-C₆)alkyl group, a linear or branched halo(C₁-C₆)alkyl group, or an aryl(C₁-C₆)alkyl group,
R₆ represents an aryl group or a heteroaryl group,
R₇ represents a cycloalkyl group, an aryl group, a heteroaryl group or a —Y₁—OR′ group,
n is an integer equal to 0, 1 or 2, [symbol]
means a single bond or a double bond,
wherein:
“aryl” means a phenyl, naphthyl, or indanyl group,
“heteroaryl” means any mono- or fused bi-cyclic group composed of from 5 to 10 ring members, having at least one aromatic moiety and containing from 1 to 3 heteroatoms selected from oxygen, sulphur and nitrogen,
“cycloalkyl” means any mono- or fused bi-cyclic non-aromatic carbocyclic group containing from 3 to 7 ring members,
“heterocycloalkyl” means any non-aromatic mono- or fused bi-cyclic group containing from 3 to 10 ring members, and containing from 1 to 3 heteroatoms selected from oxygen, sulphur and nitrogen,
wherein the aryl, heteroaryl, cycloalkyl and heterocycloalkyl groups so defined may be optionally substituted by from 1 to 4 groups selected from linear or branched (C₁-C₆)alkyl, linear or branched (C₂-C₆)alkenyl, linear or branched (C₂-C₆)alkynyl, linear or branched halo(C₁-C₆)alkyl, —Y₂—OR′, —Y₂—NR′R″, —Y₂—S(O)ₘ—R′, oxo (or N-oxide where appropriate), pentafluorosulfide, nitro, —Y₂—CN, —C(O)—R′, —C(O)—OR′, —O—C(O)—R′, —Y₂—C(O)—NR′R″, —Y₂—NR′—C(O)—R″, —Y₂—NR′—C(O)—OR″, halogen, cyclopropyl and —Y₂-heterocycloalkyl,
and wherein:
Y₁ and Y₂, independently of one another, represent a bond, a linear or branched (C₁-C₄)alkylene group, or a linear or branched halo(C₁-C₄)alkylene group,
R′ and R″, independently of one another, represent a hydrogen atom, a linear or branched (C₁-C₆)alkyl group, a linear or branched (C₂-C₆)alkenyl group, a linear or branched (C₂-C₆)alkynyl group, a linear or branched (C₁-C₆)alkoxy group, a linear or branched halo(C₁-C₆)alkyl, a linear or branched hydroxy(C₁-C₆)alkyl group, a linear or branched (C₁-C₆)alkoxy(C₁-C₆)alkyl group, a formyl group, a phenyl group, a benzyl group, a cyclopropyl group, or a cyclopropylmethyl group,
or the substituents of the pair (R′, R″), together with the nitrogen atom carrying them, form a non-aromatic ring having from 5 to 7 ring members, which ring may have, in addition to the nitrogen, a second heteroatom selected from oxygen and nitrogen, wherein the nitrogen in question may be substituted by from 1 to 2 groups selected from a hydrogen atom and a linear or branched (C₁-C₆)alkyl group,
m is an integer equal to 0, 1 and 2,
its enantiomers, diastereoisomers, or addition salts thereof with a pharmaceutically acceptable acid or base.

34. The compound according to claim 33, wherein [symbol] is a single bond.

35. The compound according to claim 33, wherein J represents an oxygen atom.

36. The compound according to claim 33, wherein R₁ represents an aryl group or a heteroaryl group.

37. The compound according to claim 36, wherein R₁ represents a phenyl group, an indanyl group, a benzodioxolyl group, a tetrahydroisoquinolyl group, an isoindolinyl group, an indazolyl group, a thiazolyl group, a pyridinyl group, a pyrrolopyridinyl group, or a pyrimidinyl group.

38. The compound according to claim 37, wherein R₁ represents a phenyl group.

39. The compound according to claim 33, wherein R₂ represents a halogen atom, a hydroxy group, or a linear or branched (C₁-C₆)alkoxy group.

40. The compound according to claim 39, wherein R₂ represents a fluorine atom, a hydroxy group, or a methoxy group.

41. The compound according to claim 33, wherein R₃ represents a halogen atom, a linear or branched (C₁-C₆)alkyl group, a linear or branched (C₂-C₆)alkynyl group, a linear or branched (C₂-C₆)alkynyl-R₇ group, an aryl group, an aryl(C₁-C₆)alkyl group or a heteroaryl(C₁-C₆)alkyl group.

42. The compound according to claim 41, wherein R₃ represents a fluorine atom; a phenyl group; a benzyl group; a —C≡CH group; a —C≡C—R₇ group, wherein R₇ represents a cycloalkyl group, an aryl group, or a heteroaryl group; or a heteroaryl(C₁-C₆)alkyl group wherein the heteroaryl moiety is pyridinyl, pyrimidinyl, pyrazinyl, pyridazinyl, thiazolyl, or imidazolyl.

43. The compound according to claim 33, wherein R₂ and R₃ are geminal groups.

44. The compound according to claim 43, wherein R₂ and R₃ each represent a fluorine atom.

45. The compound according to claim 43, wherein R₂ represents a halogen atom or a linear or branched (C₁-C₆)alkoxy group and R₃ represents a —C≡C—R₇ group wherein R₇ represents a heteroaryl group selected from imidazolyl, pyridinyl, pyrimidinyl, pyrazinyl and pyridazinyl.

46. The compound according to claim 33, wherein R₄ represents a hydrogen atom or a fluorine atom.

47. The compound according to claim 33, wherein R₅ represents a hydrogen atom.

48. The compound according to claim 33, wherein R₆ represents an aryl group or a heteroaryl group selected from pyridinyl, thienyl, oxazolyl, pyrazolyl, thiazolyl or furyl.

49. The compound according to claim 48, wherein R₆ represents an aryl group.

50. The compound according to claim 33, wherein R₇ represents a cycloalkyl group, an aryl group, or a heteroaryl group.

51. The compound according to claim 50, wherein R₇ represents a cyclopropyl group, a phenyl group, a imidazolyl group, a pyridinyl group, a pyrimidinyl group, a pyrazinyl group, or a pyridazinyl group.

52. The compound according to claim 33, which is compound of formula (I-a): 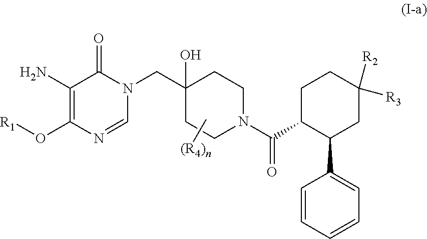
wherein R₁, R₂, R₃, R₄ and n are as defined in claim 33.

53. The compound according to claim 33, which is selected from the group consisting of:
5-amino-3-[[1-[(1R,2R)-4,4-difluoro-2-phenyl-cyclohexanecarbonyl]-4-hydroxy-4-piperidyl]methyl]-6-(3-hydroxy-5-methoxy-phenoxy)pyrimidin-4-one;
5 -amino-3-({1-[(1R,2R)-4,4-difluoro-2-phenylcyclohexane-1-carbonyl]-4-hydroxypiperidin-4-yl}methyl)-6-[4-(pyrrolidin-2-yl)phenoxy]pyrimidin-4(3H)-one;
5-amino-3-({1-[(1R,2R)-4,4-difluoro-2-phenylcyclohexane-1-carbonyl]-4-hydroxypiperidin-4-yl}methyl)-6-(4-fluoro-3-hydroxyphenoxy)pyrimidin-4(3H)-one;
5-amino-3-[[1-[(1R,2R)-4,4-difluoro-2 -phenyl-cyclohexanecarbonyl]-4-hydroxy-4-piperidyl]methyl]-6-[3-(2-piperidyl)phenoxy]pyrimidin-4-one;
5-amino-6-[4-(1-aminoethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-6-[4-(aminomethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-3-({1-[(1R,2R)-4,4-difluoro-2-phenylcyclohexane-1-carhonyl]-4-hydroxypiperidin-4-yl}methyl)-6-{4-[(methylamino)methyl]phenoxy}pyrimidin-4(3H)-one;
5-amino-6-[3-(aminomethyl)phenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-3-[[1-[(1R,2R)-4,4-difluoro-2-phenyl-cyclohexanecarbonyl]-4-hydroxy-4-piperidyl]methyl]-6-(3-hydroxyphenoxy)pyrimidin-4-one;
5-amino-6-[4-(aminomethyl)-3-fluorophenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-6-[4-(aminomethyl)-3-chlorophenoxy]-3-({1-[(1R,2R)-4,4-difluoro-2-phenylcyclohexane-1-carbonyl]-4-hydroxypiperidin-4-yl}methyl)pyrimidin-4(3H)-one;
5-amino-6-{4-[(tert-butylamino)methyl]phenoxy}-3-({1-[(1R,2R)-4,4-difluoro-2-phenylcyclohexane-1-carbonyl]-4-hydroxypiperidin-4-yl }methyl)pyrimidin-4(3H)-one;
5-amino-6-[4-(aminomethyl)phenoxy]-3-({(4S)-1-[(1R,2R)-4,4-difluoro-2-phenylcyclohexane-1 -carbonyl]-3,3-difluoro-4-hydroxypiperidin-4-yl}methyl)pyrimidin-4(3H)-one;
5-amino-3-({(4S)-1-[(1R,2R)-4,4-difluoro-2-phenylcyclohexane-1-carbonyl]-3,3-difluoro-4-hydroxypiperidin-4-yl}methyl)-6-(3-hydroxyphenoxy)pyrimidin-4(3H)-one;
5-amino-3-({(4S)-1-[(1R,2R)-4,4-difluoro-2-phenylcyclohexane-1-carbonyl]-3,3-difluoro-4-hydroxypiperidin-4-yl}methyl)-6-(4-fluoro-3-hydroxyphenoxy)pyrimidin-4(3H)-one;
5-amino-3-({1-[(1R,2R)-4,4-difluoro-2-phenylcyclohexane-1-carbonyl]-4-hydroxypiperidin-4-yl}methyl)-6-[4-(piperidin-2-yl)phenoxy]pyrimidin-4(3H)-one;
5-amino-3-[(1-{[(1R,2R,4R)-4-fluoro-2-phenyl-4-[2-(pyridin-3-yl)ethynyl]cyclohexyl]carbonyl}-4-hydroxypiperidin-4-yl)methyl]-6-(4-fluorophenoxy)pyrimidin-4-one;
5-amino-3-[(1-{[(1R,2R,4R)-4-fluoro-2-phenyl-4-[2-(pyridin-2-yl)ethynyl]cyclohexyl]carbonyl}-4-hydroxypiperidin-4-yl)methyl]-6-(4-fluorophenoxy)pyrimidin-4-one;
5-amino-3-[(1-{[(1R,2R,4R)-4-fluoro-4-[2-(5-fluoropyridin-2-yl)ethynyl]-2-phenylcyclohexyl]carbonyl}-4-hydroxypiperidin-4-yl)methyl]-6-(4-fluorophenoxy)pyrimidin-4-one;
5-amino-3-({1-[(1R,2R,4S)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4-one;
5-amino-3-({1-[(1R,2R,4R)-4-fluoro-2-phenyl-4-[2-(pyrazin-2-yl)ethynyl]cyclohexanecarbonyl]-4-hydroxypiperidin-4-yl}methyl)-6-(4-fluorophenoxy)-3,4-dihydropyrimidin-4-one;
5-amino-6-(4-fluorophenoxy)-3-[(4-hydroxy-1-{[(1R,2R,4R)-4-methoxy-2-phenyl-4-[2-(pyrimidin-5-yl)ethynyl]cyclohexyl]carbonyl}piperidin-4-yl)methyl]pyrimidin-4-one;
5-amino-3-[(1-{[(1R,2R,4R)-4-fluoro-2-phenyl-4-[2-(pyridazin-3-yl)ethynyl]cyclohexyl]carbonyl}-4-hydroxypiperidin-4-yl)methyl]-6-(4-fluorophenoxy)pyrimidin-4-one;
5-amino-6-(4-fluorophenoxy)-3-[(4-hydroxy-1-{[(1R,2R,4R)-4-methoxy-2-phenyl-4-[2-(pyridazin-3-yl)ethynyl]cyclohexyl]carbonyl}piperidin-4-yl)methyl]pyrimidin-4-one;
5-amino-6-(4-fluorophenoxy)-3-({4-hydroxy-1-[(1R,2R,4R)-4-methoxy-2-phenyl-4-[2-(pyrazin-2-yl)ethynyl]cyclohexanecarbonyl]piperidin-4-yl}methyl)-3,4-dihydropyrimidin-4-one;
5-amino-3-({1-[(1R,2R,4R)-4-fluoro-4-[2-(4-fluoropyridin-2-yl)ethynyl]-2-phenylcyclohexanecarbonyl]-4-hydroxypiperidin-4-yl}methyl)-6-(4-fluorophenoxy)-3,4-dihydropyrimidin-4-one;
5-amino-3-{[(4S)-3,3-difluoro-1-[(1R,2R,4R)-4-fluoro-4-[2-(6-methylpyridazin-3-yl)ethynyl]-2-phenylcyclohexanecarbonyl]-4-hydroxypiperidin-4-yl]methyl}-6-(4-fluorophenoxy)-3,4-dihydropyrimidin-4-one;
5-amino-3-{[(4S)-3,3-difluoro-4-hydroxy-1-{[(1R,2R,4R)-4-methoxy-2-phenyl-4-[2-(pyridazin-3-yl)ethynyl]cyclohexyl]carbonyl}piperidin-4-yl]methyl }-6-(4-fluorophenoxy)pyrimidin-4-one;
5-amino-3-{[(4S)-3,3-difluoro-4-hydroxy-1-[(1R,2R,4R)-4-methoxy-2-phenyl-4-[2-(pyridin-2-yl)ethynyl]cyclohexanecarbonyl]piperidin-4-yl]methyl}-6-(4-fluorophenoxy)-3,4-dihydropyrimidin-4-one;
5-amino-3-{[(4S)-3,3-difluoro-1-{[(1R,2R,4R)-4-fluoro-2-phenyl-4-[2-(pyridazin-3-yl)ethynyl]cyclohexyl]carbonyl}-4-hydroxypiperidin-4-yl]methyl}-6-(4-fluorophenoxy)pyrimidin-4-one;
5-amino-3-{[(4S)-3,3-difluoro-4-hydroxy-1-[(1R,2R,4R)-4-methoxy-4-[2-(5-methylpyridazin-3 -yl)ethynyl]-2-phenylcyclohexanecarbonyl]piperidin-4-yl]methyl}-6-(4-fluorophenoxy)-3,4-dihydropyrimidin-4-one;
5-amino-3-{[(4S)-3,3-difluoro-1-[(1R,2R,4R)-4-fluoro-4-[2-(5-methylpyridazin-3-yl)ethynyl]-2-phenylcyclohexanecarbonyl]-4-hydroxypiperidin-4-yl]methyl}-6-(4-fluorophenoxy)-3,4-dihydropyrimidin-4-one;
5-amino-3-{[(4S)-3,3-difluoro-4-hydroxy-1-[(1R,2R,4R)-4-methoxy-4-[2-(5-methoxypyridazin-3-yl)ethynyl]-2-phenylcyclohexanecarbonyl]piperidin-4-yl]methyl}-6-(4-fluorophenoxy)-3,4-dihydropyrimidin-4-one;
5-amino-3-{[(4S)-3,3-difluoro-4-hydroxy-1-[(1R,2R,4R)-4-methoxy-2-phenyl-4-[2-(pyrimidin-5-yl)ethynyl]cyclohexanecarbonyl]piperidin-4-yl]methyl}-6-(4-fluorophenoxy)-3,4-dihydropyrimidin-4-one.

54. A pharmaceutical composition comprising the compound according to claim 33, or an addition salt thereof with a pharmaceutically acceptable acid or base, in combination with one or more pharmaceutically acceptable excipients.

55. A method of treating a condition requiring a pro-apoptotic and/or anti-proliferative agent in a subject in need thereof, comprising administration of the compound according to claim 33, alone or in combination with one or more pharmaceutically acceptable excipients.

56. The method according to claim 55, wherein the condition is selected from cancer, auto-immune diseases, and immune system diseases.

57. The method according to claim 56, wherein the condition is selected from cancers of the bladder, brain, breast and uterus, chronic lymphoid leukemia, cancer of the colon, esophagus and liver, lymphoblastic leukemia, acute myeloid leukemia, lymphomas, melanomas, malignant haemopathies, myelomas, ovarian cancer, non-small-cell lung cancer, prostate cancer, pancreatic cancer and small-cell lung cancer.

58. A combination comprising the compound according to claim 33 and an anti-cancer agent selected from genotoxic agents, mitotic poisons, anti-metabolites, proteasome inhibitors, kinase inhibitors, protein-protein interaction inhibitors, immunomodulators, E3 ligase inhibitors, chimeric antigen receptor T-cell therapy and antibodies.

59. A pharmaceutical composition comprising the combination according to claim 58 in combination with one or more pharmaceutically acceptable excipients.

60. A method of treating cancer in a subject in need thereof, comprising administration of the combination according to claim 58, alone or in combination with one or more pharmaceutically acceptable excipients.

61. A method of treating cancer requiring radiotherapy in a subject in need thereof, comprising administration of the compound according to claim 33, alone or in combination with one or more pharmaceutically acceptable excipients.
